(19) United States
(12) Patent Application Publication
(10) Pub. No.: US 2012/0253017 A1
(43) Pub. Date: Oct. 4, 2012

(54) STEM CELL TARGETING

(76) Inventors: Victoria Ballard, King of Prussia, PA (US); Thil Dinuk Batuwangala, Cambridge (GB); Edward Coulstock, Cambridge (GB); Elena De Angelis, Cambridge (GB); Jay Edelberg, King of Prussia, PA (US); Carolyn Enever, Cambridge (GB); Steve Holmes, Cambridge (GB); Zahra Ja wad-Alami, Cambridge (GB)

(21) Appl. No.: 13/322,030

(22) PCT Filed: May 26, 2010
(86) PCT No.: PCT/EP2010/057283

§ 371 Date: Nov. 22, 2011

Related U.S. Application Data
(60) Provisional application No. 61/181,814, filed on May 28, 2009.

(51) Int. Cl. C07K 16/46

(52) U.S. Cl. 530/387.3

(57) ABSTRACT

The present invention describes an antigen-binding construct comprising a first agent which binds to a stem cell specific marker molecule and a second agent which binds to a tissue specific marker molecule. In particular, the invention describes a construct wherein the tissue specific marker is a muscle specific marker molecule. Such a construct may be used in a pharmaceutical composition for use in muscle regeneration or heart disease.

### BACKGROUND TO THE INVENTION

Cardiovascular disease is disease of the heart and/or blood vessels and is the leading cause of morbidity and mortality in the developed world. One of the main contributors to cardiovascular disease is ischaemic heart disease (IHD or myocardial ischaemia) which is characterised by the heart muscle receiving a reduced blood supply generally as a result of coronary artery disease (such as atherosclerosis of the coronary arteries). A reduced blood supply to the heart muscle can result in damage to the myocardium and death of the muscle cells which can, in turn, lead to heart failure. Heart failure can also be caused by chronic hypertension, viral infection, cardiac valve abnormalities, and genetic and other causes.

Heart failure may be treated with a range of approaches, depending on severity. In the earlier stages of heart failure, smoking cessation and physical activity may be recommended along with pharmacological interventions such as ACE inhibitors and beta-blockers. For those with more severe symptoms implantable defibrillators or pacemakers may be employed, and in extreme cases, heart transplantation may be recommended (Jessup et al., 2009, ACFF/AHA guidelines). Although current treatments reduce morbidity and mortality after a myocardial infarction (MI), these levels remain high even when treatment is conducted according to current guidelines.

More recently, approaches for cardiac tissue engineering and cell transplantation to regenerate the myocardium have begun to be developed. Many of these approaches involve the delivery of stem cells to the heart, either via intracoronary or intramyocardial injection (reviewed, for example by Fazel et al. Ann. Thorac. Surg. 2005; 79:S22238-47). These cells, largely derived from the bone marrow, are able to give rise to multiple cell types, including cardiac muscle and vascular cells, thereby making them an attractive tool for promotion of cardiac regeneration after injury. While animal studies have shown some efficacy using these approaches, little clinical benefit has been observed so far. There are many reasons why these approaches to date have shown limited cardiac functional improvement. One such reason is the ineffective homing and retention of stem cells. Indeed, a number of studies have shown that when bone marrow cells are injected into the heart after cardiac injury, the majority of these cells will end up in other organs, including the lung, spleen and liver. Thus, an improved method for targeting stem cells to damaged muscle is required.

### SUMMARY OF THE INVENTION

The present invention provides compositions and methods for targeting stem cells to tissues including muscle. In one embodiment, the invention provides compositions and methods for targeting stem cells to the heart.

In one aspect, there is provided a construct comprising a first agent which binds to a stem cell specific marker molecule and a second agent which binds to a tissue specific marker molecule. In one embodiment the construct is an antigen-binding construct. In one embodiment, the first and/or second agent is an antibody such as a monoclonal antibody. In another embodiment the first and/or second agent is an epitope-binding domain which binds an epitope on the marker molecule. In one embodiment, the epitope-binding domain is an immunoglobulin single variable domain. The construct may comprise further agents or epitope binding domains which bind additional stem cell specific marker molecules or epitopes on such stem cell specific molecules, additional tissue specific marker molecules or additional epitopes on such tissue specific marker molecules or agents or epitope binding domains which bind other molecules. Accordingly, in one embodiment, a dual targeting construct is provided, in another embodiment, a multi-targeting construct is provided.

In one embodiment, the stem cell specific marker molecule and/or the tissue specific molecule is a human molecule.

Stem cell specific marker molecules are familiar to those skilled in the art and include, for example, CD34, CD44, CD45, CD133 and CD117 (c-Kit).

Accordingly, in one embodiment, the stem cell specific marker molecule is c-Kit. In one embodiment, the agent which binds to c-Kit is an antibody such as a monoclonal antibody. In another embodiment, the agent which binds to c-Kit is an anti-c-Kit immunoglobulin single variable domain in accordance with any aspect of the invention such as those described herein. Accordingly in one embodiment there is provided a construct comprising a c-Kit dAb as described herein and an agent which which binds to a tissue specific marker molecule.

In one embodiment, the tissue specific marker molecule is a muscle specific marker molecule.

In one embodiment, the muscle specific molecule is selected from a myosin derived molecule such as a Myosin Light Chain (MLC) or a Myosin Heavy Chain, including but not limited to human ventricular myosin light chain 1 (vMLC1 (v-MLC1, vMLC-1); also referred to as cMLC or MLC 3), MLC 2 or Myosin Heavy Chain 6 (Myosin Heavy Chain, cardiac muscle alpha isoform). In one embodiment, the muscle specific marker molecule is a myocardium specific marker molecule. In one embodiment, the “myocardium-specific molecule” is a molecule that is expressed or becomes exposed only in damaged myocardium and not in healthy, intact myocardium. After ischemic injury, such as myocardial infarction, myocyte damage and necrosis occurs, resulting in the rupturing of cells and exposure of intracellular or cardiac structural proteins to the environment. vMLC1 (also known as MLC 3) is one example of such a molecule. Other myocardium specific molecules include cardiac troponin I or cardiac troponin such as cardiac troponin T, annexin and molecules which are upregulated at sites of myocardial damage such as Tenascin C and creatine kinase.

In one embodiment, the agent which binds to a muscle specific marker molecule is an anti-MLC antibody. In one embodiment, the anti-MLC antibody has high affinity for human cardiac myosin light chains. In one embodiment the anti-MLC antibody is a monoclonal antibody. Anti-MLC antibodies include antibodies which bind to human ventricular myosin light chain 1 and are described, for example, in U.S. Pat. No. 5,702,905 as monoclonal antibody 39-15 (available from ATCC HB 11709 or commercially e.g. MLM508 (Abcam)).

In one embodiment, the anti-MLC antibody in a construct in accordance with one aspect of the invention is a humanised anti-MLC antibody as described herein and in accordance with any aspect of the invention.

In another embodiment, the anti-MLC antibody is an anti-MLC immunoglobulin single variable domain antibody. Methods for generating a specific single variable domain antibody are described herein.

In one embodiment the invention provides a construct comprising an anti-MLC immunoglobulin single variable domain and an anti-c-Kit immunoglobulin single variable domain. Such a construct may be a “dAb-dAb” construct.

In another embodiment, the agent which binds to a muscle specific marker molecule binds with higher affinity than does the agent which binds to c-Kit bind to c-kit.

In one embodiment, the invention provides a construct comprising an anti-MLC monoclonal antibody and an anti-c-Kit monoclonal antibody as described herein.

In another embodiment, the invention provides a construct which comprises a monoclonal antibody (MAb) in conjunction with an immunoglobulin single variable domain (dAb). Such constructs are referred to as a “MAbdAb” (or “mAbdAb”, “mAb-dAb”). In one embodiment, the construct in accordance with the invention comprises an agent which binds to a muscle specific marker molecule, such as a monoclonal anti-MLC antibody, and an anti-c-Kit immunoglobulin single variable domain. In another embodiment, the invention provides a construct comprising a monoclonal anti-c-Kit antibody and an anti-MLC immunoglobulin single variable domain. It can be advantageous to use a construct comprising a dAb as a mAb-dAb construct can be expressed as a single molecule. In addition, using a dAb may allow a monovalent interaction with the receptor therefore reducing likelihood of receptor activation.

In one embodiment a construct in accordance with the invention is selected from any of the constructs described in Table 24. In another embodiment, the construct comprises a dAb which is a dAb from the DOM28h-94 lineage.

In one embodiment the first and/or second agent cross-reacts with the stem cell specific marker molecule or the muscle specific marker molecule from another species such as mouse, rat, dog, pig and non-human primate species.

In one embodiment, the first agent which binds a stem cell specific marker molecule and the second agent which binds to a muscle specific marker molecule are linked. Suitable linkers include chemical linkage agents such as SulfoSMCC and others available from manufacturers such as Pierce. Other suitable linkers will be familiar to those skilled in the art.

Other examples of suitable linkers include amino acid sequences which may be from 1 amino acid to about 150 amino acids in length, or from 1 amino acid to about 140 amino acids, for example, from 1 amino acid to about 130 amino acids, or from 1 to about 120 amino acids, or from 1 to about 80 amino acids, or from 1 to about 50 amino acids, or from 1 to about 20 amino acids, or from 1 to about 10 amino acids, or from about 5 to about 18 amino acids. Such sequences may have their own tertiary structure, for example, a linker of the present invention may comprise a single variable domain. The size of a linker in one embodiment is equivalent to a single variable domain. Suitable linkers may be of a size from about 1 to about 20 angstroms, for example less than about 15 angstroms, or less than about 10 angstroms, or less than about 5 angstroms.

In one embodiment of the present invention at least one of the epitope binding domains is directly attached to an Ig scaffold with a linker comprising from 1 to about 150 amino acids, for example 1 to about 20 amino acids, for example 1 to about 10 amino acids. Such linkers may be selected from any one of: A G4S linker (GGGGS; SEQ ID NO: 88); TVAAPS (SEQ ID NO: 89); ASTKGPT (SEQ ID NO: 90); ASTKGPS (SEQ ID NO: 91); EPKSCDKTHTCPPCP (SEQ ID NO: 92); ELQLEESCAEAQDGELDG (SEQ ID NO: 93), “AST” (SEQ ID NO: 94), STGGGGGS (SEQ ID NO: 95), STGGGGGSGGGGS (SEQ ID NO: 96), STGPPPPPS (SEQ ID NO: 97), STGPPPPPPPPPPS (SEQ ID NO: 98), ‘STG’ (serine, threonine, glycine; SEQ ID NO: 99), ‘GSTG’ (SEQ ID NO: 100) or ‘RS’ (SEQ ID NO: 101). In one embodiment, the linker is selected from STG, GGGGS and PPPPPS (SEQ ID NO: 483). In one embodiment, the linker may be one which reduces the potential for interactions between the dAb and the Fc domain due to steric constraints thereby increasing the opportunity for the Fc region to participate in its normal interactions. In this embodiment, the linker may be the stalk region from a protein such as human glycoprotein VI (GPVI), for example: STGSRDPYLWSAPSDPLELVVTGTSVTPSRLPTEPPSSVAEFSEATAELTVSFTNK VFTTETSRSITTSPKESDSPAGPARQYYTKGNGSTG (SEQ ID NO: 484). Linkers of use in the antigen binding constructs of the present invention may comprise alone or in addition to other linkers, one or more sets of GS residues, for example ‘GSTVAAPS’ (SEQ ID NO: 102) or ‘TVAAPSGS’ (SEQ ID NO: 103) or ‘GSTVAAPSGS’ (SEQ ID NO: 104). In another embodiment there is no linker between the epitope binding domain, for example the dAb, and the Ig scaffold. In another embodiment the epitope binding domain, for example a dAb, is linked to the Ig scaffold by the linker ‘TVAAPS’ (SEQ ID NO: 89). In another embodiment the epitope binding domain, for example a dAb, is linked to the Ig scaffold by the linker ‘TVAAPSGS’ (SEQ ID NO: 103). In another embodiment the epitope binding domain, for example a dAb, is linked to the Ig scaffold by the linker ‘GS’ (SEQ ID NO: 105).

Suitable methods for generating a MAbdAb construct in accordance with the invention are described herein. Various positions for the dAb attachment to the MAb form an embodiment of this aspect of the invention. Such positions for dAb attachment are exemplified in FIG. 15 and also include attachment of the dAb to a variable domain of a MAb.

In another embodiment, a construct comprising a dAb-dAb may be linked via Fc region or a heavy chain constant region as described, for example, in EP 1864998.

In one embodiment a construct wherein one of the targeting antibodies is a monoclonal antibody or dAb linked to an Fc domain will be of sufficient size to reduce the rate of clearance of the construct from the blood i.e. to provide an extended half life (compared to the dAb alone). Additional methods for half-life extension and methods for determining the same are described, for example in WO 2008096158. Such methods include generating protease resistance, linking to serum proteins such as serum albumin, AlbudAbs® and so forth.

In one embodiment, the construct comprises an inactivated Fc or alternative IgG isotype that does not induce ADCC.

In another aspect there is provided a nucleotide sequence encoding a construct in accordance with the first aspect of the invention.

In a further aspect of the invention there is provided an antigen binding protein which binds Myosin Light Chain (MLC). In one embodiment the antigen binding protein which binds MLC is one which binds a cardiac isoform of MLC, for example ventricular MLC (vMLC, vMLC-1, MLC-3) or human ventricular MLC (HVMLC). Accordingly, in one embodiment, the antigen binding protein binds human ventricular myosin light chain 1 (vMLC1).

In one embodiment, the antigen binding protein in accordance with this aspect of the invention is an antibody related to the mouse monoclonal antibody, 39-15 (ATCC HB11709) which binds vMLC1. Accordingly, in one embodiment, the invention provides an antigen binding protein which binds vMLC1 and which comprises a heavy or light chain CDR3 sequence of 39-15 as set out of SEQ ID NO: 13 or SEQ ID NO: 17, or variants thereof which contain 1, 2 or 3 amino acid substitutions in CDR3. In one embodiment any such variants also bind vMLC1.

In one embodiment, the antigen binding protein in accordance with the invention comprises the following CDRs: CDRH1 (SEQ ID NO: 18) CDRH2 (SEQ ID NO: 19), CDRH3 (SEQ ID NO: 20), CDRL1 (SEQ ID NO: 14), CDRL2 (SEQ ID NO: 15), CDRL3 (SEQ ID NO: 16).

In another embodiment, the antigen binding protein binds to both human vMLC1 and to another MLC1 derived from a different species such as mouse, dog or cynomolgus monkeys (cyno). In one embodiment, the antigen binding protein in accordance with the invention binds to both mouse and human vMLC1. In the context of the present invention, such cross reactivity between vMLC1 from humans and other species allows the same antibody construct to be used in an animal disease model as well as in humans.

In one embodiment, the antigen binding protein is an antibody such as a humanized or chimaeric antibody. In one embodiment, the MLC antigen binding proteins of the present invention include non-murine equivalents of 39-15 such as humanized forms as described herein. In one embodiment an antigen binding protein in accordance with the invention comprises a Fab, Fab′, F(ab′)₂, Fv, diabody, triabody, tetrabody, miniantibody, isolated VH, isolated VK or dAb.

In one embodiment, the heavy chain variable regions may be formatted together with light chain variable regions to allow binding to MLC in the conventional immunoglobulin manner (for example human IgG, IgA, IgM etc.) or in any other “antibody-like” format that binds to human MLC (for example single chain Fv, diabodies, Tandabs etc (for a summary of alternative “antibody” formats see Holliger and Hudson, Nature Biotechnology, 2005, Vol. 23, No. 9, 1126-1136).

In one embodiment, the antigen binding protein in accordance with the invention comprises a V_{H} domain selected from SEQ ID NOs: 22, 25, 28 or 31 paired with a light chain variable region to form an antigen binding unit which binds to MLC. In another embodiment the antigen binding protein in accordance with the invention comprises a Vk domain selected from SEQ ID NOs: 34 or 37 paired with a heavy chain variable region to form an antigen binding unit which binds to MLC. Other suitable pairings are exemplified herein. In a further embodiment there is provided an antigen binding protein comprising a V_{H} domain selected from SEQ ID NOs: 22, 25, 28 or 31 and a Vk domain selected from SEQ ID NOs: 34 or 37. In one embodiment, the heavy chain has a sequence as set out in SEQ ID NO: 31 and the light chain has a sequence as set out in SEQ ID NO: 37. In other embodiments, there is provided a combination of any of the V_{H} domains or Vκ domains described herein with any light or heavy chain variable region. In one embodiment, the V_{H} domains or Vκ domains are any of the sequences as set out in FIG. 5.

In one embodiment, the antigen binding protein in accordance with the invention binds to human MLC, for example human cardiac isoforms of MLC such as HVMLC and HVMLC-1, with high affinity as measured by Biacore in the region of about 0.1 pM to about 100 nM, for example about 0.1 pM to about 100 pM.

In another aspect, there is provided a nucleic acid molecule encoding an antigen binding protein or antibody in accordance with the invention. There is also provided a host cell transformed or transfected with such a nucleic acid molecule. In another aspect there is provided a first and second vector wherein said first vector comprises a nucleic acid molecule encoding a heavy chain of an antigen binding protein or antibody in accordance with the invention and said second vector comprises a nucleic acid molecule encoding a light chain of an antigen or antibody in accordance with the invention.

In another aspect, the invention provides an anti-c-Kit immunoglobulin single variable domain. In one embodiment, an anti-c-Kit immunoglobulin single variable domain in accordance with the first aspect is one which binds to c-Kit with a dissociation constant (Kd, KD, K_{D}) in the range of about 10 pM to about 10 micromolar, for example about 100 pM to about 10 micromolar, about 10 nM to about 1 micromolar, or about 1 nM to about 100 nM.

In another aspect, the invention provides an isolated polypeptide comprising an anti-c-Kit immunoglobulin single variable domain. In one embodiment, the isolated polypeptide comprises an amino acid sequence that is at least about 70% identical to at least one amino acid sequence encoded by a nucleic acid having a sequence as set out in any of SEQ ID NOs: 39-87, 224-246, 270-282, 297-300, 383-384, 383-384 or 476 and which binds to c-Kit. In another embodiment, the isolated polypeptide comprises an amino acid sequence that is at least 70% identical to at least one amino acid sequence as set out in any of SEQ ID NOs: 148 to 163, 247-269, 283-295. 301-305, 477-482 and which binds to c-Kit. In one embodiment, the isolated polypeptide comprises an amino acid sequence that is at least 70% identical to a DOM28h-94 lineage amino acid sequence. Suitably, the isolated polypeptide comprises an amino acid sequence that is at least 70% identical to at least one amino acid sequence as set out in any of SEQ ID NOs: 302-305, 457, 458 or 482.

In one aspect, the invention provides an isolated polypeptide comprising an amino acid sequence encoded by a nucleic acid having a sequence as set out in any of SEQ ID NOs: 39-87, 224-246, 270-282, 297-300, 383-384, 383-384 or 476.

In another aspect, the invention provides an isolated polypeptide encoded by a nucleotide sequence that is at least about 60% identical to the nucleotide sequence selected from the group consisting of: any of the nucleic acid sequences set out in and of FIG. 6, 16, 17 or 20 (SEQ ID NOs: 39-87, 224-246, 270-282, 297-300, 383-384, 383-384 or 476) and which binds to c-Kit. In one embodiment the isolated polypeptide in accordance with any aspect of the invention binds to human c-Kit. In another embodiment, the polypeptide binds both human c-Kit and to c-Kit from another species such as mouse, dog or cyno. In one embodiment, the polypeptide binds to both human and mouse c-Kit.

In another aspect there is provided an anti-c-Kit immunoglobulin single variable domain comprising an amino acid sequence that is at least about 90% identical to the amino acid sequence of any one amino acid sequence encoded by a nucleic acid having a sequence as set out in any of SEQ ID NOs: 39-87, 224-246, 270-282, 297-300, 383-384 or 476. In one embodiment, the anti-c-kit immunoglobulin single variable domain comprises an amino acid sequence encoded by any of the nucleic acid sequences set out in SEQ ID NOs: 39-87, 224-246, 270-282, 297-300, 383-384 or 476. In one embodiment, the anti-c-Kit immunoglobulin single variable domain comprises an amino acid sequence that is identical to the amino acid sequence encoded by any one of the nucleic acid sequences identified as DOM28h-5 (SEQ ID NO: 39), DOM28h-43 (SEQ ID NO: 51), DOM28h-33 (SEQ ID NO: 49), DOM28h-94 (SEQ ID NO: 65), DOM28h-66 (SEQ ID NO: 58), DOM28h-110 (SEQ ID NO: 70), DOM28h-84 (SEQ ID NO: 62), DOM28m-23 (SEQ ID NO: 81), DOM28m-7 (SEQ ID NO: 78), DOM28m-52 (SEQ ID NO: 84), DOM28h-79 (SEQ ID NO: 61), DOM28h-7 (SEQ ID NO: 41), DOM28h-20 (SEQ ID NO: 45), DOM28h-26 (SEQ ID NO: 48), DOM28h-78 (SEQ ID NO: 60), DOM28h-73 (SEQ ID NO: 59), DOM28h-54 (SEQ ID NO: 55), DOM28h-113 (SEQ ID NO: 72), DOM28h-115 (SEQ ID NO: 73) and DOM28m-73 (SEQ ID NO: 86). In one embodiment the anti-c-Kit immunoglobulin single variable domain in accordance with any aspect of the invention binds to human c-Kit. In another embodiment, the anti-c-Kit immunoglobulin single variable domain binds both human c-Kit and to c-Kit from another species such as mouse, dog or monkeys such as cynomolgus monkeys (cyno). In one embodiment, the anti-c-Kit immunoglobulin single variable domain binds to both human and mouse c-Kit.

In another aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR1 sequence that is at least about 50% identical to the CDR1 sequence in any one of the amino acid sequences encoded by a nucleic acid having a sequence as set out in any of SEQ ID NOs: 39-87, 224-246, 270-282, 297-300, 383-384 or 476.

In a further aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR2 sequence that is at least about 50% identical to the CDR2 sequence in any one of the amino acid sequences encoded by a nucleic acid having a sequence as set out in any of SEQ ID NOs: 39-87, 224-246, 270-282, 297-300, 383-384 or 476.

In another aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR3 sequence that is at least about 50% identical to the CDR3 sequence in any one of the amino acid sequences encoded by a nucleic acid having a sequence as set out in any of SEQ ID NOs: 39-87, 224-246, 270-282, 297-300, 383-384 or 476.

In a further aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR1 sequence that is at least 50% identical to a CDR1 sequence in any one of the amino acid sequences encoded by a nucleic acid having a sequence as set out in any of SEQ ID NOs: 39-87, 224-246, 270-282, 297-300, 383-384 or 476 and comprises a CDR2 sequence that is at least about 50% identical to a CDR2 sequence in any one of the amino acid sequences encoded by a nucleic acid having a sequence as set out in any of SEQ ID NOs: 39-87, 224-246, 270-282, 297-300, 383-384 or 476.

In another aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR1 sequence that is at least about 50% identical to the CDR1 sequence in any one of the amino acid sequences encoded by a nucleic acid having a sequence as set out in any of SEQ ID NOs: 39-87, 224-246, 270-282, 297-300, 383-384 or 476 and comprises a CDR3 sequence that is at least about 50% identical to the CDR3 sequence in any one of the amino acid sequences encoded by a nucleic acid having a sequence as set out in any of SEQ ID NOs: 39-87, 224-246, 270-282, 297-300, 383-384 or 476.

In yet another aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR2 sequence that is at least about 50% identical to the CDR2 sequence in any one of the amino acid sequences encoded by a nucleic acid having a sequence as set out in any of SEQ ID NOs: 39-87, 224-246, 270-282, 297-300, 383-384 or 476 and comprises a CDR3 sequence that is at least about 50% identical to the CDR3 sequence in any one of the amino acid sequences encoded by a nucleic acid having a sequence as set out in any of SEQ ID NOs: 39-87, 224-246, 270-282, 297-300, 383-384 or 476.

In another aspect there is provided an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476 that is modified at no more than about 25 amino acid positions and comprises a CDR1 sequence that is at least about 50% identical to the CDR1 sequence in any one of the amino acid sequences encoded by a nucleic acid having a sequence as set out in any of SEQ ID NOs: 39-87 and comprises a CDR2 sequence that is at least about 50% identical to the CDR2 sequence in any one of the amino acid sequences encoded by a nucleic acid having a sequence as set out in any of SEQ ID NOs: 39-87, 224-246, 270-282, 297-300, 383-384 or 476 and comprises a CDR3 sequence that is at least 50% identical to the CDR3 sequence in any one of the amino acid sequences encoded by a nucleic acid having a sequence as set out in any of SEQ ID NOs: 39-87, 224-246, 270-282, 297-300, 383-384 or 476.

In a further aspect there is provided an anti-c-Kit immunoglobulin single variable domain comprising a CDR3 sequence that is at least about 50% identical to a CDR3 sequence selected from the group consisting of: the CDR3 sequence in any one of the amino acid sequences encoded by a nucleic acid having a sequence as set out in any of SEQ ID NOs: 39-87. 224-246, 270-282, 297-300, 383-384 or 476.

In another aspect there is provided an anti-c-Kit immunoglobulin single variable domain comprising a CDR3 sequence selected from the group consisting of: the CDR3 sequence in any one of the amino acid sequences encoded by a nucleic acid having a sequence as set out in any of SEQ ID NOs: 39-87, 224-246, 270-282, 297-300, 383-384 or 476.

In yet another aspect there is provided an anti-c-Kit immunoglobulin single variable domain comprising at least one CDR selected from the group consisting of: CDR1, CDR2, and CDR3, wherein the CDR1, CDR2, or CDR3 is identical to a CDR1, CDR2, or CDR3 sequence in any one of the amino acid sequences encoded by a nucleic acid having a sequence as set out in any of SEQ ID NOs: 39-87, 224-246, 270-282, 297-300, 383-384 or 476. In one embodiment, an anti-c-Kit immunoglobulin single variable domain in accordance with the invention comprises at least one CDR selected from the CDR sequences set out in FIG. 7. In one aspect there is provided a nucleic acid molecule comprising a nucleic acid sequence encoding an anti-c-Kit immunoglobulin single variable domain in accordance with the invention. In one embodiment, the nucleotide sequence comprises a nucleic acid sequence as set out in any of SEQ ID NOs: 39-87, 224-246, 270-282, 297-300, 383-384 or 476.

In another aspect there is provided a ligand that has binding specificity for c-Kit and inhibits the binding of an anti-c-Kit immunoglobulin single variable domain comprising an amino acid sequence encoded by a nucleic acid having a sequence as set out in any of SEQ ID NOs: 39-87, 224-246, 270-282, 297-300, 383-384 or 476.

The structure of c-Kit along with signaling through Stem Cell Factor (SCF) binding is described, for example, by Ronnstrand; Cellular and Molecular Life Sciences, 61 (2004), 2535-2548 and by Yuzawa et al. Cell 130 (2007), 323-334. Stem Cell Factor binds to the extracellular domain of c-Kit resulting in tyrosine kinase activation.

In one embodiment, the anti-c-Kit immunoglobulin single variable domain in accordance with the invention binds to c-Kit in such a way that c-Kit receptor binding and/or activation by SCF is not substantially inhibited.

In another embodiment, the anti-c-Kit immunoglobulin single variable domain in accordance with the invention binds to c-Kit in such a way that c-Kit receptor binding and/or activation by SCF is substantially inhibited.

In one embodiment, inhibition of SCF binding to c-Kit can be determined in a competitive binding assay as described herein. Other assays for determining c-Kit activation will be familiar to those skilled in the art and include assays which measure downstream signaling components as described, for example, by Ronnstrand as referred to above. Other suitable assays include assays for phosphorylation such as that provided by MSD, catalogue number K11119D-2.

In one embodiment, there is provided anti-c-Kit immunoglobulin single variable domains which bind to c-Kit and are not competitive for SCF binding. In this embodiment, the anti-c-Kit immunoglobulin single variable domain may be selected from an amino acid sequence encoded by the nucleic acid sequence set out in any of DOM28h-5 (SEQ ID NO: 39), DOM28h-33 (SEQ ID NO: 49), DOM28h-43 (SEQ ID NO: 51), DOM28h-66 (SEQ ID NO: 58), DOM28h-84 (SEQ ID NO: 62), DOM28h-94 (SEQ ID NO: 65), DOM28h-110 (SEQ ID NO: 70), DOM28m-7 (SEQ ID NO: 78), DOM28m-23 (SEQ ID NO: 81) and DOM28m-52 (SEQ ID NO: 84). Other suitable non-competitive anti-c-Kit immunoglobulin single variable domains are exemplified herein.

In another embodiment, there is provided anti-c-Kit immunoglobulin single variable domains which bind to c-Kit and are competitive for SCF binding. In this embodiment, the anti-c-Kit immunoglobulin single variable domain may be selected from an amino acid sequence encoded by the nucleic acid sequence set out in any of DOM28h-7 (SEQ ID NO: 41), DOM28h-20 (SEQ ID NO: 45), DOM28h-26 (SEQ ID NO: 48), DOM28h-54 (SEQ ID NO: 55), DOM28h-73 (SEQ ID NO: 59), DOM28h-78 (SEQ ID NO: 60) and DOM28h-79 (SEQ ID NO: 61). Other suitable competitive anti-c-Kit immunoglobulin single variable domains are exemplified herein.

In one embodiment, single variable domains of the present invention show cross-reactivity between human c-Kit and c-Kit from another species such as mouse, dog or cyno. In one embodiment, the single variable domains of the present invention show cross-reactivity between human and mouse c-Kit. In this embodiment, the variable domains specifically bind human and mouse c-Kit. In one embodiment variable domains which are cross reactive for human and mouse c-Kit are selected from an amino acid sequence encoded by the nucleic acid sequence set out in any of DOM28h-5 (SEQ ID NO: 39), DOM28h-94 (SEQ ID NO: 65), DOM28m-7 (SEQ ID NO: 78), DOM28m-23 (SEQ ID NO: 81) and DOM28m-52 (SEQ ID NO: 84). Other cross reactive variable domains are exemplified herein. As described above, cross reactivity is particularly useful, since drug development typically requires testing of lead drug candidates in animal systems, such as mouse models, before the drug is tested in humans. The provision of a drug that can bind to a human protein as well as the species homologue such as the equivalent mouse protein allows one to test results in these systems and make side-by-side comparisons of data using the same drug. This avoids the complication of needing to find a drug that works against, for example, a mouse c-Kit and a separate drug that works against human c-Kit, and also avoids the need to compare results in humans and mice using non-identical or surrogate drugs.

Optionally, the binding affinity of the immunoglobulin single variable domain for at least mouse c-Kit and the binding affinity for human c-Kit differ by no more than a factor of about 5, about 10, about 50 or about 100.

In another aspect of the invention, there is provided a method for producing an antigen binding construct (e.g, dual-specific ligand, multispecific ligand), an anti-MLC antibody or an anti-c-Kit immunoglobulin single variable domain, polypeptide or ligand in accordance with the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

Reference is made to WO200708515, page 161, line 24 to page 189, line 10 for details of disclosure that is applicable to embodiments of the present invention. This disclosure is hereby incorporated herein by reference as though it appears explicitly in the text of the present disclosure and relates to the embodiments of the present invention, and to provide explicit support for disclosure to incorporate into claims below. This includes disclosure presented in WO200708515, page 161, line 24 to page 189, line 10 providing details of the “Preparation of Immunoglobulin Based Ligands”, “Library vector systems”, “Library Construction”, “Combining Single Variable Domains”, “Characterisation of Ligands”, “Structure of Ligands”, “Skeletons”, “Protein Scaffolds”, “Scaffolds for Use in Constructing Ligands”, “Diversification of the Canonical Sequence” and “Therapeutic and diagnostic compositions and uses”, as well as definitions of “operably linked”, “naive”, “prevention”, “suppression”, “treatment”, “allergic disease”, “Th2-mediated disease”, “therapeutically-effective dose” and “effective”.

In one aspect there is provided an anti-c-Kit immunoglobulin single variable domain, polypeptide or ligand in accordance with the invention for use in targeting c-Kit for therapy of a disease or disorder associated with c-Kit receptor activation. In one embodiment the anti-c-Kit immunoglobulin single variable domain is one which competes with SCF in a competitive binding assay so as to inhibit SCF activation of c-Kit. Suitable competitive dAbs are disclosed herein.

Diseases or disorders associated with c-Kit activity include mast cell disorders and cancers. In particular, c-Kit activity has been implicated to be involved in tumour angiogenesis. Accordingly, targeting c-Kit may allow inhibition of tumour angiogenesis in an anti-cancer treatment. In addition, c-Kit and SCF autocrine loops have been identified (where a tumour expresses both c-Kit and SCF) in a number of cancers including small cell lung carcinomas, colorectal carcinoma, breast carcinoma, gynaecological tumours and neuroblastomas (see Ronnstrand; Cellular and Molecular Life Sciences, 61 (2004), 2535-2548).

In another aspect there is provided a pharmaceutical composition comprising an immunoglobulin single variable domain polypeptide or ligand in accordance with the invention.

In one embodiment, an anti-c-Kit immunoglobulin single variable domain, polypeptide or ligand in accordance with the invention may be attached to a device such as a stent. Suitable such devices are described, for example, in WO 03/065881. In one embodiment, the anti-c-Kit immunoglobulin single variable domain, polypeptide or ligand in accordance with the invention may be attached to the surface of the stent such that it is available for stem cell homing. In one embodiment in this embodiment, the c-Kit immunoglobulin single variable domain is one which binds c-Kit non-competitively i.e. is non-competitive for SCF activation of c-Kit.

In another aspect, there is provided an antigen-binding construct in accordance with the invention for use in targeting stem cells to a target tissue. In one embodiment the target tissue expresses a tissue specific marker molecule. In one embodiment, the antigen-binding construct is for use in recruiting stem cells to a target tissue in order to regenerate that target tissue. Accordingly, the construct of the present invention is for use in the treatment of a diseased target tissue. In one embodiment the construct in accordance with the invention is for use in the treatment of muscle disease. In one embodiment, there is provided and antigen-binding construct in accordance with the invention for use in the treatment of heart disease.

In a further aspect there is provided a pharmaceutical composition comprising an antigen-binding construct in accordance with the invention. In one embodiment, cytokine therapy can be used to mobilise bone marrow stem cell and progenitor cells. Cytokine therapy is described, for example, by Kang et al. Lancet (2004), 363; 751-6. Suitable cytokines include SCF, G-CSF, SDF-1, AMD3100 (commercial name=Mozobil, VEGF, FGF) and DPP-IV inhibitors.

In another aspect there is provided a method of treating heart disease comprising administering an antigen-binding construct in accordance with the invention. In one embodiment the method further comprises administering a cytokine, in one embodiment selected from SCF, G-CSF, SDF-1, AMD3100, VEGF, FGF and DPP-IV inhibitors.

In another embodiment, bone marrow cells can be extracted from the patient to be treated. In one embodiment, bone marrow cells can be extracted from the patient's sternum or iliac crest or cells may be isolated from the blood. Unfractionated bone marrow cells may be used for subsequent systemic/local delivery or specific cell populations may be isolated using cell sorting techniques (for example, FACS or magnetic bead immunoselection). Cells may also be cultured in vitro prior to injection back to the patient to promote a number of mechanisms such as increasing cell number by treatment with mitotic agents, increasing cell function with factors such as VEGF and statins to increase survival, differentiation or angiogenic capacity, for example. Cells may also be genetically engineered to modulate gene expression of, for example, survival factors or pro-regenerative factors. Cells may be purified based on selection for cell surface markers using magnetic cell sorting techniques (for example, see Losordo et al. Circulation 2007 Jun. 26; 115(25):3165-72). Cell clusters such as cardiospheres may be generated in vitro (as described, for example, in Barile et al., Nature Clinical Practice, February 2007, Vol. 4 Supplement 1). Other cells for use in accordance with the invention include haemangioblasts, mesenchymal stem cells, haematopoietic stem cells or endothelial progenitor cells. Accordingly, in one embodiment there is provided a method for treating heart disease comprising extracting bone marrow cells from a patient, treating said cells in vitro and returning said cells to the patient prior to administering a construct.

Cells may be administered to the patient using intravenous administration, or, for example, through intramyocardial administration or intracoronary delivery via a catheter. In one embodiment, cells may be administered locally during surgical intervention.

In one embodiment, the construct in accordance with the invention recruits stem cells to the target tissue such as muscle. In one embodiment, the construct recruits stem cells to the myocardium. In one embodiment, c-Kit+ cells are recruited.

In one embodiment the stem cells are cells which can generate myoblasts or myocytes such that muscle can be repaired. In one embodiment the stem cells can generate vascular cells (including endothelial and smooth muscle cells) that will repair damaged vasculature, which in itself will promote survival of the muscle and myocyte differentiation from stem cells. In one embodiment, the stem cells can repair the myocardium. In particular, stem cells which are targeted by molecules of the invention are cells which can differentiate into cardiomyocytes, vascular endothelial cells or smooth muscle cells. In another embodiment, the stem cells can generate myoblasts or myocytes such that damage to skeletal muscle can be repaired. Thus, in one embodiment, stem cells are adult stem cells such as haematopoietic stem cells, mesenchymal stem cells, cardiac stem cells, endothelial progenitor cells, induced pluripotent stem cells (iPS). In another embodiment, stem cells are embryonic stem cells. Besides the action of stem cells to differentiate into cardiovascular cell types, these stem cells also have the ability to act in a paracrine manner, secreting growth factors, cytokines and other molecules that can act at the site of injury to promote cell survival, cell repair, tissue regeneration, angiogenesis and myocardial regeneration. In one embodiment, the stem cells are haematopoietic stem cells. Stem cells for use in the present invention may be derived from the patient themselves (i.e. autologous) or may be allogeneic (i.e. derived from somebody else). In one embodiment, the stem cells are CD34+ cells. In another embodiment, the stem cells can be any mammalian stem cell including, for example, stem cells from a primate, such as a human or stem cells from a rodent, a cat, a pig, a sheep, a dog, a cow or a horse.

In another embodiment, the stem cells may be genetically modified such that they encompass transduced genes for gene therapy.

Another embodiment provides a method for treating muscle disease or heart disease further comprising administering a compound to enhance stem cell survival, differentiation or proliferation. Suitable compounds include VEGF, FGF, statins, SDF-1, CXCR4 (described for example by Tan et al Cardiovascular Res. (Advance Access published on Feb. 24, 2009; doi: doi:10.1093/cvr/cvp044)) or SDF-1betaP2G. Such compounds improve the ability of these cells to contribute to cardiac regeneration and prevent the long-term damage observed after myocardial injury as reviewed, for example, in Ballard and Edelberg, Circulation Research 2007, 100(8): 1116-27.

### BRIEF DESCRIPTION OF THE DRAWINGS

FIG. 1 shows amino acid and nucleic acid sequences for human and mouse vMLC1-(6×HIS tag).

FIG. 2 shows amino acid sequences of human, mouse, dog and cyno c-KIT ECD-hIgG1 Fc fusion (c-KIT ECD (extracellular domain) in bold).

FIG. 3 shows amino acid of human and mouse SCF-6×HIS tag.

FIG. 4 shows anti-MLC antibody Mouse Kappa chain (Vk gene in BOLD; CDR sequences underlined) and anti-MLC antibody Mouse Heavy IgG1 chain (V_{H} gene in BOLD; CDR sequences underlined).

FIG. 5 shows humanised 39-15 mAb V genes (CDR sequences underlined).

FIG. 6 shows nucleic acid and amino acid sequences for dAbs which bind c-kit.

FIG. 7 shows predicted CDR sequences from the corresponding amino acid sequences for selected dAbs. Using Kabat numbering the CDRs are determined as follows: (VH-CDR1 (30-35), VH-CDR2 (50-56), VH-CDR3 (94-102), VK-CDR1 (26-34), VK-CDR2 (49-56), VK-CDR3 (89-97).

FIG. 8 shows BIAcore binding traces of dAbs that bind human c-kit non-competitively. dAb binding was assessed on biotinylated human c-kit (His-tagged) immobilized on a streptavidin chip. The traces allow visual comparison of the relative off-rates and on-rates of the dAbs and fitting of the curves using kinetic models allows calculation of the affinity constants for the dAbs.

FIGS. 9 & 10 show the results of Competitive Receptor Binding Assays (RBA). In this assay dAbs are assessed to determine whether or not they can inhibit the interaction between human c-kit and human stem cell factor (SCF). Those dAbs that are competitive with SCF inhibit the interaction reducing the signal as the dAb concentration increases, whereas those dAbs that are non-competitive with SCF have no effect and therefore the signal remains constant as the dAb concentration increases.

FIG. 11a & 11b show binding of non-competitive dAbs to KU812 cells by flow cytometry. This assay determines whether the dAbs can bind specifically to c-kit displayed on the cell surface by looking at the binding to the KU812 cell line which has been shown to be c-kit+ve. 2 pt curves (100-500 nM) are shown in FIG. 11a, 2 pt curves (80-400 nM) are shown in FIG. 11b.

FIGS. 12a & 12b show binding of non-competitive dAbs to Jurkat cells by flow cytometry. This assay determines whether the dAbs are binding specifically or non-specifically to cells by looking at binding to the Jurkat cell line which has been shown to be c-kit-ve. 2 pt curves (100-500 nM) are shown in FIG. 12a, 2 pt curves (80-400 nM) are shown in FIG. 12b.

FIG. 13 shows binding of competitive dAbs to KU812 by flow cytometry. This assay determines whether the dAbs can bind specifically to c-kit displayed on the cell surface by looking at the binding to the KU812 cell line which has been shown to be c-kit+ve. 3 pt curves (400 nM-2 uM-10 uM) are shown.

FIG. 14 shows binding of competitive dAbs to Jurkat cells by flow cytometry. This assay determines whether the dAbs are binding specifically or non-specifically to cells by looking at binding to the Jurkat cell line which has been shown to be c-kit-ve. 3 pt curves (400 nM-2 uM-10 uM) are shown.

FIG. 15 shows binding of panel of dAbs to c-kit+ve gated mouse bone marrow cells by flow cytometry. This assay determined whether dAbs can bind to murine bone marrow cells which have been sorted on the basis of being cKIT+ve. 2 pt curves are shown (5 uM-10 uM).

FIG. 16 shows nucleic acid and amino acid sequences for dAbs that bind c-kit.

FIG. 17 shows nucleic acid and amino acid sequences for dAbs that bind c-kit.

FIG. 18 shows BIAcore results of 13 dAbs that are active in cells and compatible as a mAbdAb.

FIG. 19 shows epitope mapping via sequence-structure comparisons.

FIG. 20 shows nucleic acid and amino acid sequences for dAbs that bind c-kit.

FIG. 21 shows a schematic diagram illustrating different antibody formats.

FIG. 22 shows a schematic diagram illustrating the construction of a mAbdAb heavy chain (top illustration) or a mAbdAb light chain (bottom illustration).

FIG. 23 shows schematic illustrations of mAb-dAbs described in Example 5.

FIG. 24 shows a schematic diagram illustrating cloning of Dummy mAb-cKIT dAb mAb-dAbs.

FIG. 25 shows nucleic acid and amino acid sequences of constructs described in Example 5.

FIG. 26 shows epitope analysis of c-kit dAbs.

FIG. 27 shows a BIAcore example of typical BIAcore epitope mapping experiment where the epitopes are not overlapping for 2B8 the commercial antibody and 4552 (DOM28m-107 in a dummy framework Mab).

FIG. 28 shows BIAcore example of a typical BIAcore epitope mapping experiment where the epitopes are partially overlapping for the dummy framework Mab 4505 (DOM28m-7) and 4503 (DOM28h-94).

FIG. 29 shows a comparison of bispecific mAb-dAbs, control molecules and DOM28h-94 affinity matured clones in 10% mouse serum.

FIG. 30 exemplifies cell surface staining, cell surface and intracellular staining and intracellular staining patterns.

FIG. 31 shows a Table of a list of sequences identified herein.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g, in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

Nomenclature and Abbreviations:

Nomenclature and abbreviations used herein include: Monoclonal antibody (MAb, mAb); Monoclonal antibodies (mAbs); Domain antibody (dAb); Domain antibodies (dAbs); Heavy Chain (H chain); Light chain (L chain); Heavy chain variable region (V_{H}); Light chain variable region (V_{L}); kappa light chain variable region (Vk); Human IgG1 constant heavy region 1 (CH1); Human IgG1 constant heavy region 2 (CH2); Human IgG1 constant heavy region 3 (CH3); Light chain/kappa light chain constant region (CL/CK); and complementarity determining region (CDR)—of heavy chain (CDRH);—of light chain (CDRL); regions 1, 2, 3 (CDR1, CDR2, CDR3).

Target Tissues and Tissue Specific Marker Molecules:

Suitable target tissues include muscle tissue, including the myocardium and skeletal muscle, epithelial tissue, skin, connective tissue, hepatic tissue, neuronal tissue, heart or cardiac tissue and articular tissue. Tissue specific markers for each of these tissues are known by those skilled in the art. In one embodiment, tissue specific markers include markers of inflammation, components of scar tissue or markers which are specific to tissues such as muscle tissue, including the myocardium, epithelial tissue, skin, connective tissue, hepatic tissue, neuronal tissue, cardiac tissue and articular tissue. In one embodiment, the invention provides compositions and methods for targeting stem cells to the heart tissue including myocardial tissue, fibroblasts, coronary vasculature and proteins in the interstitial space or basement membrane.

Muscle Specific Marker Molecules:

Myosins:

Myosins are a large family of motor proteins which are found in the muscle sarcomere and are responsible for actin-based motility. Myosin molecules are composed of heavy and light chains interlinked in a three dimensional structure. A cytosolic precursor pool of light chain molecules has been described in muscle cells and it is thought that these leak out into the circulation upon myocardial damage, for example (as described, for example in U.S. Pat. No. 5,702,905).

In muscle, the myosin light chains (MLCs) in a myosin molecule are found in pairs. Cardiac and skeletal MLCs are immunologically distinct. Cardiac MLC is present in myocardium and myocardial infarctions (as described, for example, by Lyn et al. Physiol. Genomics 2000; 2:93-100; Mair et al. Clin Chim Acta 1994; 229:153-159 and Khaw et al. J. Clin. Invest. 1976; 58: 439-446). When the tissue membrane is damaged in myocardial infarctions, they become accessible to anti-MLC antibodies. Myosin Light Chains include MLC-1, MLC-2, MYL, MYL-2/3, human ventricular myosin light chain (vMLC), vMLC-1 (UniProtKB/Swiss-Prot entry P08590). U.S. Pat. No. 5,702,905 describes a mouse monoclonal antibody to human ventricular myosin light chain which has high affinity for the cardiac isoforms of myosin light chains. vMLC (also known as MLC-1, MLC-3) is expressed in the heart muscle, skeletal muscle, vascular smooth muscle, umbilical artery smooth muscle cells and in muscle tissue in kidney, colon, fallopian tubes, rectum, seminal vesicle, prostate, skin, intestinal endothelium, pancreas, adipose tissue, retinal endothelial cells and urinary bladder epithelium. See, for example, Bicer and Reiser, J Muscle Res Cell Motil. 2004; 25(8):623-33.

As used herein “MLC” also includes a portion or fragment of a MLC. MLCs include naturally occurring or endogenous mammalian MLC proteins and to proteins having an amino acid sequence which is the same as that of a naturally occurring or endogenous corresponding mammalian MLC protein (e.g, recombinant proteins, synthetic proteins (i.e., produced using the methods of synthetic organic chemistry)). Accordingly, as defined herein, the term includes mature MLC protein, polymorphic or allelic variants, and other isoforms of MLC and modified or unmodified forms of the foregoing (e.g, lipidated, glycosylated).

Stem-Cell Specific Marker Molecules:

Stem-cell specific marker molecules include those molecules which are expressed on stem cells. Such molecules include: CD30, Nestin, Stro-1, PSA-NCam, p75, Neurotrophin, CD34, Sca-1, ABCG2, CD133 and c-Kit. c-Kit (also referred to as CD117 and SCFR (stem cell factor receptor); human c-Kit is described in UniProtKB/Swiss-Prot record P10721)) is a cell and membrane associated tyrosine kinase receptor. Stem Cell Factor (SCF) is a glycoprotein that signals through binding c-Kit and this signaling pathway plays a key role in hematopoiesis acting both as a positive and negative regulator, often in synergy with other cytokines. A soluble shed c-Kit receptor may play a role in regulating SCF. In one embodiment, the agent which binds to a stem cell specific marker molecule may be a receptor binding protein or growth factor such as SCF.

c-Kit (also referred to as c-KIT, cKIT, c-kit, ckit, cKit) is expressed on pluripotent hematopoietic stem cells which are the precursors to mature cells belonging to lymphoid and erythroid lineages. Expression of c-Kit on stem and progenitor cells from the bone marrow and on cardiac stem cells and the role of these cells in myocardial repair is described, for example, by Fazel et al. Journal of Clinical Investigation, 116 (2006), 7, 1865-1876. c-Kit is an early stem cell marker which is found on a significant portion of the stem cell population, being expressed by approximately 1% of circulating white blood cells. Cells expressing c-kit have been shown to give rise to both cardiomyocytes and vascular cell types. In addition, the absence of c-kit in animal models leads to impairment in cardiac repair after MI, suggesting that these cells play a key role in cardiovascular regeneration. c-Kit+ cells are found in the bone marrow and are subsequently mobilized to the bloodstream after injury or administration of a mobilizing agent (reviewed, for example, by Bearzi et al. PNAS (2007) 104; 35; 14068-14073).

As used herein “c-Kit” also includes a portion or fragment of c-Kit. c-Kit includes naturally occurring or endogenous mammalian c-Kit proteins and to proteins having an amino acid sequence which is the same as that of a naturally occurring or endogenous corresponding mammalian c-Kit protein (e.g, recombinant proteins, synthetic proteins (i.e., produced using the methods of synthetic organic chemistry)).

Accordingly, as defined herein, the term includes mature c-Kit protein, polymorphic or allelic variants, and other isoforms of c-Kit and modified or unmodified forms of the foregoing (e.g, lipidated, glycosylated). Mammalian c-Kit used include rat c-Kit (also referred to as rc-Kit, rcKIT, rc-kit) and mouse/murine c-Kit (also referred to as mcKIT, mc-Kit, mc-kit).

Immunoglobulin:

As used herein, “immunoglobulin” refers to a family of polypeptides which retain the immunoglobulin fold characteristic of antibody molecules, which contain two 13 sheets and, usually, a conserved disulphide bond.

Domain:

As used herein “domain” refers to a folded protein structure which retains its tertiary structure independently of the rest of the protein. Generally, domains are responsible for discrete functional properties of proteins and in many cases may be added, removed or transferred to other proteins without loss of function of the remainder of the protein and/or of the domain.

Immunoglobulin Single Variable Domain:

The phrase “immunoglobulin single variable domain” or single antibody variable domain refers to an antibody variable domain (V_{H}, V_{HH}, V_{L}) or binding domain that specifically binds an antigen or epitope independently of different or other V regions or domains. Such an “immunoglobulin single variable domain” is a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain. An immunoglobulin single variable domain can be present in a format (e.g, homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

In all aspects of the invention, the or each immunoglobulin single variable domain is independently selected from antibody heavy chain and light chain single variable domains, e.g. V_{H}, V_{L} and V_{HH}.

Antibody:

As used herein an “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from, for example, serum, B-cells, hybridomas, transfectomas, yeast or bacteria.

Antibody Format:

In one embodiment, the antibody, immunoglobulin single variable domain, polypeptide or ligand in accordance with the invention can be provided in any antibody format. As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g, a Fv fragment (e.g, single chain Fv (scFv), a disulfide bonded Fv), a Fab fragment, a Fab′ fragment, a F(ab′)₂ fragment), a single antibody variable domain (e.g, a dAb, V_{H}, V_{HH}, V_{L}), and modified versions of any of the foregoing (e.g, modified by the covalent attachment of polyethylene glycol or other suitable polymer or a humanized V_{HH}). In one embodiment, alternative antibody formats include alternative scaffolds in which the CDRs of any molecules in accordance with the invention can be grafted onto a suitable protein scaffold or skeleton, such as an affibody, a SpA scaffold, an LDL receptor class A domain, an avimer (see, e.g, U.S. Patent Application Publication Nos. 2005/0053973, 2005/0089932, 2005/0164301) or an EGF domain. Further, the ligand can be bivalent (heterobivalent) or multivalent (heteromultivalent) as described herein. In other embodiments, a “Universal framework” may be used wherein “Universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity through variation in the hypervariable regions alone.

If desired, an “antibody” can further comprise one or more additional moieties that can each independently be a peptide, polypeptide or protein moiety or a non-peptidic moiety (e.g, a polyalkylene glycol, a lipid, a carbohydrate). For example, the ligand can further comprise a half-life extending moiety (e.g, a polyalkylene glycol moiety, a moiety comprising albumin, an albumin fragment or albumin variant, a moiety comprising transferrin, a transferrin fragment or transferrin variant, a moiety that binds albumin, a moiety that binds neonatal Fc receptor). Suitable half-life extending moieties are described, for example, in WO2008096158. Another approach is to include an additional binding moiety such as an antibody or immunoglobulin single variable domain which binds to a peptide, polypeptide or protein moiety such as serum albumin, as described, for example in EP1517921, WO03002609, WO04003019, WO2008096158, WO04058821 and WO2007080392. Suitable Camelid V_{HH} that bind serum albumin include those disclosed in WO 2004041862 (Ablynx N.V.) and in WO2007080392

By “anti-MLC” or “anti-c-Kit” with reference to an immunoglobulin single variable domain, polypeptide, ligand, fusion protein or so forth is meant a moiety which recognises and binds MLC or c-Kit. In particular, reference to “anti-MLC” encompasses a moiety which binds any MLC variant including vMLC-1 and so forth.

Epitope:

An “epitope” is a unit of structure conventionally bound by an immunoglobulin V_{H}/V_{L}, pair. Epitopes define the minimum binding site for an antibody, and thus represent the target of specificity of an antibody. In the case of a single domain antibody, an epitope represents the unit of structure bound by a variable domain in isolation.

Epitope Binding Domain:

The term “Epitope-binding domain” refers to a domain that specifically binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than the natural ligand.

Binding:

Binding is indicated by a dissociation constant (Kd). Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof.

Binding Affinity:

Binding affinity is optionally determined using surface plasmon resonance (SPR) and Biacore (Karlsson et al., 1991), using a Biacore system (Uppsala, Sweden). The Biacore system uses surface plasmon resonance (SPR, Welford K. 1991, Opt. Quant. Elect. 23:1; Morton and Myszka, 1998, Methods in Enzymology 295: 268) to monitor biomolecular interactions in real time, and uses surface plasmon resonance which can detect changes in the resonance angle of light at the surface of a thin gold film on a glass support as a result of changes in the refractive index of the surface up to 300 nm away. Biacore analysis conveniently generates association rate constants, dissociation rate constants, equilibrium dissociation constants, and affinity constants. Binding affinity is obtained by assessing the association and dissociation rate constants using a Biacore surface plasmon resonance system (Biacore, Inc.). A biosensor chip is activated for covalent coupling of the target according to the manufacturer's (Biacore) instructions. The target is then diluted and injected over the chip to obtain a signal in response units of immobilized material. Since the signal in resonance units (RU) is proportional to the mass of immobilized material, this represents a range of immobilized target densities on the matrix. Dissociation data are fit to a one-site model to obtain k_{off}+/−s.d. (standard deviation of measurements). Pseudo-first order rate constant (Kd's) are calculated for each association curve, and plotted as a function of protein concentration to obtain kₒₙ+/−s.e. (standard error of fit). Equilibrium dissociation constants for binding, Kd's, are calculated from SPR measurements as k_{off}/kₒₙ.

CDRs:

The antigen binding proteins, antibodies and immunoglobulin single variable domains (dAbs) described herein contain complementarity determining regions (CDR1, CDR2 and CDR3). The locations of CDRs and frame work (FR) regions and a numbering system have been defined by Kabat et al. (Kabat, E. A. et al., Sequences of Proteins of Immunological Interest, Fifth Edition, U.S. Department of Health and Human Services, U.S. Government Printing Office (1991)). The amino acid sequences of the CDRs (CDR1, CDR2, CDR3) of the V_{H} (CDRH1 etc.) and V_{L} (CDRL1 etc.) (V_{K}) dAbs disclosed herein will be readily apparent to the person of skill in the art based on the well known Kabat amino acid numbering system and definition of the CDRs. According to the Kabat numbering system, the most commonly used method based on sequence variability, heavy chain CDR-H3 have varying lengths, insertions are numbered between residue H100 and H101 with letters up to K (i.e. H100, H100A . . . H100K, H101). CDRs can alternatively be determined using the system of Chothia (based on location of the structural loop regions) (Chothia et al., (1989) Conformations of immunoglobulin hypervariable regions; Nature 342, p877-883), according to AbM (compromise between Kabat and Chothia) or according to the Contact method (based on crystal structures and prediction of contact residues with antigen) as follows. See http://www.bioinforg.uk/abs/ for suitable methods for determining CDRs.

Once each residue has been numbered, one can then apply the following CDR definitions:

Kabat:
CDR H1: 31-35/35A/35B
CDR H2: 50-65
CDR H3: 95-102
CDR L1: 24-34
CDR L2: 50-56
CDR L3: 89-97

Chothia:

CDR H1: 26-32
CDR H2: 52-56
CDR H3: 95-102
CDR L1: 24-34
CDR L2: 50-56
CDR L3: 89-97

| | |
|---|---|
| (using Kabat numbering): | (using Chothia numbering): |

AbM:

| | |
|---|---|
| CDR H1: 26-35/35A/35B | 26-35 |
| CDR H2: 50-58 | — |
| CDR H3: 95-102 | — |
| CDR L1: 24-34 | — |
| CDR L2: 50-56 | — |
| CDR L3: 89-97 | — |

Contact

| | |
|---|---|
| CDR H1: 30-35/35A/35B | 30-35 |
| CDR H2: 47-58 | — |
| CDR H3: 93-101 | — |
| CDR L1: 30-36 | — |
| CDR L2: 46-55 | — |
| CDR L3: 89-96 | — |

(“—” means the same numbering as Kabat)

Competes:

As referred to herein, the term “competes” means that the binding of a first target (e.g., c-Kit) to its cognate target binding domain (e.g., immunoglobulin single variable domain) is inhibited in the presence of a second binding domain (e.g., immunoglobulin single variable domain) that is specific for said cognate target. For example, binding may be inhibited sterically, for example by physical blocking of a binding domain or by alteration of the structure or environment of a binding domain such that its affinity or avidity for a target is reduced. See WO2006038027 for details of how to perform competition ELISA and competition BIACore experiments to determine competition between first and second binding domains, the details of which are incorporated herein by reference to provide explicit disclosure for use in the present invention.

Linking dAbs to IgG:

Domain antibodies of use in the present invention can be linked at the C-terminal end of the heavy chain and/or the light chain of conventional IgGs. In addition some dAbs can be linked to the C-terminal ends of both the heavy chain and the light chain of conventional antibodies.

In constructs where the N-terminus of dAbs are fused to an antibody constant domain (either CO or CL), a peptide linker may help the dAb to bind to antigen. Indeed, the N-terminal end of a dAb is located closely to the complementarity-determining regions (CDRS) involved in antigen-binding activity. Thus a short peptide linker acts as a spacer between the epitope-binding, and the constant domain of the protein scaffold, which may allow the dAb CDRs to more easily reach the antigen, which may therefore bind with high affinity.

The surroundings in which dAbs are linked to the IgG will differ depending on which antibody chain they are fused to:

When fused at the C-terminal end of the antibody light chain of an IgG scaffold, each dAb is expected to be located in the vicinity of the antibody hinge and the Fc portion. It is likely that such dAbs will be located far apart from each other. In conventional antibodies, the angle between Fab fragments and the angle between each Fab fragment and the Fc portion can vary quite significantly. It is likely that—with mAbdAbs—the angle between the Fab fragments will not be widely different, whilst some angular restrictions may be observed with the angle between each Fab fragment and the Fc portion.

When fused at the C-terminal end of the antibody heavy chain of an IgG scaffold, each dAb is expected to be located in the vicinity of the C_{H}3 domains of the Fc portion. This is not expected to impact on the Fc binding properties to Fc receptors (e.g. FcγRI, II, III an FcRn) as these receptors engage with the C_{H}2 domains (for the FcγRI, II and III class of receptors) or with the hinge between the C_{H}2 and C_{H}3 domains (e.g. FcRn receptor). Another feature of such antigen-binding constructs is that both dAbs are expected to be spatially close to each other and provided that flexibility is provided by provision of appropriate linkers, these dAbs may even form homodimeric species, hence propagating the ‘zipped’ quaternary structure of the Fc portion, which may enhance stability of the construct.

Such structural considerations can aid in the choice of the most suitable position to link an epitope-binding domain, for example a dAb, on to a protein scaffold, for example an antibody.

Linkers:

Protein scaffolds of the present invention may be linked to epitope-binding domains by the use of linkers. Examples of suitable linkers include amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

In one embodiment of the present invention at least one of the epitope binding domains is directly attached to the Ig scaffold with a linker comprising from 1 to 150 amino acids, for example 1 to 20 amino acids, for example 1 to 10 amino acids.

Such linkers may be selected from any one of those set out in SEQ ID NO: 3 to 8, for example the linker may be ‘TVAAPS’, or the linker may be ‘GGGGS’, or multiples of such linkers. Linkers of use in the antigen-binding proteins of the present invention may comprise alone or in addition to other linkers, one or more sets of GS residues, for example ‘GSTVAAPS’ (SEQ ID NO: 102) or ‘TVAAPSGS’ (SEQ ID NO: 103) or ‘GSTVAAPSGS’ (SEQ ID NO: 104), or multiples of such linkers. In one embodiment the epitope binding domain is linked to the Ig scaffold by the linker ‘(PAS)ₙ(GS)ₘ’. In another embodiment the epitope binding domain is linked to the Ig scaffold by the linker ‘(GGGGS)ₙ(GS)ₘ’. In another embodiment the epitope binding domain is linked to the Ig scaffold by the linker ‘(TVAAPS)ₙ(GS)ₘ’. In another embodiment the epitope binding domain is linked to the Ig scaffold by the linker ‘(GS)ₘ(TVAAPSGS)ₙ’. In another embodiment the epitope binding domain is linked to the Ig scaffold by the linker ‘(GS)ₘ(TVAAPS)ₚ(GS)ₘ’. In another embodiment the epitope binding domain is linked to the Ig scaffold by the linker ‘(PAVPPP)ₙ(GS)ₘ’. In another embodiment the epitope binding domain is linked to the Ig scaffold by the linker ‘(TVSDVP)ₙ(GS)ₘ’. In another embodiment the epitope binding domain is linked to the Ig scaffold by the linker ‘(TGLDSP)ₙ(GS)ₘ’. In all such embodiments, n=1-10, m=0-4 and p=2-10.

Examples of such linkers include (PAS)ₙ(GS)ₘ wherein n=1 and m=1, (PAS)ₙ(GS)ₘ wherein n=2 and m=1, (PAS)ₙ(GS)ₘ wherein n=3 and m=1, (PAS)ₙ(GS)ₘ wherein n=4 and m=1, (PAS)ₙ(GS)ₘ wherein n=2 and m=0, (PAS)ₙ(GS)ₘ wherein n=3 and m=0, (PAS)ₙ(GS)ₘ wherein n=4 and m=0.

Examples of such linkers include (GGGGS)ₙ(GS)ₘ wherein n=1 and m=1, (GGGGS)ₙ(GS)ₘ wherein n=2 and m=1, (GGGGS)ₙ(GS)ₘ wherein n=3 and m=1, (GGGGS)ₙ(GS)ₘ wherein n=4 and m=1, (GGGGS)ₙ(GS)ₘ wherein n=2 and m=0, (GGGGS)ₙ(GS)ₘ wherein n=3 and m=0, (GGGGS)ₙ(GS)ₘ wherein n=4 and m=0.

Examples of such linkers include (GS)ₘ(TVAAPS)ₚ wherein p=1 and m=1, (GS)ₘ(TVAAPS)ₚ wherein p=2 and m=1, (GS)ₘ(TVAAPS)ₚ wherein p=3 and m=1, (GS)ₘ(TVAAPS)ₚ wherein p=4 and m=1), (GS)ₘ(TVAAPS)ₚ wherein p=5 and m=1, or (GS)ₘ(TVAAPS)ₚ wherein p=6 and m=1.

Examples of such linkers include (TVAAPS)ₙ(GS)ₘ wherein n=1 and m=1, (TVAAPS)ₙ(GS)ₘ wherein n=2 and m=1, (TVAAPS)ₙ(GS)ₘ wherein n=3 and m=1, (TVAAPS)ₙ(GS)ₘ wherein n=4 and m=1, (TVAAPS)ₙ(GS)ₘ wherein n=2 and m=0, (TVAAPS)ₙ(GS)ₘ wherein n=3 and m=0, (TVAAPS)ₙ(GS)ₘ wherein n=4 and m=0.

Examples of such linkers include (GS)ₘ(TVAAPSGS)ₙ wherein n=1 and m=1, (GS)ₘ(TVAAPSGS)ₙ wherein n=2 and m=1, (GS)ₘ(TVAAPSGS)ₙ wherein n=3 and m=1, or (GS)ₘ(TVAAPSGS)ₙ wherein n=4 and m=1, (GS)ₘ(TVAAPSGS)ₙ wherein n=5 and m=1, (GS)ₘ(TVAAPSGS)ₙ wherein n=6 and m=1, (GS)ₘ(TVAAPSGS)ₙ wherein n=1 and m=0, (GS)ₘ(TVAAPSGS)ₙ wherein n=2 and m=10, (GS)ₘ(TVAAPSGS)ₙ wherein n=3 and m=0, or (GS)ₘ(TVAAPSGS)ₙ wherein n=0.

Examples of such linkers include (TVAAPSGS)ₚ(GS)ₘ wherein p=2 and m=1, (TVAAPSGS)ₚ(GS)ₘ wherein p=3 and m=1, (TVAAPSGS)ₚ(GS)ₘ wherein p=4 and m=1, (TVAAPSGS)ₚ(GS)ₘ wherein p=2 and m=0, (TVAAPSGS)ₚ(GS)ₘ wherein p=3 and m=0, (TVAAPSGS)ₚ(GS)ₘ wherein p=4 and m=0.

Examples of such linkers include (PAVPPP)ₙ(GS)ₘ wherein n=1 and m=1, (PAVPPP)ₙ(GS)ₘ wherein n=2 and m=1 (SEQ ID NO: 65), (PAVPPP)ₙ(GS)ₘ wherein n=3 and m=1, (PAVPPP)ₙ(GS)ₘ wherein n=4 and m=1, (PAVPPP)ₙ(GS)ₘ wherein n=2 and m=0, (PAVPPP)ₙ(GS)ₘ wherein n=3 and m=0, (PAVPPP)ₙ(GS)ₘ wherein n=4 and m=0.

Examples of such linkers include (TVSDVP)ₙ(GS)ₘ wherein n=1 and m=1 (SEQ ID NO: 67), (TVSDVP)ₙ(GS)ₘ wherein n=2 and m=1, (TVSDVP)ₙ(GS)ₘ wherein n=3 and m=1, (TVSDVP)ₙ(GS)ₘ wherein n=4 and m=1, (TVSDVP)ₙ(GS)ₘ wherein n=2 and m=0, (TVSDVP)ₙ(GS)ₘ wherein n=3 and m=0, (TVSDVP)ₙ(GS)ₘ wherein n=4 and m=0.

Examples of such linkers include (TGLDSP)ₙ(GS)ₘ wherein n=1 and m=1, (TGLDSP)ₙ(GS)ₘ wherein n=2 and m=1, (TGLDSP)ₙ(GS)ₘ wherein n=3 and m=1, (TGLDSP)ₙ(GS)ₘ wherein n=4 and m=1, (TGLDSP)ₙ(GS)ₘ wherein n=2 and m=0, (TGLDSP)ₙ(GS)ₘ wherein n=3 and m=0, (TGLDSP)ₙ(GS)ₘ wherein n=4 and m=0.

In another embodiment there is no linker between the epitope binding domain and the Ig scaffold. In another embodiment the epitope binding domain is linked to the Ig scaffold by the linker ‘TVAAPS’ (SEQ ID NO: 89). In another embodiment the epitope binding domain, is linked to the Ig scaffold by the linker ‘TVAAPSGS’ (SEQ ID NO: 103). In another embodiment the epitope binding domain is linked to the Ig scaffold by the linker ‘GS’ (SEQ ID NO: 105). In another embodiment the epitope binding domain is linked to the Ig scaffold by the linker ‘ASTKGPT’ (SEQ ID NO: 91).

Homology:

Sequences similar or homologous (e.g, at least about 70% sequence identity) to the sequences disclosed herein are also part of the invention. In some embodiments, the sequence identity at the amino acid level can be about 75%, 80%, 85%, 90%, 91%, 92%, 93%, 94%, 95%, 96%, 97%, 98%, 99% or higher. At the nucleic acid level, the sequence identity can be about 60%, 65%, 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g, very high stringency hybridization conditions), to the complement of the strand. The nucleic acids may be present in whole cells, in a cell lysate, or in a partially purified or substantially pure form.

As used herein, the terms “low stringency,” “medium stringency,” “high stringency,” or “very high stringency” conditions describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and optionally (4) very high stringency hybridization conditions are 0.5M sodium phosphate, 7% SDS at 65° C., followed by one or more washes at 0.2×SSC, 1% SDS at 65° C.

Calculations of “homology” or “sequence identity” or “similarity” between two sequences (the terms are used interchangeably herein) are performed as follows. The sequences are aligned for optimal comparison purposes (e.g, gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least about 30%, optionally at least about 40%, optionally at least about 50%, optionally at least about 60%, and optionally at least about 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are optionally prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Nucleic Acid Molecules, Vectors, Host Cells and Protein Expression of Constructs:

The invention also provides isolated and/or recombinant nucleic acid molecules encoding ligands (single variable domains, fusion proteins, polypeptides, dual-specific ligands and multispecific ligands) as described herein.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g, plasmids, phagemids), expression control elements, host cells and methods for producing recombinant host cells of the invention are well-known in the art.

The antigen binding constructs of the present invention may be produced by transfection of a host cell with an expression vector comprising the coding sequence for the antigen binding construct of the invention. An expression vector or recombinant plasmid is produced by placing these coding sequences for the antigen binding construct in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding construct light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding construct may reside on a single vector, for example in two expression cassettes in the same vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding construct of the invention. The antigen binding construct which includes the association of both the recombinant heavy chain and/or light chain is screened from culture by appropriate assay, such as ELISA or RIA. Similar conventional techniques may be employed to construct other antigen binding constructs.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding constructs of the present invention. Host cells useful for the cloning and other manipulations of these cloning vectors are also conventional. However, cells from various strains of E. coli may be used for replication of the cloning vectors and other steps in the construction of antigen binding constructs of this invention.

Examples of host cells or cell lines for the expression of the antigen binding constructs of the invention include mammalian cells such as NSO, Sp2/0, CHO (e.g., ATCC Accession No. CRL-9096, CHO DG44 (Urlaub, G. and Chasin, L A., Proc. Natl. Acad. Sci. USA, 77(7):4216-4220 (1980)))), COS such as COS-1 (ATCC Accession No. CRL-1650) and COS-7 (ATCC Accession No. CRL-1651), HEK, 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NSO cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).), a fibroblast cell (e.g., 3T3), and myeloma cells. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of fungal or yeast cells known to those skilled in the art are also available as host cells (e.g., Pichia pastoris, Aspergillus sp., Saccharomyces cerevisiae, Schizosaccharomyces pombe, Neurospora crassa), as well as insect cells, e.g. Drosophila and Lepidoptera and viral expression systems (e.g., Drosophila Schnieder S2 cells, Sf9 insect cells (WO 94/26087 (O'Connor)). See, e.g. Miller et al., Genetic Engineering, 8:277-298, Plenum Press (1986) and references cited therein

The general methods by which the vectors may be constructed, the transfection methods required to produce the host cells of the invention, and culture methods necessary to produce the antigen binding construct of the invention from such host cell may all be conventional techniques. Typically, the culture method of the present invention is a serum-free culture method, usually by culturing cells serum-free in suspension. Likewise, once produced, the antigen binding constructs of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies is described in WO 99/58679 and WO 96/16990.

Yet another method of expression of the antigen binding constructs may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

In a further aspect of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an antigen binding construct of the present invention which method comprises the steps of;
(a) providing a first vector encoding a heavy chain of the antigen binding construct;
(b) providing a second vector encoding a light chain of the antigen binding construct;
(c) transforming a mammalian host cell (e.g. CHO) with said first and second vectors;
(d) culturing the host cell of step (c) under conditions conducive to the secretion of the antigen binding construct from said host cell into said culture media;
(e) recovering the secreted antigen binding construct of step (d).

Once expressed by the desired method, the antigen binding construct is then examined for in vitro activity by use of an appropriate assay. Presently conventional ELISA assay formats or BIAcore are employed to assess qualitative and quantitative binding of the antigen binding construct to its target. Additionally, other in vitro assays may also be used to verify neutralizing efficacy prior to subsequent human clinical studies performed to evaluate the persistence of the antigen binding construct in the body despite the usual clearance mechanisms.

Advantageously, the antigen binding construct of the present invention can be expressed as a single molecule in a cell expression system. In one embodiment, where the antigen binding construct is a dual targeting construct which forms a mAbdAb molecule, the heavy chain of the mAb is expressed a single molecule comprising the dAb. For example, where the mAbdAb construct in accordance with the present invention is a monoclonal antibody which binds MLC linked to an anti-c-Kit immunoglobulin single variable domain, the anti-c-Kit immunoglobulin single variable domain is expressed as part of the anti-MLC antibody heavy chain. In another embodiment, the anti-c-Kit immunoglobulin single variable domain is expressed as part of the anti-MLC antibody light chain.

Advantageously, such an expression construct can be produced more efficiently than a molecule in which the two antigen binding components are linked using a chemical linker. This is because, when a chemical linker is used, the final product obtained will comprise a mixed population of molecules representing incomplete chemical linkage reactions. That is where binding component A is mixed with binding component B and linkage agent x is added to ensure chemical cross-linking, the reaction mixture obtained after the linkage reaction will comprise, A, B, x, A-x and B-x as well as the desired compound A-x-B. Accordingly, using this in a manufacturing process will require a purification step to remove all the partially reacted components and obtain just the desired compound A-x-B.

An in vitro expression system for the expression of a dual targeting construct provides a manufacturing system as all the molecules obtained therefrom will be the desired compound. Such a system provides a simplified manufacturing process which provides a more homogeneous population of products and provides a more routine production process which can satisfy safety requirements.

Treatment:

The dose and duration of treatment relates to the relative duration of the molecules of the present invention in the human circulation, and can be adjusted by one of skill in the art depending upon the condition being treated and the general health of the patient. It is envisaged that repeated dosing (e.g. once every 3 days, once a week or once every two weeks) over an extended time period (e.g. four to six months) maybe required to achieve maximal therapeutic efficacy. Ideal dosing would be a single administration within the first week after myocardial infarction (i.e. post-MI).

The mode of administration of the therapeutic agent of the invention may be any suitable route which delivers the agent to the host. The antigen binding constructs, immunoglobulin single variable domains and pharmaceutical compositions of the invention are particularly useful for parenteral administration, i.e., subcutaneously (s.c.), intrathecally, intraperitoneally, intramuscularly (i.m.), intravenously (i.v.), or intranasally or during surgical procedures.

Therapeutic agents of the invention may be prepared as pharmaceutical compositions containing an effective amount of the antigen binding construct or immunoglobulin single variable domains of the invention as an active ingredient in a pharmaceutically acceptable carrier. In one embodiment the prophylactic agent of the invention is an aqueous suspension or solution containing the antigen binding construct in a form ready for administration. In one embodiment the suspension or solution is buffered at physiological pH. The compositions for parenteral administration will comprise a solution of the antigen binding construct of the invention or a cocktail thereof dissolved in a pharmaceutically acceptable carrier. In one embodiment the carrier is an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding construct of the invention in such pharmaceutical formulation can vary widely, i.e., from less than about 0.5%, usually at or at least about 1% to as much as about 15 or about 20% by weight and will be selected primarily based on fluid volumes, viscosities, etc., according to the particular mode of administration selected.

Thus, a pharmaceutical composition of the invention for intramuscular injection could be prepared to contain about 1 mL sterile buffered water, and between about 1 ng to about 100 mg, e.g. about 50 ng to about 30 mg or about 5 mg to about 25 mg, of an antigen binding construct of the invention. Similarly, a pharmaceutical composition of the invention for intravenous infusion could be made up to contain about 250 ml of sterile Ringer's solution, and about 1 to about 30 or about 5 mg to about 25 mg of an antigen binding construct of the invention per ml of Ringer's solution. Actual methods for preparing parenterally administrable compositions are well known or will be apparent to those skilled in the art and are described in more detail in, for example, Remington's Pharmaceutical Science, 15th ed., Mack Publishing Company, Easton, Pa. For the preparation of intravenously administrable antigen binding construct formulations of the invention see Lasmar U and Parkins D “The formulation of Biopharmaceutical products”, Pharma. Sci. Tech. today, page 129-137, Vol. 3 (3 Apr. 2000); Wang, W “Instability, stabilisation and formulation of liquid protein pharmaceuticals”, Int. J. Pharm 185 (1999) 129-188; Stability of Protein Pharmaceuticals Part A and B ed Ahern T. J., Manning M. C., New York, N.Y.: Plenum Press (1992); Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, “Mannitol-sucrose mixtures-versatile formulations for protein lyophilization”, J. Pharm. Sci, 91 (2002) 914-922; and Ha, E Wang W, Wang Y. J. “Peroxide formation in polysorbate 80 and protein stability”, J. Pharm Sci, 91, 2252-2264,(2002) the entire contents of which are incorporated herein by reference and to which the reader is specifically referred.

In one embodiment the therapeutic agent of the invention, when in a pharmaceutical preparation is present in unit dose forms. The appropriate therapeutically effective dose will be determined readily by those of skill in the art. Suitable doses may be calculated for patients according to their weight, for example suitable doses may be in the range of about 0.01 to about 20 mg/kg, for example about 0.1 to about 20 mg/kg, for example about 1 to about 20 mg/kg, for example about 10 to about 20 mg/kg or for example about 1 to about 15 mg/kg, for example about 10 to about 15 mg/kg. To effectively treat conditions of use in the present invention in a human, suitable doses may be within the range of about 0.01 to about 1000 mg, for example about 0.1 to about 1000 mg, for example about 0.1 to about 500 mg, for example about 500 mg, for example about 0.1 to about 100 mg, or about 0.1 to about 80 mg, or about 0.1 to about 60 mg, or about 0.1 to about 40 mg, or for example about 1 to about 100 mg, or about 1 to about 50 mg, of an antigen binding construct of this invention, which may be administered parenterally, for example subcutaneously, intravenously or intramuscularly. Such dose may, if necessary, be repeated at appropriate time intervals selected as appropriate by a physician.

The antigen binding constructs described herein can be lyophilized for storage and reconstituted in a suitable carrier prior to use. This technique has been shown to be effective with conventional immunoglobulins and art-known lyophilization and reconstitution techniques can be employed.

Diseases:

Diseases that can be treated by the pharmaceutical compositions of the invention include diseases in which the heart muscle, skeletal muscle, vascular smooth muscle, umbilical artery smooth muscle cells and in muscle tissue in kidney, colon, fallopian tubes, rectum, seminal vesicle, skin, retinal endothelial cells or urinary bladder epithelium are damaged. In one embodiment, diseases that can be treated include cardiovascular disease; Myocardial infarction, chronic heart failure, ischemic heart disease, chronic ischemic or non-ischemic cardiomyopathy, hypertension, coronary artery disease, diabetic heart disease, hemorrhagic stroke, thrombotic stroke, embolic stroke, limb ischaemia, peripheral vascular disease or another disease in which tissue has become ischaemic. In one embodiment, spinal cord injury may be treated. In another embodiment, muscular diseases or muscle disorders may be treated. Muscular diseases/muscle disorders include sarcopenia, Muscular Dystrophy, Spinal Muscular Atrophy, for example.

In one embodiment, the disease is myocardial infarction, in particular, acute myocardial infarction (AMI). In myocardial infarction, myocytes in the myocardium are damaged by oxygen deprivation through insufficiency in the blood supply.

Successful treatment can be measured by an improvement in cardiac functional parameters, including ejection fraction, fractional shortening, left ventricular end systolic and diastolic volume and regional wall motion or cardiac morphological measurements such as reduction in infarct size. These can be measured by echocardiography or MRI, nuclear imaging. Additionally, success can be measured by a reduction in adverse events, including hospitalization, subsequent MI and death and improvement in quality of life and exercise tolerance.

The invention is further described in the following examples, for the purposes of illustration only.

### EXAMPLES

### Example 1

### Cloning and Expression of Proteins

1. Cloning and Expression of Recombinant Mouse and Human vMLC-1

The genes for ventricular myosin light chain 1 (vMLC1) (UniProt accession numbers P08590 (Human), P09542 (mouse)) were synthesised using PCR to also incorporate a C-terminal GlySer(His)₆ tag.

The following PCR primers were used:

TABLE 1

| | | |
|---|---|---|
| SCT016 | GCGCGGATCCACCGGCATGGCGCCGAAAAAACCG | Mouse |
| | GAACCG | vMLC1 |
| | (SEQ ID NO: 106) | 5′ primer |
| SCT017 | GCGCAAGCTTATTAATGATGATGATGATGATGAG | Mouse |
| | AACCGCTCGCCATAATATGTTTCACGAACGC | vMLC1 |
| | (SEQ ID NO: 107) | 3′ primer |
| SCT018 | GCGCGGATCCACCGGCATGGCACCAAAAAAGCCG | Human |
| | GAACCG | vMLC1 |
| | (SEQ ID NO: 108) | 5′ primer |
| SCT019 | GCGCAAGCTTATCAGCTGCTCATGATGTG | Human |
| | (SEQ ID NO: 109) | vMLC1 |
| | | 3′ primer |
| SCT021 | GCGCAAGCTTATTAATGATGATGATGATGATGAG | Human |
| | AACCGCTGCTCATGATGTG | vMLC1 |
| | (SEQ ID NO: 485) | 3′ primer |

The PCR products were digested with BamHI and HindIII and ligated into the vector pDOM50, a mammalian expression vector which is a pTT5 derivative with an N-terminal V-J2-C mouse IgG secretory leader sequence to facilitate expression/secretion into the cell media. The secretory leader sequence is as follows:

(amino acid)
(SEQ ID NO: 110)
METDTLLLWVLLLWVPGSTG
(nucleotide):
(SEQ ID NO: 111)
ATGGAGACCGACACCCTGCTGCTGTGGGTGCTGCTGCTGTGGGTGCCCG
GATCCACCGGGC.

Expression of the protein was performed as described:

Plasmid DNA was prepared using QIAfilter megaprep (Qiagen). 1 μg DNA/ml was transfected with 293-Fectin into HEK293E cells and grown in serum free media. The protein was expressed in culture for 5 days and purified from culture supernatant using protein A affinity resin and eluted with 100 mM glycine pH2 and neutralised with 1/5 volume 1M Tris pH8.0. The proteins were buffer exchanged into PBS.

The vMLC1-6×HIS proteins were purified on Ni-NTA resin (Qiagen) according to the manufacturer's instructions. Following elution from the column, the protein was buffer exchanged into PBS.

To biotinylate the vMLC1, the protein was reacted with a 3-fold molar excess of NHS-LC-biotin (Pierce) overnight at room temperature in PBS according to manufacturer's instructions. The protein was then dialysed extensively into fresh PBS.

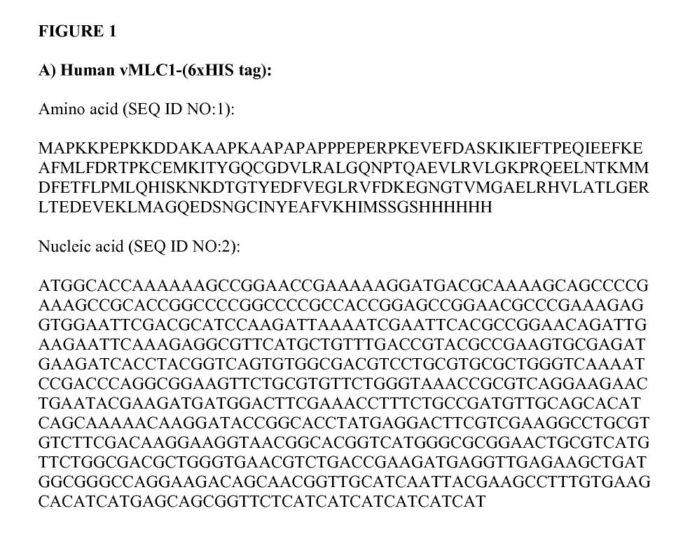

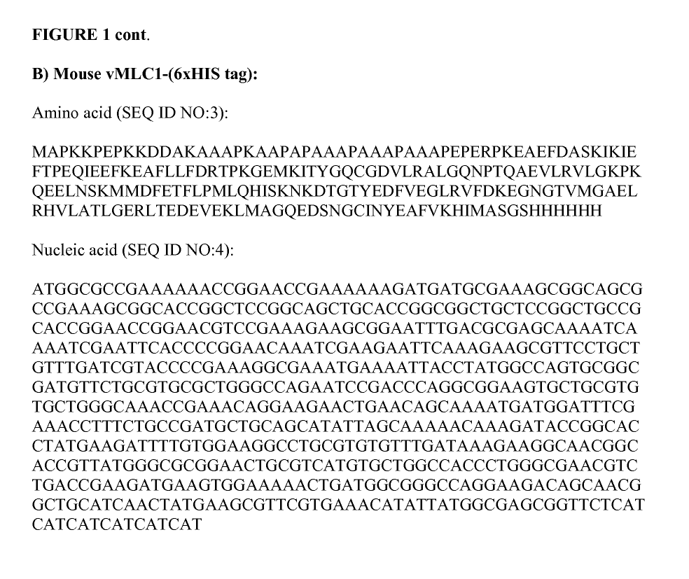

The amino acid and nucleic acid sequences of the resultant HIS-tagged proteins are given in FIG. 1.

### 2. Cloning and Expression of Recombinant Mouse, Human, Cyno, Dog C-Kit

The extra-cellular domains (ECD) of cKIT from human (UniProt accession number: P10721)(hcKIT), mouse (PO5532)(mcKIT), dog (097799) and cynomologus monkey (cloned from cynomologus monkey breast cDNA library (BioChain Institute)) were synthesised by PCR amplification using primers SCT001 and SCT002 (human and cynomologus), SCT014 and SCT028 (mouse) and SCT067 and SCT002 (dog).

TABLE 2

| | | |
|---|---|---|
| SCT001 | GCGCGGATCCACCGGCCAACCATCTGTGAGTCCA | 5′ human/ |
| | GGGG | cyno |
| | (SEQ ID NO: 112) | c-Kit ECD |
| | | primer |
| SCT002 | GCGCGCTAGCAGGAGTGAACAGGGTGTGGGG | 3′ human/ |
| | (SEQ ID NO: 113) | cyno |
| | | c-Kit ECD |
| | | primer |
| SCT014 | GGTACCGGATCCACCGGCCAGCCCAGCGCCAGC | 5′ mouse |
| | (SEQ ID NO: 114) | c-Kit ECD |
| | | primer |
| SCT028 | GCGCGCTAGCGGGGGTGAACAGGGTGTG | 3′ mouse |
| | (SEQ ID NO: 115) | c-Kit ECD |
| | | primer |
| SCT067 | CGCGCCGGATCCACCGGCAGCCAGCCCAGCGTG | 5′ dog |
| | (SEQ ID NO: 116) | c-Kit |
| | | ECD primer |

The PCR products were digested with BamHI and NheI and ligated into pDOM38 (a modified pDOM50 mammalian expression vector which provides a 3′ human IgG1-Fc). DNA was transfected with into HEK293E cells, expressed and purified using protein A affinity resin as described above.

To biotinylate the cKIT-Fc proteins, they were reacted with a 3-fold molar excess of NHS-LC-biotin (Pierce) overnight at room temperature in PBS (according to manufacturer's instructions). The protein was then dialysed extensively into fresh PBS.

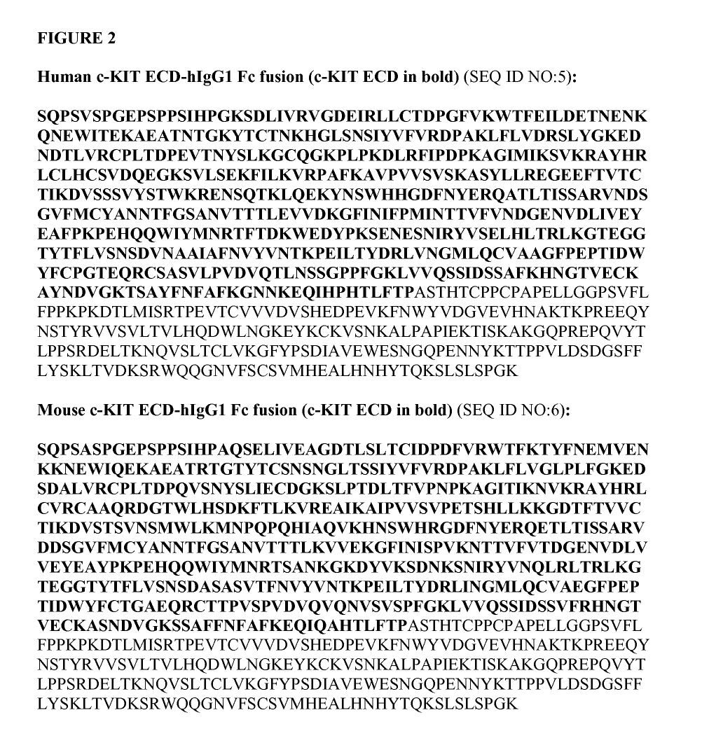

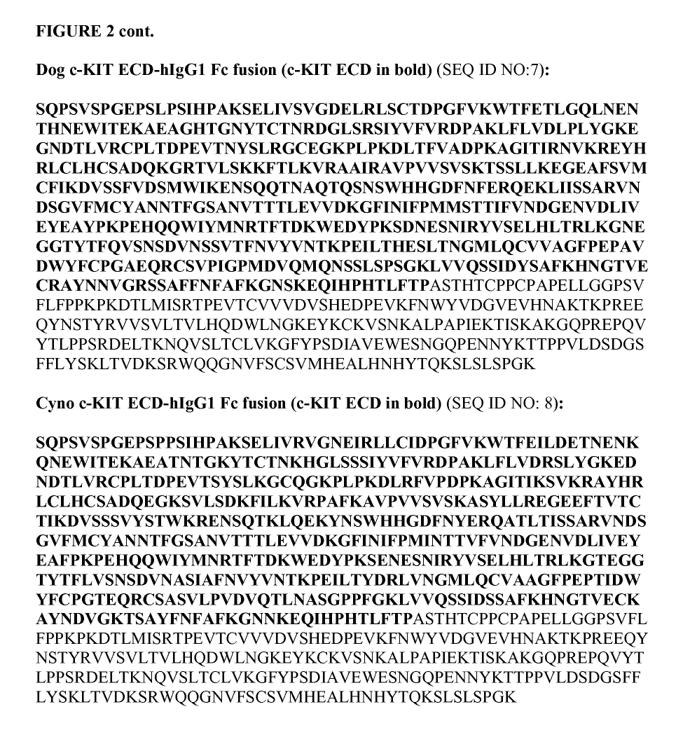

The amino acid sequences of the resultant cKIT ECD-Fc fusion proteins are given in FIG. 2.

His-tagged proteins were generated by expression in pDOM50.

### 3. Cloning and Expression of Recombinant Human and Mouse Stem Cell Factor (SCF).

Human and mouse, soluble Stem Cell Factor (SCF) (Uniprot accession number P21583 (human) (hSCF), P20826 (mouse) (mSCF)) were synthesised to produce full length constructs with BamHI and HindIII cloning sites. The synthesised gene was digested from the holding vector and ligated into the pDOM50 mammalian expression vector. The DNA was transfected into HEK293E cells, expressed and purified as described above for vMLC-1.

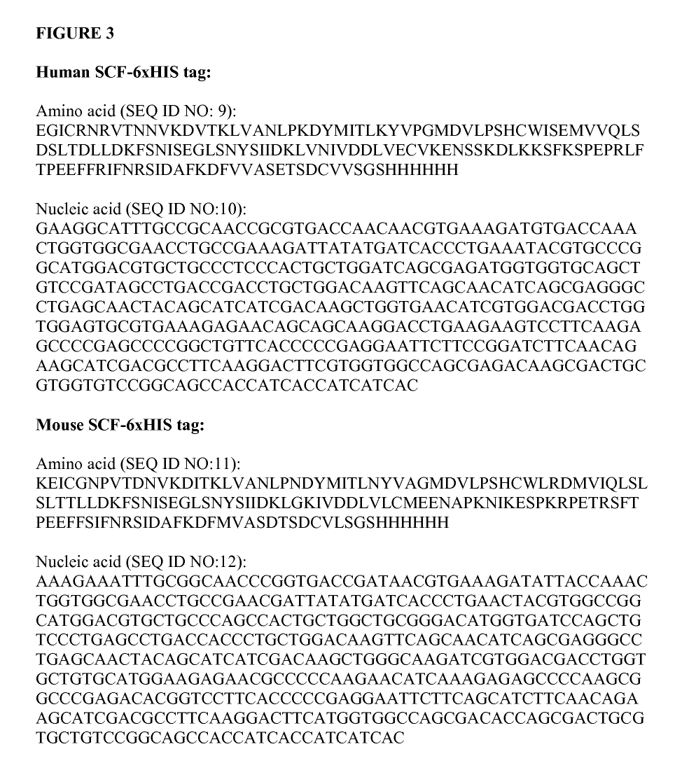

The amino acid and nucleic acid sequences for each of the human and mouse his-tagged proteins are given in FIG. 3.

4. Cloning of rcKIT and rSCF ECD

Synthetic genes of rat cKIT ECD (rcKIT) (Uniprot accession number Q63116) and rat SCF (rSCF) (Uniprot accession number P21581) were synthesised. The SCF construct was designed to incorporate a GlySerHis(₆)-tag at the C-terminus. The genes were ligated into pDOM38 and pDOM50 respectively.

5. Functional Test of Human and Mouse cKIT and SCF Proteins

Human and murine SCF were covalently attached to a CM5 BIAcore chip (GE Healthcare) in the presence of acetate pH 4 for both antigens. cKIT from various species were diluted to 1 μM in HBS-EP buffer (GE Healthcare) and run on the BIAcore in a 2-fold serial dilution to 1 nM. The table below shows the binding of human, mouse and cyno cKIT to human and mouse SCF.

TABLE 3

| Protein | K_{D} hSCF | K_{D} mSCF |
|---|---|---|
| Human cKIT ECD-Fc | 4.64 nM | 17.7 nM |
| Mouse cKIT ECD-Fc | Poor data, cannot fit | Poor data, cannot fit |
| Cyno cKIT ECD_Fc | 9.78 nM | 15.0 nM |
| Human cKIT ECD-H6 | NB | NB |
| Mouse cKIT ECD-H6 | NB | NB |
| Cyno cKIT ECD-H6 | 369 nM | 867 nM |
| α-human SCF mAb | 10.1 nM | 40.7 nM |
| α-mouse SCF mAb | NB | 10.9 nM |

The BIAcore data showed that the 6×HIS (HIS₆) tagged proteins (ECD-H6) do not bind (indicated as “NB”) to SCF on BIAcore, however the Fc versions do. mSCF appeared not to bind mouse cKIT. ‘Poor data, cannot fit’ or ‘bad fit’ refers to inability to fit BIAcore curves according to the standard fitting algorithms for 1:1 Langmuir model or an alternative bivalent model where this is deemed necessary. Anti-human and anti-mouse SCF monoclonal antibodies (mAb) were used as controls (R&D systems).

5.1 RT-PCR of cKIT Positive Cell Lines MC/9

To confirm whether the sequence of mcKIT obtained from P05532 corresponds to mouse cKIT expressed on cells, RT PCR was performed on RNA extracted from MC/9 cells (ATCC #CRL-8306) using standard procedures. Sequencing results of 7 randomly picked clones revealed them all to contain a E207A mutation compared to the sequence given in P05532. Mapping position 207 on the structure of mouse cKIT-SCF complex revealed that this sits in the SCF binding region. Therefore the inability of mcKIT to bind mSCF by BIAcore in the assay described above could be attributed to this.

5.2 mcKIT GNNK Isoforms

Murine cKIT exists in vivo in two isoforms. They differ by an insertion of a “GNNK” motif at the juxtamembrane region in the extracellular domain (Voytyuk et al., 2003, Journal of Biological Chemistry, 278 (11) 9159-9166). To investigate whether this has an effect on mcKIT binding to SCF, mcKIT GNNK was cloned into the mammalian expression vector pDOM38. Briefly, the GNNK⁻ mouse cKIT-hIgG1Fc (pDOM38-mcKIT ECD) was PCR amplified using the primers SCT027 and SCT090 (see table below) to generate a GNNK mouse cKIT construct. Following PCR purification with a Qiagen QIAquick PCR purification kit, the PCR product was ligated into the pCR-2,1-TOPO vector (Invitrogen). Colonies were sequenced with M13 forward and reverse primers. Clone 6F was chosen on the basis of correct sequence with the exception of an Ala-Val mutation which was repaired by mutagenesis with primers SCT093 and SCT094. The insert was the double digested with NheI and BamHI to release the mcKIT GNNK construct and this was ligated into the mammalian expression vector pDOM38.

TABLE 4

| Name | | |
|---|---|---|
| of | | |
| Primer | Sequence | Description |
| SCT027 | GCCCGGATCCACCGGCTCTCA | mcKIT GNNK+ |
| | GCCCAGCGCC | primer (forward) |
| | (SEQ ID NO: 117) | cloning into |
| | | pDOM38 |
| SCT090 | GCGCGCTAGCGGGGGTGAAC | mcKIT GNNK+ |
| | AGGGTGTGGGCCTGGATCTG | primer (reverse) |
| | CTCCTTGTTGTTGCCTTTGAA | cloning into |
| | GGCGAAGTTGAAGAAGGC | pDOM38 |
| | (SEQ ID NO: 118) | |
| SCT093 | GAGCTGATCGTGGAGGCCGG | A29V mutagenesis |
| | CGACACCCTGAGC | primer for mcKIT |
| | (SEQ ID NO: 119) | GNNK (forward) |
| SCT094 | GCTCAGGGTGTCGCCGGCCTC | A29V mutagenesis |
| | CACGATCAGCTC | primer for mcKIT |
| | (SEQ ID NO: 120) | GNNK (Reverse) |

mcKIT GNNK was verified by sequencing and the DNA was prepared via an Endo-free plasmid mega prep kit (Qiagen). 250 ug of DNA was transfected into 250 ml of HEK2936e cells and grown for 5 days at 37° C. The cells were spun, media collected and clarified via filtration and mixed with 1 ml of protein A streamline resin for purification. After an overnight incubation at 4° C., the resin was packed into a column and washed with 30 ml of sterile PBS, 10 ml of 10 mM Sodium Citrate buffer pH 6 and eluted and neutralised with 6.4 ml of 10 mM Sodium Citrate buffer pH 3 and 1 M Sodium Citrate buffer pH 6 respectively.

### 5.3 Site Specific Mutagenesis on Position 207

To investigate whether position 207 is important for mcKIT-mSCF binding, QuickChange™ mutagenesis (Stratagene) was carried out to mutate position 207 from a glutamic acid to an alanine residue. Primers used were E207A reverse mcKIT and E207A for mcKIT (given in the table below).

TABLE 5

| Name | | |
|---|---|---|
| of | | |
| Primer | Sequence | Description |
| E207A | GGCCTTGATGCCGCCCGCSCT | QuickChange ™ |
| reverse | TTCAGG | primer for |
| mcKIT | (SEQ ID NO: 121) | E207A reverse |
| E207A | CCCTGAAAGTGCGGGCGGCC | QuickChange ™ |
| for | ATCAAGGCC | primer for |
| mcKIT | (SEQ ID NO: 122) | E207A forward |

A QuickChange™ PCR was carried out with the above primers (1 ul at 100 uM) and according to manufacturer's instructions, with the addition of 0.5 ul of formamide in a 50 ul reaction. Pfu turbo was the polymerase of choice (Stratagene), and the PCR programme involved a denaturation step for 30 seconds, annealing at 55° C. for 30 seconds and an elongation step at 68° C. for 20 minutes. After 18 cycles products were checked on an agarose gel. Samples were purified with a Qiagen PCR purification kit and parental template was digested with 4 ul of dpnI for 2 hours at 37° C. DNA was ethanol precipitated and eluted in 5 ul of water. The entire 5 ul was transformed into HB2151 cells and clones were verified by sequencing. mcKIT GNNK E207A was also prepared for transfection and expressed and purified in the same way as described above for the mcKIT GNNK isoform.
5.4 Binding of mcKIT GNNK and E207A to mSCF by BIAcore

Human, murine and rat Stem Cell Factor (SCF) were coated onto a CM5 chip at 100 ng/ml in acetate pH 4.5. The chip was made purposefully with a high density (approximately 2000 RUs per SCF) in order to mimic the dimerisation event that occurs when cKIT binds to its ligand SCF.

mcKIT GNNK+ and mcKIT GNNK+E207A were run over the BIAcore at 12 concentrations from 1 uM down to 17 nM to get accurate KD results. Results showed that mcKIT GNNK+ can partially bind mSCF and rSCF, but not hSCF. In addition the level of this binding is very similar to mcKIT GNNK−. mcKIT GNNK+E207A however, can bind only mcKIT and rcKIT but at 26 and 81 nM respectively. Hence this single amino acid modification is instrumental in the correct binding of mcKIT to mSCF/rSCF.

Nonetheless, mcKIT obtained from P05532 is used in the assays described herein as dAbs were selected for their ability to bind mcKIT.

### Example 2

### Anti-MLC Antibody

### 1. Sequencing and Cloning of Recombinant Anti-MLC Antibody (39-15)

The N-termini of the 39-15 mAb (ATCC# HB11709) was determined by Edman sequencing as follows:

Briefly, the kappa chain (Vκ) or heavy chain (V_{H}) was treated with pyroglutamate aminopeptidase (PGAP) from Pyrococcus furiosus (Sigma; Cat# P6236). 20 μL of mAb at 0.25 mg/mL in PBS was used to resuspend 0.01 units of lyophilised PGAP as supplied. The protein suspension was incubated at 75° C. overnight. The treated mAb was then subjected to reducing SDS-PAGE, Western blotting and Edman sequencing.

The N-terminal sequence of the kappa chain (Vκ) was identified as:

(SEQ ID NO: 123)
DIVMSQSPSSLAVSA . . .

The N-terminal sequence of the heavy chain (V_{H}) was identified as:

(SEQ ID NO: 124)
xVQLQQSGAELASPGA . . .

Total cell RNA was extracted from 39-15 hybridoma cells (ATCC HB11709) using the Invitrogen PureLink micro-to-midi kit (Cat#12183-018) according to the manufacturer's instructions.

Variable domains were obtained by RT-PCR of the hybridoma RNA using the Promega AccessQuick RT-PCR system (Cat#9PIA170) with a pool of light chain and heavy chain primers taken from primer sets. DNA multiple sequence alignments of the leader sequences of the κ light chain and the _{H} chain V genes were used to design the primer sets. Primers were manually designed from the alignments to fit the following rules: 1) Minimise the degeneracy as much as possible (less than 100 sequences most desirable, less than 1000 if possible), but at the same time limit the number of degenerate primers required; 2) At least one primer must exist that has: a) No mismatches in the three 3′ bases, and preferentially no mismatches in the 3′ half of the sequence; b) No more than three mismatches across the sequence.

PCR products were purified and ligated into the TOPO-TA cloning vector pCR2.1-TOPO (Invitrogen Cat# K4500-01) according to the manufacturer's instructions.

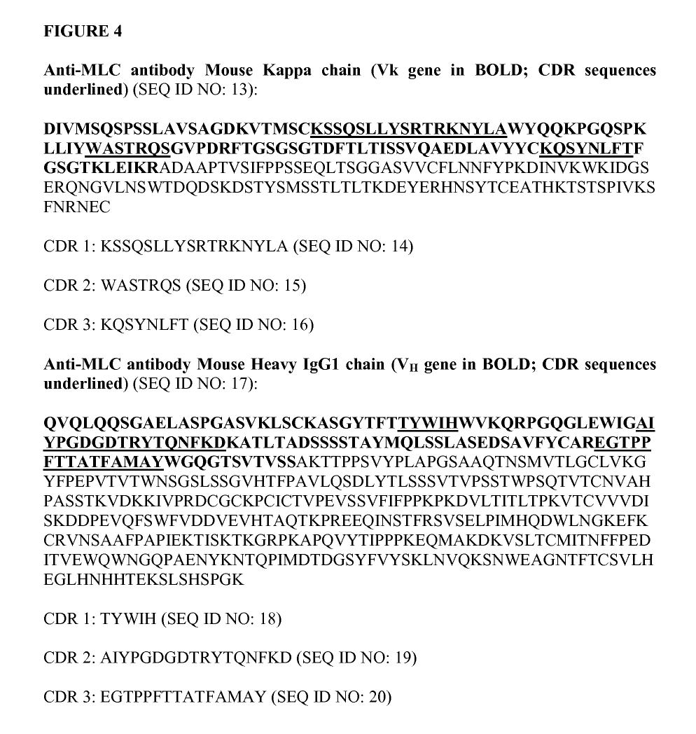

Colonies were sequenced and first residue (x) for the heavy chain was shown to be Gln. The constant regions for mouse kappa and mouse IgG1 heavy chains were sequenced. The sequences are given in FIG. 4.

To generate recombinant anti-MLC mAb (39-15), sequenced V_{H} and Vκ-gene fragments were linked to the mouse IgG1 heavy or light chains respectively by SOE (single overlap extension PCR) according to the method of Horton et al. Gene, 77, p61 (1989)).

Briefly, PCR amplification of the V gene and constant domain sequences were carried out separately using overlapping primers. The primers used are as follows: —

TABLE 6

| 39-15 Vκ SOE | GCGCGGATCCACCGGCGACATTGTGATGTCAC |
|---|---|
| fragment 5′ | (SEQ ID NO: 125) |
| 39-15 Vκ SOE | GGATACAGTTGGTGCAGCATC |
| fragment 3′ | (SEQ ID NO: 126) |
| mouse IgG Cκ SOE | GATGCTGCACCAACTGTATCC |
| fragment 5′ | (SEQ ID NO: 127) |
| mouse IgG Cκ SOE | GCGCAAGCTTACTAACACTCATTCCTGTTG |
| fragment 3′ | (SEQ ID NO: 128) |
| 39-15 V_{H} SOE | GCGCGGATCCACCGGCCAGGTGCAGCTCCAGC |
| fragment 5′ | (SEQ ID NO: 129) |
| 39-15 V_{H} SOE | GGCCAGTGGATAGACAGATGGGGGTGTCG |
| fragment 3′ | (SEQ ID NO: 130) |
| mouse IgG C_{H} | CGACACCCCCATCTGTCTATCCACTGGCC |
| SOE fragment 5′ | (SEQ ID NO: 131) |
| mouse IgG C_{H} | GCGCAAGCTTATCATTTACCAGGAG |
| SOE fragment 3′ | (SEQ ID NO: 132) |

The fragments were purified separately and subsequently assembled in a SOE (single overlap extension PCR extension) reaction using only the flanking primers: 39-15 Vκ SOE fragment 5′, mouse IgG Cκ SOE fragment 3′, 39-15 V_{H} SOE fragment 5′ and mouse IgG C_{H} SOE fragment 3′.

The assembled PCR product was digested using the restriction enzymes BamHI and HindIII and the gene ligated into the corresponding sites in pDOM50.

Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using protein A affinity resin and eluted with 100 mM glycine pH2 and neutralised with 1/5 volume 1M Tris pH8.0. The proteins were buffer exchanged into PBS.

To determine the binding affinity (K_{D}) of the recombinant anti-MLC mAb (31-15) to vMLC-1 compared to the anti-MLC mAb purified from the hybridoma; purified mAbs were analysed by BIAcore on immobilised human vMLC-1 and mouse vMLC-1 (generated as described above) over a concentration range from 833 nM to 7 nM in 2-fold serial dilutions.

TABLE 7

| | | | Dissociation rate | |
|---|---|---|---|---|
| | vMLC | On-rate (1/Ms) | (1/s) | K_{D} (pM) |
| anti-MLC | human | 3.50E+6 | 1.10E−6 | 0.3 |
| (Hybridoma) | | | | |
| recombinant | human | 3.81E+6 | 1.78E−5 | 4.7 |
| anti-MLC | | | | |
| anti-MLC | mouse | 1.31E+06 | 3.07E−05 | 23.5 |
| (Hybridoma) | | | | |
| recombinant | mouse | 1.56E+06 | 3.46E−05 | 22.2 |
| anti-MLC | | | | |

### 2. Generating Mouse/Human Chimera and Affinity Determination.

### Cloning of Anti-MLC (Anti-vMLC-1) Chimera

A mouse/human anti-MLC mAb chimera was made. The mouse anti-MLC V genes were amplified by PCR using the following primers:

TABLE 8

| | |
|---|---|
| mouse anti-MLC | GCGCGGATCCACCGGCCAGGTGCAGCTCCAGC |
| V_{H} gene 5′ | (SEQ ID NO: 133) |
| mouse anti-MLC | CGCGCTAGCTAGCTGAGGAGACGGTGACTGAGG |
| V_{H} gene 3′ | (SEQ ID NO: 134) |
| mouse anti-MLC | TGCCCGGGTCGACCGGCGACATTGTGATG |
| Vκ gene 5′ | (SEQ ID NO: 135) |
| mouse anti-MLC | GCGCTCCGTACGTTTTATTTCCAACTTTGTCCCC |
| Vκ gene 3′ | (SEQ ID NO: 136) |

The PCR products were digested with BamHI/NheI (Vh) and SalI/BsiWI (Vk) and ligated into pDOM40, a mammalian expression vector derived from pDOM50 containing human IgG1 CH1-Ch3 domains or pDOM 39, mammalian expression vector derived from pDOM50 containing human IgG c kappa domains respectively.

Affinity was determined to be similar to the recombinant or hybridoma produced mouse mAb.

### Example 3

### Humanization of Anti-MLC

To humanize the mAb, 2 human Vκ genes (IGKV1-39, IGKV1-4) and 4 human V_{H} genes (IGHV1-3, IGHV1-46, IGHV1-8, IGHV5-51) were identified as having suitable properties to act as human framework scaffolds. The mouse V genes were aligned against the framework sequences of human V genes. The complimentary determining regions (CDRs) of the anti-MLC mAb were identified using the Kabat numbering system and grafted into the human scaffolds. For the J-region minigenes after CDRH3 which are absent in the original human scaffolds, the most similar sequence was chosen by comparing the mouse J-region in Kabat vol. I. For the light chain this is JK2 sequence FGQGTKLEIKR (SEQ ID NO: 137) and for the heavy chain this is JK4 sequence WGQGTLVTVSS (SEQ ID NO: 138).

Humanised variable domains were obtained through PCR assembly using overlapping oligos according to the method described by Stemmer et al. Gene 164(1):49-53, 1995. Restriction sites were installed by PCR of the V-genes (V_{H} BamHI/NheI-Vκ SalI/BsiWI) with the primers:

TABLE 9

| | | |
|---|---|---|
| SCT061 | gcccggatcCACCGGCCAGGTGCAGCTGGTGC | Humanised |
| | AG | clones 1-3, |
| | (SEQ ID NO: 139) | 1-46 and |
| | | 1-8 Vh |
| | | BamHI fwd |
| SCT062 | gcccggatcCACCGGCGAGGTCCAGCTGGTGC | Humanised |
| | AG | clone 5-51 |
| | (SEQ ID NO: 140) | V_{H} BamHI |
| | | fwd |
| SCT063 | gtggtgctagcGCTGCTCACGGTCACCAGGG | Humanised |
| | (SEQ ID NO: 141) | clones V_{H} |
| | | NheI rev |
| SCT064 | gcccgggtcgaccggtGACATCCAGATGACCC | Humanised |
| | AGAGCCC | clone 1-39 |
| | (SEQ ID NO: 142) | V_{H} SalI fwd |
| SCT065 | gcccgggtcgaccggtGACATCGTGATGACCC | Humanised |
| | AGTCTCC | clone 4-1 |
| | (SEQ ID NO: 143) | V_{H} SalI fwd |
| SCT066 | gtggtcgtacgCTTGATCTCCAGCTTGG | Humanised |
| | (SEQ ID NO: 144) | clones Vκ |
| | | BsiWI rev |

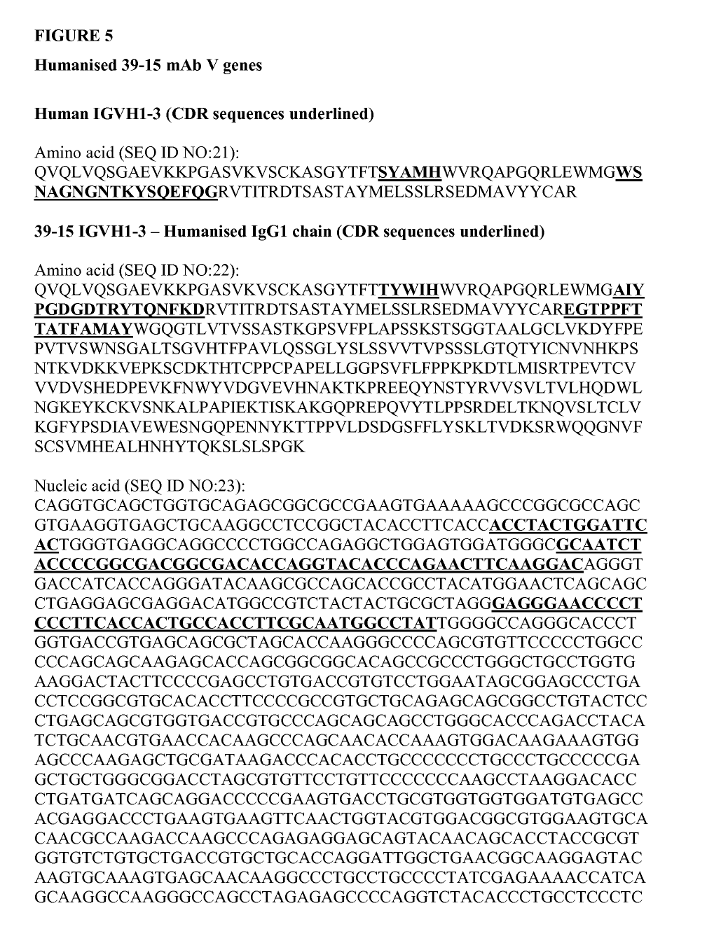

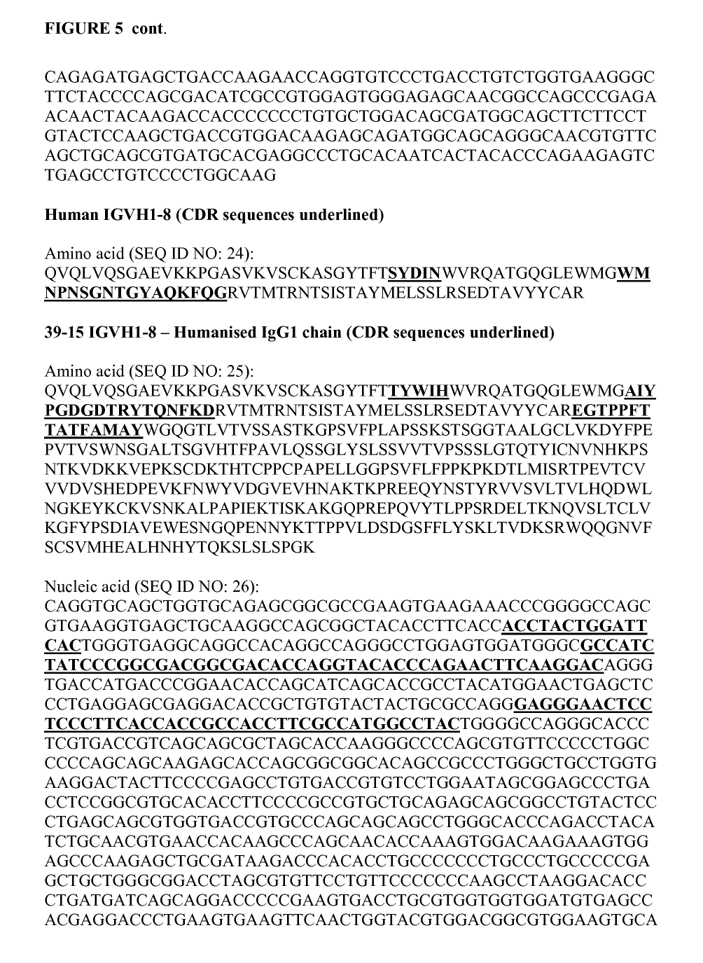

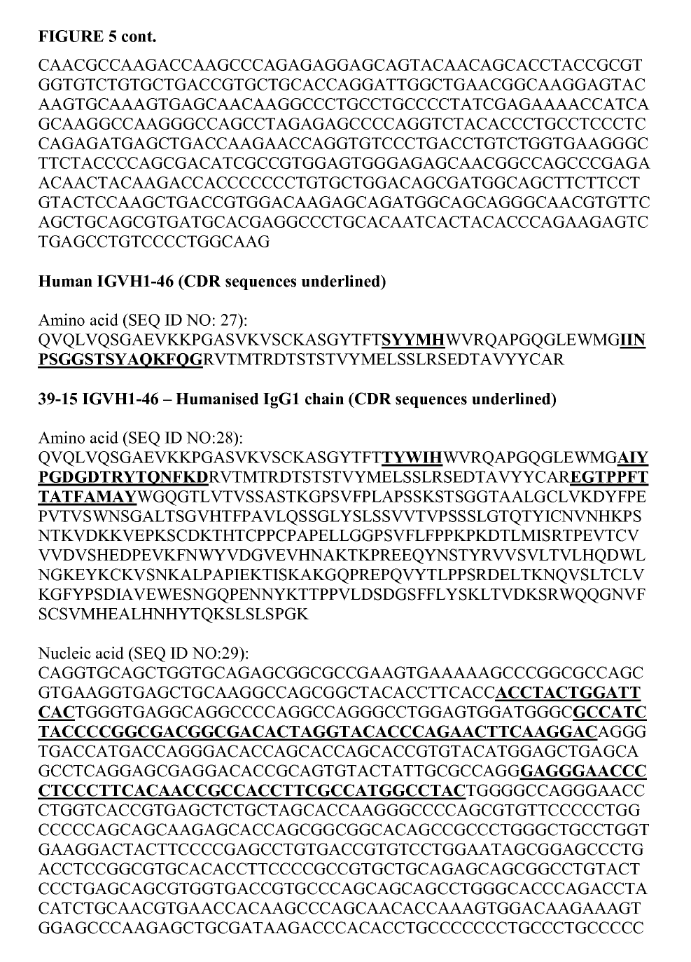

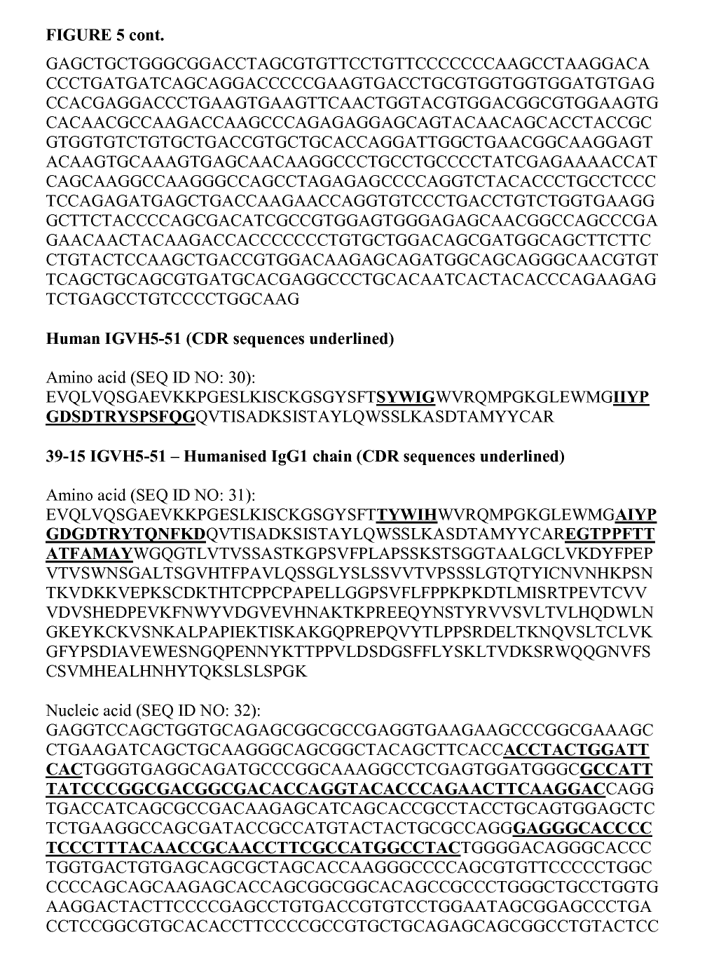

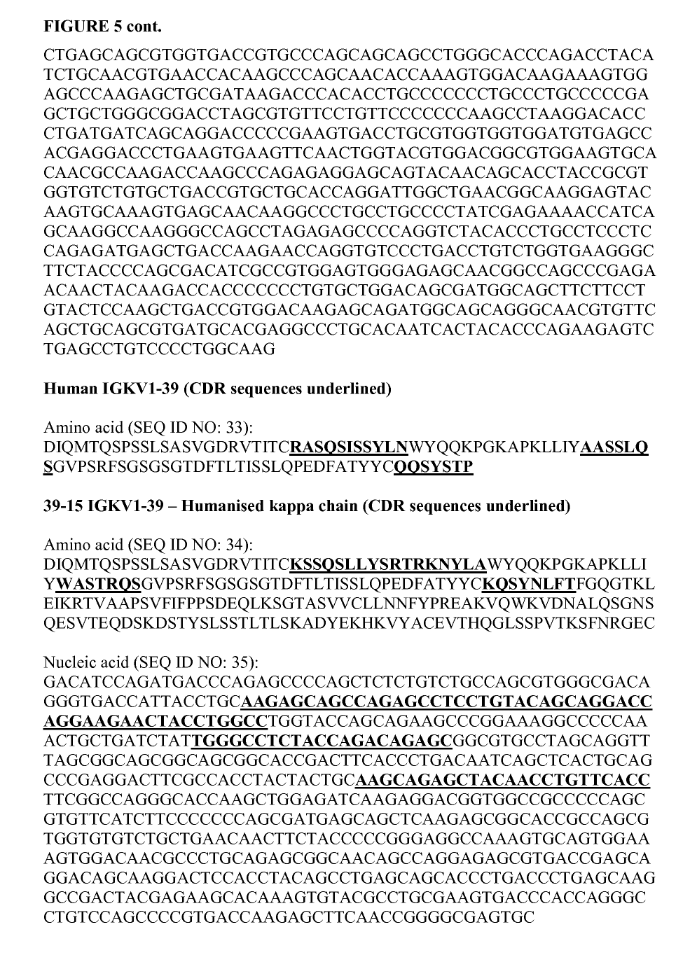

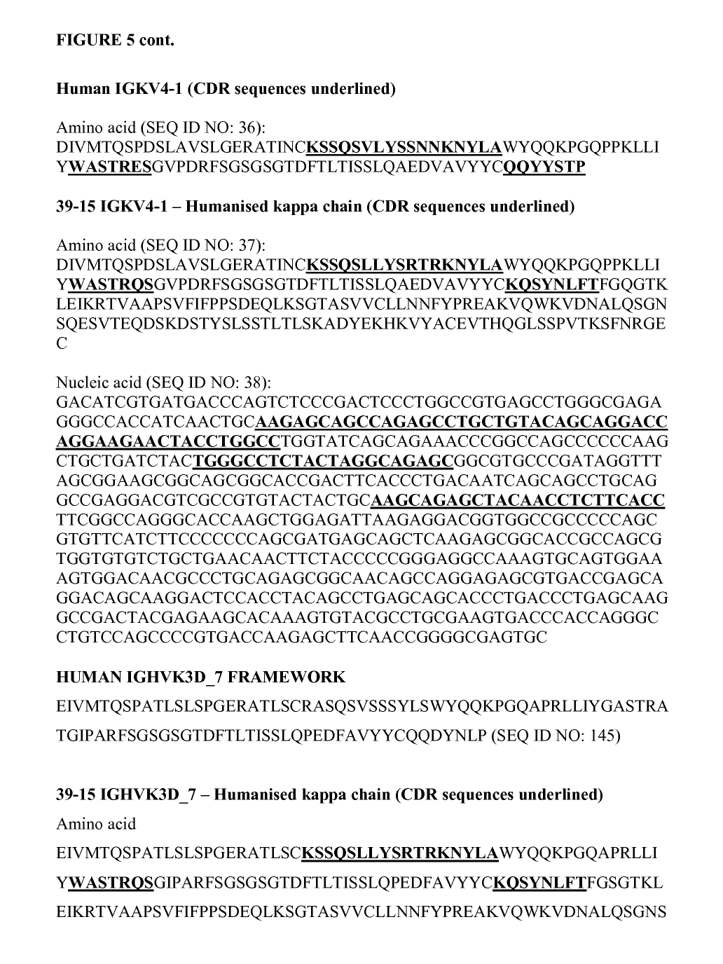

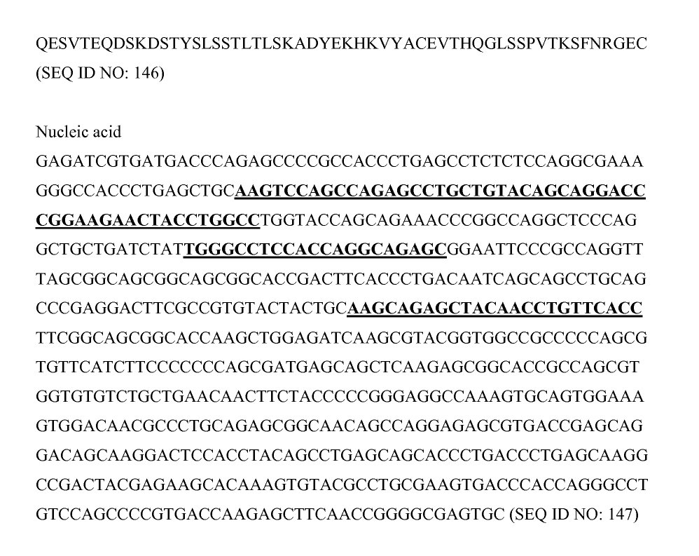

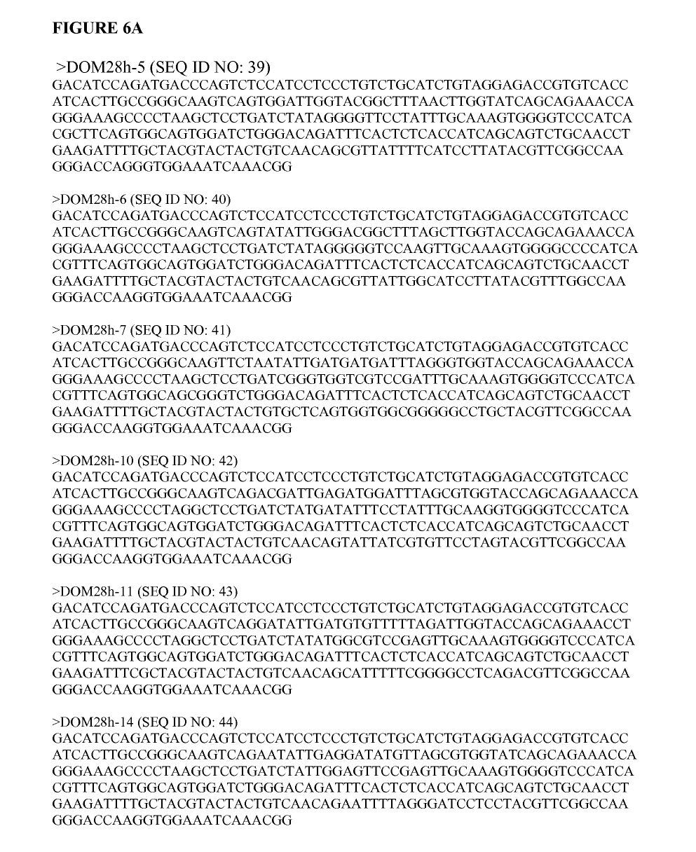

The V genes were then digested and ligated into pDOM39 and pDOM40; mammalian vectors (based on the pDOM50 vector that already contain the constant regions for human kappa chain (Ck) and human IgG1 heavy chain (CH1, CH2 and CH3) respectively) such that each light chain (IGKV1-39; IGKV4-1) was paired with each heavy chain (IGVH1-3, IGVH1-8; IGVH1-46; IGVH5-51) to create eight humanised mAbs. The amino acid and nucleic acid sequences of the heavy and light chains are set out in FIG. 5. The underlined and bold parts show the CDR sequences, CDR1, CDR2 and CDR3 consecutively.

The DNA was transfected into HEK293E cells and expressed and purified as described above.

### Humanised 39-15 mAb V Genes:

Affinity of all 8 humanised mAbs was determined by BIAcore as described above. The results are shown in the following table (ND=binding not determined):

TABLE 10

| | | | | K_{D} |
|---|---|---|---|---|
| Antigen | Protein | ka (1/Ms) | kd (1/s) | (pM) |
| Human | 1-39:1-3 | ND | 2.17e−5 | — |
| | 1-39:1-8 | ND | 3.66E−05 | ND |
| | 1-39:1-46 | ND | 3.41E−05 | ND |
| | 1-39:5-51 | ND | 9.56e−6 | — |
| | 4-1:1-3 | 1.84E+06 | 1.52E−05 | 8.25 |
| | 4-1:1-8 | 1.46E+06 | 1.87E−05 | 12.8 |
| | 4-1:1-46 | 1.99E+06 | 1.63E−05 | 8.18 |
| | 4-1:5-51 | 1.57E+06 | 2.65E−06 | 1.69 |
| Mouse | 1-39:1-3 | ND | 8.8e−5 | — |
| | 1-39:1-8 | ND | 1.36e−4 | — |
| | 1-39:1-46 | ND | 1.22e−4 | — |
| | 1-39:5-51 | ND | 4.02e−5 | — |
| | 4-1:1-3 | 1.67E+06 | 6.60E−05 | 39.6 |
| | 4-1:1-8 | 1.32E+06 | 7.78E−05 | 58.9 |
| | 4-1:1-46 | 1.79E+06 | 7.65E−05 | 42.6 |
| | 4-1:5-51 | 1.42E+06 | 2.16E−05 | 15.3 |

The 1-39 light chain appears to impair binding whereas the 4-1 light chain preserves binding. The 1-39 partnered mAbs were difficult to generate K_{D} values for as the on rates appeared to increase with concentration.

The IGVK4 pairings are better than IGVK1-39 in terms of affinity and properties on BIAcore. The rank order in affinity of the IGVK4-1 pairings are 5-51, 1-3 and 1-46 tied, and finally 1-8. This is consistent over both antigens. Importantly, 4-1:5-51 is as potent as the murine parent mAb.

Light chain from 3D-7 (IGVK3D-7) was also synthesized following the method described for 4-1Vκ. The sequences of the human 3D-7 framework and 39-15 humanized Kappa chain are given in FIG. 5.

### Example 4

### Methods for Selecting dAbs

A) Naïve Selections for Anti-Human C-Kit and Anti-Mouse C-Kit dAbs

Domantis' 4G and 6G naïve phage libraries, phage libraries displaying antibody single variable domains expressed from the GAS1 leader sequence (see WO2005093074) for 4G and additionally with heat/cool preselection for 6G (see WO04101790) were divided into seven pools (identified as 4VH11-13, 4VH14-15, 4VH16-17, 4VH18-19, 4K, 6H, 6K). Library aliquots were of sufficient size to allow 10-fold over representation of each library. Library aliquots were incubated with antigen in 2% Marvel-PBS and incubated for one hour before capture on streptavidin or protein G or anti-Fc Dynabeads (Invitrogen), washed with 0.1% Tween-PBS and PBS and eluted with 1 mg/ml Trypsin. Phage were infected into log phase TG1 cells (Gibson, T. J., (1984) Studies on the Epstein-Barr virus genome. PhD thesis. University of Cambridge) and the infected cells were plated on tetracycline plates (15 μg/ml tetracycline). Cells infected with the phage were then grown up in 2×Ty with tetracycline overnight at 37° C. before the phage were precipitated from the culture supernatant using PEG-NaCl and used for subsequent rounds of selection.

Selection took place against both biotinylated human and mouse c-kit (His tagged) and human and mouse c-kit-Fc fusions as described above. The concentrations of antigen were decreased from 1 μM to 10 nM as the rounds progressed and the titres increased as the rounds progressed. Some selections were carried out in the presence of 5 μM glycated BSA (SIGMA) to reduce the number of carbohydrate binders in the outputs.

### Screening Strategy

After 3 rounds of selection, the dAb genes from each library pool were subcloned from the pDOM4 phage vector into the pDOM10 soluble expression vector.

In each case after selection a pool of phage DNA from appropriate round of selection is prepared using a QIAfilter midiprep kit (Qiagen), the DNA is digested using the restriction enzymes Sal1 and Not1 and the enriched dAb genes are ligated into the corresponding sites in pDOM10 the soluble expression vector which expresses the dAb with a flag tag.

The pDOM10 vector is a pUC119-based vector. Expression of proteins is driven by the LacZ promoter. A GAS1 leader sequence (see WO 2005/093074) ensures secretion of isolated, soluble dAbs into the periplasm and culture supernatant of E. coli. dAbs are cloned SalI/NotI in this vector, which appends a flag tag at the C-terminus of the dAb.

The ligated DNA is used to electro-transform E. coli HB 2151 cells which are then grown overnight on agar plates containing the antibiotic carbenicillin. The resulting colonies are individually assessed for antigen binding.

The antigen binding of individual dAb clones was assessed either by ELISA or on BIAcore. The ELISA assay took the following format. Human or mouse c-kit (His tagged or Fc fusion) or was coated at 1 μg/ml onto a Maxisorp (NUNC) plate overnight at 4° C. The plate was then blocked with 2% Tween-PBS, followed by incubation with dAb supernatant diluted 1:1 with 0.1% Tween-PBS, followed by detection with 1:5000 anti-flag (M2)-HRP (SIGMA) (all steps at room temperature). The binding of the dAb supernatant to a control antigen (glycated BSA or Fc (SIGMA)) was also analysed at the same time. Those dAbs that showed specific binding to c-kit were streaked out and sequenced.

Screening by BIAcore took place using dAb supernatant expressed as above diluted 1:1 with HBS-EP BIAcore running buffer. Each dAb was then injected over a blank flow cell and a flow cell coated with biotinylated human or mouse c-kit on a SA chip. Any dAb that showed specific binding to c-kit was again streaked out and sequenced.

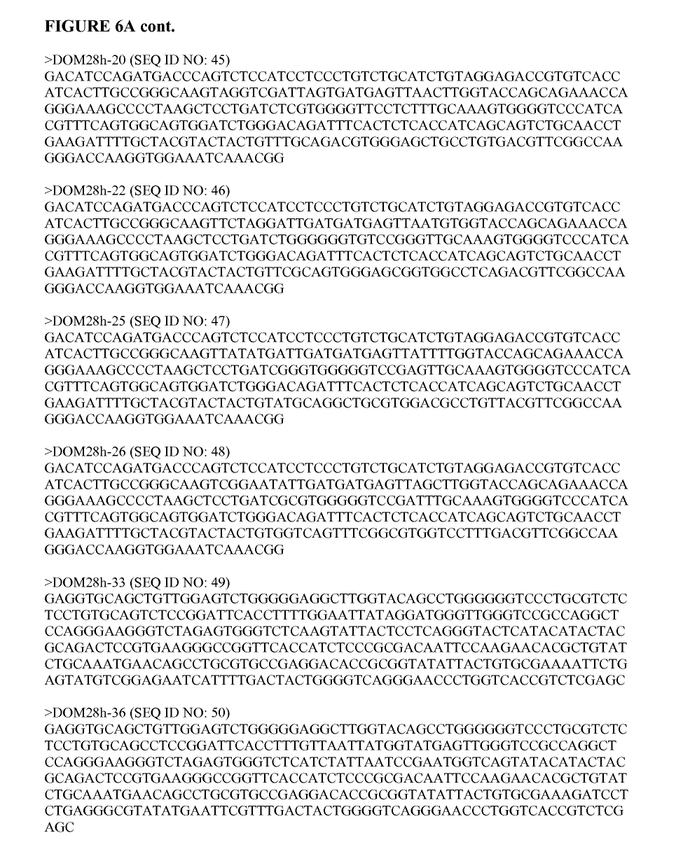

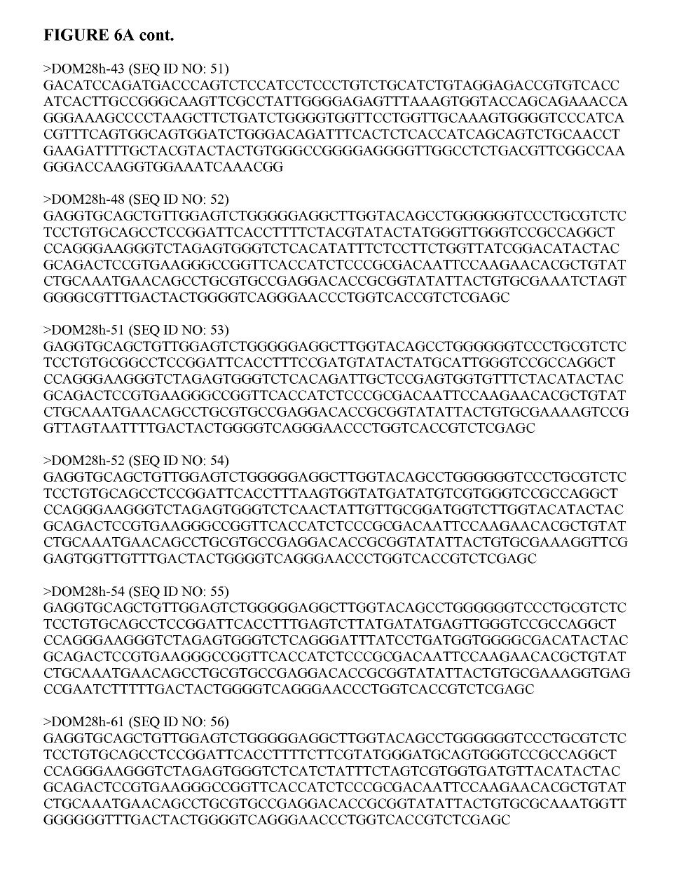

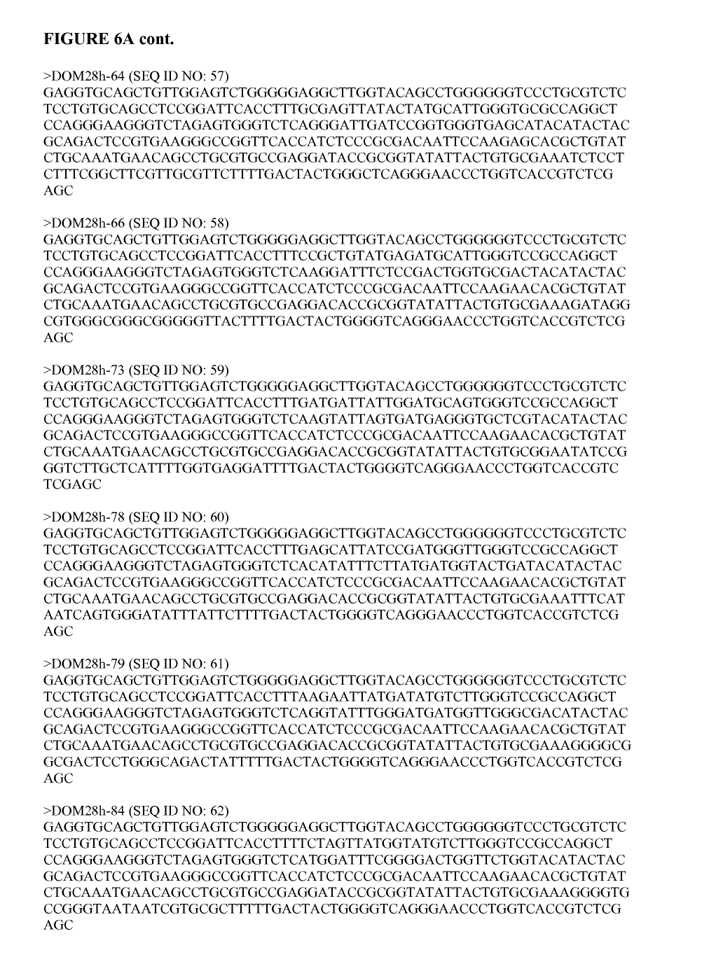

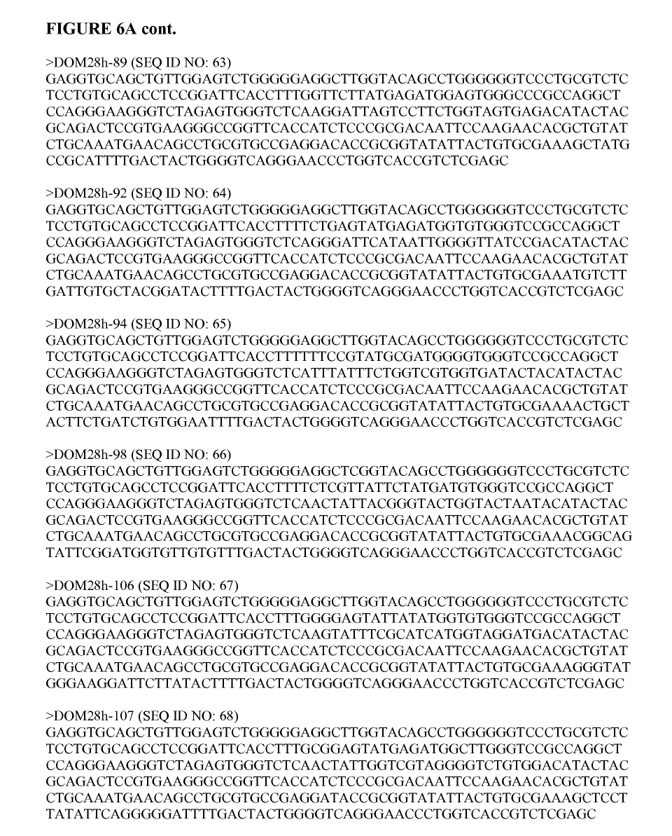

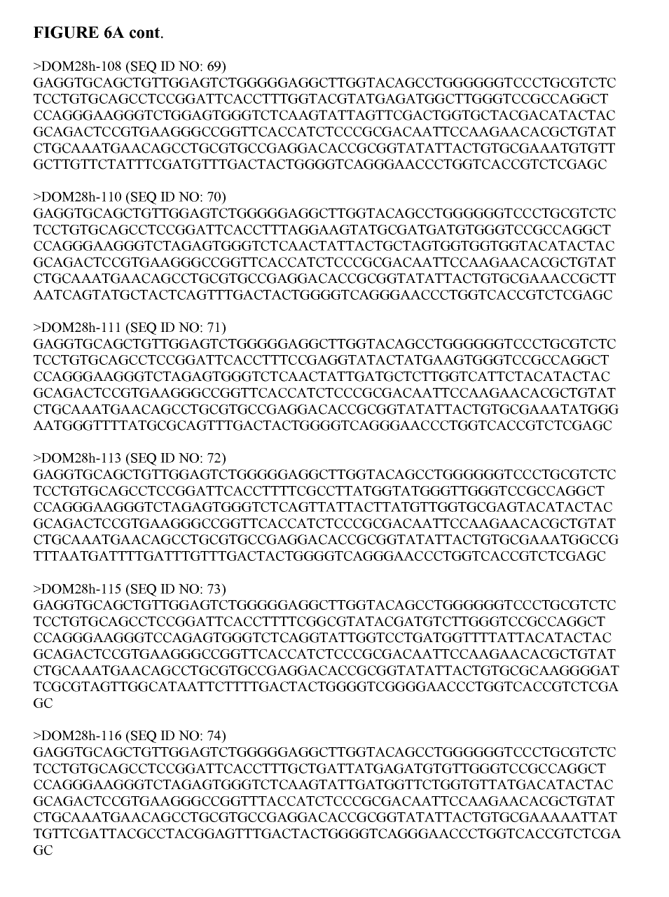

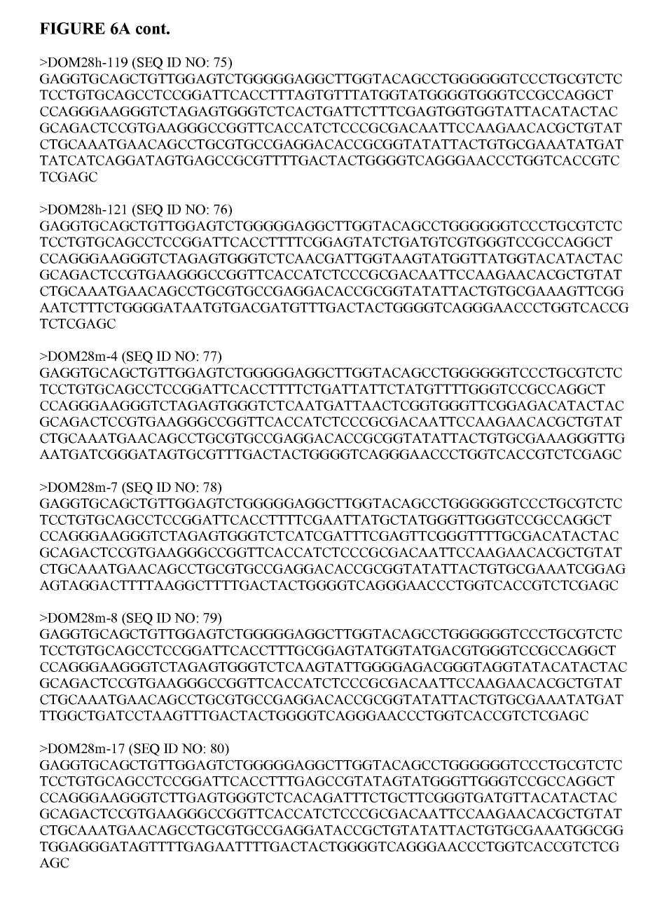

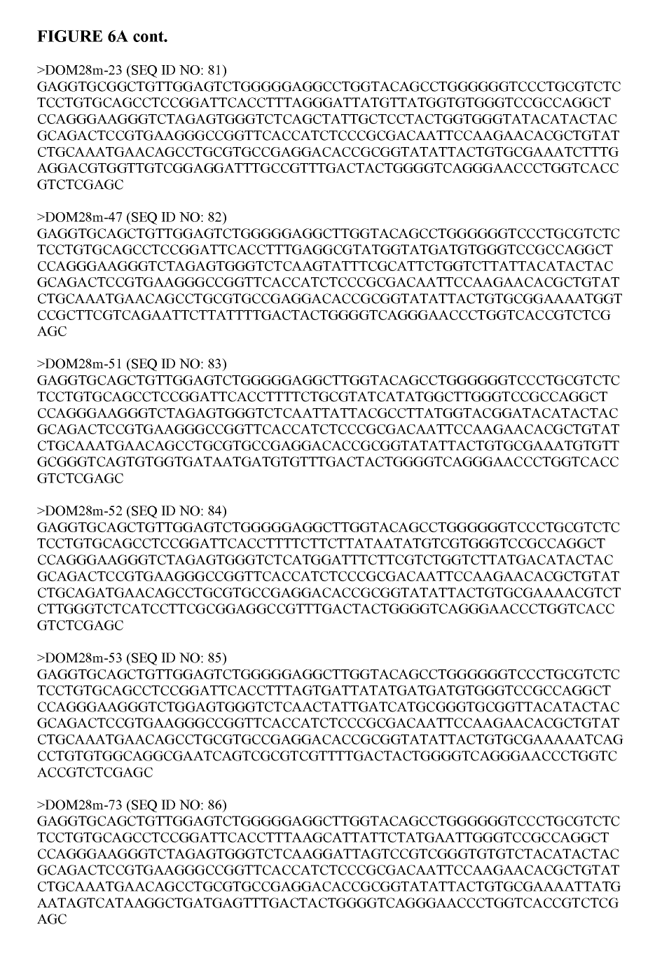

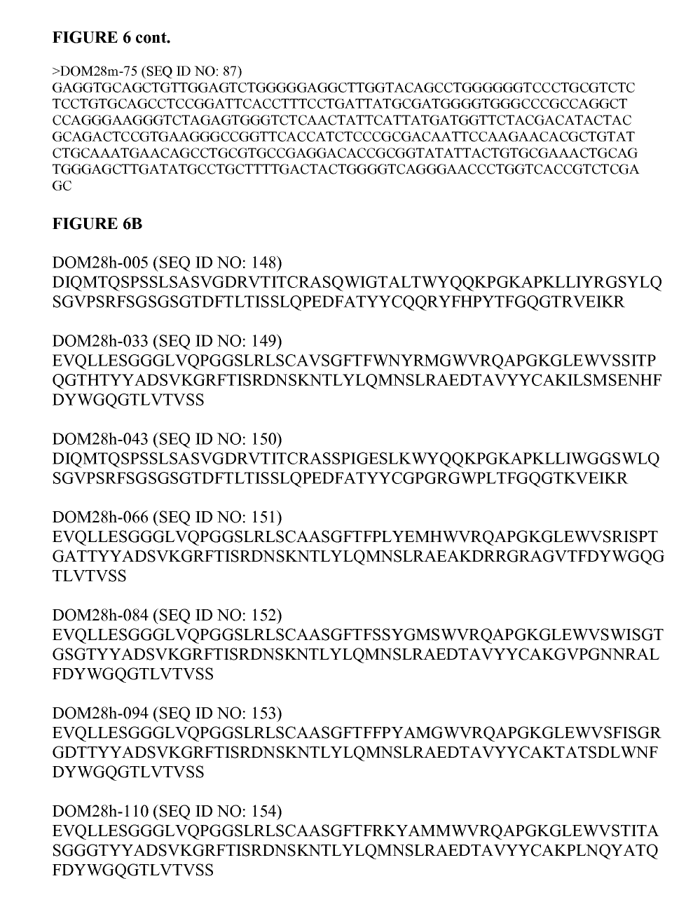

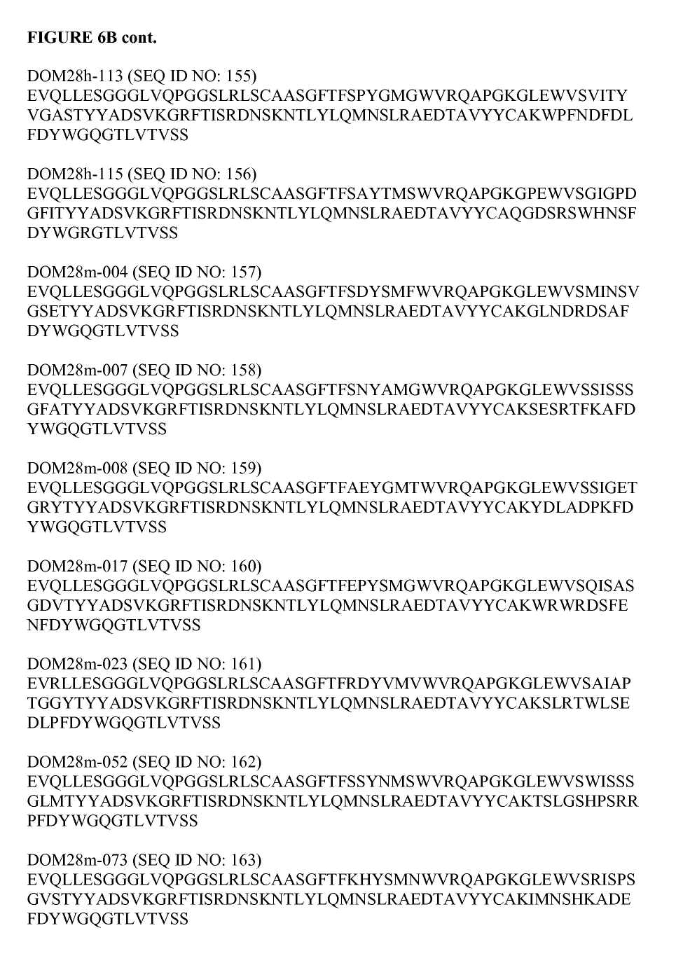

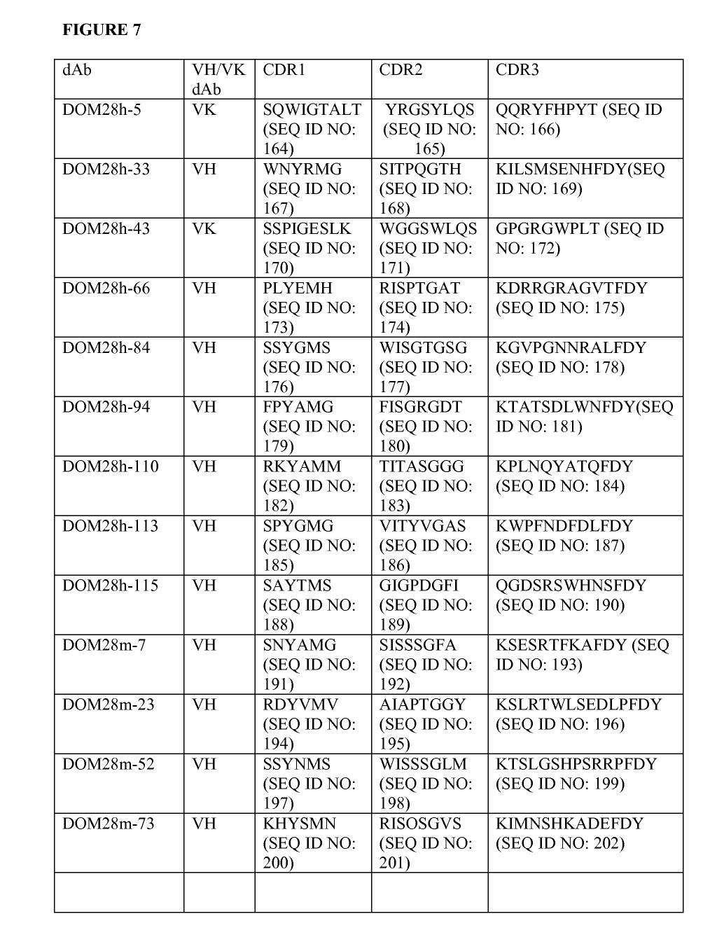

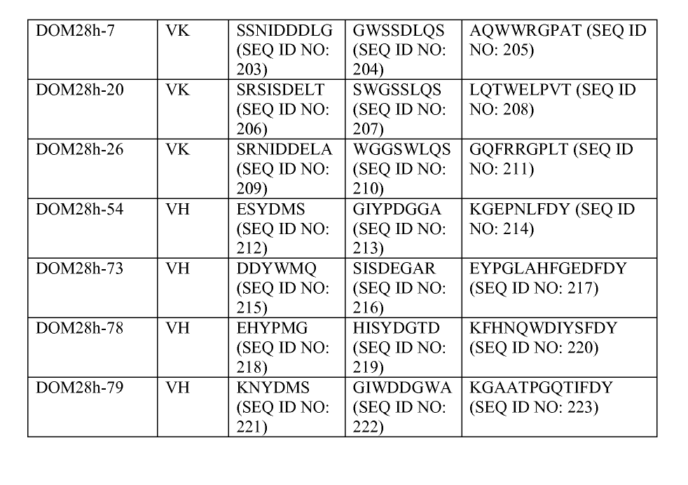

All unique dAb clones were expressed in 50 ml cultures (OnEX plus carbenicillin) overnight at 37° C. and purified on protein A (VH dAbs) or protein L (VK dAbs). Purified dAbs were passed over a SA BIAcore chip coated with either human or mouse c-kit at 1 μM to identify those dAbs that bound specifically to either human or murine c-kit. The nucleic acid sequences of those dAbs are set out in FIG. 6A and SEQ ID NOS: 39 to 87 along with the amino acid sequences in FIG. 6B. The CDR sequences of the corresponding amino acid sequences of a group of clones, as determined by Kabat, are set out in FIG. 7.

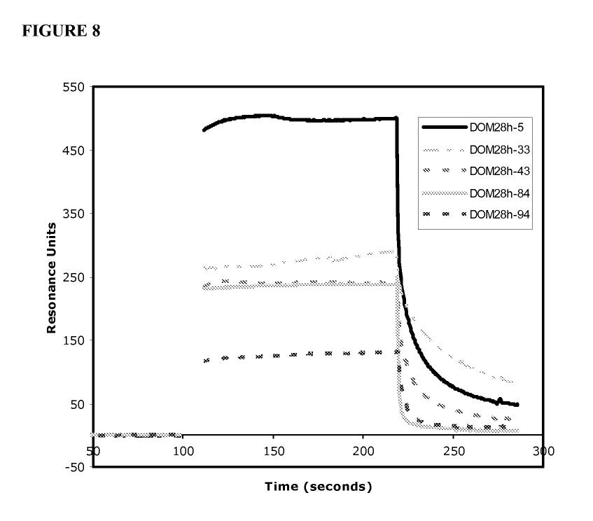

FIG. 8 shows an illustrative BIAcore trace. The approximate affinities of selected dAbs to c-kit from different species are shown in the tables below. Table 11 represents dAbs that bind non-competitively to human c-kit, Table 12 represents dAbs that bind competitively to human c-kit as determined the competitive receptor binding assay described below and Table 13 describes mouse c-kit binding dAbs.

TABLE 11

| dAb | KD (human) | KD (mouse) | KD (cyno) |
|---|---|---|---|
| DOM28h-5 | 533 nM | Not determined | 550 nM |
| DOM28h-33 | 48 nM | Not determined | 98.5 nM |
| DOM28h-43 | 239 nM | Not determined | 345 nM |
| DOM28h-66 | 2.9 uM | Not determined | Not determined |
| DOM28h-84 | 896 nM | Not determined | 10.2 uM |
| DOM28h-94 | 21.8 uM | 1.5 uM | 19.2 uM |
| DOM28h-110 | 3.3 uM | Not determined | Not determined |

TABLE 12

| dAb | KD (human) |
|---|---|
| DOM28h-7 | 1.4 uM |
| DOM28h-20 | 1 uM |
| DOM28h-26 | 621 nM |
| DOM28h-54 | 490 nM |
| DOM28h-73 | 1.3 uM |
| DOM28h-78 | 233 nM |
| DOM28h-79 | 218 nM |

TABLE 13

| dAb | KD (human) | KD (mouse) | KD (cyno) |
|---|---|---|---|
| DOM28m-7 | 7.2 uM | Not determined | 29 uM |
| DOM28m-23 | 2.9 uM | 2.8 uM | 2.72 uM |
| DOM28m-52 | Not determined | Not determined | Not determined |

Those dAbs that bound specifically to human or mouse c-kit were then analysed in the Competitive Receptor Binding Assay.

The Competitive Receptor Binding Assay was carried out as follows. Sphero streptavidin polystyrene beads were coated with 1 μg/ml biotinylated human c-kit. This was carried out by washing 200 μl beads 3× with PBS, incubating the beads with 1 μg/ml biotinylated human c-kit at room temperature with rotation for >1 hr and then washing the beads again 3× with PBS before resuspending the beads in 500 μl PBS. 10 μl 1:10 c-kit coated beads (all dilutions were carried out in 0.1% BSA-PBS) were then mixed with 10 μl 1:100 R&D human stem cell factor (20 μg/ml stock), 1:1000 anti-human stem cell factor IgG (Alexa Fluor 647 labeled) and 10 μl dAb (dilution series starting at 10 μM) in a 384 well clear bottomed FMAT plate and left to incubate for 6 hours before being read on the AB8200 FMAT.

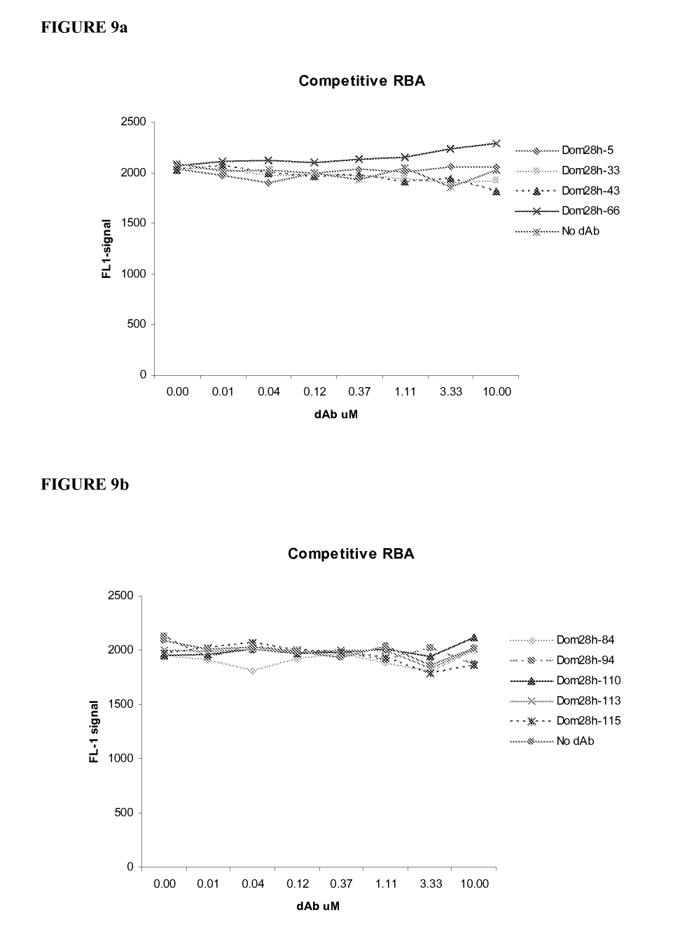

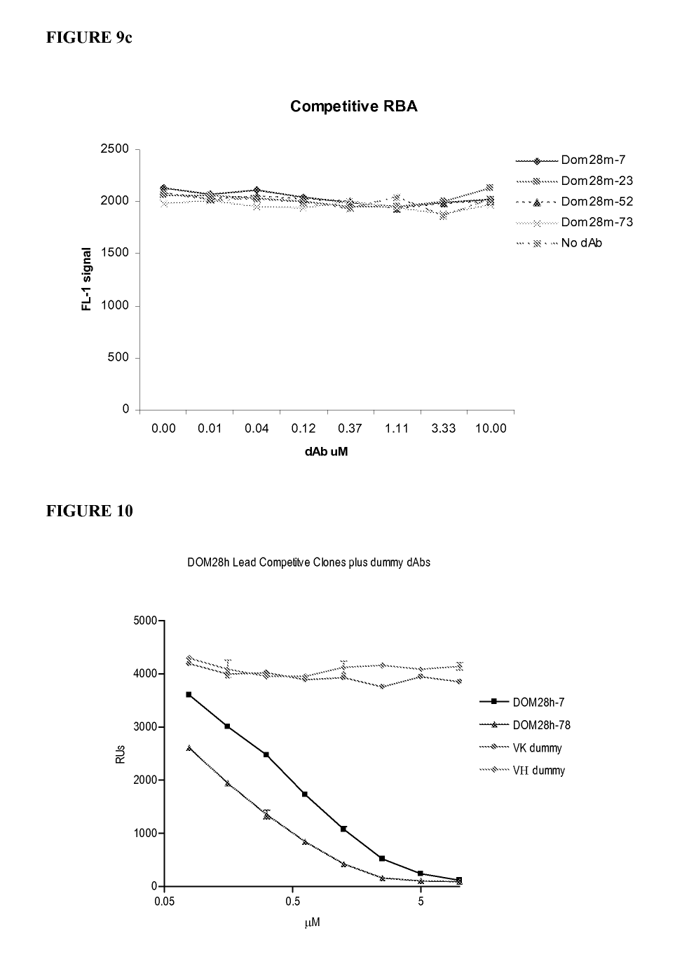

Analysis of the data revealed that certain dAbs are competitive with Stem Cell Factor for binding to c-kit while certain dAbs are not. Data from these assays are shown in FIGS. 9a-c and 10. In these Figures it can be seen that “competitive” dAbs (DOM28h-7 & DOM28h-78) inhibit the binding of human stem cell factor (SCF) to human biotinylated c-kit and therefore as the concentration of dAbs increases the binding signal decreases whereas with “non-competitive” dAbs there is no inhibition of the SCF-c-kit interaction and therefore the binding signal remains constant across all dAb concentrations.

Both dAbs that were competitive and non-competitive with stem cell factor for binding to c-kit were analyzed using flow cytometry to determine whether they bound selectively to cell lines displaying c-kit on their cell surface.

Briefly, cells are harvested, and washed in PBS/5% FCS. Cells are divided between the appropriate number of wells at a concentration of 1×10⁵ cells per well. The cells are incubated with the appropriate concentration of dAb for 30 mins-1 hr at 4° C. The cells are washed with PBS/5% FCS and incubated with 1:500 Secondary antibody (mouse anti-FLAG, Sigma) for 30 mins-1 hr at 4° C. The cells are washed again with PBS/5% FCS buffer and incubated with 1:500 tertiary antibody (Goat anti-mouse FITC sigma) for 30 mins-1 hr at 4° C. The cells are washed with PBS/5% FCS and resuspended in 200 ul PBS/2.5% FCS before analysis by flow cytometry (FACS Canto II, using FACS Diva software).

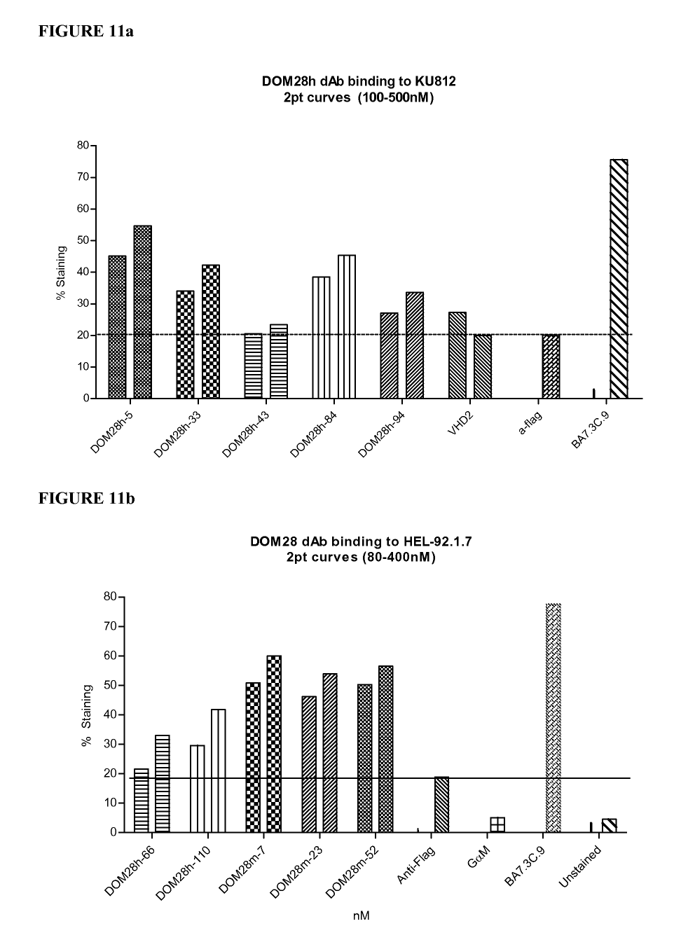

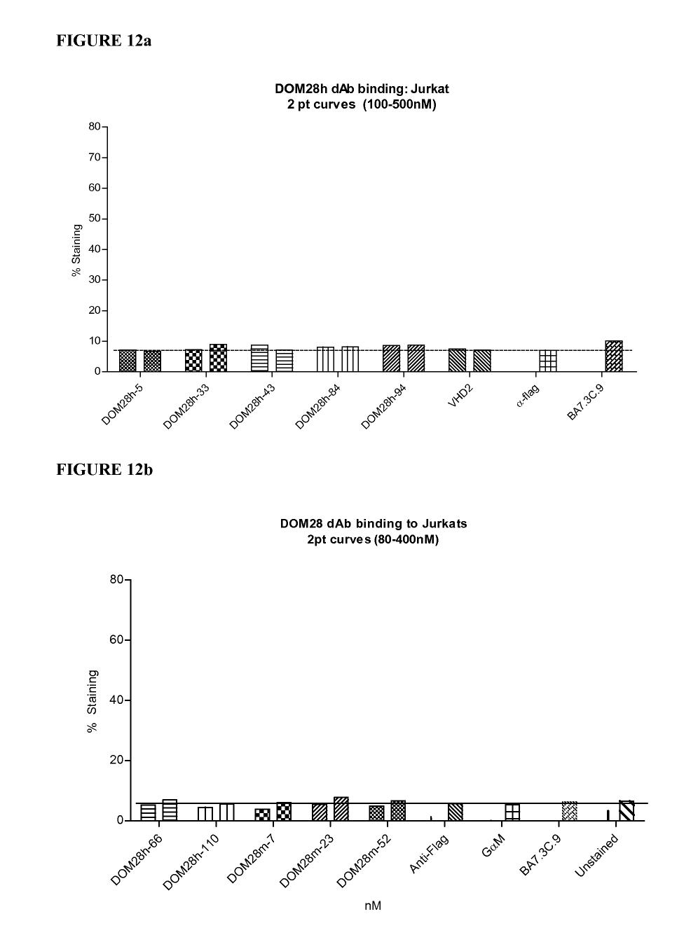

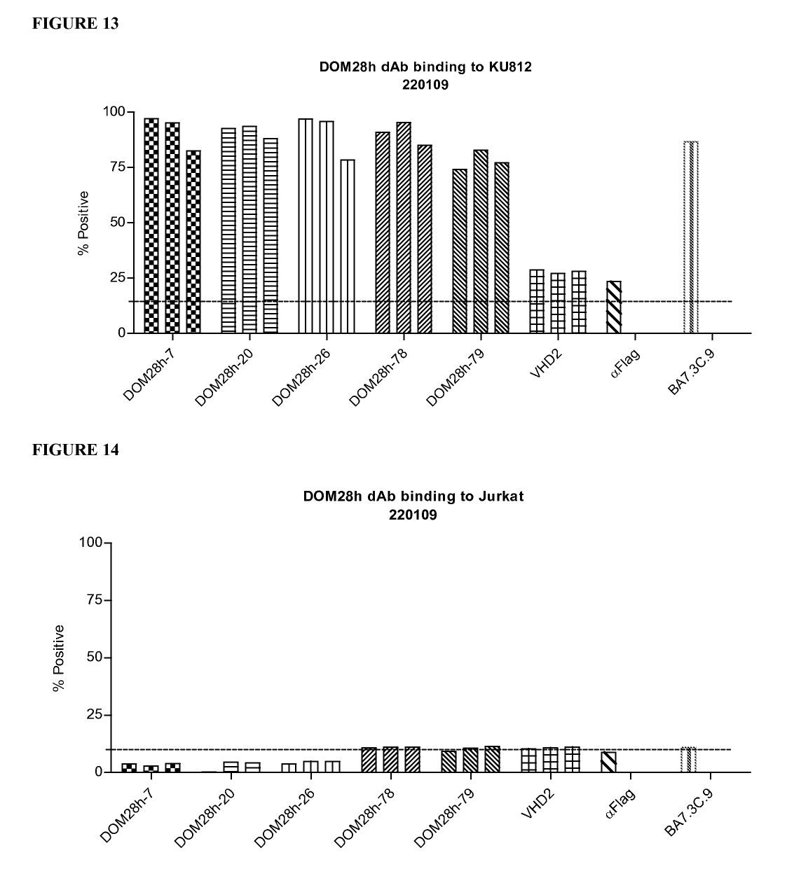

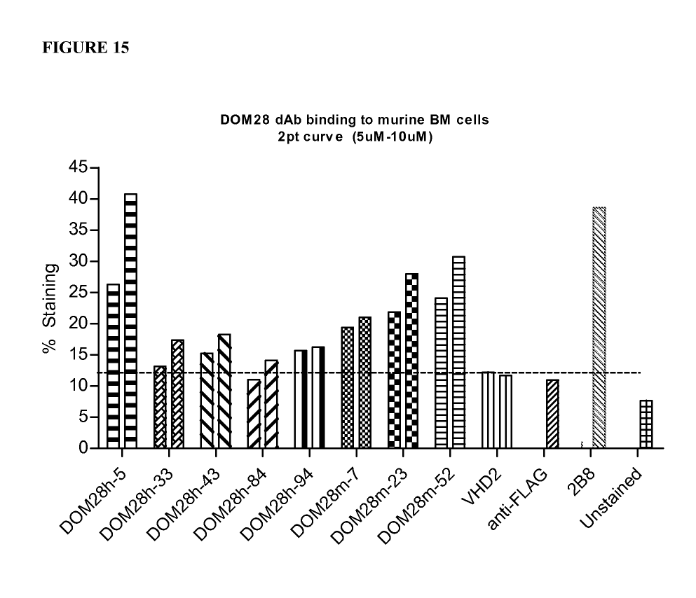

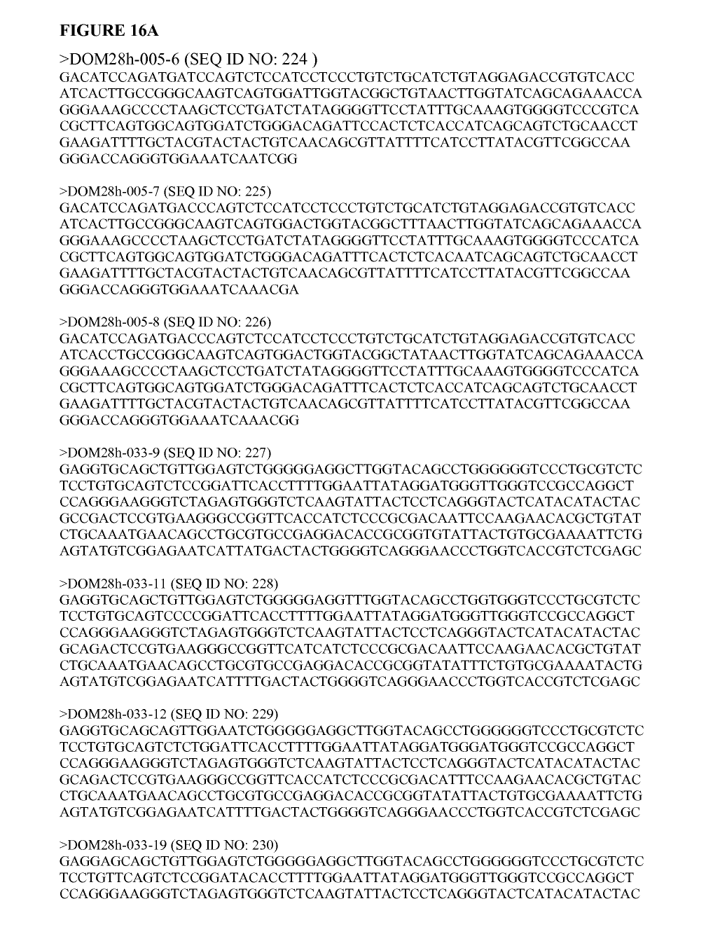

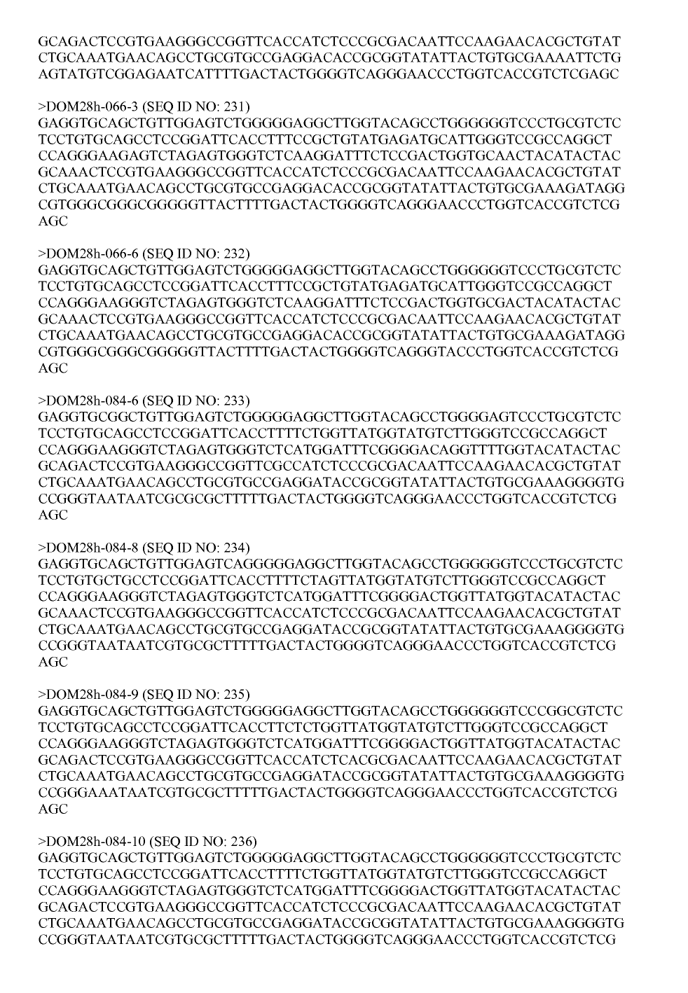

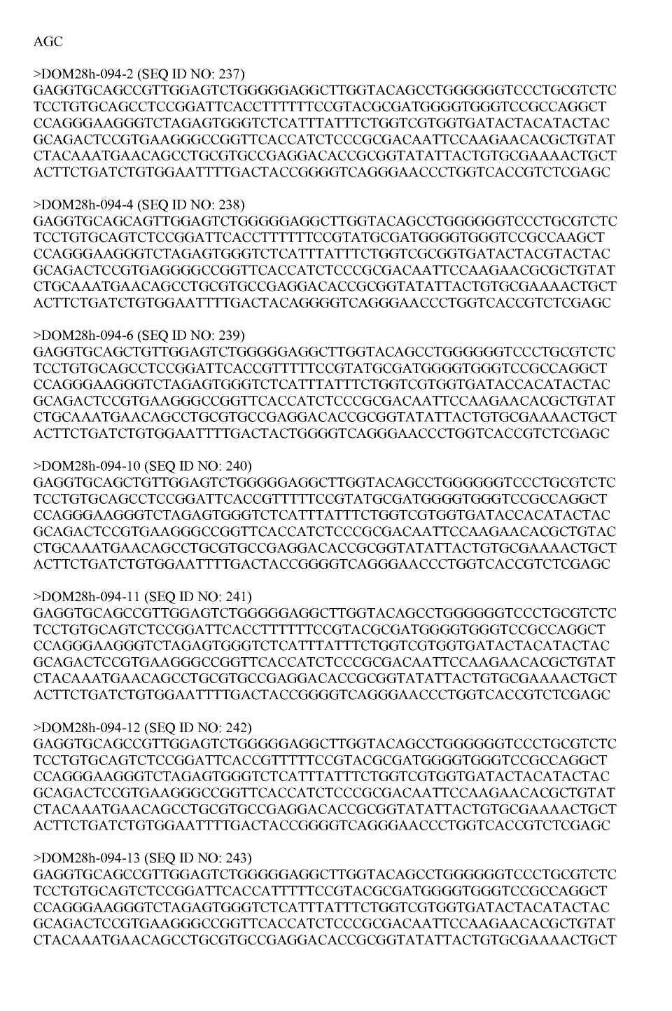

Three cell lines were used as human c-kit positive lines (KU812 (Biocat #117278), KG1 (ATCC #CCL-246, Biocat #122882) and HEL 92.1.7 (Biocat #49486)) and one as a negative control human line (Jurkat (Biocat #114406)). Due to the absence of a c-kit positive mouse cell line, initially, dAbs were tested against c-kit gated, primary mouse bone marrow cells with the mouse cell line L929 (HPA (ECACC) #85011425) as a negative control. Binding of non-competitive dAbs to KU812 cells is shown in FIGS. 11a & 11b and to Jurkat cells in FIGS. 12a & 12b. Binding of competitive dAbs to KU812 cells is shown in FIG. 13 and to Jurkat cells in FIG. 14. Binding was also analysed against c-kit+ve-gated primary murine bone marrow cells and the binding of dAbs to these cells is shown in FIG. 15.

dAbs were analysed by SEC-MALLS to determine whether they were monomeric or formed higher order oligomers in solution. SEC MALLS (size exclusion chromatography with multi-angle-LASER-light-scattering) is a non-invasive technique for the characterizing of macromolecules in solution. Briefly, proteins (at concentration of 1 mg/mL in buffer Dulbecco's PBS) are separated according to their hydrodynamic properties by size exclusion chromatography (column: TSK3000; S200). Following separation, the propensity of the protein to scatter light is measured using a multi-angle-LASER-light-scattering (MALLS) detector. The intensity of the scattered light while protein passes through the detector is measured as a function of angle. This measurement taken together with the protein concentration determined using the refractive index (RI) detector allows calculation of the molar mass using appropriate equations (integral part of the analysis software Astra v.5.3.4.12).). Table 14 describes to SEC-MALLS analysis of human and mouse c-kit dAbs. Further SEC MALLs analysis of the mouse c-kit dAbs is shown in Table 22.

TABLE 14
SEC-MALLS of dAbs

| | Mean Molar | | |
|---|---|---|---|
| | mass over | | |
| Name | main peak | | In-solution state |
| DOM28h-5 | 13.9 | kDa | monomer (97%), |
| | 50 | kDa | tetramer (<3%) |
| DOM28h-33 | 15.4 | kDa | monomer (98%) |
| | 37.7 | | dimer (2%) |
| DOM28h-43 | 13.3 | kDa | monomer (99%) |
| | 42 | kDa | dimer/tetramer? (1%) |
| DOM28h-66 | 15 | kDa | monomer (40%), |
| | 35 | kDa | dimer (55%), |
| | 69 | kDa | tetramer (3.5%) |
| DOM28h-84 | 20 | kDa | M/D equilibrium (95%) |
| | 52 | kDa | dimer/tetramer(5%) |
| | 66.5 | kDa | oligomer higher order multimer (15%) |
| DOM28h-94 | 18.6 | kDa | monomer (70%) |
| | 92 | kDa | dimer (10%) |
| | 8300 | kDa | tri/tetramer (20%) |
| DOM28h-110 | 17.6 | kDa | monomer (85%), |
| | 48 | kDa | dimer (15%) |
| DOM28m-7 | 15.8 | kDa | monomer (75%), |
| | 62 | kDa | tetramer (20%), |
| | 93 | kDa | hexamer (5%) |
| DOM28m-23 | 16 | kDa | monomer (80%), |
| | 32 | kDa | dimer (15%), |
| | 125 | kDa | hexamer (5%) |
| DOM28m-52 | 17 | kDa | monomer (80%), |
| | 23 | kDa | dimer (20%) |
| DOM28h-7 | 21.6 | kDa | monomer-dimer equilibrium (100%) |
| DOM28h-20 | 21 | kDa | monomer-dimer equilibrium (95%) |
| | 37 | kDa | dimer-tetramer eq (5%) |
| DOM28h-78 | 16 | kDa | monomer (75%), |
| | 39 | kDa | dimer (9%), |
| | 56 | kDa | tetramer (16%), |
| | 100 | kDa | octamer (<1%) |

dAbs were also analysed by Differential Scanning calorimetry (DSC) to determine the apparent Tm. Briefly, the protein is heated at a constant rate of 18° C./hr (at 1 mg/mL in PBS) and a detectable heat change associated with thermal denaturation measured. The transition midpoint (ₐₚₚTₘ) is determined, which is described as the temperature where 50% of the protein is in its native conformation and the other 50% is denatured. Here, DSC determined the apparent transition midpoint (appTm) as most of the proteins examined do not fully refold. The higher the Tm, the more stable the molecule. The software package used was Origin® v7.0383.

TABLE 15
DSC data

| Name | app Tm 1, ° C. | app Tm 2, ° C. |
|---|---|---|
| DOM28h-5 | 71.1 | 73.7 |
| DOM28h-33 | 56 | 61.6 |
| DOM28h-43 | ~48 | ~55 |
| DOM28h-84 | 64.2 | 66.4 |
| DOM28h-94 | 68.7 | 74.4 |
| DOM28h-110 | 63.9 | 66.8 |
| DOM28m-7 | 67.5 | 69.8 |
| DOM28h-7 | 56.7 | 59.1 |
| DOM28h-20 | 53.5 | 54.5 |

B) Affinity Maturation of Anti-Human C-Kit dAbs

Affinity Maturation Libraries:

To identify dAbs with higher affinity for human and mouse cKIT, error-prone libraries were created using DOM28h-5, DOM28h-33, DOM28h-43, DOM28h-66, DOM28h-84, DOM28h-94 and DOM28h-110 parental dAbs (see FIG. 6 for the sequences of these dAbs). The error prone libraries were generated in the pDOM4 vector. Vector pDOM4, is a derivative of the Fd phage vector in which the gene III signal peptide sequence is replaced with the yeast glycolipid anchored surface protein (GAS) signal peptide. It also contains a c-myc tag between the leader sequence and gene III, which puts the gene III back in frame. This leader sequence functions well both in phage display vectors but also in other prokaryotic expression vectors and can be universally used.

For error-prone maturation libraries, plasmid DNA encoding the dAb to be matured was amplified by PCR, using the GENEMORPH® II RANDOM MUTAGENESIS KIT (random, unique mutagenesis kit, Stratagene). The product was digested with Sal I and Not I and used in a ligation reaction with cut phage vector pDOM4. The ligation produced was then used to transform E. coli strain TB1 by electroporation and the transformed cells plated on 2×TY agar containing 15 μg/ml tetracycline, yielding library sizes of >2×10⁸ clones.

The seven error-prone libraries had mutation rates of between approximately 2 and 5 amino acids per dAb and an average size of 3.9×10⁸.

Selections:

Selection took place against biotinylated human cKIT (His tagged) in solution as described above. The concentration of antigen was decreased from 100 nM to 1 nM as the rounds progressed and the phage titres increased as the rounds progressed.

Screening Strategy and Affinity Determination:

In each case after round 2 or 3 of selection a pool of phage DNA from that round is prepared using a QIAfilter midiprep kit (Qiagen), the DNA is digested using the restriction enzymes Sal1 and Not1 and the enriched v genes are ligated into the corresponding sites in pDOM10, the soluble expression vector which expresses the dAb with a flag tag. The ligated DNA is used to electro-transform E. coli HB 2151 cells which are then grown overnight on agar plates containing the antibiotic carbenicillin. The resulting colonies are individually assessed for antigen binding. In each case at least 96 clones were tested for binding to biotinylated human cKIT and mouse cKIT by BIAcore™ (surface plasmon resonance). Soluble dAb fragments were produced in bacterial culture in ONEX culture media (Novagen) overnight at 37° C. in 96 well plates. The culture supernatant containing soluble dAb was centrifuged and analysed by BIAcore for binding to high density human cKIT and mouse cKIT SA chips and each compared to the appropriate parental dAb from the library was created. Clones were found that showed improvements in affinity to human cKIT (and mouse cKIT) by off-rate screening. All clones that showed an improvement in off-rate were sequenced revealing unique dAb sequences.

Unique dAbs were expressed as bacterial supernatants in 2.5 L shake flasks in Onex media at 37° C. for 24 hrs at 250 rpm. dAbs were purified from the culture media by absorption to protein A or L agarose followed by elution with 10 mM glycine pH2.0. The binding affinity (K_{D}) to human cKIT and mouse cKIT by BIAcore was determined by passing purified dAbs over the BIAcore at 1000 and 500 nM. K_{D} values for binding to human and mouse c-Kit are shown in Table 16 and Table 17 respectively. All DOM28h-94 derivatives have improved affinity to both human and mouse cKIT where as all the other derivatives (DOM28h-5, DOM28h-33, DOM28h-66, DOM28h-84 and DOM28h-110) have improved affinity only to human cKIT.

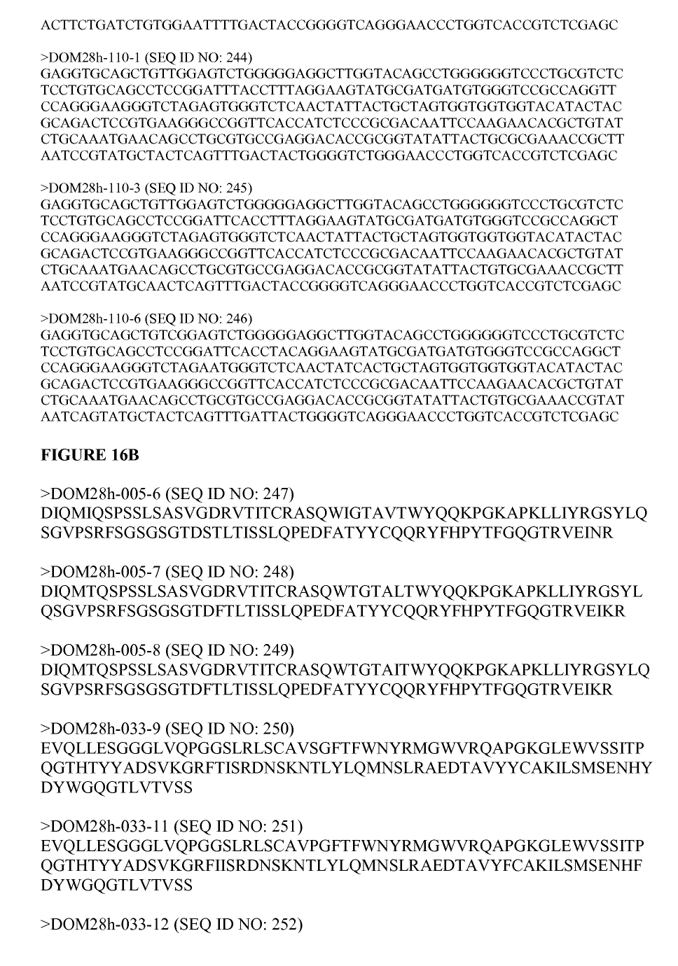

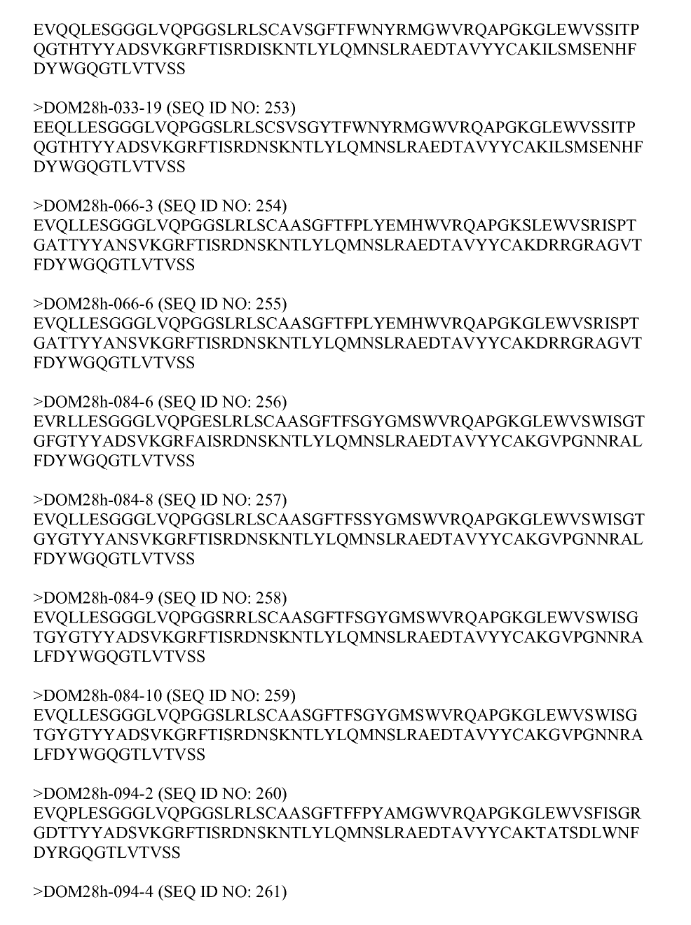

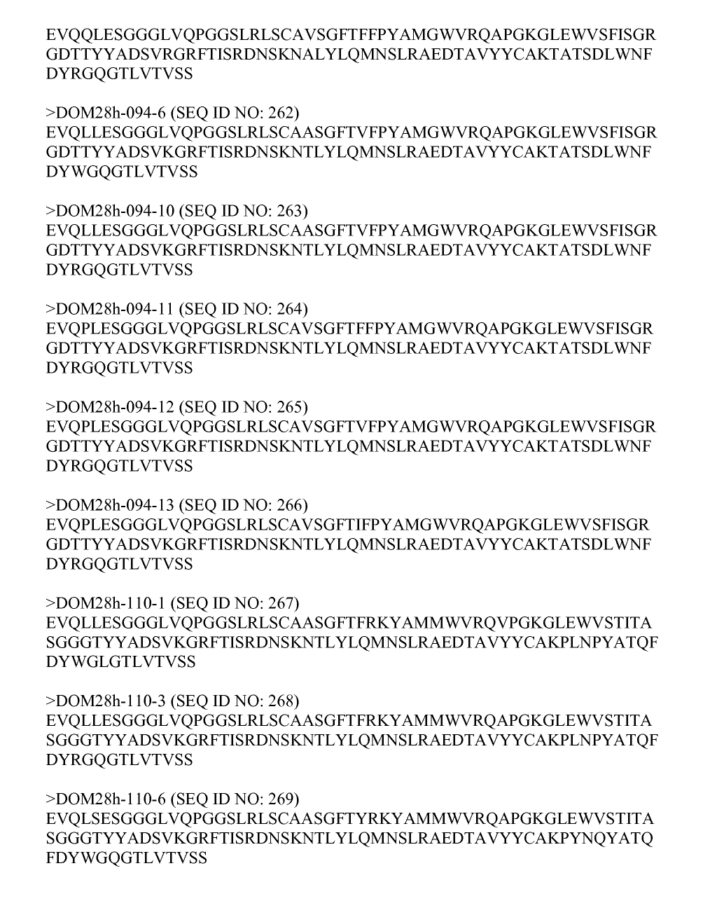

The nucleotide and amino acid sequences of these clones are shown in FIGS. 16A and B.

All DOM28h-94 derivatives had improved affinity to both human and mouse cKIT where as all the other derivatives (DOM28h-5, DOM28h-33, DOM28h-66, DOM28h-84 and DOM28h-110) had improved affinity only to human cKIT.

The nucleotide and amino acid sequences of these clones are shown in FIGS. 16A and B.

TABLE 16

| | | | Affinity (K_{D}) to |
|---|---|---|---|
| | | | human cKIT |
| dAb | Ka | Kd | (nM) |
| DOM28h-5 | 1.37E+06 | 0.0731 | 533 nM |
| DOM28h-5-6 | 9.16E+04 | 4.71E−03 | 52 nM |
| DOM28h-5-7 | 8.67E+04 | 0.0114 | 132 nM |
| DOM28h-5-8 | 8.03E+04 | 8.86E−03 | 110 nM |
| DOM28h-33 | 4.68E+05 | 0.0224 | 48 nM |
| DOM28h-33-9 | 2.47E+05 | 0.0118 | 48 nM |
| DOM28h-33- | 3.24E+05 | 0.0117 | 36 nM |
| 11 | | | |
| DOM28h-33- | 2.24E+05 | 0.0143 | 64 nM |
| 12 | | | |
| DOM28h-33- | 1.58E+04 | 2.34E−03 | 148 nM |
| 19 | | | |
| DOM 28h-66 | 1.42E+05 | 0.412 | 2900 nM |
| DOM 28h-66-3 | 6.45E+05 | 0.0586 | 91 nM |
| DOM 28h-66-6 | 6.11E+05 | 0.0609 | 100 nM |
| DOM 28h-84 | 3.22E+05 | 0.289 | 896 nM |
| DOM 28h-84-6 | 5.22E+06 | 0.029 | 6 nM |
| DOM 28h-84-8 | 9.77E+05 | 0.0242 | 25 nM |
| DOM 28h-84-9 | 3.35E+06 | 0.0398 | 12 nM |
| DOM 28h-84- | 5.86E+04 | 2.95E−03 | 50 nM |
| 10 | | | |
| DOM28h-94 | 1.55E+04 | 0.337 | 21800 nM |
| DOM 28h-94-2 | 6.82E+04 | 0.0159 | 233 nM |
| DOM28h-94-4 | 9.24E+04 | 0.0125 | 135 nM |
| DOM28h-94-6 | 3.92E+04 | 0.0121 | 309 nM |
| DOM28h-110 | 2.06E+05 | 0.674 | 3300 nM |
| DOM28h-110-1 | 6.29E+04 | 0.0616 | 980 nM |
| DOM28h-110-3 | 6.70E+05 | 0.202 | 302 nM |
| DOM28h-110-6 | 8.81E+03 | 0.0372 | 4220 nM |

DOM28h-94 Variants

Analysis of the DOM28h-94 sequences that exhibited the greatest improvements in binding to mouse cKIT revealed a series of consensus mutations that were potentially involved in improving the affinity of the dAb. These were L4P, A24V, F29V/I and W110R. dAbs with different combinations of these mutations were created by site-directed mutagenesis as follows:

DOM28h-94-10: F29V W110R

DOM28h-94-11: L4P A24V W110R

DOM28h-94-12: L4P A24V F29V W110R

DOM28h-94-12: L4P A24V F291 W110R

The binding of two of these dAbs (DOM28h-94-10 & DOM28h-94-12) to mouse cKIT was analysed by BIAcore and approximate kinetic constants from this analysis are also shown in Table 17.

TABLE 17

| | | | Affinity (K_{D}) to |
|---|---|---|---|
| | | | mouse cKIT |
| dAb | Ka | Kd | (nM) |
| DOM28h-94 | 8.84E+04 | 0.135 | 1500 nM |
| DOM 28h-94-2 | 2.80E+04 | 4.38E−03 | 157 nM |
| DOM28h-94-4 | 7.64E+04 | 3.38E−03 | 44 nM |
| DOM28h-94-6 | 2.08E+04 | 5.22E−03 | 251 nM |
| DOM28h-94-10 | 2.19E+04 | 2.45E−03 | 112 nM |
| DOM28h-94-11 | nd | nd | nd |
| DOM28h-94-12 | 2.02E+05 | 2.22E−03 | 11 nM |
| DOM28h-94-13 | nd | nd | nd |

affinity measurements that were not determined are represented by ‘nd’.

The minimum identity to parent (at the amino acid level) of the clones selected was 96% (DOM28h-5-6: 96%, DOM28h-5-7: 99%, DOM28h-5-8: 98%).

The minimum identity to parent (at the amino acid level) of the clones selected was 98% (DOM28h-33-9: 99%, DOM28h-33-11: 98%, DOM28h-33-12: 98%, DOM28h-33-19: 99%).

The minimum identity to parent (at the amino acid level) of the clones selected was 98% (DOM28h-66-3: 98%, DOM28h-66-6: 99%).

The minimum identity to parent (at the amino acid level) of the clones selected was 96% (DOM28h-84-6: 96%, DOM28h-84-8: 98%, DOM28h-84-9: 98%, DOM28h-84-10: 98%).

The minimum identity to parent (at the amino acid level) of the clones selected was 96% (DOM28h-94-2: 98%, DOM28h-94-4: 96%, DOM28h-94-6: 99%, DOM28h-94-10: 98%, DOM28h-94-11: 98%, DOM28h-94-12: 97%, DOM28h-94-13: 97%).

The minimum identity to parent (at the amino acid level) of the clones selected was 98% (DOM28h-110-1: 98%, DOM28h-110-3: 98%, DOM28h-110-6: 98%).

C) Further Screening for Mouse C-Kit Binding dABs

Further mcKIT binding dAbs were generated and characterised. The selection outputs from round 3 set out in Table 13 above (i.e. DOM 28m-7, DOM 28m-23 and DOM 28m-52) were sub-cloned once again from the pDOM4 phage vector into the pDOM10 expression vector for soluble dAb expression with C-terminal FLAG tag. Phage DNA from the outputs of round 3 were prepared as described above.

Individual colonies were picked and sequenced to ensure no loss of sequence diversity from the library after cloning, and re-picked into a 96 well plate format. A total of 13 plates or 1200 clones were analysed. The cells were grown in 2.2 ml deep well plates in 2×TY with Overnight Express™ auto induction media cocktails (Merck). Cells were grown at 30° C. for 72 hours in an Infors high-speed shaker with humidity.

The supernatants from the 96 well plate expressions were mixed 1:1 with HBP-EP BIAcore running buffer (GE Healthcare) and ran over the BIAcore on a CM5 mcKIT coated chip.

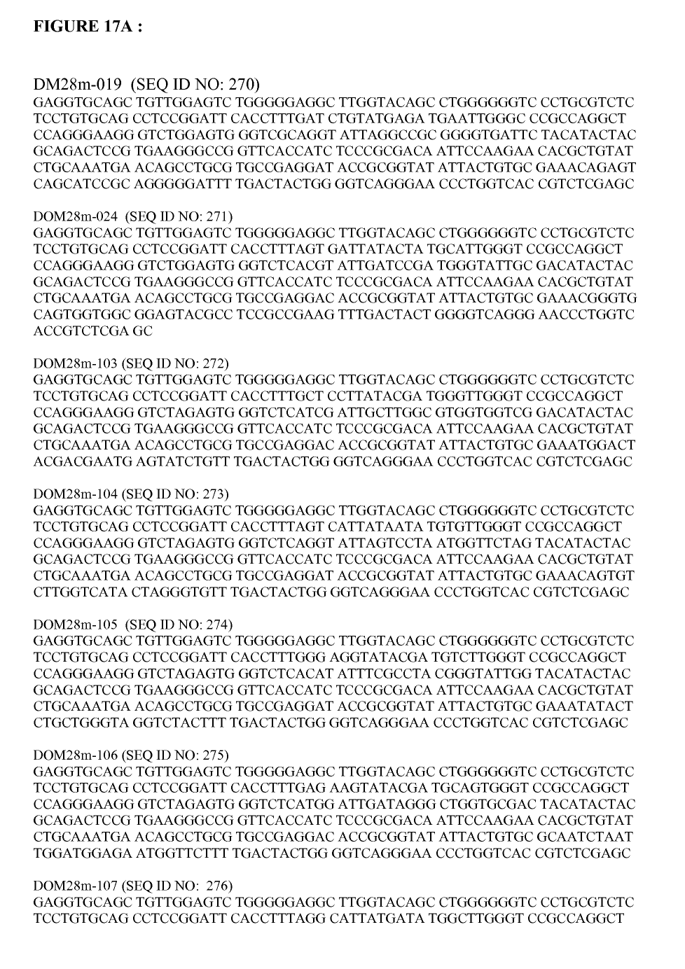

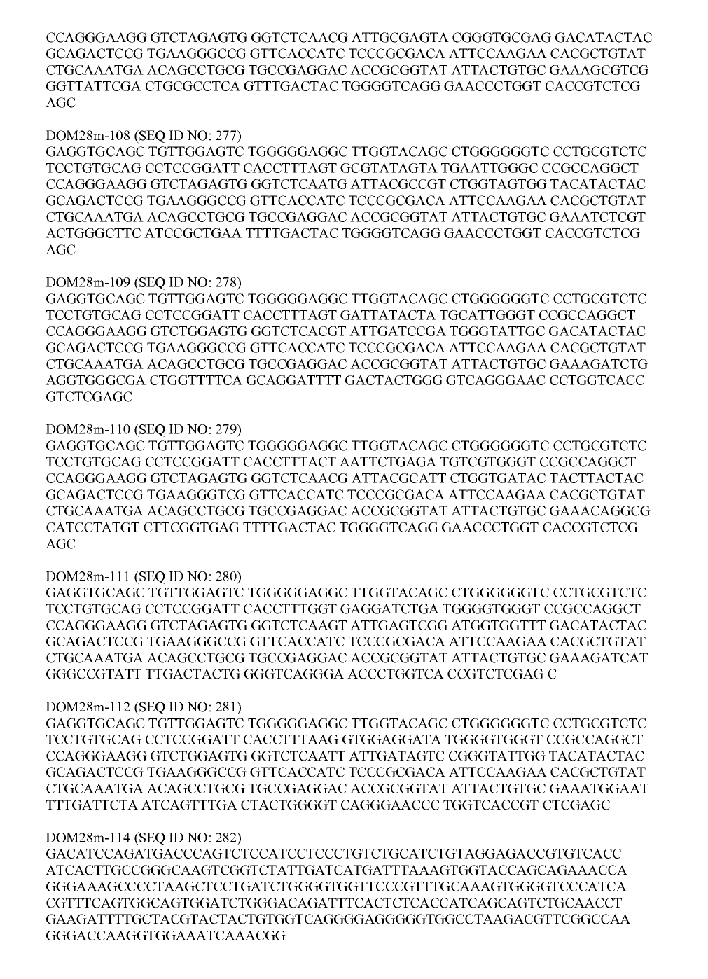

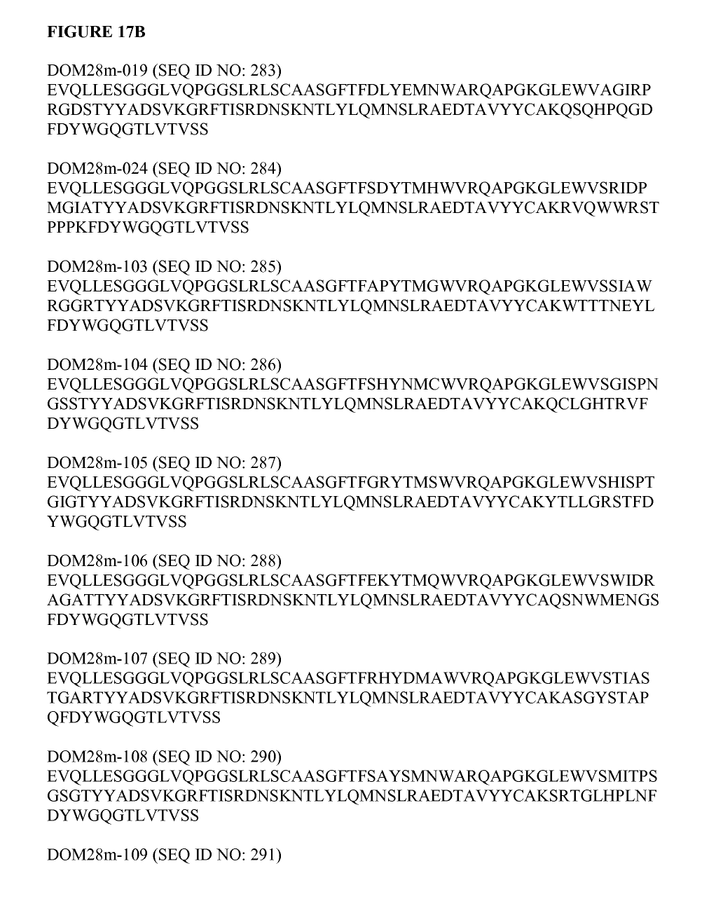

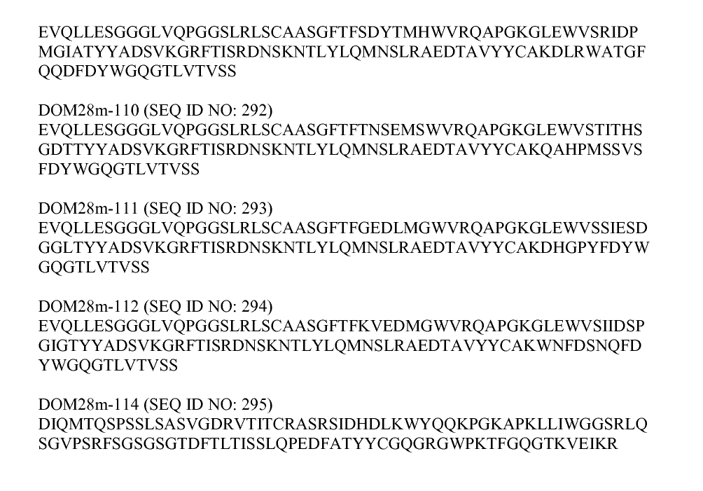

A further 38 clones were identified. Duplicate clones, those that bound the Fc domain introduced into the recombinant c-kit protein for expression purposes and those with sequencing errors were eliminated to give a list of 19 dAbs, the sequences of which are set out in FIGS. 6A and 6B and FIGS. 17A and B, based on their BIAcore binding to mcKIT. These clones were grown in 50 ml of 2×TY with Overnight Express™ auto induction media cocktails (Merck) and grown, as in the screening, for 72 hours at 30° C. in an Infors shaker incubator as above. The 50 ml supernatants were mixed with 1.5 ml of protein A or protein L resin and left to bind with rotation for 3 hours at room temperature. The resins with supernatants were packed into a column and samples were column purified by washing with 30 ml of PBS, 30 ml of 10 mM TRIS-HCL pH 8, and eluting the dAbs with 10 ml of 0.1M glycine pH 2. Samples were neutralised with 2.5 ml of 1M TRIS-HCL pH 8 and concentrated down to approximately 1 ml and dialysed into PBS for expression, biophysical and binding analysis.

The dAbs were tested for their binding to mcKIT and their cross reactivity to hcKIT, the data of which is shown in the table below. All binding data was performed on a BIAcore 2000 instrument at 1 uM of dAb concentration. Clones DOM28m-7 and DOM28m-23 were previously analysed for their binding to mcKIT and hckit (Table 12), and the KD values are roughly in agreement (within the 10 fold error expected on BIAcore). Clones were ranked according to cell binding data both in dAb and mAbdAb formats (Tables 20, 26 and 30).

TABLE 18

| | KD | | Ranking and |
|---|---|---|---|
| Name of dAb | (mcKIT) | KD (hcKIT) | Reasoning |
| DOM28m-4 | 11 uM | No binding | X IC |
| DOM28m-7 | 4 uM | weak | Y MD 4 |
| DOM28m-8 | 3 uM | No binding | X WB |
| DOM28m-17 | 6 uM | weak | Y MD 5 |
| DOM28m-19 | 14 uM | No binding | X IC |
| DOM28m-23 | 22 uM | 6 uM | Y MD 9 |
| DOM28m-24 | 210 nM | 33 nM | X NS |
| DOM28m-103 | 226 nM | 8 uM | X WB |
| DOM28m-104 | 1 uM | No binding | Y MD 3 |
| DOM28m-105 | Weak | No binding | X NS |
| DOM28m-106 | 9 uM | No binding | Y MD 6 |
| DOM28m-107 | 60 nM | 45 nM | Y MD 1 |
| DOM28m-108 | Weak | No binding | X IC |
| DOM28m-109 | 11 uM | No binding | Y MD 7 |
| DOM28m-110 | 3 uM | No binding | X IC |
| DOM28m-111 | 1 uM | No binding | X IC |
| DOM28m-112 | 11 uM | No binding | Y MD 8 |
| DOM28m-73 | 9 uM | No binding | X NS |
| DOM28m-114 | 1 uM | 920 nM | Y MD 2 |

IC—inconsistent cell binding data
WB—weak binding on cells as dAb and as mAbdAb
Y—Clone considered to be good
MD—mAbdAb data suggests clone is a good specific binder
NS—cell binding data suggests non specificity
X—Clone discarded
Weak—weak binding observed

D) Binding of C-Kit dABs to Cells Expressing C-Kit

Positive binding anti-human c-KIT and anti-mouse c-kit dAbs were tested to confirm binding to c-KIT expressed on human (HEL-92.1.7 (Biocat #49486) and KU812 (Biocat #117278)) using a method modified from that described in Example 4. Two mouse cell lines expressing murine c-KIT were identified (MC/9 (ATCC #CRL-8306) and EML (ATCC #CRL-11691)) and these cell lines were subsequently used in flow cytometry assays. Cell lines (Jurkat and HeLa (Biocat #113348)) that did not express c-KIT were included to confirm specificity.

c-KIT dAbs were diluted to appropriate concentration (4× final concentration) in 25 μl PBS with 2.5% FBS (FACS buffer) and added to 25 μl 40 μg/ml (4×) anti-FLAG-BIO (Sigma #F9291) for 1 hr at room temperature to allow the reagents to pre-complex. Cells were counted and washed in FACS buffer. 50 μl cells were added to the dAb/anti-FLAG complex at a density of 1×10⁶ cells per well and left for 1 hr at 4′C. Cells were spun at 1200 rpm for 3 min at 4′C and washed with ice-cold FACS buffer twice before incubation with 100 μl 1.25 μg/ml streptavidin-PE (Biolegend #405204) for 40 mins at 4′C. Cells were spun and washed in FACS buffer again before being resuspended in 200 μl PBS. Samples were analysed for dAb binding on the FACS Canto II flow cytometer (BD Biosciences). All data was analysed using Flow Jo software (Tree Star). Profiles were generated for all the affinity matured clones and used to score whether the dAbs bind to c-kit expressed on mouse MC/9 and EML cells. A control cell line (HELA (HeLa)) that did not express c-kit was included in this assay. All binding of the c-KIT dAbs (YES' or ‘NO’) was expressed relative to that for a negative control molecule (Vk and V_{H}-2 dummy dAbs).

Table 19 shows DOM 28h-94-2, DOM 28h-94-4 and DOM 28h94-6 all bound to MC/9 cells better than the parent DOM 28h-94 clone but none of the DOM 28h-94 lineage bound to EML cells. There was no binding to the c-kit negative HeLa cell line.

TABLE 19
Table summarising the binding of h94 affinity matured lineage
to HELA, MC/9 and EML cells.

| c-kit dAb | HELA | MC/9 | EML |
|---|---|---|---|
| DOM 28h94 | NO | NO | NO |
| DOM 28h94-2 | NO | YES | NO |
| DOM 28h 94-4 | NO | YES | NO |
| DOM 28h 94-6 | NO | YES | NO |
| DOM 28h 94-8 | NO | NO | NO |

Table 20 shows 13 of the anti-mouse c-kit dAbs to bind to c-kit expressed on cells.

TABLE 20
Table summarizing the binding data for the mouse c-kit dAbs

| | | | HEL- | | | |
|---|---|---|---|---|---|---|
| c-kit dAb | Jurkats | HELA | 92.17 | KU812 | MC/9 | EML |
| DOM28m4 | 1/2* | NT | YES | NT | YES | YES |
| DOM28m7 | NO | NT | YES | YES | NO | 2/4* |
| DOM28m8 | NO | NT | YES | YES | 1/2* | 1/2* |
| DOM28m17 | NO | 1/2* | YES | NT | YES | YES |
| DOM28m23 | NO | NO | YES | YES | 1/3* | 2/3* |
| DOM28m47 | NO | NT | YES | NT | NO | YES |
| DOM28m52 | NO | NT | YES | NT | YES | YES |
| DOM28m53 | N0 | NT | YES | NT | YES | YES |
| DOM28m73 | YES | NT | YES | NT | YES | YES |
| DOM28m24 | NO | NT | YES | YES | YES | YES |
| DOM28m103 | NO | NT | YES | YES | YES | YES |
| DOM28m104 | NO | NT | YES | YES | YES | YES |
| DOM28m104 | NT | NO | NT | NT | YES | NO |
| DOM28m106 | NB | NT | YES | YES | YES | YES |
| DOM28m107 | NO | NT | YES | YES | NO | NO |
| DOM28m109 | NO | NT | YES | YES | YES | YES* |
| DOM28m110 | NO | NT | NT | NT | YES | NT |
| DOM28m111 | NO | NT | YES | NT | NO | YES |
| DOM28m112 | NO | NO | YES | YES | 1/2* | 1/2* |
| DOM28m114 | NO | NO | YES | YES | YES | 1/2* |
| DOM28h94 | NT | NO | NT | NT | NO | NO |

NT = not tested; Yes = binding; No = no binding
*= inconclusive data (dAb inconsistently binds e.g. 1 out of 2 occasions (1/2*) or 1 out of 3 occasions (1/3*)).

E) Species Cross-Reactivity of Anti-Mouse c-KIT Clones

13 dAbs were analysed further for their binding. Cross reactivity with rat cKIT antigen revealed that different dAbs could be categorised based on their cross reactivity. The table below highlights the cross species BIAcore data.

TABLE 21

| | | | KD - rat |
|---|---|---|---|
| | KD - mouse | KD - human | cKIT |
| Name of dAb | cKIT nM | cKIT nM | nM |
| DOM28m-7 | 3000 | 0 | 30 (BF) |
| DOM28m-17 | 6000 | 0 | 43 (BF) |
| DOM28m-23 | 22000 | 6000 | 25 (BF) |
| DOM28m-24 | 210 | 33 | 75 |
| DOM28m-103 | 230 | 8000 | 500 |
| DOM28m-104 | 1000 | 0 | 0 |
| DOM28m-105 | weak | 0 | 0 |
| DOM28m-107 | 60 | 0 | 0 |
| DOM28m-108 | weak | 0 | 0 |
| DOM28m-109 | 11000 | 0 | 1200 |
| DOM28m-112 | 11000 | 0 | 0 |
| DOM28m-114 | 1000 | 920 | 85 |
| DOM28m-73 | 9000 | 0 | 0 |

*BF denotes a bad fit on BIAcore;
weak—weak binding observed

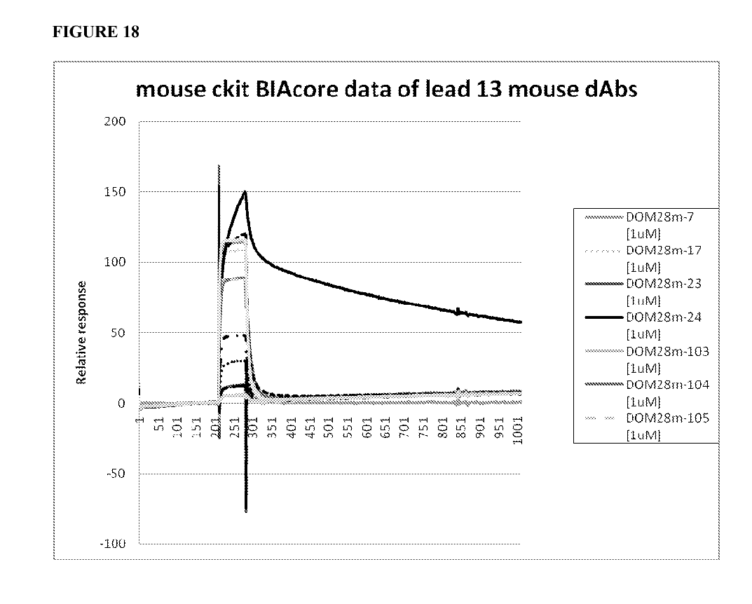

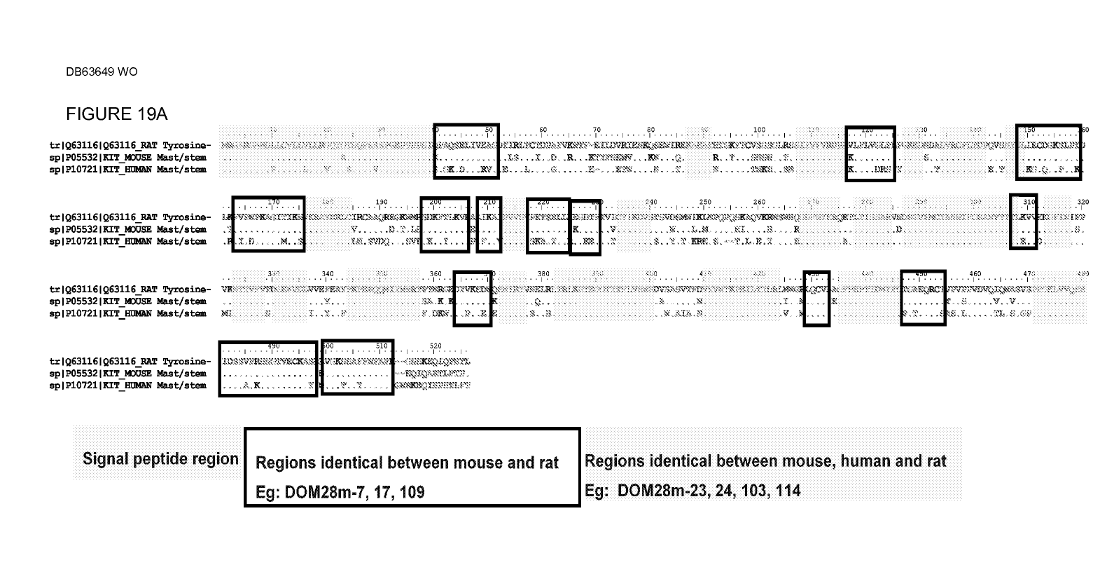

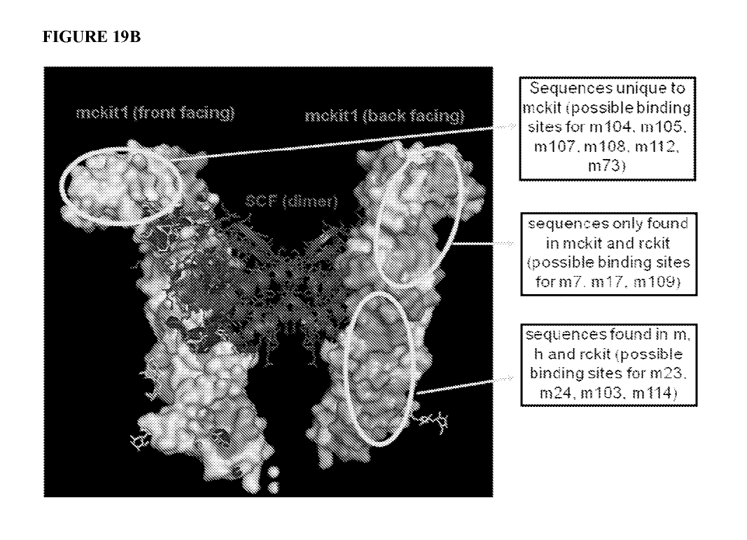

This analysis gave broad insights into epitopes of binding based on sequence-structure alignments of mouse, human and rat cKIT (FIGS. 18 and 19). FIG. 19 shows a sequence alignment of the mouse, human and rat cKIT. Based on this alignment, the areas of similarities between all 3 species can be mapped onto the structure of cKIT giving approximate indications to where the various epitopes may lie.

These 13 dAb clones were analysed for their biophysical properties by SEC MALLS and DSC. For SEC MALLS, proteins are separated on the SEC column at 1 mg/ml in PBS buffer and the refractive index of molecules eluted gives accurate measures of molecular mass. DSC was carried out as described above. Clones DOM28m-7 and DOM28m-23 were previously tested on SEC MALLS (Table 14) and the data is consistent with the table below. DOM28m-7 was also tested on DSC once before (Table 15) and is in agreement with the table below

TABLE 22

| | SEC MALLS data | | | |
|---|---|---|---|---|
| | Mean Molar | | DSC data | |
| | Mass over | In-solution | App Tm1, | App Tm2, |
| Name of dAb | main peak | State | ° C. | ° C. |
| DOM28m-7 | 16 kDa | 75% Monomer | 66.9 | 69.5 |
| DOM28m-17 | 16.6 kDa | 70% Monomer | 60.9 | 69.9 |
| DOM28m-23 | 16.8 kDa | 87% Monomer | 64 | 68.9 |
| DOM28m-24 | ND | | ND | ND |
| DOM28m-103 | 14.24 kDa | Monomer | 57.3 | 59.2 |
| | 15.34 kDa | | | |
| DOM28m-104 | 4.42 kDa | Monomer | 74.1 | 76 |
| | 15.3 kDa | | | |
| DOM28m-105 | ND | | 55.3 | 56.7 |
| DOM28m-107 | 6.12 kDa | Monomer | 59.9 | 69 |
| | 15.1 kDa | | | |
| DOM28m-108 | 15 kDa | Monomer and | 56.1 | 70.2 |
| | 50 kDa | Oligomer | | |
| DOM28m-109 | ND | | 56.6 | 64.2 |
| DOM28m-112 | 14.6 kDa | Monomer | 58.2 | 65.3 |
| DOM28m-114 | 10.89 kDa | Monomer | 61.9 | 66.9 |
| | 13.32 kDa | | | |
| DOM28m73 | 16 kDa | 75% Monomer | 53.4 | 57.8 |

ND—not determined

Mouse dAb/C-Kit Phosphorylation Assay in the Presence and Absence of SCF

No competitive receptor binding assay was available for detection of mc-kit binding dAbs, therefore an alternative assay platform was utilized to confirm that the 13 mc-kit binding dAbs did not compete with mouse SCF binding to mouse c-kit. The effect of dAbs on one of the signaling cascades following mouse SCF binding to mouse c-KIT expressing cells was examined in a mouse c-KIT phosphorylation MesoScaleDiscovery (MSD) assay.

Mouse c-KIT positive cell lines, MC/9 and EML, and human HeLa cells (a c-KIT negative control line) were used. The dAbs were added to cells at the appropriate concentration with or without 150 ng/ml mouse SCF for 5 min at room temperature. The cells were then lysed using Cell Signaling lysis buffer (catalog #9803 Cell Signaling Technology) on ice and run in a mouse c-kit phosphorylation assay. Briefly, an anti-mouse c-kit antibody (eBioscience #14-1171)) was coated at 1 μg/ml on to a blank standard bind MSD plate (catalog #L15XA-3/L11XA-3) overnight at 4′C. The plate was washed and blocked with 3% BSA in PBS for 1 hr at room temperature. The plate was then washed 3 times in PBS with 0.5% Tween-20. 25 ul of cell lysate was added for 1 hr at room temperature and washed as before. The bound antigen was detected with 0.5 μg/ml anti-phosphotyrosine kinase-SULPHO tagged MSD antibody (MSD #R32AP) for 1 hr at room temperature. The plate was washed again and 150 μl of 1×MSD read buffer (catalog #R92TC) was added before being read on the MSD imager.

For EML cells, modifications were made to this protocol. The cells were starved of mouse SCF from the culture media 48 hr prior to the phosphorylation assay and the amount of SCF used was increased to 1 μg/ml. Commercial anti-cKIT antibodies were included as controls; clone 2B8 (eBioscience catalog #14-1171) is a non-neutralising mAb that should not affect c-kit phosphorylation, whereas clone ACK45 (BD Pharmingen catalog #553868) is a neutralising mAb which may decrease c-kit phosphorylation in the presence of mouse SCF.

The data generated confirmed that, in the presence of SCF, c-kit dAbs did not completely reduce c-kit phosphorylation to baseline levels (i.e. MSD signal in absence of SCF) with the exception of DOM28m-104 in MC/9 cells. In the absence of SCF, the dAbs did not affect c-kit phosphorylation. Control behaved as expected.

Affinity Maturation of mcKIT dAbs

7 mouse cKIT dAbs (DOM28m-7, DOM28m-17, DOM28m-23, DOM28m-104, DOM28m-107, DOM28m-109, DOM28m-112), along with the human/mouse cross reactive dAb, DOM28h-94, were taken forward for affinity maturation. These were chosen based on the results of the cell binding mAbdAb data (Section 5.1.2(e). Error prone PCR was carried out with a starting template concentration of 50 pg. Errors were introduced throughout the gene with the GeneMorph Random Mutagenesis kit with Mutazyme Polymerase (Stratagene). The error prone PCRs were performed with biotinylated 3′ and 5′ primers for more efficient purification of PCR fragments. 3 ug of error prone inserts were then digested with the concentrated forms of SalI and NotI restriction enzymes (New England Biolabs) to enhance digestion efficiency. The digested error prone inserts were purified on streptavidin beads. The phage vector, pDOM4 was also digested with concentrated forms of SalI and NotI restriction enzymes, as with the error prone inserts, however an additional digest with PstI (New England Biolabs) ensured that all vector was digested. pDOM4 is a phage vector based on the commercial phage vector fd-tet, and ensures that the dAb libraries are cloned in frame with the gene III phage surface protein for phage display. The prepared inserts and vector were ligated in a 3:1 insert to vector molar ratio. 5 ug of vector were used to generate libraries all in the region of 10⁸. Such library sizes were possible by making freshly prepared TB1 competent cells. Cells were scraped and grown in 150 ml 2×TY supplemented with tetracycline for phage harvest. Between 0 and 4 amino acid mutations were seen on the protein level, with an average of 1.3 mutations per gene.

The 8 dAb libraries were selected against biotinylated mouse cKIT in soluble selections. Two batches of selections were performed with 100 nM, 10 nM and 2.5 nM of biotinylated mcKIT and 100 nM, 50 nM and 5 nM of biotinylated mcKIT. Phage based ELISA and sequencing after rounds 2 and 3 helped determine the progression of selections. After the third round of selections, the libraries outputs were subcloned into the vector pDOM10 as described previously.

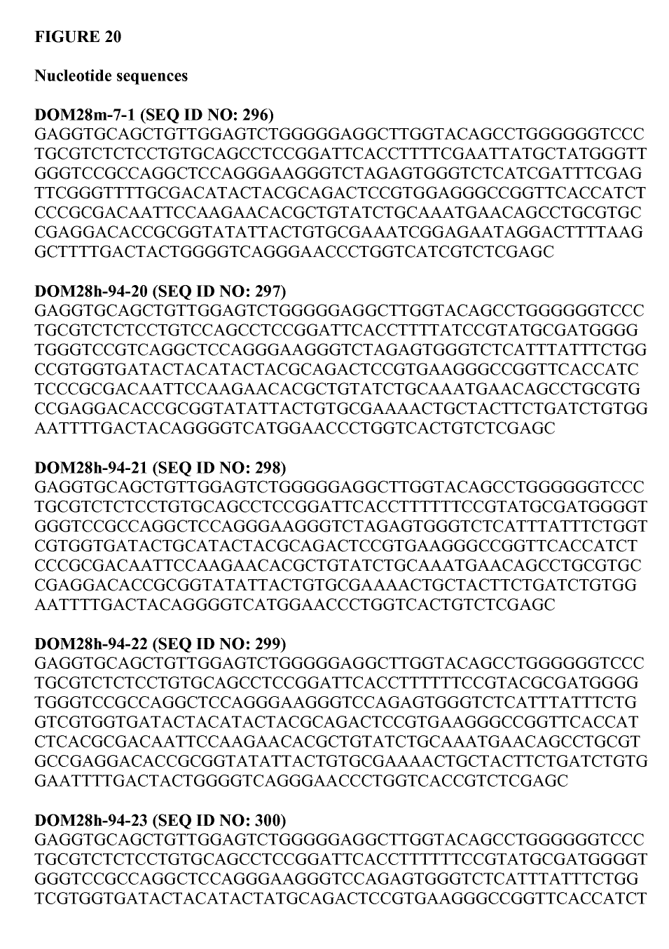

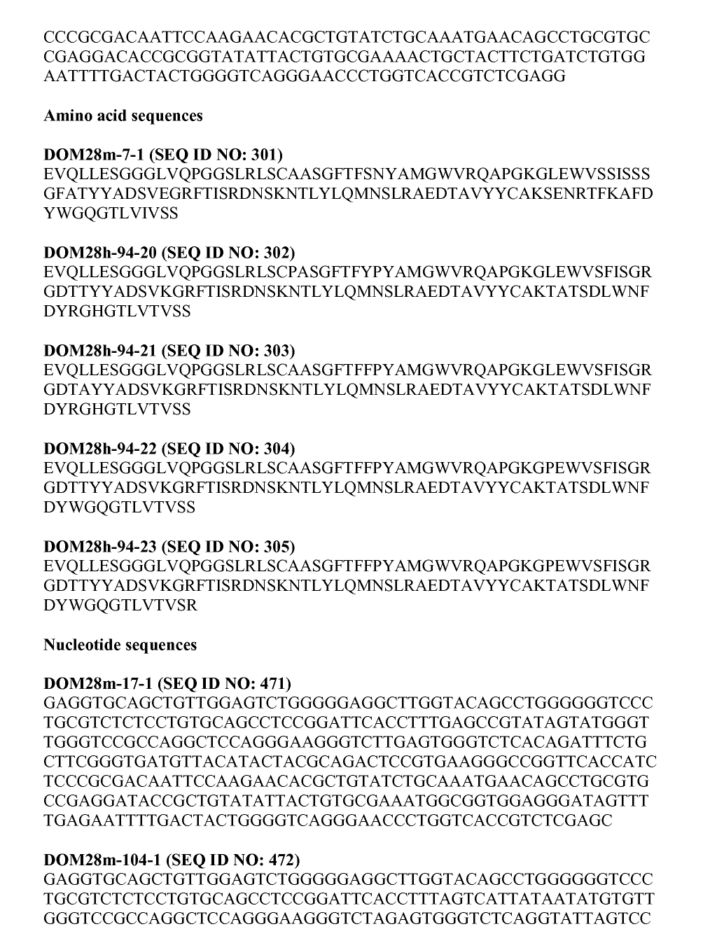

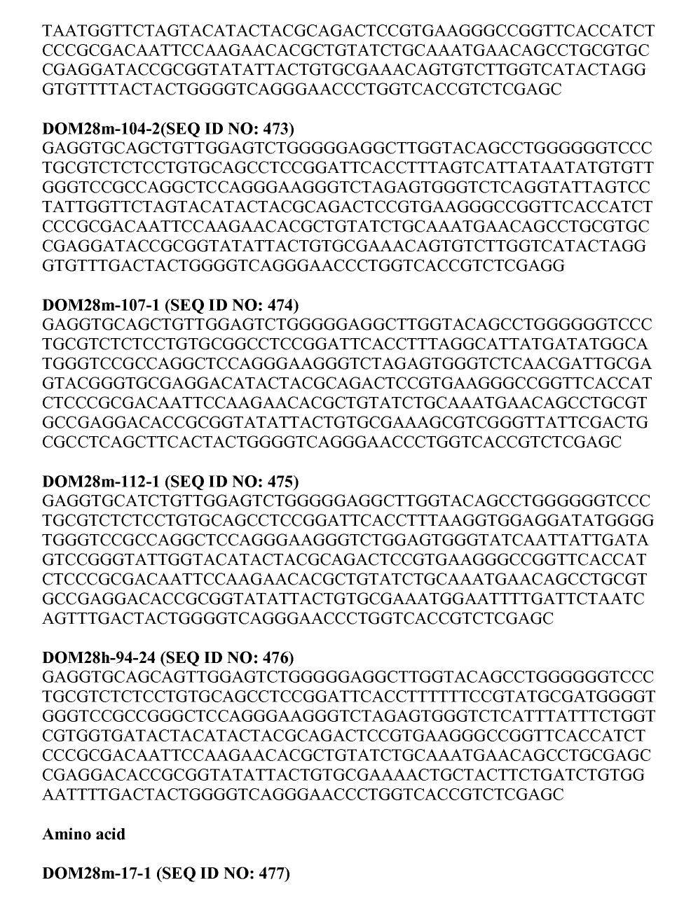

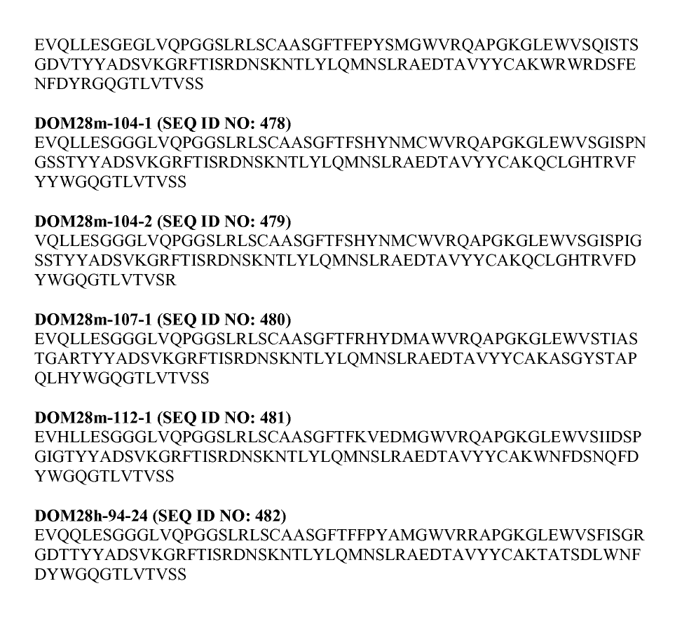

Improved binders compared to parent dAbs were screened by BIAcore as supernatants. In total 16 plates, resulting in approximately 1500 single clones were grown in 1 ml of 2×TY supplemented with Autoinduction cocktails (as described above) and grown for 72 hrs at 30° C. The supernatants were mixed 1:1 with BIAcore HBS-EP buffer (GE Healthcare) and run with regeneration points at the end of injections with glycine pH 2. A total of 11 clones with improved BIAcore off rates were identified and sequenced. The majority of these came from the DOM28h-94 lineage, however positives were found in many other different lineages. The KD values of these clones along with their affinity matured counterparts are given in Table 23 (dAbs where binding was too weak for KD values to be determined are described as “weak”, dAbs where the KD measurement was not able to be correctly fitted using BIAcore software is described as “bad fit”, dAbs where the KD value was not determined are designated “ND”. The sequences of these 11 clones are given in FIG. 20.

TABLE 23

| | | | Fold | |
|---|---|---|---|---|
| | KD - mckit | KD - rckit | improvement | |
| Name of Clone | (nM) | (nM) | over parent | Rank |
| DOM28m-7-1 | 1000 | Weak | x3 | 6 |
| DOM28m-17-1 | 200 | ND | x30 | 3 |
| DOM28m-104-1 | Bad fit | ND | — | |
| DOM28m-104-2 | 145 | ND | x7 | 1 |
| DOM28m-107-1 | 150 | ND | x1.5 | 2 |
| DOM28m-112-1 | Bad fit | ND | — | |
| DOM28h-94-20 | 500 | 200 | x14 | 4 |
| DOM28h-94-21 | 2000 | 2000 | x4 | 7 |
| DOM28h-94-22 | 3000 | 1000 | x2 | 8 |
| DOM28h-94-23 | 1000 | Weak | x7 | 5 |
| DOM28m-94-24 | 9000 | ND | — | 9 |

### Example 5

### Generation of Dual Targeting mAbdAbs

Dual targeting mAbdAbs are constructed in the following way. In one embodiment, the mAb is an MLC mAb as described herein and the dAb is an anti-c-kit dAb as described herein. Expression constructs are generated by grafting a sequence encoding a domain antibody on to a sequence encoding a heavy chain or a light chain (or both) of a monoclonal antibody such that when expressed the dAb is attached to the C-terminus of the heavy or light chain. Linker sequences may be used to join the domain antibody to heavy chain CH3 or light chain CK. Suitable linker sequences include STG (SEQ ID NO: 99); STGGGGGS (SEQ ID NO: 95); STGGGGGSGGGGS (SEQ ID NO: 96); TVAAPS (SEQ ID NO: 89); GS (SEQ ID NO: 105); GSTVAAPS (SEQ ID NO: 102); STGPPPPPS (SEQ ID NO: 97); STGPPPPPPPPPPS (SEQ ID NO: 98); AST (SEQ ID NO: 94); or ASTKGPS (SEQ ID NO: 91). In other constructs the domain antibody may be joined directly to the heavy or light chain with no linker sequence.

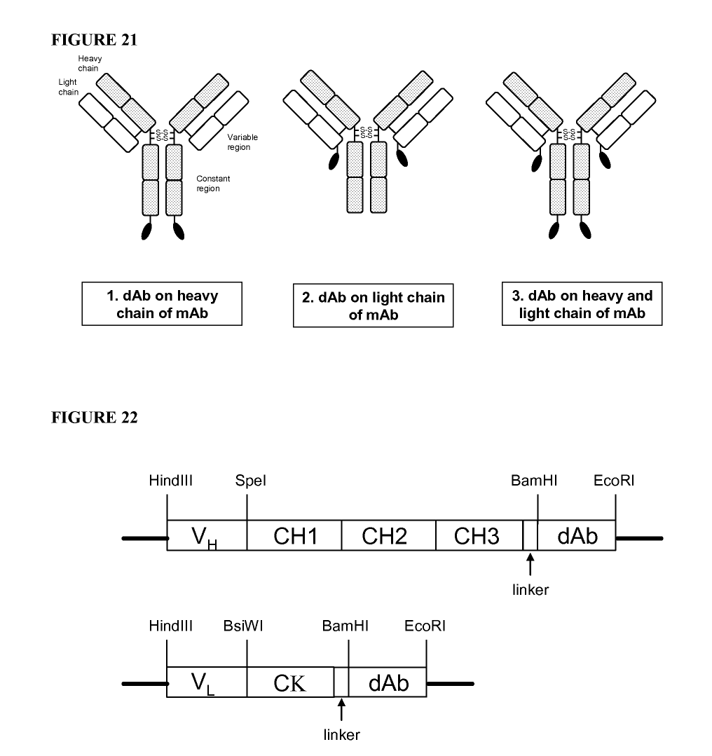

A general schematic diagram of mAbdAb constructs is shown in FIG. 21 (the mAb heavy chain is drawn in grey; the mAb light chain is drawn in white; the dAb is drawn in black). mAbdAb types 1 and 2 are tetravalent constructs, mAbdAb type 3 is a hexavalent construct.

A schematic diagram illustrating the construction of a mAbdAb heavy chain (top illustration) or a mAbdAb light chain (bottom illustration) is shown in FIG. 22.

Note that for the heavy chain the term ‘V_{H}’ is the monoclonal antibody variable heavy chain sequence; ‘CH1, CH2 and CH3’ are human IgG1 heavy chain constant region sequences; ‘linker’ is the sequence of the specific linker region used; ‘dAb’ is the domain antibody sequence. For the light chain the term ‘V_{L}’ is the monoclonal antibody variable light chain sequence; ‘CK’ is the human light chain constant region sequence; ‘linker’ is the sequence of the specific linker region used; ‘dAb’ is the domain antibody sequence.

DNA expression constructs are made de novo by oligo build or derived from existing constructs (as described above) by restriction cloning or site-directed mutagenesis.

These constructs (mAbdAb heavy or light chains) are cloned into mammalian expression vectors (Rln, Rld or pTT vector series) using standard molecular biology techniques. A mammalian amino acid signal sequence may be used in the construction of these constructs.

For expression of mAbdAbs where the dAb is joined to the C-terminal end of the heavy chain of the monoclonal antibody, the appropriate heavy chain mAbdAb expression vector is paired with the appropriate light chain expression vector for that monoclonal antibody. For expression of mAbdAbs where the dAb is joined to the C-terminal end of the light chain of the monoclonal antibody, the appropriate light chain mAbdAb expression vector is paired with the appropriate heavy chain expression vector for that monoclonal antibody.

For expression of mAbdAbs where the dAb is joined to the C-terminal end of the heavy chain of the monoclonal antibody and where the dAb is joined to the C-terminal end of the light chain of the monoclonal antibody, the appropriate heavy chain mAbdAb expression vector is paired with the appropriate light chain mAbdAb expression vector.

mAbdAbs may be expressed transiently in CHOK1 cell supernatants and analysed for activity in MLC and c-Kit binding ELISAs.

### 5.1. Experimental

### 5.1.1 Introduction

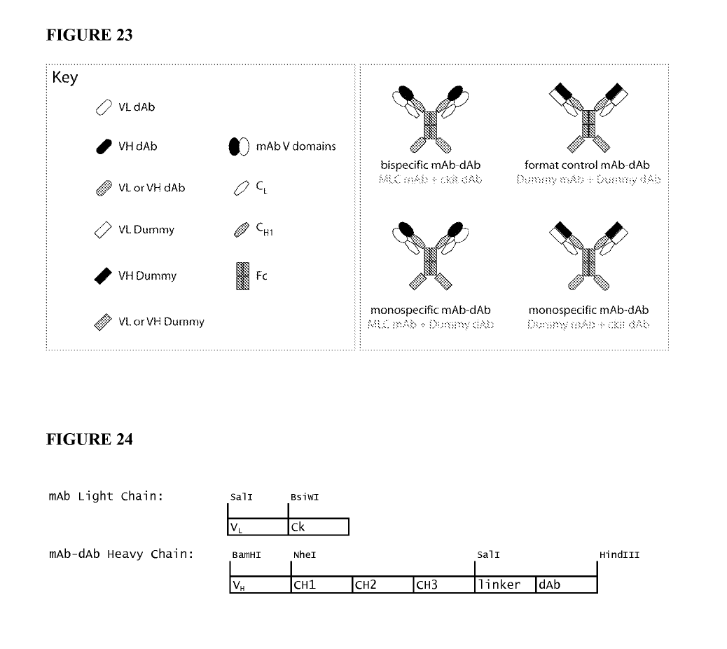

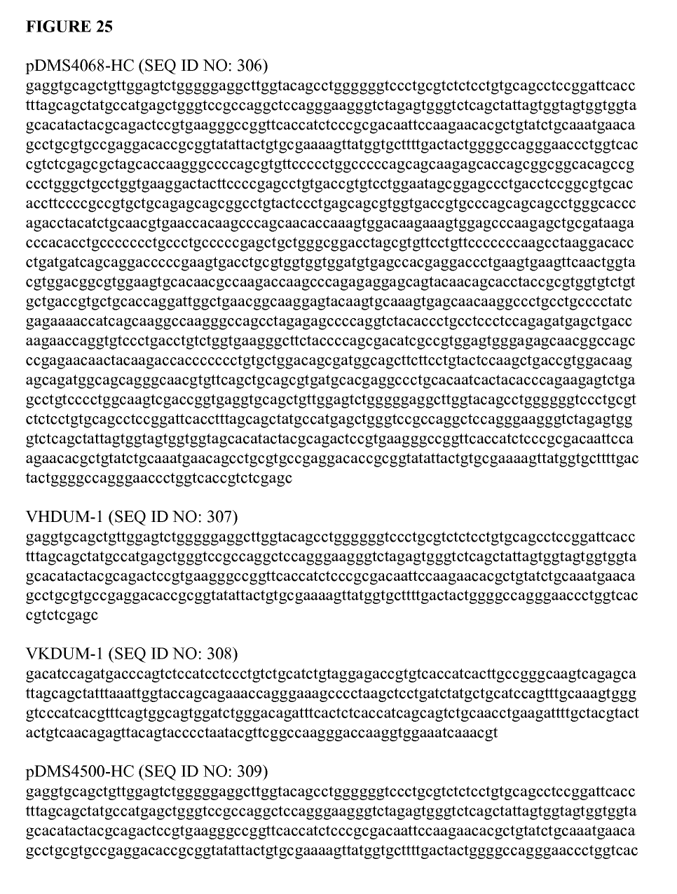

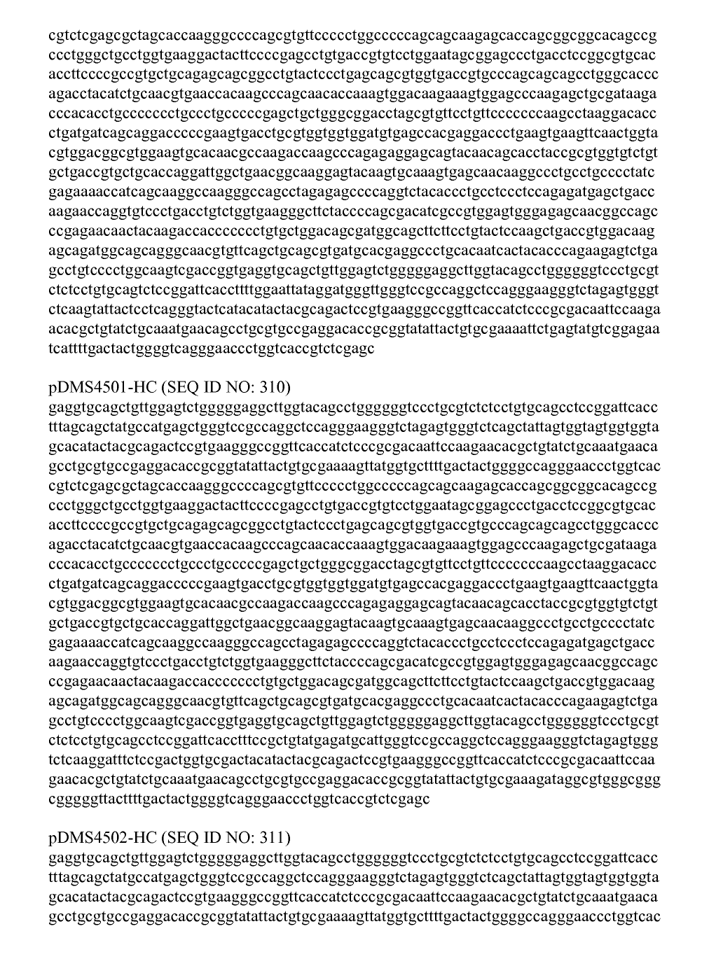

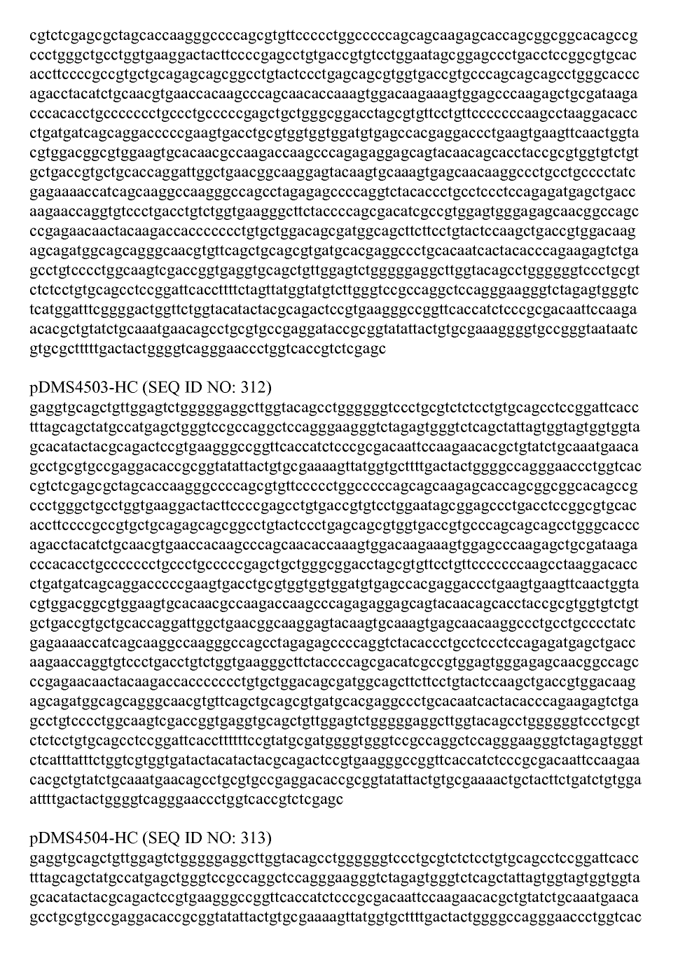

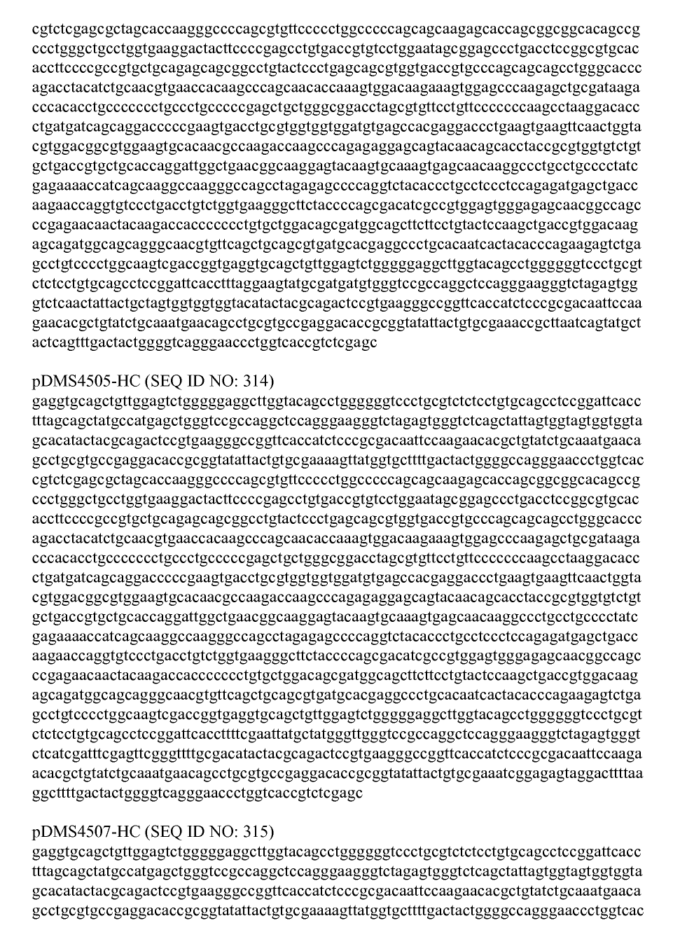

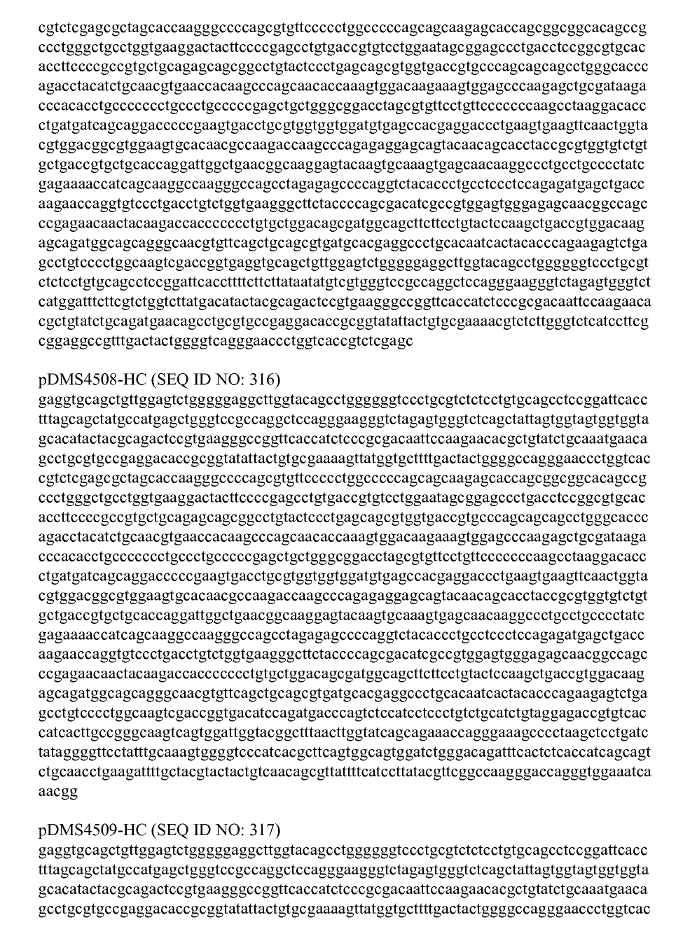

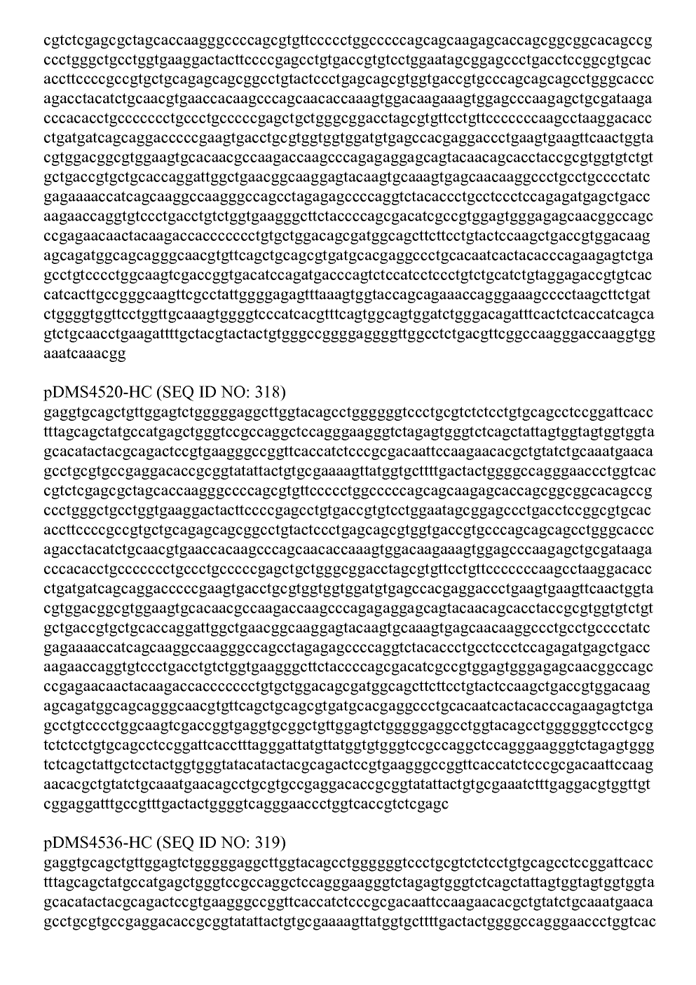

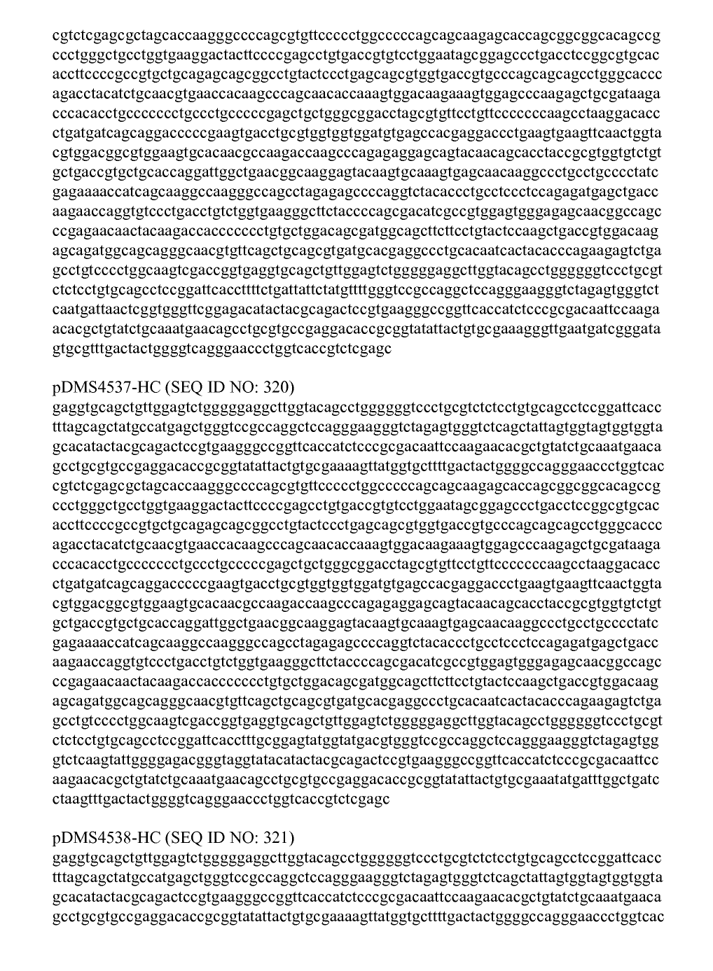

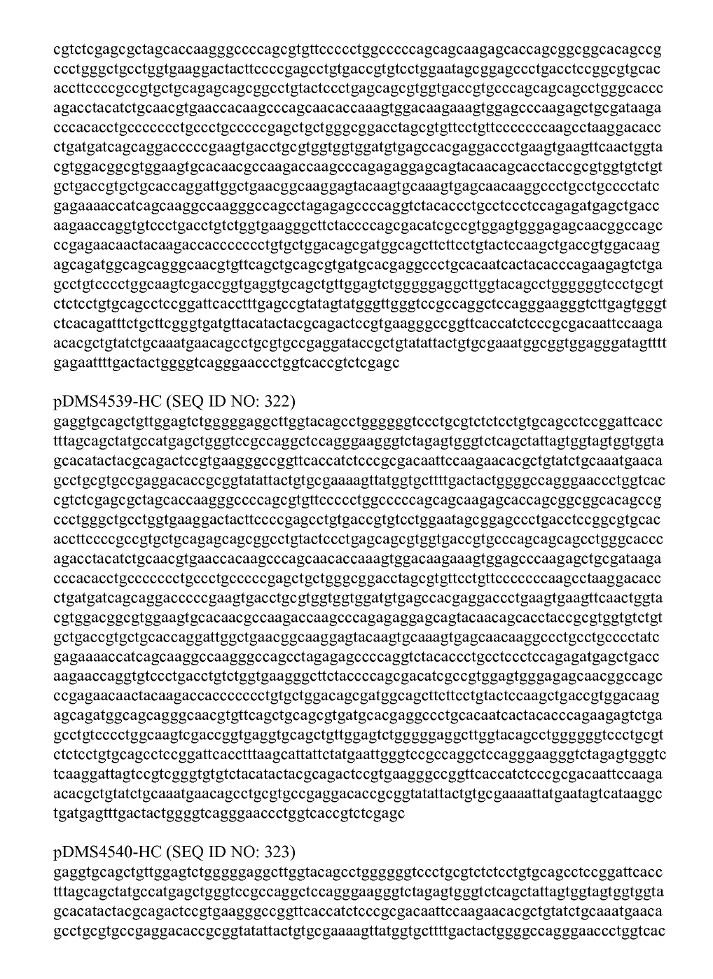

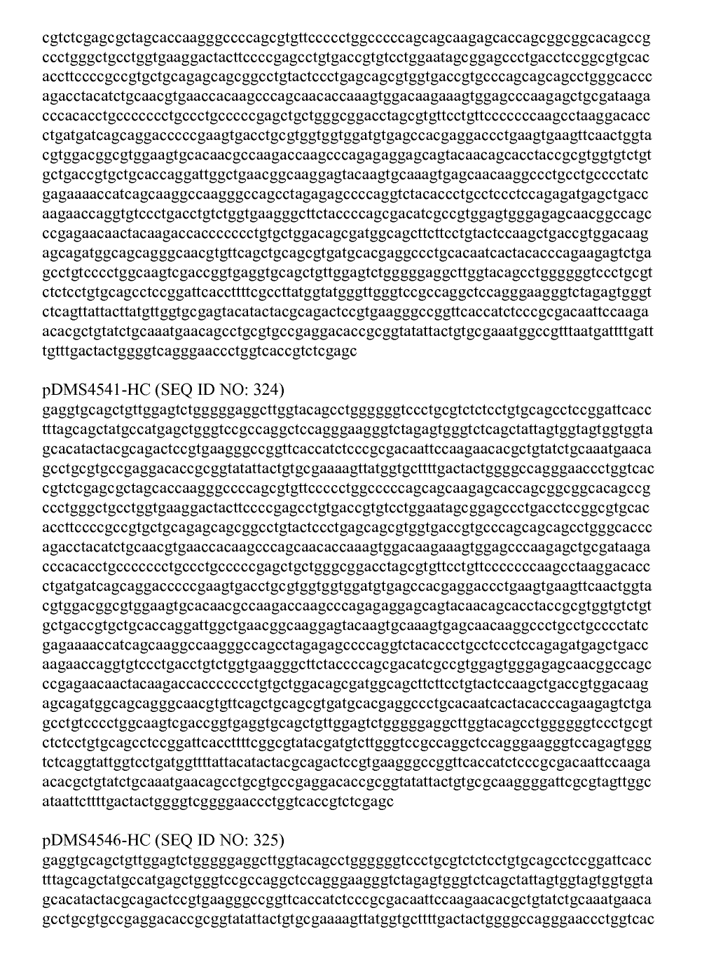

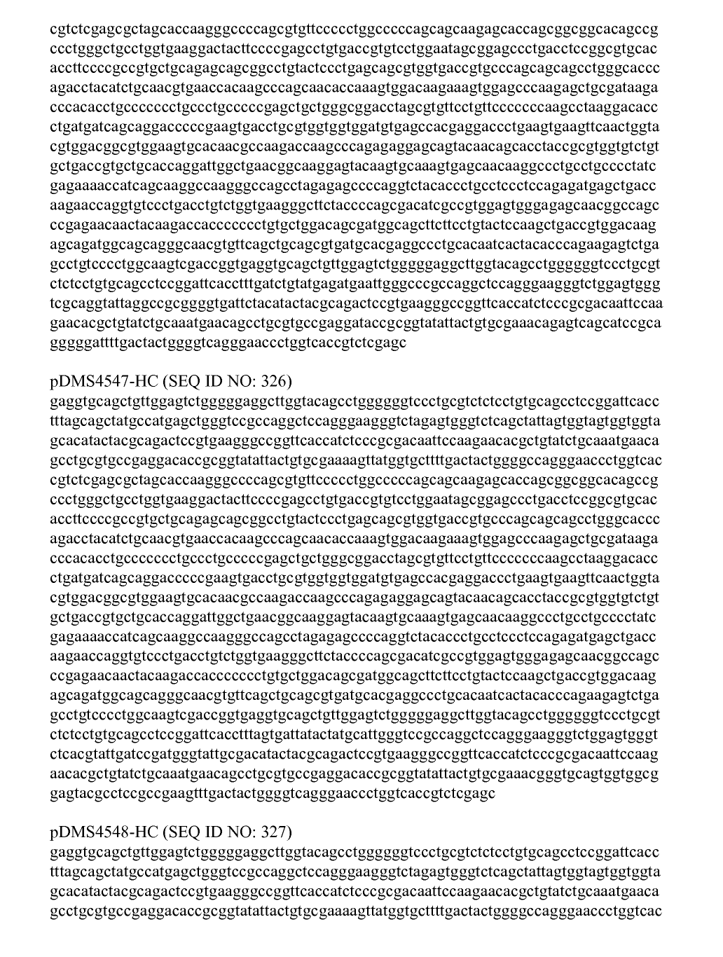

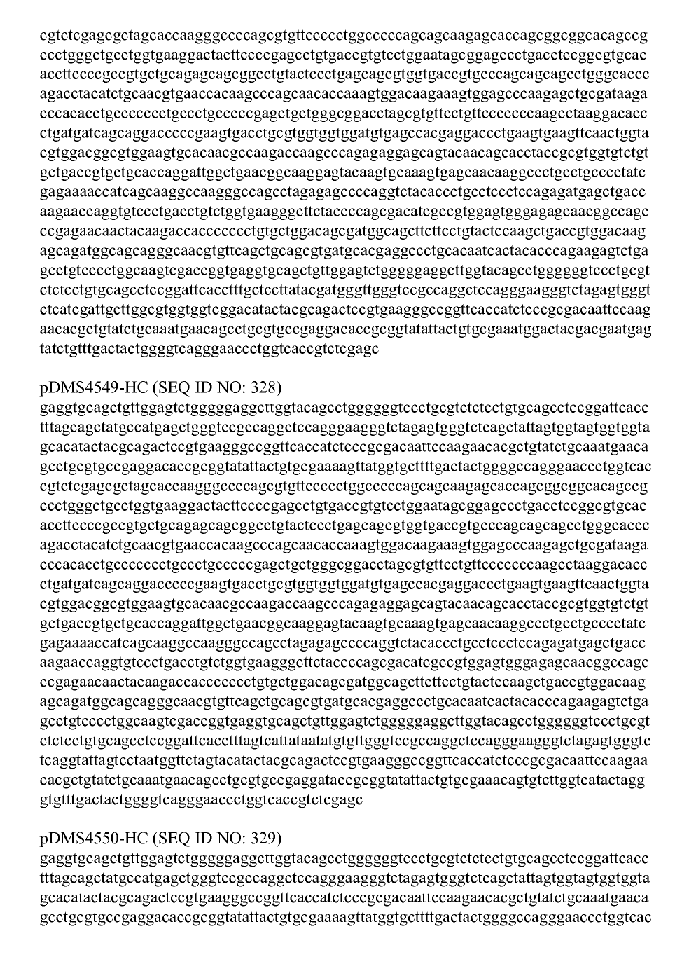

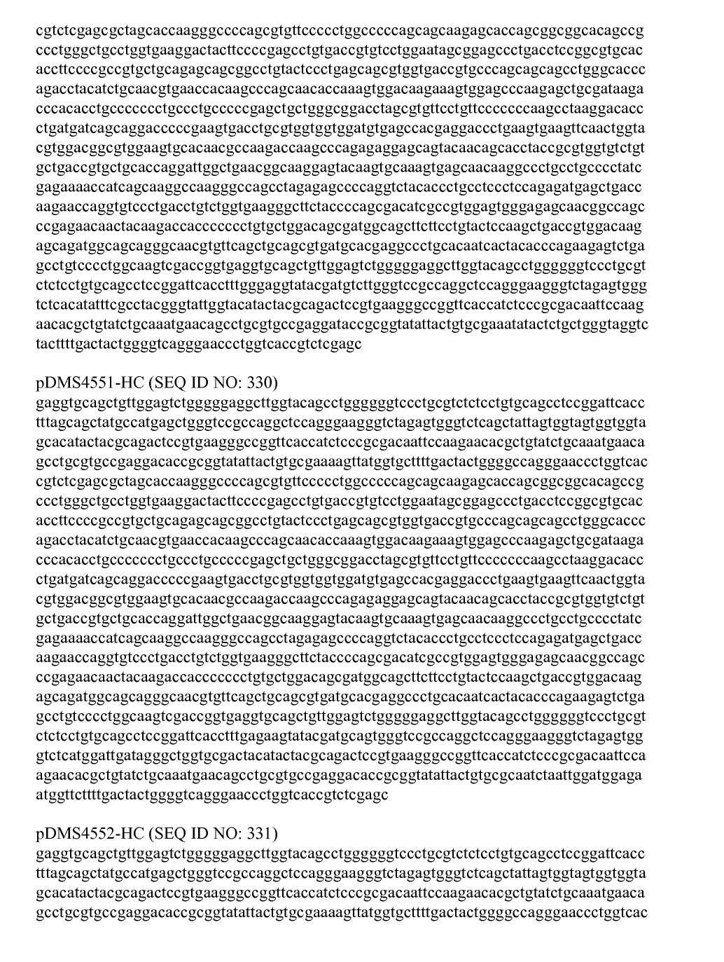

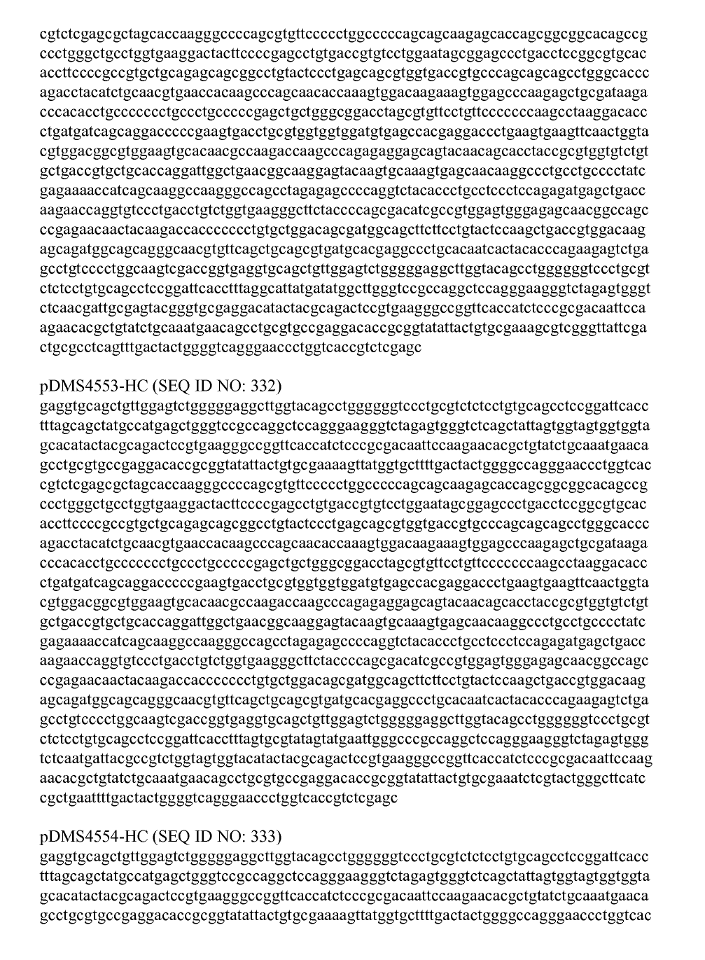

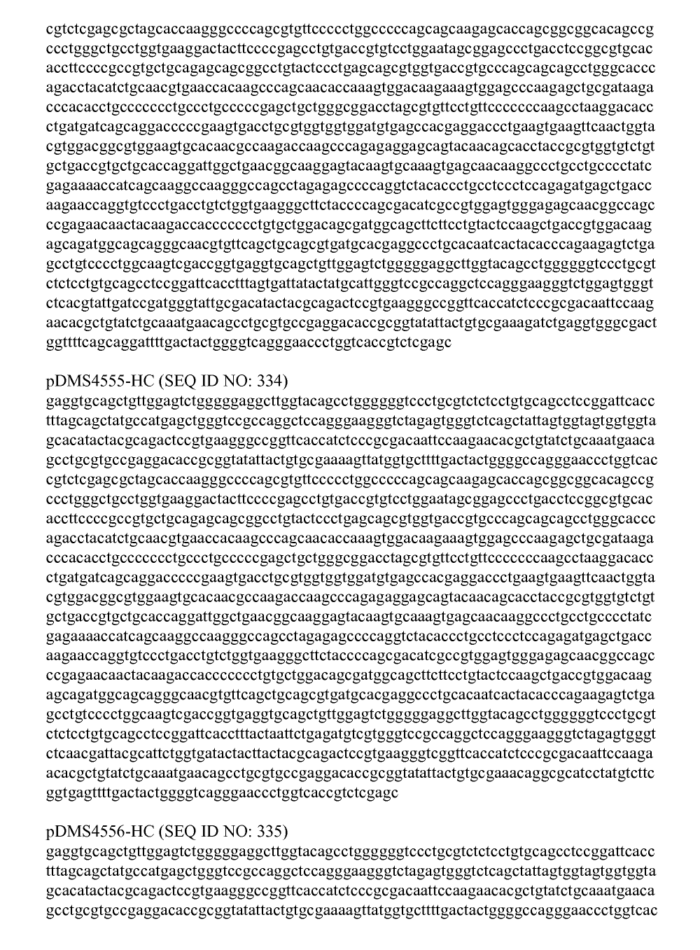

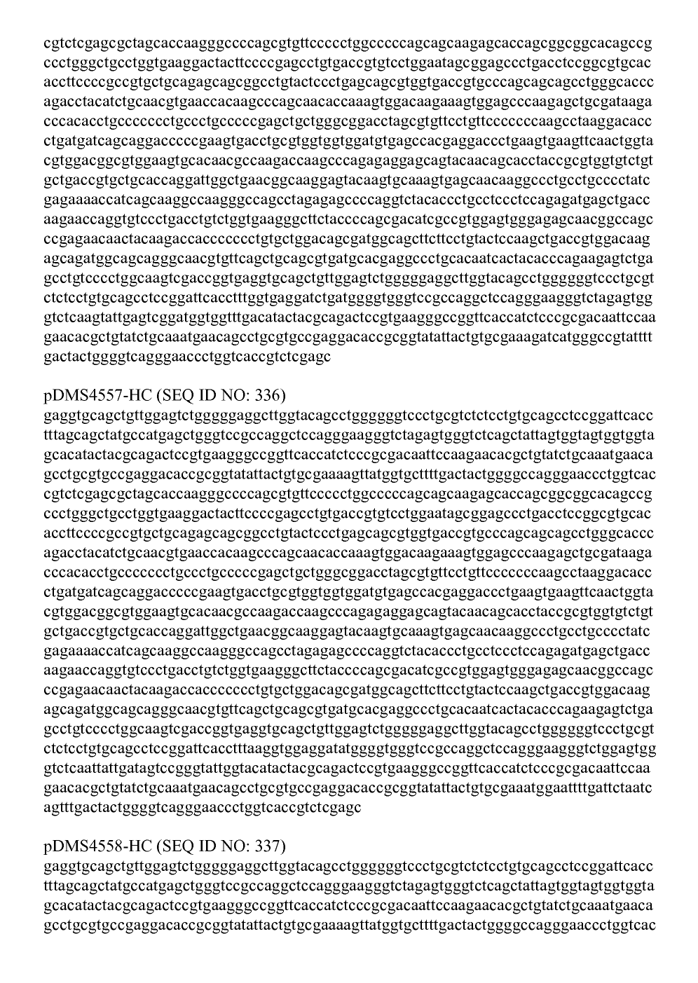

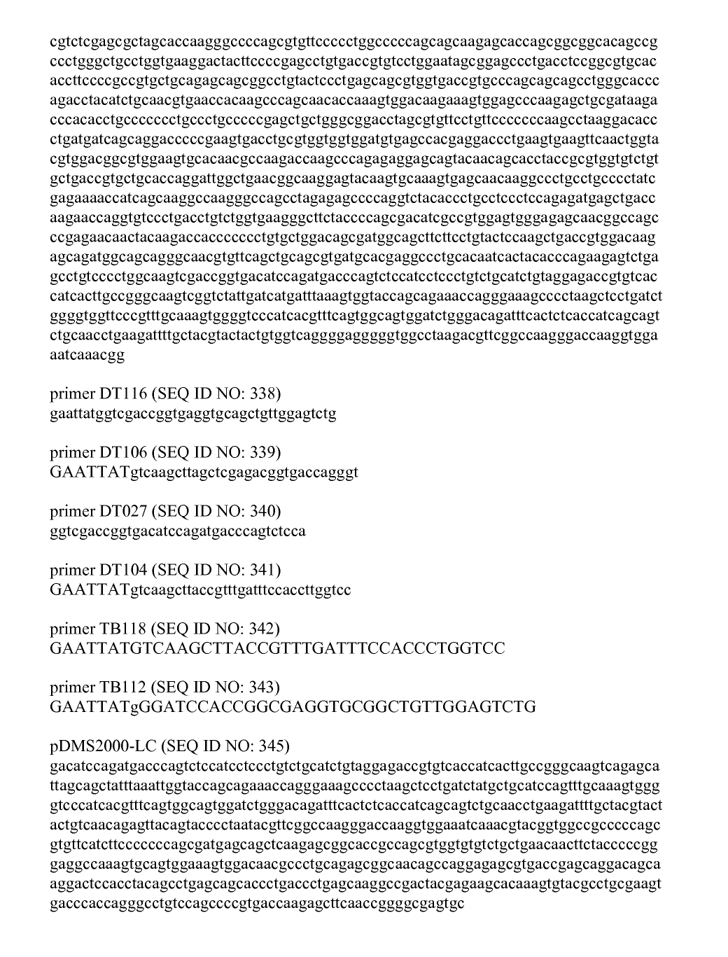

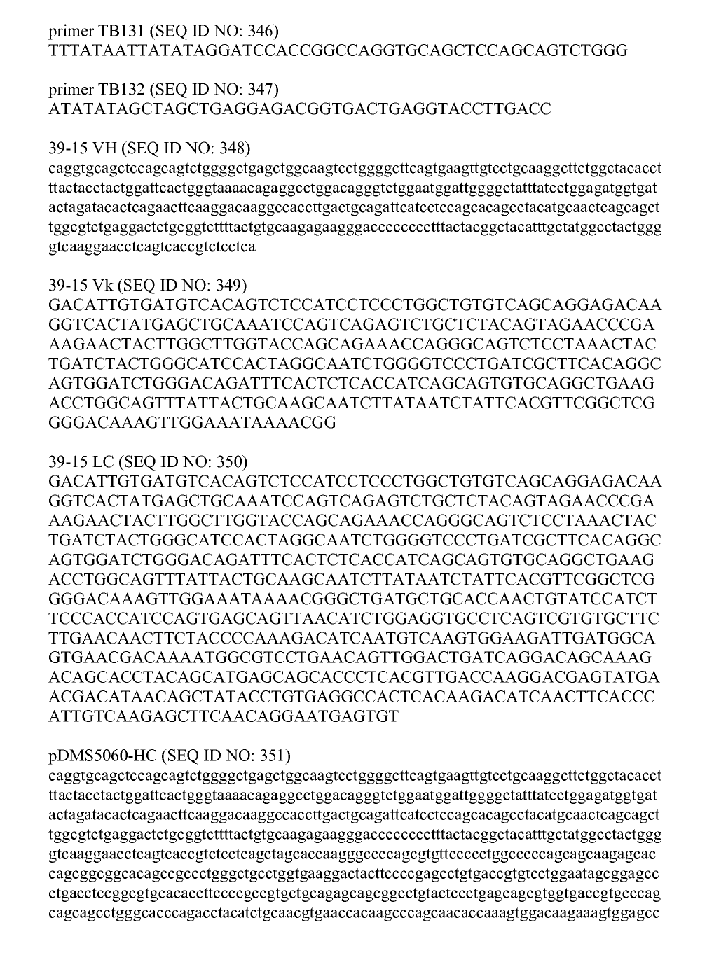

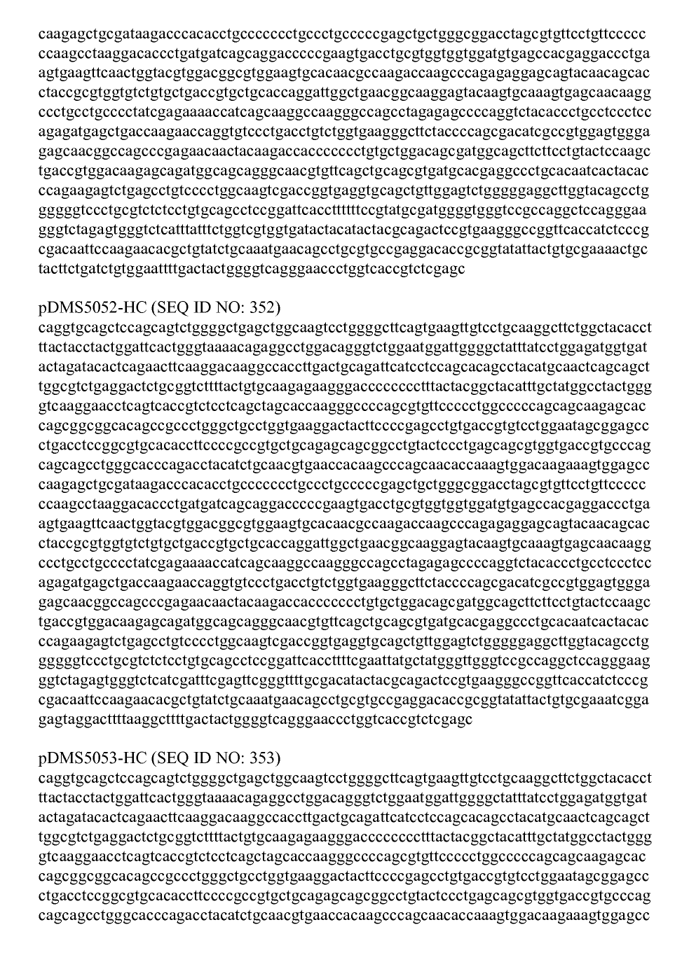

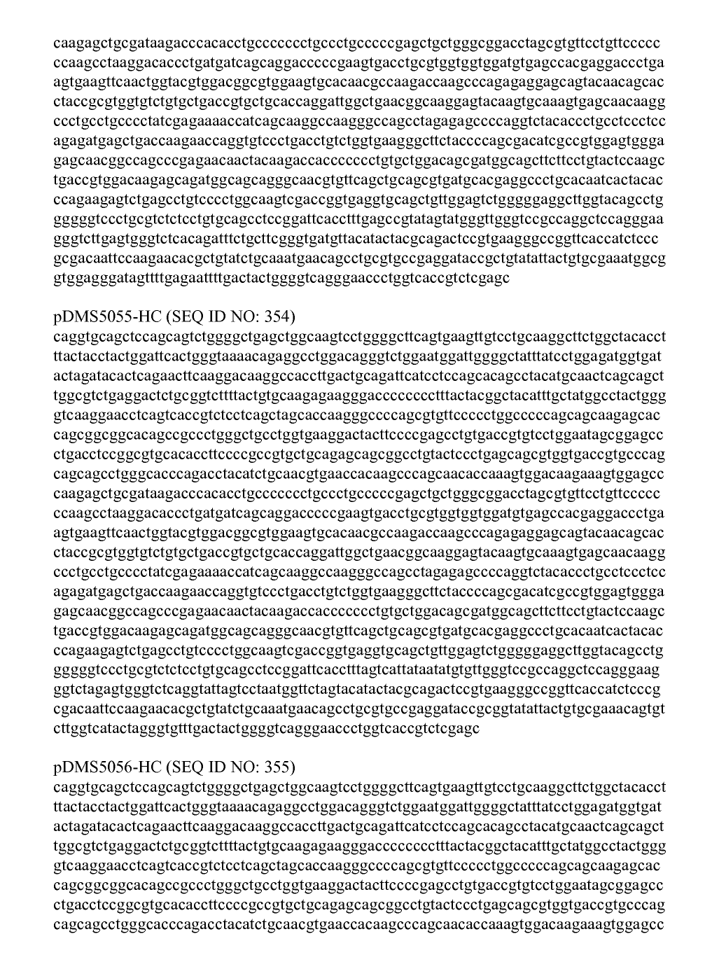

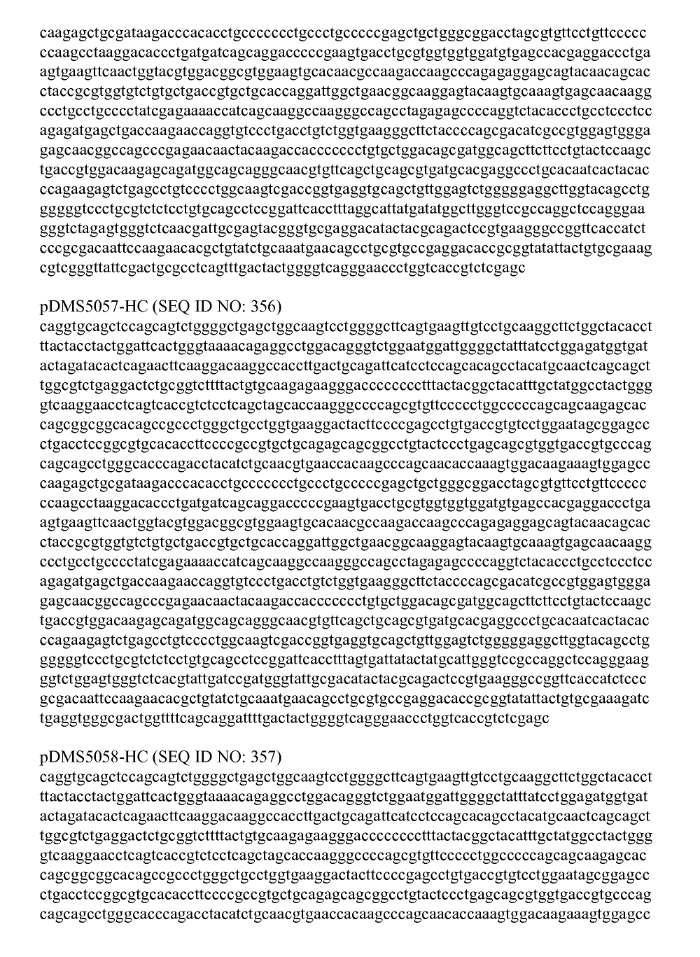

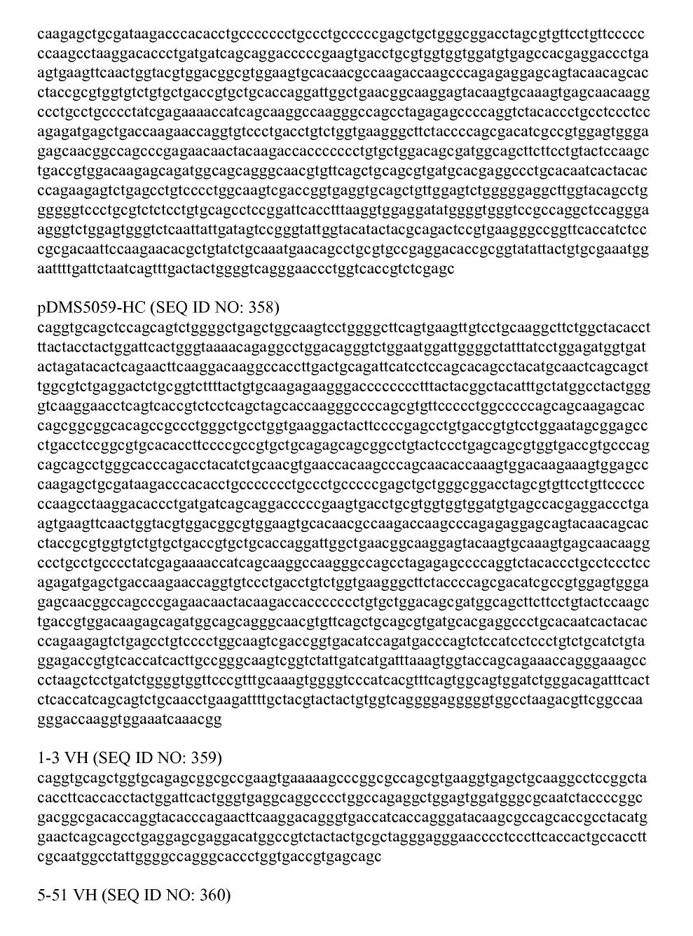

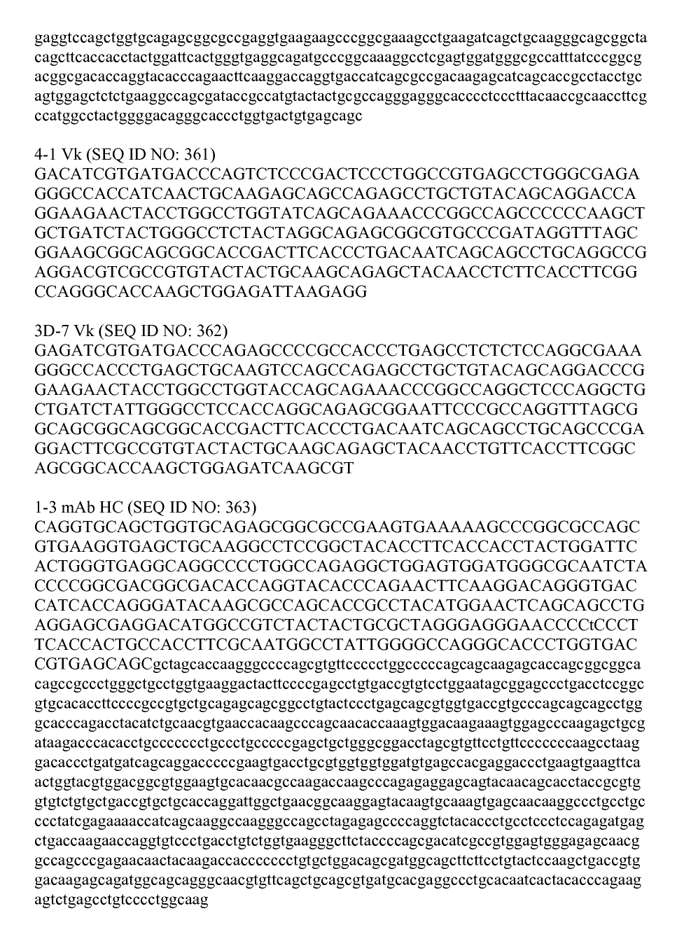

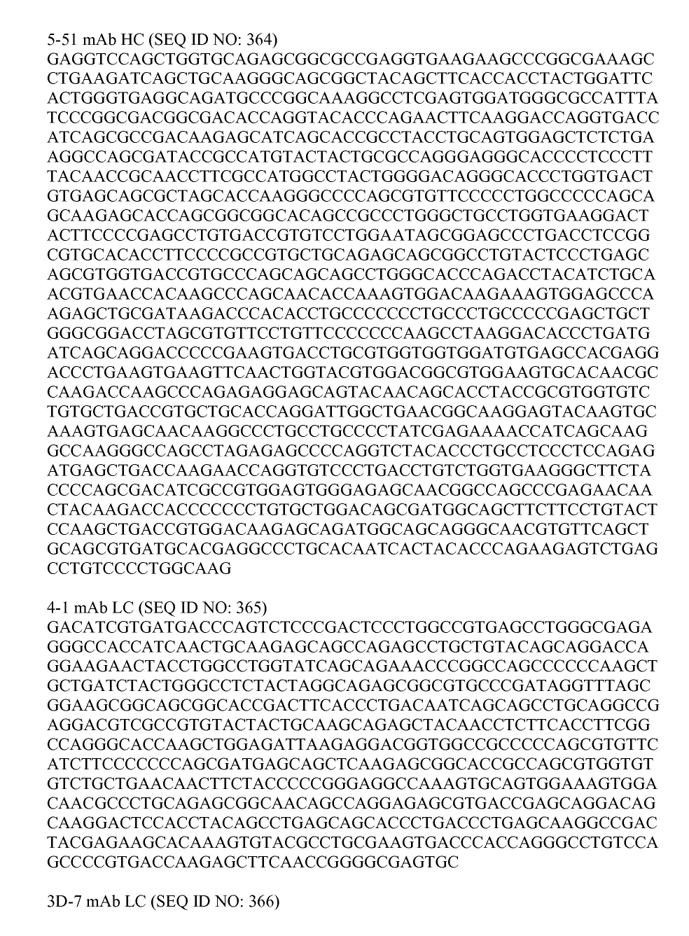

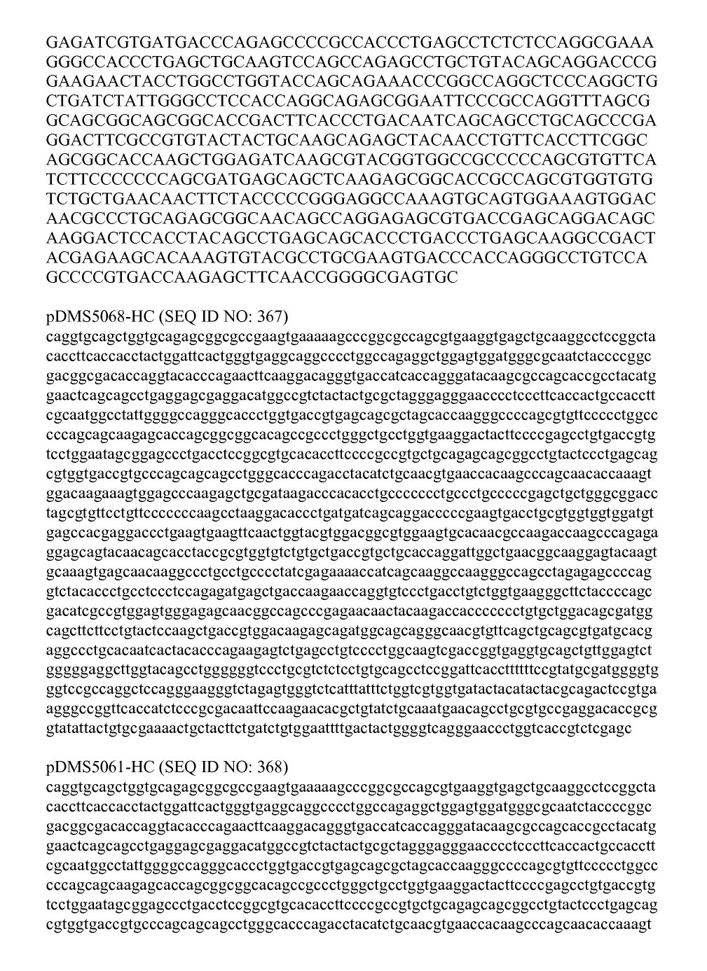

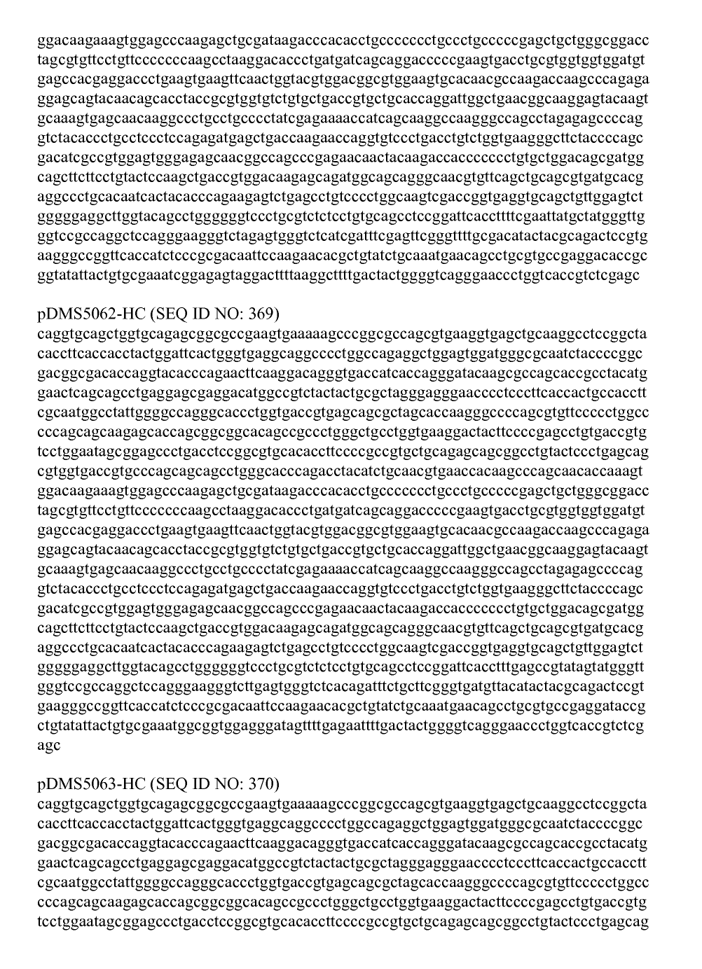

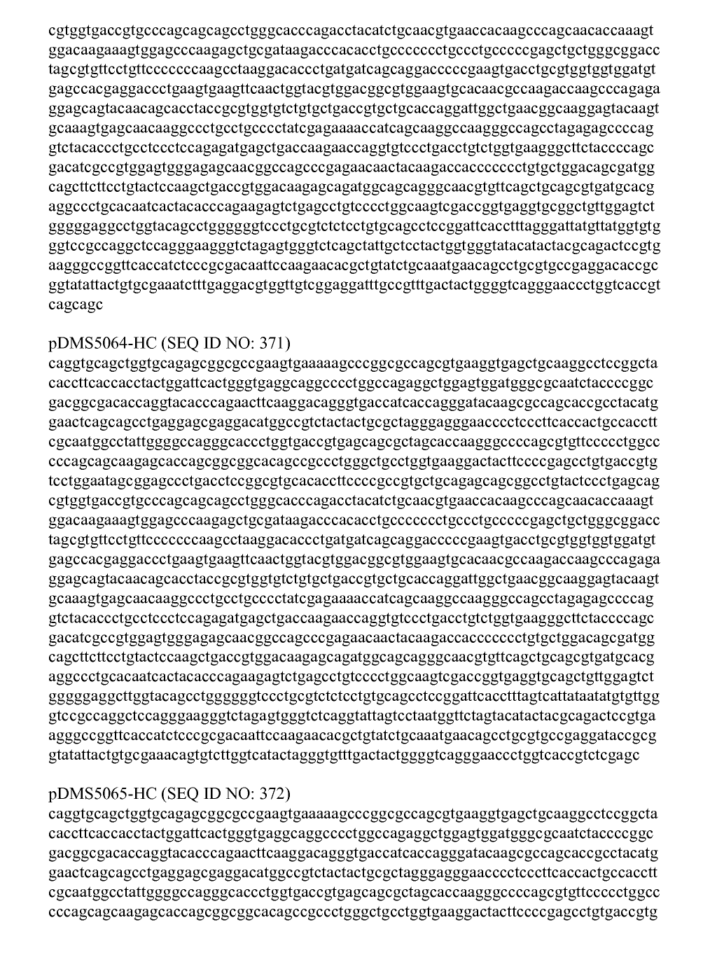

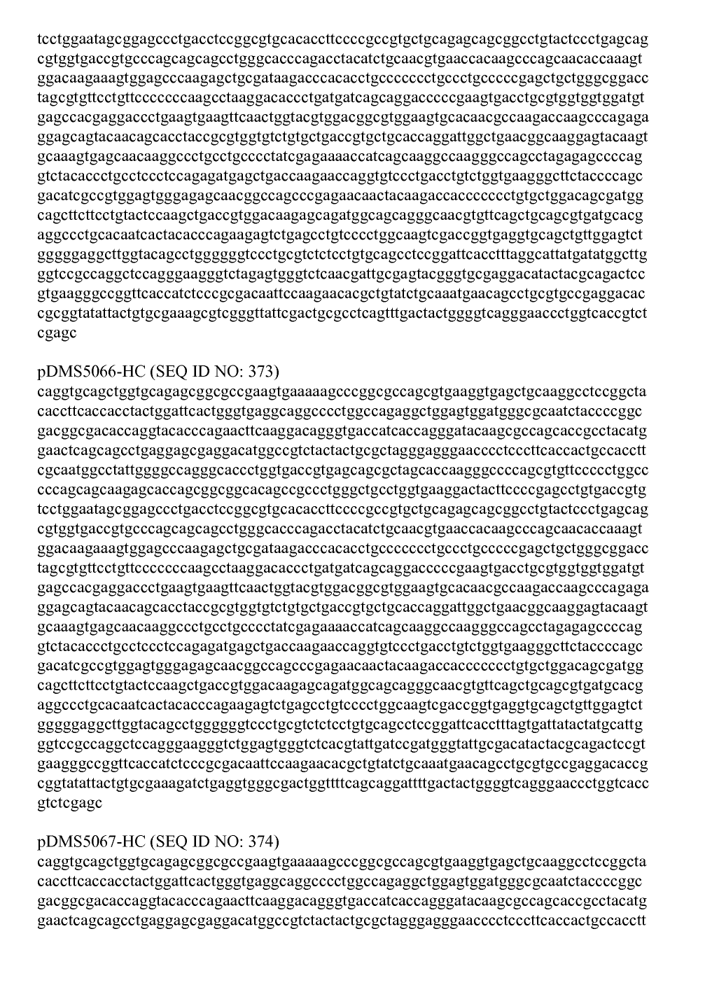

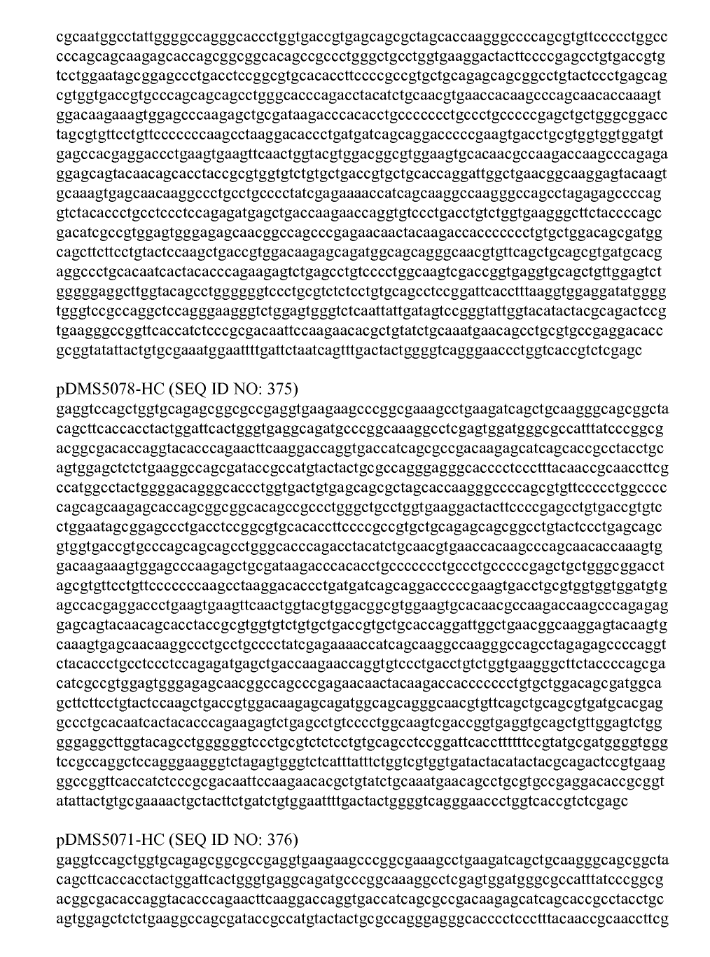

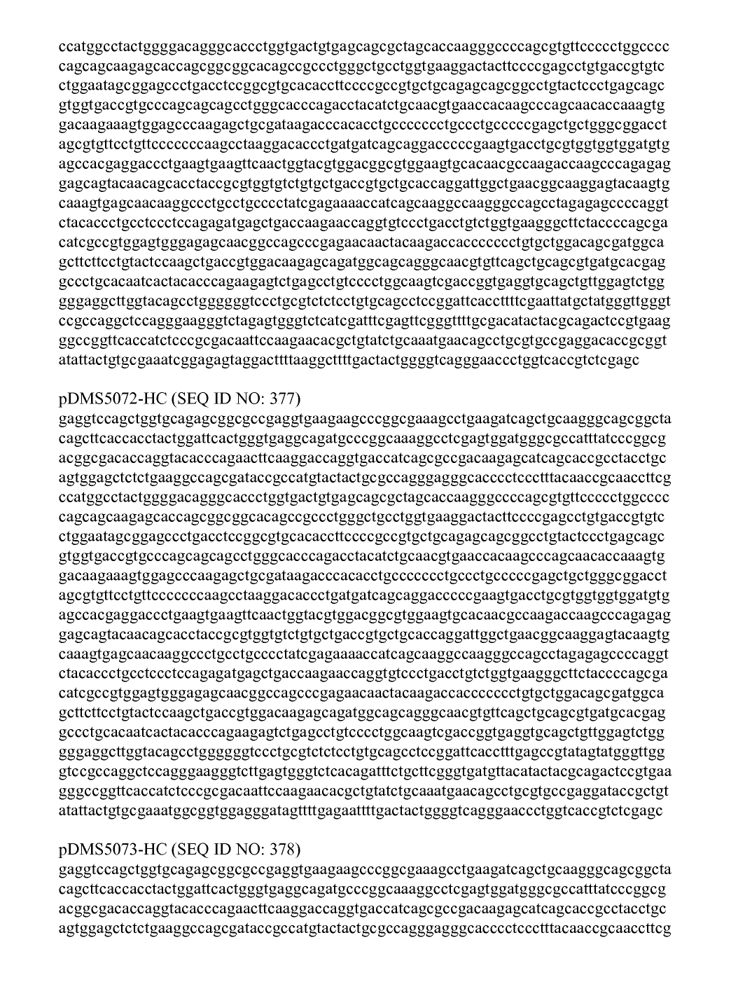

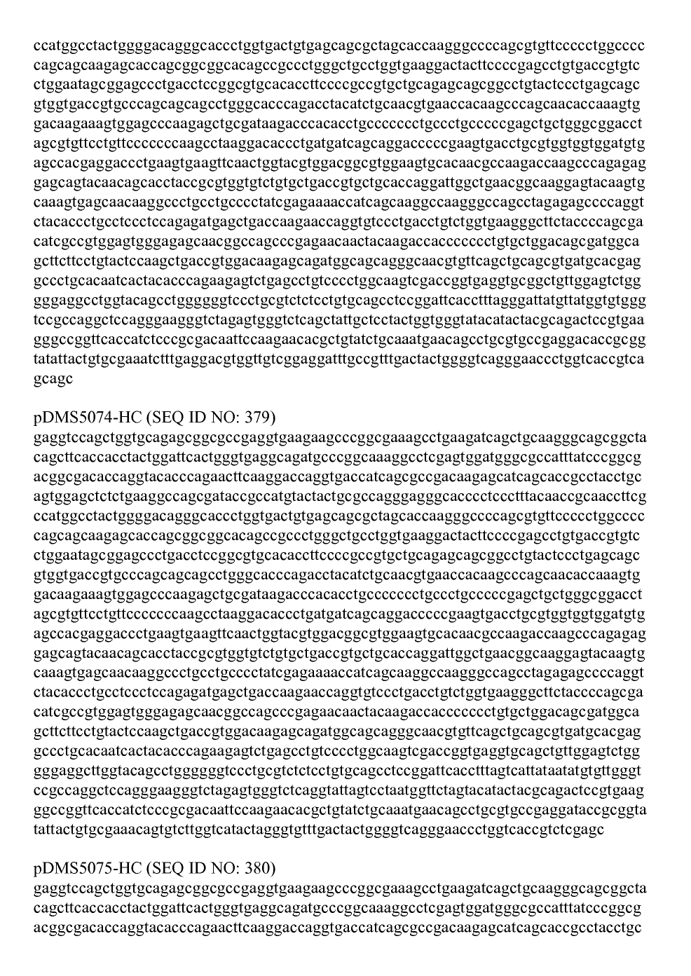

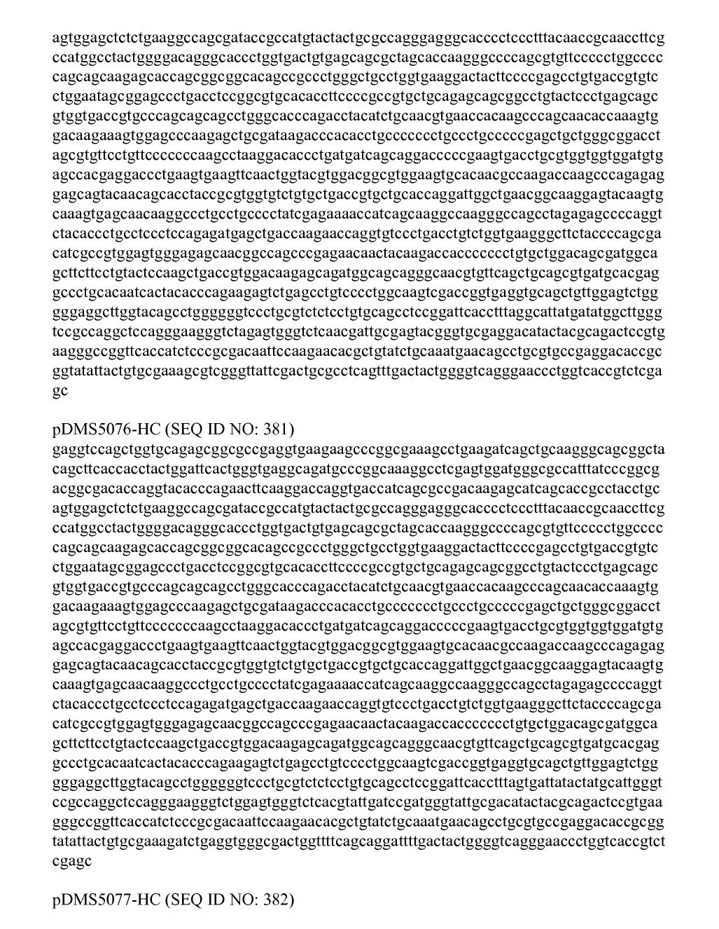

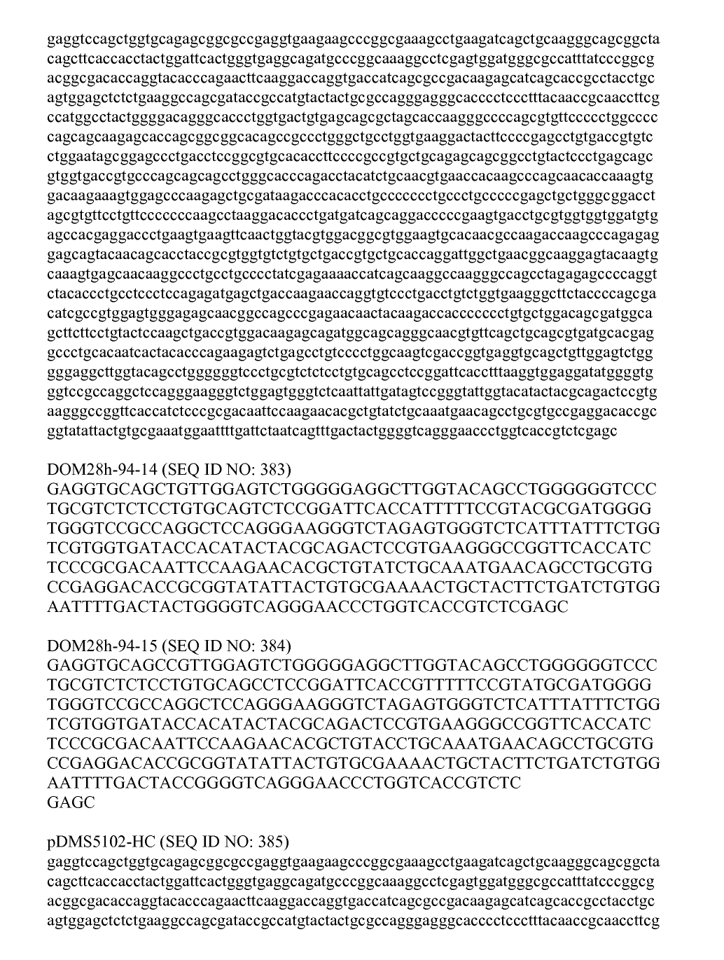

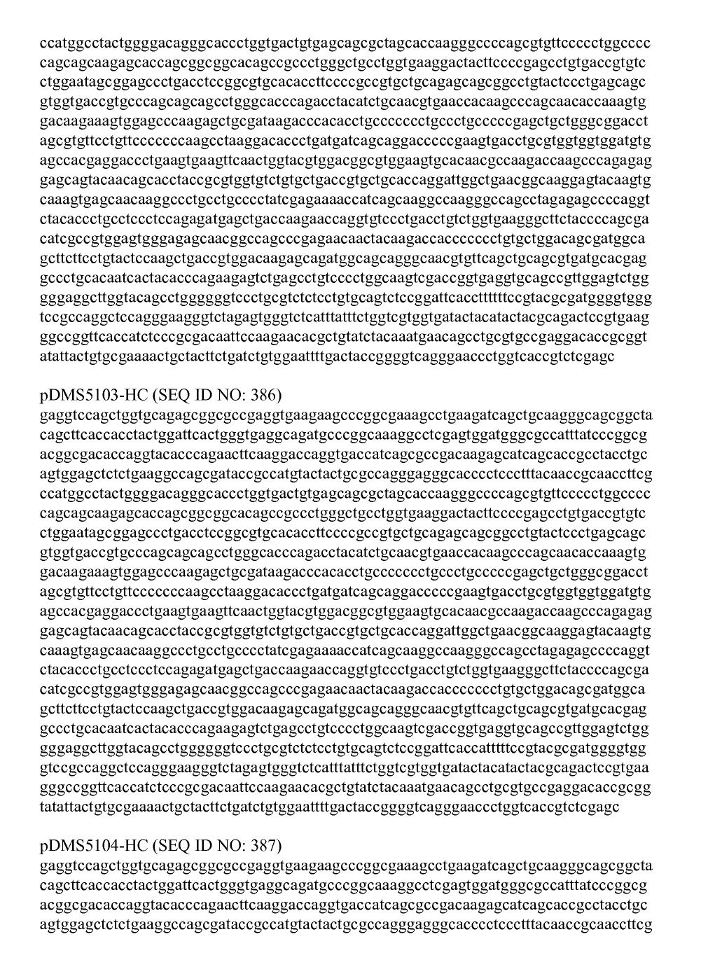

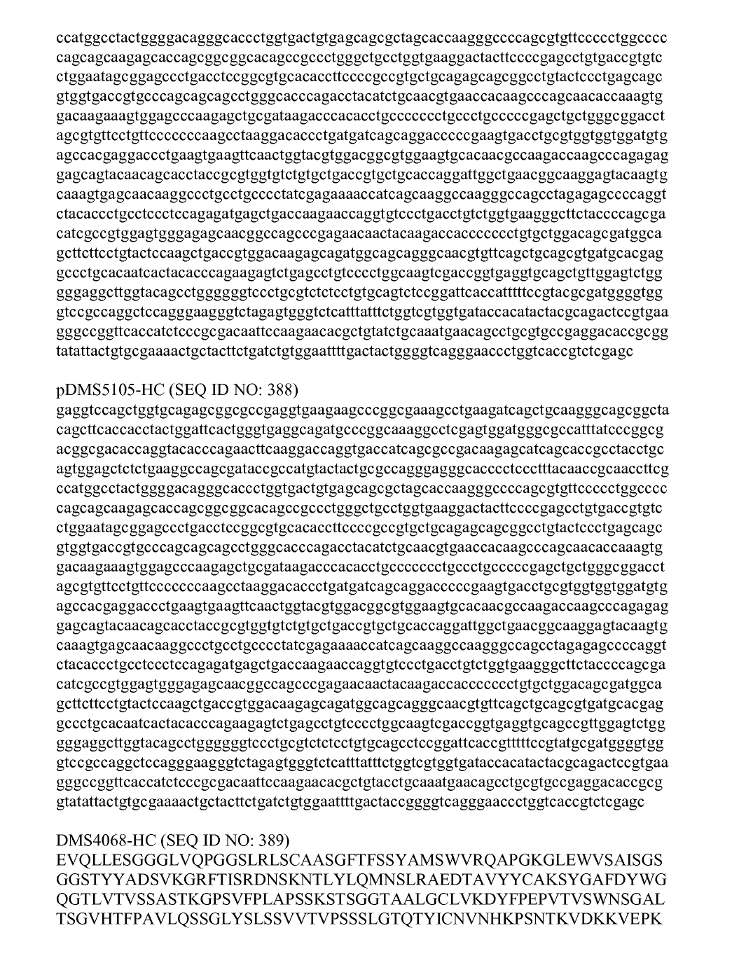

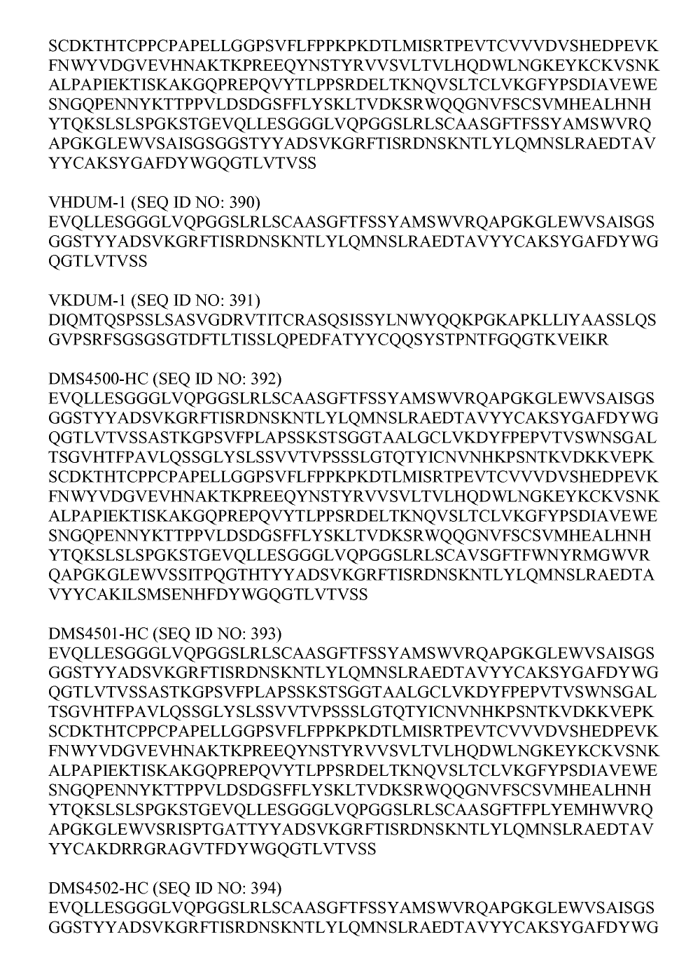

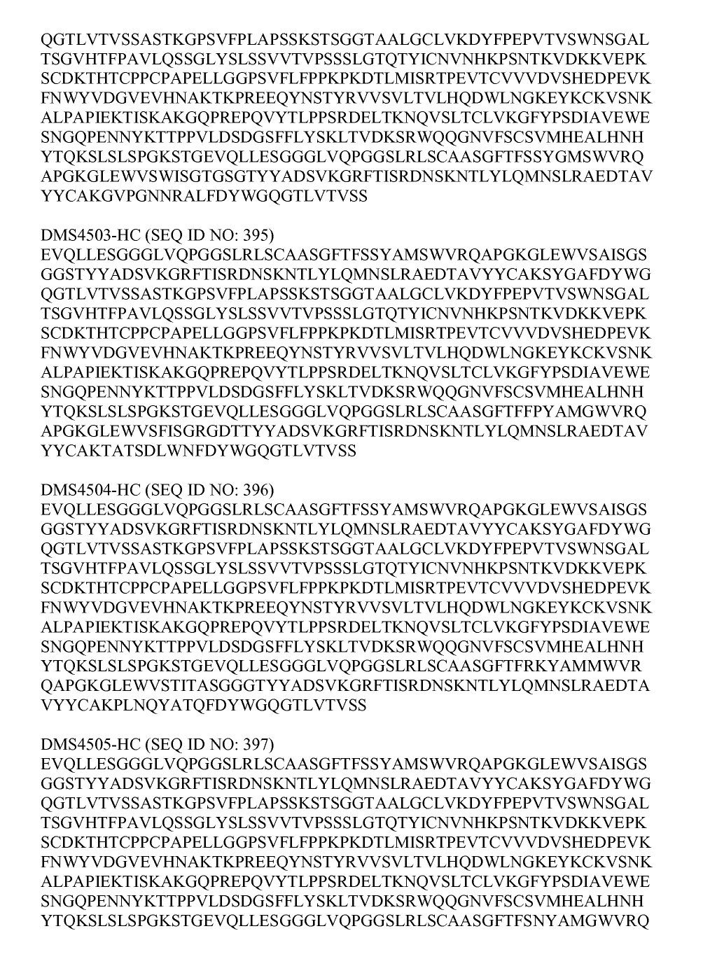

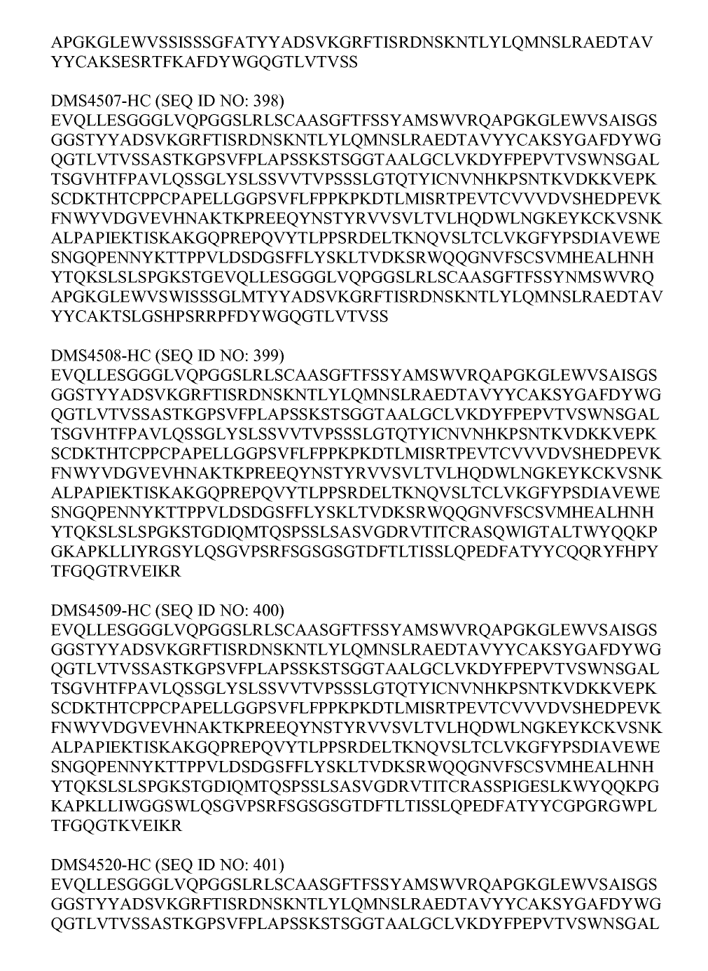

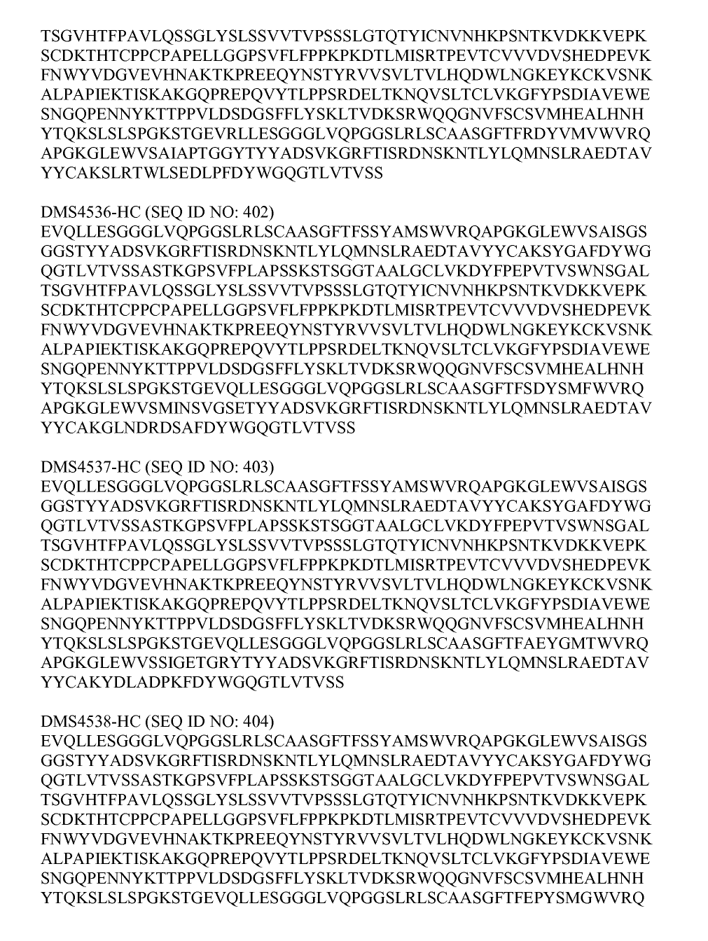

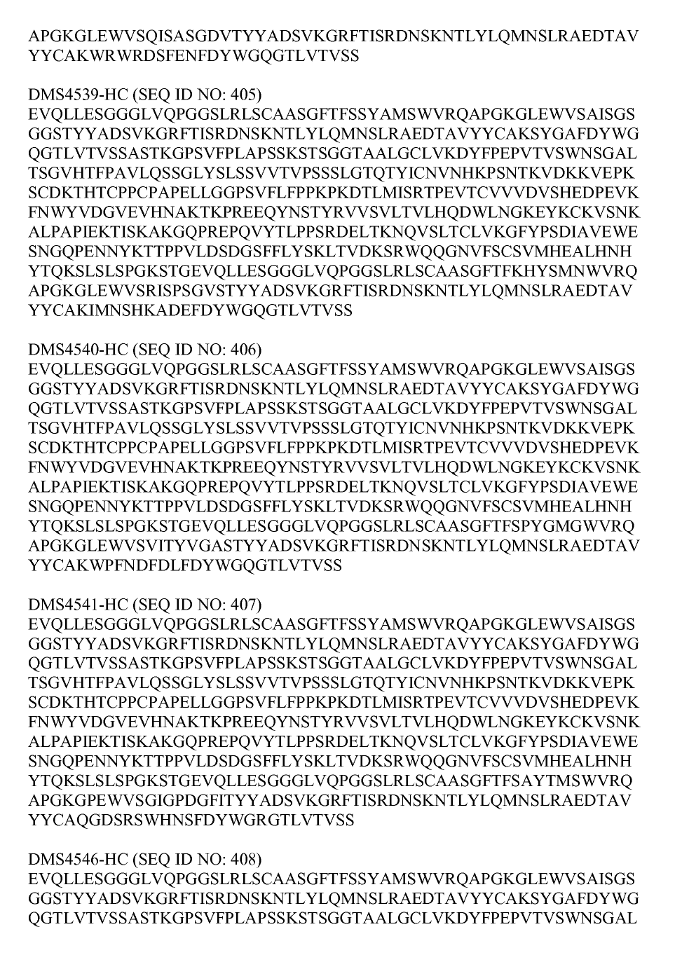

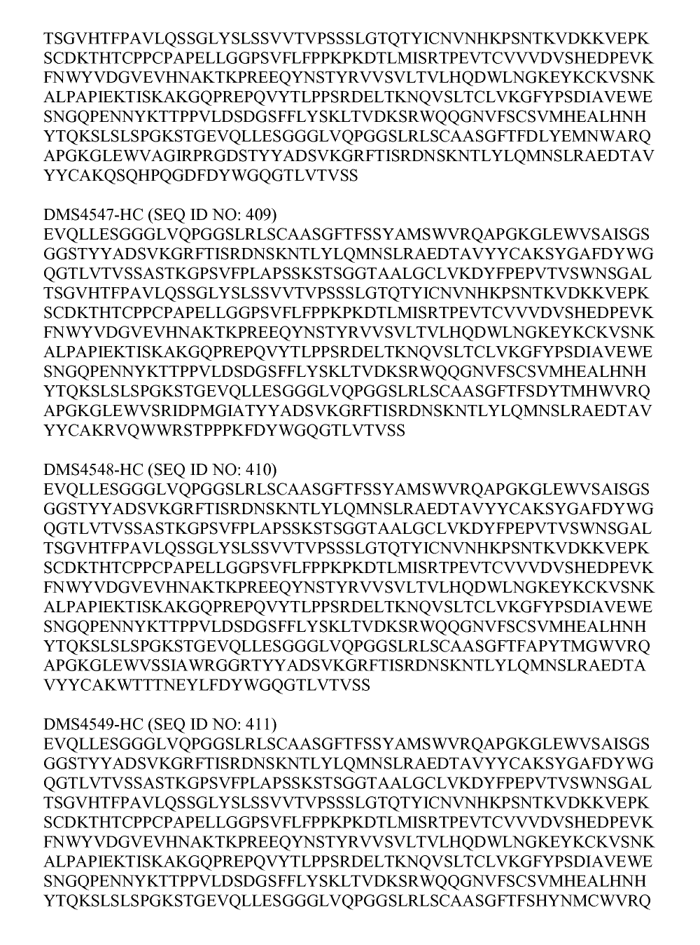

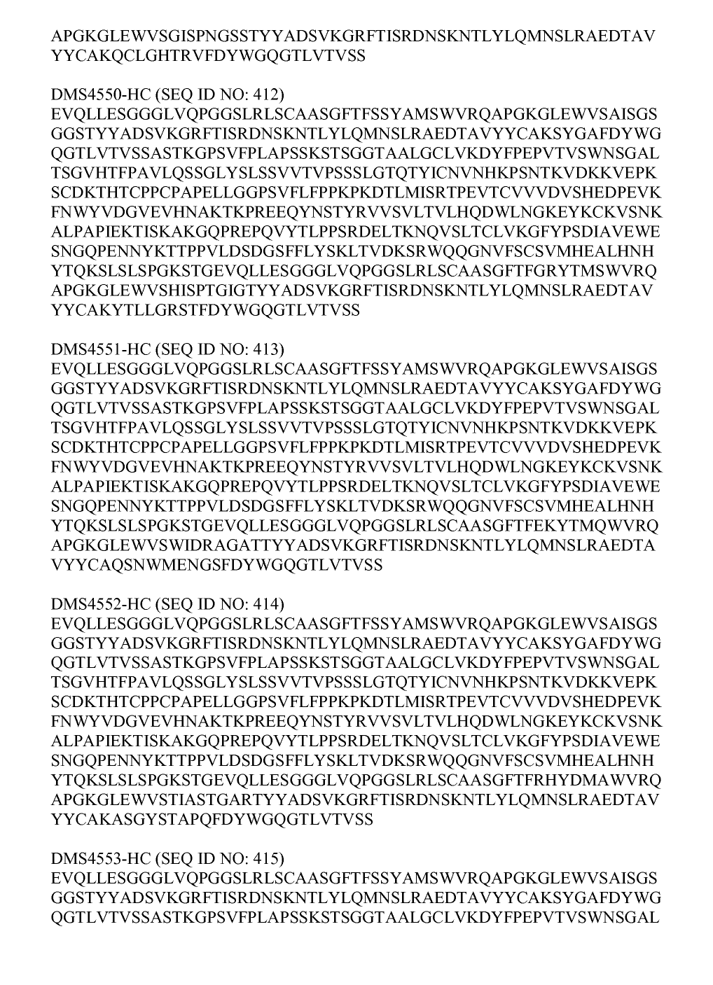

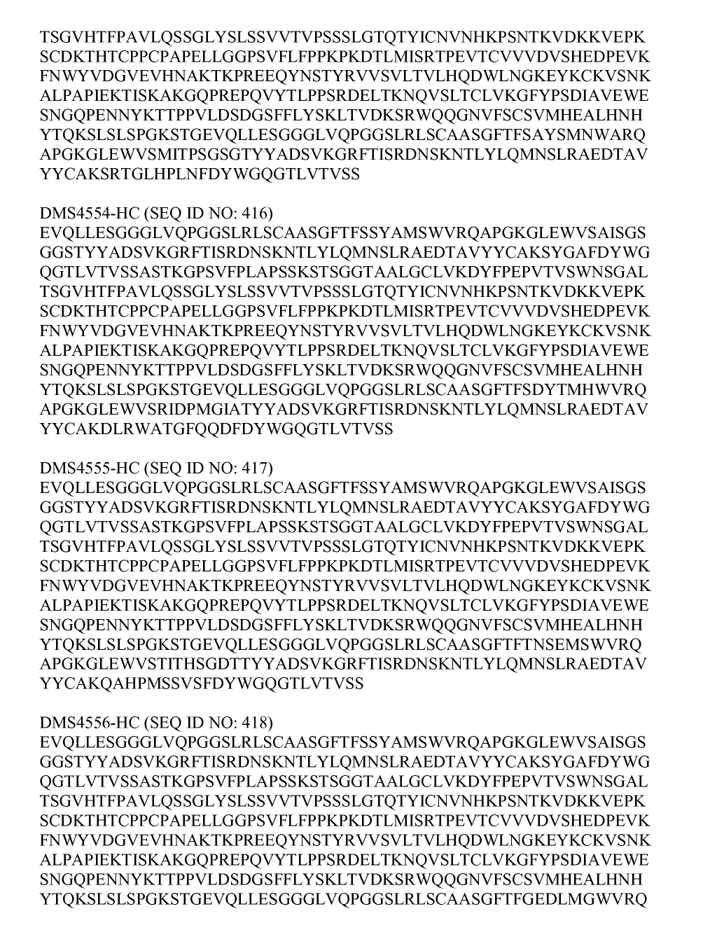

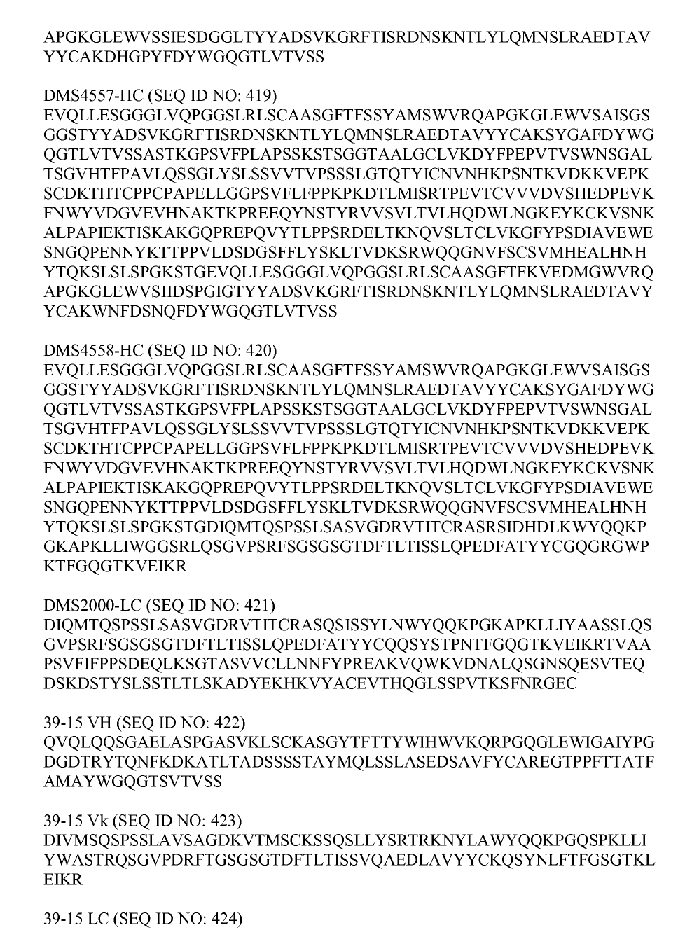

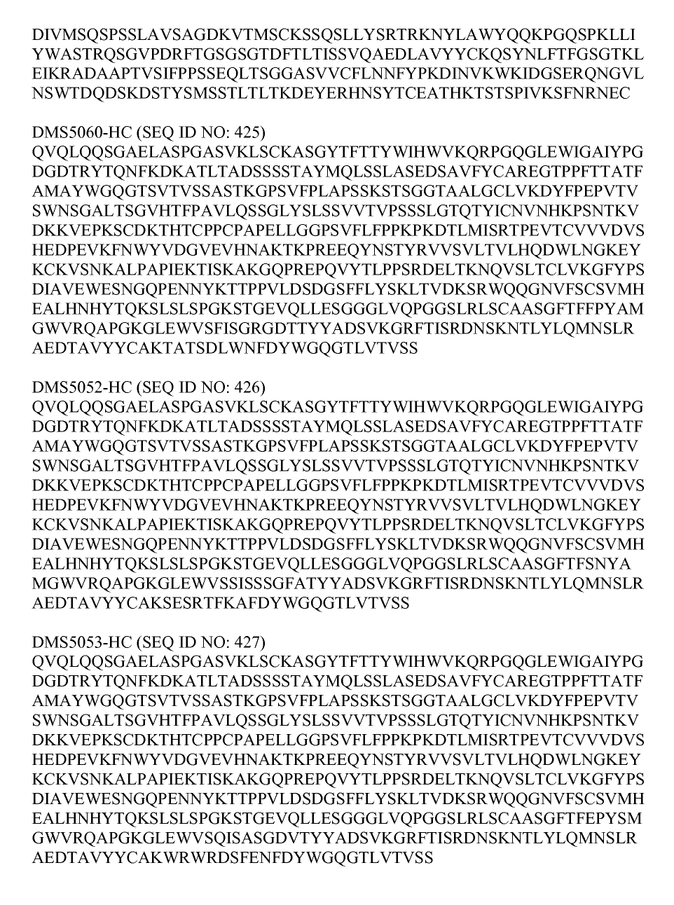

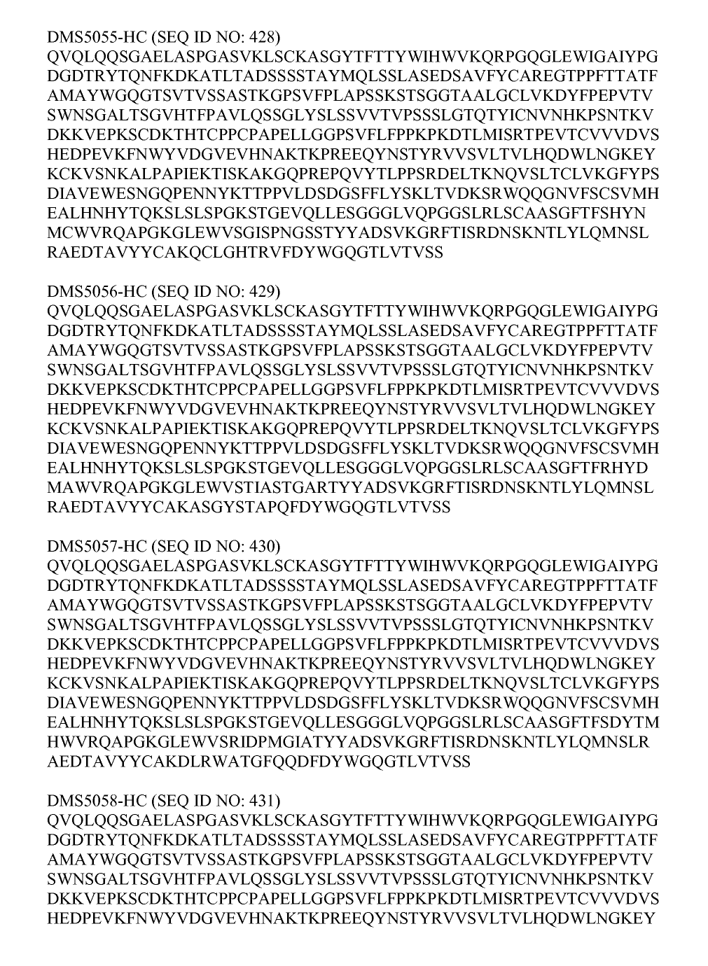

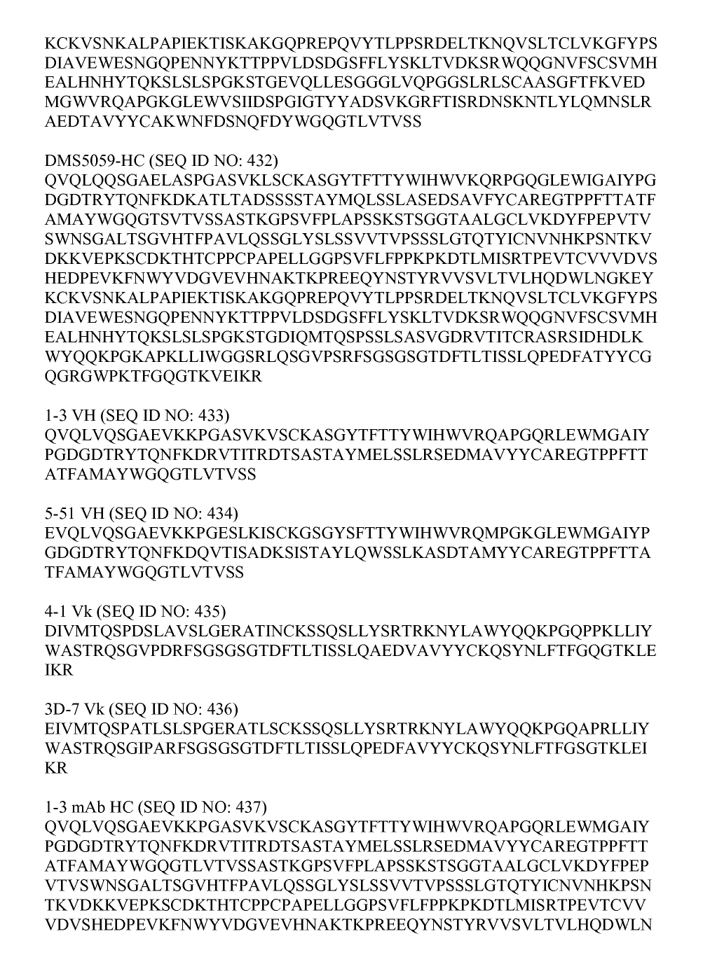

mAb-dAb molecules were constructed by combining a standard mAb light chain and modified mAb heavy chains where dAbs were fused to the C-termini. The overall architecture of bispecific mAb-dAbs, monospecific and format control molecules are illustrated on FIG. 23. All constant regions for mAb-dAbs described here were of the human IgG1 isotype.

The overall strategy to construct bispecific anti-vMLC/anti-c-kit mAb-dAbs was to first format anti-c-KIT dAbs from selections as mAb-dAb by fusion to a dummy mAb framework to generate dummy mAb-c-KIT dAb type mAb-dAbs. Both human and mouse c-kit binding dAbs were examined.

The dAbs with desired properties in that format were then formatted into a bispecific format where the mAb portion contained V domains from the anti-vMLC mouse mAb 39-15 to make the chimeric mAb-c-KIT dAb mAb-dAbs. Finally c-kit dAbs were combined with various humanized anti-vMLC mAbs. A list of the mAb-dAb constructs described herein is given in Table 24.

| | | Heavy Chain | | | | | Light Chain | | |
|---|---|---|---|---|---|---|---|---|---|
| | | Heavy | Constant | | | | Light | Constant | |
| mAb-dAb | Protein | Chain V | Heavy | | C-ter dAb | | Chain V | Light | |
| Type | DMS ID | domain | Regions | linker | Fusion | DNA ID: | domain | Regions | DNA ID: |
| Dummy | 4500 | VHDUM-1 | CH1-CH2-CH3 | STG | DOM28h-033 | pDMS4500-HC | VKDUM-1 | Ck | pDMS2000-LC |
| mAb-cKIT | 4501 | | | | DOM28h-066 | pDMS4501-HC | | | |
| dAb | 4502 | | | | DOM28h-084 | pDMS4502-HC | | | |
| | 4503 | | | | DOM28h-094 | pDMS4503-HC | | | |
| | 4504 | | | | DOM28h-110 | pDMS4504-HC | | | |
| | 4505 | | | | DOM28m-007 | pDMS4505-HC | | | |
| | 4507 | | | | DOM28m-052 | pDMS4507-HC | | | |
| | 4508 | | | | DOM28h-005 | pDMS4508-HC | | | |
| | 4509 | | | | DOM28h-043 | pDMS4509-HC | | | |
| | 4520 | | | ST | DOM28m-023 | pDMS4520-HC | | | |
| | 4536 | | | STG | DOM28m-004 | pDMS4536-HC | | | |
| | 4537 | | | | DOM28m-008 | pDMS4537-HC | | | |
| | 4538 | | | | DOM28m-017 | pDMS4538-HC | | | |
| | 4539 | | | | DOM28m-073 | pDMS4539-HC | | | |
| | 4540 | | | | DOM28h-113 | pDMS4540-HC | | | |
| | 4541 | | | | DOM28h-115 | pDMS4541-HC | | | |
| | 4546 | | | | DOM28m-019 | pDMS4546-HC | | | |
| | 4547 | | | | DOM28m-024 | pDMS4547-HC | | | |
| | 4548 | | | | DOM28m-103 | pDMS4548-HC | | | |
| | 4549 | | | | DOM28m-104 | pDMS4549-HC | | | |
| | 4550 | | | | DOM28m-105 | pDMS4550-HC | | | |
| | 4551 | | | | DOM28m-106 | pDMS4551-HC | | | |
| | 4552 | | | | DOM28m-107 | pDMS4552-HC | | | |
| | 4553 | | | | DOM28m-108 | pDMS4553-HC | | | |
| | 4554 | | | | DOM28m-109 | pDMS4554-HC | | | |
| | 4555 | | | | DOM28m-110 | pDMS4555-HC | | | |
| | 4556 | | | | DOM28m-111 | pDMS4556-HC | | | |
| | 4557 | | | | DOM28m-112 | pDMS4557-HC | | | |
| | 4558 | | | | DOM28m-114 | pDMS4558-HC | | | |
| Chimeric | 5060 | 39-15 VH | CH1-CH2-CH3 | STG | DOM28h-094 | pDMS5060-HC | 39-15 Vk | Ck | 39-15 Vk-hCk |
| MLC mAb- | 5052 | | | | DOM28m-007 | pDMS5052-HC | | | |
| cKIT dAb | 5053 | | | | DOM28m-017 | pDMS5053-HC | | | |
| | 5055 | | | | DOM28m-104 | pDMS5055-HC | | | |
| | 5056 | | | | DOM28m-107 | pDMS5056-HC | | | |
| | 5057 | | | | DOM28m-109 | pDMS5057-HC | | | |
| | 5058 | | | | DOM28m-112 | pDMS5058-HC | | | |
| | 5059 | | | | DOM28m-114 | pDMS5059-HC | | | |
| Humanized | 5068 | 1-3 VH | CH1-CH2-CH3 | STG | DOM28h-094 | pDMS5068-HC | 4-1 Vk | Ck | 4-1 LC |
| MLC mAb- | 5061 | | | | DOM28m-007 | pDMS5061-HC | | | |
| cKIT dAb | 5062 | | | | DOM28m-017 | pDMS5062-HC | | | |
| | 5078 | 5-51 VH | | | DOM28h-094 | pDMS5078-HC | 3D-7 | Ck | 3D7 LC |
| | 5071 | | | | DOM28m-007 | pDMS5071-HC | | | |
| | 5072 | | | | DOM28m-017 | pDMS5072-HC | | | |
| | 5073 | | | ST | DOM28m-023 | pDMS5073-HC | | | |
| | 5074 | | | STG | DOM28m-104 | pDMS5074-HC | | | |
| | 5075 | | | | DOM28m-107 | pDMS5075-HC | | | |
| | 5076 | | | | DOM28m-109 | pDMS5076-HC | | | |
| | 5077 | | | | DOM28m-112 | pDMS5077-HC | | | |
| | 5088 | 1-3 VH | | | DOM28h-094 | pDMS5068-HC | 4-1 Vk | Ck | 4-1 LC |
| | 5081 | | | | DOM28m-007 | pDMS5061-HC | | | |
| | 5082 | | | | DOM28m-017 | pDMS5062-HC | | | |
| | 5098 | 5-51 VH | | | DOM28h-094 | pDMS5078-HC | 3D-7 | Ck | 3D7 LC |
| | 5091 | | | | DOM28m-007 | pDMS5071-HC | | | |
| | 5092 | | | | DOM28m-017 | pDMS5072-HC | | | |
| | 5102 | 5-51 VH | CH1-CH2-CH3 | STG | DOM28h-94-11 | pDMS5102-HC | 4-1 Vk | Ck | 4-1 LC |
| | 5103 | | | | DOM28h-94-13 | pDMS5103-HC | | | |
| | 5104 | | | | DOM28h-94-14 | pDMS5104-HC | | | |
| | 5105 | | | | DOM28h-94-15 | pDMS5105-HC | | | |
| Control | 4068 | VHDUM-1 | CH1-CH2-CH3 | STG | VHDUM-1 | pDMS4068-HC | VKDUM-1 | Ck | pDMS2000-LC |
| mAb-dAb | 4069 | VHDUM-1 | | | VKDUM-1 | pDMS4069-HC | | | |
| | 4572 | VHDUM-2 | | | VHDUM-2 | pDMS4572-HC | | | |
| | 4573 | 39-15 VH | | | VHDUM-2 | pDMS4573-HC | | | |
| | 4579 | 5-51 VH | | | VHDUM-2 | pDMS4579-HC | 4-1 Vk | Ck | 4-1 LC |

5.1.2 Dummy mAb-cKIT dAb mAb-dAbs

### a) Construction

Dummy mAb-dAb heavy chain and (mAb) light chain expression cassette templates had been previously constructed (as described in WO2009/068649). Restriction sites for cloning are shown below in FIG. 24. cKIT dAbs were fused to the c-terminus of the heavy chain using SalI and HindIII cloning sites. It should be noted that introduction of the site results in SalI coding a 3-residue linker of ‘STG’ (serine, threonine, glycine) between the mAb and the dAb. The starting template for cloning heavy chain of mAb-dAbs was pDMS4068-HC (SEQ ID NO: 306) which had been constructed as follows: VHDUM-1 (SEQ ID NO: 307) was amplified by PCR using primers DT116 (SEQ ID NO: 338) and DT106 (SEQ ID NO: 339). This PCR product was inserted using SalI and HindIII ends into a vector backbone which contained VHDUM-1_CH1_CH2_CH3 in the expression cassette (FIG. 24) to make pDMS4068-HC. This construct contained the VHDUM-1 (VH dummy) dAb (SEQ ID NO: 307) in place of the “VH” between BamHI-NheI and “dAb” SalI-HindIII sites as illustrated in FIG. 24.

The light chain contained VKDUM-1 (Vk dummy, SEQ ID NO: 308) between SalI and BsiWI sites in place of “VL” as illustrated in FIG. 24.

29 different cKIT dAbs were constructed in this format. In brief, cKIT dAb inserts were amplified by PCR and ligated into pDMS4068-HC backbone which had the c-terminal dAb excised using SalI-HindIII sites. Primers used for PCR are Primer DT116: (SEQ ID NO: 338); Primer DT106: (SEQ ID NO: 339); Primer DT027: (SEQ ID NO: 340); Primer DT104: (SED ID NO: 341); Primer TB118: (SEQ ID NO: 342) and Primer TB112: (SEQ ID NO: 343). Primer pairs were chosen according to class of dAb (VH or Vk) and whether or not the dAbs contained framework changes on primer annealing regions.

Sequence verified clones were selected and large scale plasmid DNA preps were made using Qiagen Maxi or Mega Prep kits following the manufacturer's protocols. mAb-dAbs were expressed in mammalian HEK293-6E cells (Biocat #120363) using transient transfection techniques by co-transfection of light chain (SEQ ID NO: 345) and heavy chains (SEQ ID NOs: 309 to 337). It was observed that all clones containing the DOM28m-23 dAb (pDMS4520-HC) had a shortened linker ‘ST’ (serine, threonine) rather than ‘STG’ as described above.

### b) Purification, SDS-PAGE Analysis and SEC Analysis

Dummy mAb-cKIT dAb mAb-dAbs were purified from clarified expression supernatants using Protein-A affinity chromatography according to established protocols. Concentrations of purified samples were determined by spectrophotometry from measurements of light absorbance at 280 nm.

SDS-PAGE analysis of the purified sample showed non-reduced samples running at ˜175 kDa whilst reduced samples showed two bands running at ˜25 and ˜60 kDa corresponding light chain and dAb-fused heavy chain respectively.

For size exclusion chromatography (SEC) analysis the mAb-dAb concentrations were adjusted to 1.0 mg·ml⁻¹ and applied onto an S-200 10/300 GL column (GE Healthcare) attached to an HPLC system pre-equilibrated and running in PBS at 0.5 ml/min.

mAb-dAbs were scored on a scale of 5 to 1 (5=good; 1=poor) taking into account (a) total elution as % of sample applied to column, (b) area of main peak as % of all peaks, (c) % elution in main peak and (d) symmetry of main peak as criteria for performance on SEC.

c) BIAcore Affinity to cKIT and Cell Binding Properties

Mouse cKIT was coupled on a BIAcore CM5 chip (GE Healthcare) in the presence of acetate pH 4.5 to aim for approximately 1750 RUs on the chip (“high density chip”). A second, lower density chip was made for mcKIT on a streptavidin coated BIAcore chip (GE Healthcare) to aim for approximately 750 RUs on the chip.

Using a positive control anti mouse cKIT antibody (2B8), the high density chip only gave a response of 100 for 2B8 instead of the theoretical response of 4000 RUs. This indicates that the number of active cKIT molecules on the surface of a BIAcore chip is less than theoretically expected.

Rat cKIT was coupled to a CM5 chip on acetate pH 5.5, and as with mcKIT was able to bind positive rat cKIT binding dAbs. Finally, Myosin Light Chain was coupled to a streptavidin chip.

mAbdAbs were diluted to 1 uM in HBS-EP buffer (GE Healthcare) and injected across the different BIAcore chips. The chip was regenerated by a single injection of glycine at pH 2.

### d) Cell Binding

mAb-dAbs were tested for binding to c-kit expressed on the cell surface of c-kit positive mouse (MC/9 and EML) and human (HEL-92.1.7 and KU812) cell lines. The negative control cell lines which did not express c-kit were Jurkat and HeLa. Briefly, cells were counted and washed in PBS with 2.5% FBS (FACS buffer). Cells were added to a 96-well plate at a density of ˜5×10⁵ cells per well. The cells were incubated with the mAb-dAbs at the appropriate concentration for 1 hr at 4° C. The cells were spun and washed with FACS buffer 2 times. The cells were then incubated with 2 μg·ml⁻¹ anti-human FAb Alexa-488 antibody (Invitrogen #A11013) for 40 mins at 4° C. The cells were washed again with FACS buffer and resuspended in 200 μl PBS with 50 nM Topro-3 Iodide dead-cell dye (Invitrogen #T3605) before analysis on the Canto II flow cytometer using Flow Jo software as described above.

### e) Results

The majority of dummy mAb-cKIT dAb mAb-dAbs did bind to either human and/or mouse c-kit expressing cells. The dummy mAb-cKIT dAb mAb-dAbs were ranked based on desired properties (SEC, BIAcore affinity and cell binding). Mouse specific c-KIT binding dAbs exhibiting desirable properties were progressed for examination in pre-clinical studies. 8 cKIT dAbs namely DOM28h-094, DOM28m-007, DOM28m-017, DOM28m-023, DOM28m-104, DOM28m-107, DOM28m-109 and DOM28m-112 (all VH dAbs) were selected for progression to the next stages to make bispecific mAb-dAbs by fusing these dAbs to a chimeric anti-MLC mAb. DOM28m-114 was also picked as a Vk dAb.

5.1.3 Chimeric anti-vMLC mAb-cKIT dAb mAb-dAbs

a) Construction

Chimeric anti-MLC mAb-cKIT dAb mAb-dAbs were made by taking the dummy mAb-cKIT dAb mAb-dAbs described above and swapping the VH and Vk dummy from the Fab Variable domains for the VH and Vk regions from the anti-vMLC mouse mAb 39-15 (SEQ ID NO: 348 and 349 respectively) (as described above in Example 3).

The VH dummy coding region from the dummy mAb-cKIT dAb was excised by digestion with BamHI and NheI; the 39-15 VH insert was amplified by PCR using the primers TB131 and TB132 (SEQ ID NO: 346 and 347) and ligated into the aforementioned backbones using BamHI and NheI ends. The resulting 7 chimeric heavy chains are summarised in Table 24 (SEQ ID NOs: 351 to 358).

The 39-15 chimeric light chain expression cassette (39-15 VK—human Ck, SEQ ID NO: 350) was constructed as described above (see Example 3).

Sequence verified clones were selected and large scale plasmid DNA preps were made using Qiagen Maxi or Mega Prep kits following the manufacturer's protocols. mAb-dAbs were expressed in mammalian HEK293-6E cells using transient transfection techniques by co-transfection of light chain (SEQ ID NO: 350) and heavy chains (SEQ IDs 351 to 358).

b) Chimeric anti-vMLC mAb-cKIT dAb mAb-dAbs were purified and analysed by SDS-PAGE, as described above. Non-reduced samples ran at ˜175 kDa whilst reduced samples showed two bands running at ˜25 and ˜60 kDa corresponding to light chain and dAb-fused heavy chain respectively.

SEC analysis was performed as described above scoring on a scale of 5 to 1 where 5 is good and 1 is poor.

TABLE 25
Summary of SEC results for chimeric
anti-vMLC mAb-cKIT dAb mAb-dAbs

| DMS | |
|---|---|
| ID | SEC Rating |
| 5052 | 2 |
| 5053 | 3 |
| 5055 | 2 |
| 5056 | 1 |
| 5057 | 3 |
| 5058 | 3 |
| 5059 | 1 |
| 5060 | 3 |

c) BIAcore studies were carried out as described above.

d) Cell Binding (“full format”)

mAb-dAbs were tested for binding to c-KIT expressed on the cell surface of mouse cell lines substantially as described above but with a modified detection system. The molecules were detected for binding to c-KIT via the MLC mAb portion. Briefly, following incubation with the chimera or humanised MLC mAb-cKIT dAb molecule, the cells were then incubated with 0.5 μg·ml⁻¹ biotinylated mouse vMLC antigen for 30 mins at 4° C. The cells were washed again with FACS buffer 2 times and incubated with 1 μg·ml⁻¹ strep-PE for 30 mins at 4° C. The cells were then washed in FACS buffer again and resuspended in 200 μl PBS with 50 nM Topro-3 Iodide dead-cell dye before analysis on the Canto II flow cytometer. All data was analysed using Flow Jo software.

The c-KIT positive cell lines included MC/9 and EML mouse cells. The negative control cell line was human HeLa cells. This experiment confirmed that all the chimeric MLC mAb-cKIT dAb molecules bound to c-kit expressed on mouse cells.

TABLE 26
Cell binding and BIAcore results for chimeric
anti-vMLC mAb-cKIT dAb mAb-dAbs (BF = bad fit).

| | | | BIAcore | | BIAcore | | BIAcore | | | |
|---|---|---|---|---|---|---|---|---|---|---|
| | | | KD for mouse | | KD for mouse | | KD for human | | BIAcore | |
| mAb-dAb | HELA | MC/9 | c-kit | | c-kit-Fc | | c-kit | | KD for MLC | |
| 5052 | NO | YES | 80 | nM | 36 | nM | 27 | nM | 47 | pM [BF] |
| 5053 | NO | YES | 58 | nM [BF] | 103 | nM [BF] | 36 | μM | 3 | pM [BF] |
| 5055 | NO | YES | 30 | nM | 22 | nM [BF] | 72 | nM | 124 | pM [BF] |
| 5056 | NO | YES | 19 | nM | 13 | nM [BF] | NB | | 48 | pM [BF] |
| 5057 | NO | YES | 205 | nM | 71 | nM | 318 | nM | 116 | pM [BF] |
| 5058 | NO | YES | 46 | nM | 26 | nM | NB | | 42 | pM [BF] |
| 5059 | YES | YES | 12 | nM [BF] | 10 | nM [BF] | 8 | nM [BF] | 10 | nM [BF] |

| | | | | | | | | | |
|---|---|---|---|---|---|---|---|---|---|
| 5060 | NO | YES | 14 | nM | 13 | nM | 36 | nM | 26M [BF] |

NB = no binding by BIAcore; YES = cell binding; NO = no cell binding.

e) Dummy and Chimera mAb-dAb/C-Kit Phosphorylation Assay in the Presence and Absence of SCF

To confirm that the mAb-dAbs did not interfere with SCF signalling via c-KIT, the mAb-dAbs (DMS 4069, 4503, 4505, 4538, 4549, 4552, 4554, 4557, 4558, 4572, 4573, 5060, 5052, 5053, 5055, 5056, 5057 and 5058) were tested in the mouse c-KIT phosphorylation MSD assay in MC/9 and EML cells. This assay was carried out as described above, except that 500 ng/ml mouse SCF was added to the cells and the chimera MLC mAb was included to control for any off-target effects caused by the mAb portion of the chimera MLC mAb-cKIT dAbs.

None of the molecules inhibited the phosphorylation of c-kit by SCF to baseline levels. There was also no significant effect on c-kit phosphorylation in the absence of SCF.

5.1.4 Humanized Anti-vMLC mAb-cKIT dAb mAb-dAbs
a) Construction of Humanized anti-vMLC mAb-cKIT dAb mAb-dAbs

Humanized anti-MLC mAb-cKIT dAb mAb-dAbs were made by taking the dummy mAb-cKIT dAb mAb-dAbs described above and swapping the VH and Vk dummy from the Fab Variable domains for the VH and Vk regions from the humanized anti-vMLC mAbs as described above in Example 3. 2 humanized VH sequences; 1-3 and 5-51 (SEQ ID NO: 359 and 360 respectively); and 2 humanized Vk sequences; 4-1 and 3D-7 (SEQ ID NO: 361 and 362 respectively); were combined to make 4 different humanized anti-MLC VH-Vk pairings.

Humanized anti-MLC mAb-cKIT dAb heavy chain expression cassettes were constructed by taking pDMS4503-HC, pDMS4505-HC, pDMS4538-HC, pDMS4549-HC, pDMS4552-HC, pDMS4554-HC, pDMS4557-HC and pDMS4520-HC (Table 24) and swapping the VH dummy coding region with the humanized anti-MLC VH regions 1-3 and 5-51 from expression cassettes of 1-3 mAb heavy chain and 5-51 mAb heavy chain (SEQ ID NOs: 363 and 364 respectively).

Humanized anti-vMLC mAb-cKIT dAb heavy chains (except pDMS5063-HC and pDMS5073-HC) were constructed by excising humanized anti-MLC VH regions 1-3 and 5-51 with BamHI and NheI and ligating these excised VH inserts into pDMS4503-HC, pDMS4505-HC, pDMS4538-HC, pDMS4549-HC, pDMS4552-HC, pDMS4554-HC and pDMS4557-HC backbones which had the VH dummy coding region removed with BamHI and NheI. pDMS5063-HC and pDMS5073-HC were constructed by (a) excising the DOM28m-23 coding regions with SalI and HindIII from pDMS4520-HC; (b) excising CH1-CH2-CH3 coding regions from pDMS4068-HC (SEQ ID NO: 306) with BamHI and SalI; (c) removing the entire mAb-dAb HC coding region from pDMS4068-HC with BamHI and HindIII; and then ligating inserts from (a) and (b) with either 1-3 or 5-51 into the vector backbone from (c) in a 4-fragment ligation. Humanized anti-vMLC mAb-cKIT dAb mAb-dAb heavy chains having SEQ ID NOs: 367 to 382 were generated.

b) Determining Optimal Humanized Anti-vMLC VH-Vk Pairing to Construct Humanized Bispecific mAb-dAb

### i) Construction of Subset of 12 Different Test Parings

To determine the best humanized anti-vMLC VH-Vk pairing in terms of biophysical properties a selection of 6 different mAb-dAb heavy chains listed in Table 24 were combined with 4-1 and 3D7 light chains (4-1 VK—human Ck, SEQ ID NO: 365 and 3D7 VK—human Ck, SEQ ID NO: 366 respectively). The different pairings resulted in Humanized anti-vMLC mAb-cKIT dAb heavy chain and light chain pairings to give mAb-dAbs identified as DMS 5068, 5061, 5062, 5078, 5071 and 5072 as well as DMS 5088, 5081, 5082, 5098, 5091 and 5092 (see Table 24).

Sequence verified clones were selected and large scale plasmid DNA preps were made using Qiagen Maxi or Mega Prep kits following the manufacturer's protocols. mAb-dAbs were expressed in mammalian HEK293-6E cells using transient transfection techniques by co-transfection of pairings.

### ii) Purification and SEC Analysis of the Subset of 12 Different Pairings

The 12 humanized anti-vMLC mAb-cKIT dAb mAb-dAbs were purified from clarified expression supernatants using Protein-A affinity chromatography according to established protocols. SDS-PAGE analysis showed non-reduced samples running at ˜175 kDa whilst reduced samples showed two bands running at ˜25 and ˜60 kDa corresponding to light chain and dAb-fused heavy chain respectively. Under non-reducing conditions DMS5061, DMS5062 and DMS5068 show an additional high molecular weight band running at ˜260 kDa.

For size exclusion chromatography (SEC) analysis the mAb-dAb concentrations were adjusted to 0.5 mg·ml⁻¹ (with the exception of DMS5081, DMS5082, DMS5088, DMS5091 and DMS5092 which were run at 0.2, 0.2, 0.2, 0.1 and 0.4 mg·ml⁻¹ respectively) and applied onto an S-200 10/300 GL column (GE Healthcare) attached to an HPLC system pre-equilibrated and running in PBS at 0.5 ml/min.

Humanized anti-vMLC mAb-cKIT dAb mAb-dAbs were scored on a scale of 5 to 1 (5=good; 1=poor) taking into account (a) total elution as % of sample applied to column, (b) area of main peak as % of all peaks, (c) % elution in main peak and (d) symmetry of main peak as criteria for performance on SEC.

TABLE 27
Summary of SEC results for 12 different pairings
(— indicates a zero rating)

| mAb-dAb ID | SEC Rating |
|---|---|
| DMS5061 | 1 |
| DMS5062 | 3 |
| DMS5068 | 3 |
| DMS5071 | 1 |
| DMS5072 | 3 |
| DMS5078 | 3 |
| DMS5081 | 1 |
| DMS5082 | 1 |
| DMS5088 | 0 |
| DMS5091 | 0 |
| DMS5092 | 0 |
| DMS5098 | 0 |

iii) Selection Of Best Humanized Anti-vMLC VH-Vk Pairing Based On 12 Test Combinations

Out of the 12 combinations expressed the humanized anti-vMLC mAb-cKIT dAb molecules with the 5-51 VH and 4-1 Vk pairings, DMS5071, DMS5072 and DMS5078 gave the best SDS-PAGE and SEC results. Although DMS5061, DMS5062 and DMS5068 gave comparable to SEC ratings, the presence of the additional higher molecular weight band on non-reducing SDS-PAGE ruled out the 1-3 VH and 4-1 Vk pairings.

c) Expression, Purification and SEC/MALLS Analysis of Humanized Anti-vMLC mAb-cKIT dAb mAb-dAbs

Sequence verified clones of light and heavy chain constructs listed in Table 28 below were selected and large scale plasmid DNA preps were made using Qiagen Maxi or Mega Prep Kit following the manufacturer's protocols. mAb-dAbs were expressed in mammalian HEK293-6E cells using transient transfection techniques by co-transfection of light and heavy chains listed in Table 28.

TABLE 28
Humanized anti-vMLC mAb-cKIT dAb molecules with the
5-51 VH and 4-1 Vk pairings selected for expression

| anti- | | humanized MLC | humanized anti- | |
|---|---|---|---|---|
| MLC | anti-cKIT | mAb-cKIT dAb | MLC Vk (Light | mAb- |
| VH | dAb | heavy chain ID | Chain SEQ ID) | dAb ID |
| 5-51 | DOM28h- | pDMS5078-HC | 4-1 | DMS5078 |
| | 094 | (SEQ ID NO: 375) | (SEQ ID NO: | |
| | DOM28m- | pDMS5071-HC | 365) | DMS5071 |
| | 007 | (SEQ ID NO: 376) | | |
| | DOM28m- | pDMS5072-HC | | DMS5072 |
| | 017 | (SEQ ID NO: 377) | | |
| | DOM28m- | pDMS5073-HC | | DMS5073 |
| | 023 | (SEQ ID NO: 378) | | |
| | DOM28m- | pDMS5074-HC | | DMS5074 |
| | 104 | (SEQ ID NO: 379) | | |
| | DOM28m- | pDMS5075-HC | | DMS5075 |
| | 107 | (SEQ ID NO: 380) | | |
| | DOM28m- | pDMS5076-HC | | DMS5076 |
| | 109 | (SEQ ID NO: 381) | | |
| | DOM28m- | pDMS5077-HC | | DMS5077 |
| | 112 | (SEQ ID NO: 382) | | |

The 8 humanized anti-vMLC mAb-cKIT dAb mAb-dAbs, DMS5071, DMS5072, DMS5073, DMS5074, DMS5075, DMS5076, DMS5077 and DMS5078, were purified from clarified expression supernatants by affinity chromatography using mAb Select HiTrap columns (GE Healthcare) according to established protocols. Concentrations of purified samples were determined by spectrophotometry from measurements of light absorbance at 280 nm. SDS-PAGE analysis of the purified sample shows non-reduced sample running at ˜175 kDa whilst reduced sample shows two bands running at ˜25 and ˜60 kDa corresponding light chain and dAb-fused heavy chain respectively.

mAb-dAbs were characterized for their solution state by SEC-MALLS (size-exclusion chromatography—multi-angle laser light scattering). Purified DMS5071, DMS5072, DMS5073, DMS5074, DMS5075, DMS5076, DMS5077 and DMS5078 were buffer exchanged into PBS, filtered and concentrations adjusted to 1.0 mg·ml⁻¹. RSA was purchased from Sigma (Fisher Scientific) and used without further purification (Batch number: KJ139812).

### Size-Exclusion Chromatography and Detector Set-Up:

Shimadzu LC-20AD Prominence HPLC system with an autosampler (SIL-20A) and SPD-20A Prominence UV/Vis detector was connected to Wyatt Mini Dawn Treos (MALLS, multi-angle laser light scattering detector) and Wyatt Optilab rEX DRI (differential refractive index) detector. The detectors were connected in the following order—LS-UV-RI. Both RI and LS instruments operated at a wavelength of 488 nm. An S-200 10/300 GL column (GE Healthcare) column was used (silica-based HPLC column) with mobile phase of PBS. The flow rate used is 0.5 ml/min. Proteins were prepared in buffer to a concentration of 1 mg/ml and injection volume was 100 μl.

### Detector Calibration:

The light-scattering detector was calibrated with toluene according to manufacturer's instructions.

Detector Calibration with BSA:

The UV detector output and RI detector output were connected to the light scattering instrument so that the signals from all three detectors could be simultaneously collected with the Wyatt ASTRA software. Several injections of BSA in a mobile phase of PBS (1 ml/min) are run over a An S-200 10/300 GL column (GE Healthcare) column with UV, LS and RI signals collected by the Wyatt software. The traces were then analysed using ASTRA software, and the signals were normalised aligned and corrected for band broadening following manufacturer's instructions. Calibration constants were then averaged and input into the template which is used for future sample runs.

### Absolute Molar Mass Calculations.

100 μl of each sample were injected onto a pre-equilibrated column (S-200 10/300 GL column (GE Healthcare)). After the SEC column the sample passes through 3 on-line detectors—UV, MALLS (multi-angle laser light scattering) and DRI (differential refractive index) allowing absolute molar mass determination. The dilution that takes place on the column is about 10 fold, and the concentration at which in-solution state was determined as appropriate.

The basis of the calculations in ASTRA as well as of the Zimm plot technique, which is often implemented in a batch sample mode is the equation from Zimm [J. Chem. Phys. 16, 1093-1099 (1948)]:

The calculations are performed automatically by ASTRA software, resulting in a plot with molar mass determined for each of the slices [Astra manual].

### SEC/MALLS Results:

SEC/MALLS analysis showed that all 8 bispecific mAb-dAbs DMS5071, DMS5072, DMS5073, DMS5074, DMS5075, DMS5076, DMS5077 and DMS5078 had monomeric solution states with the molecular weights calculated by MALLS closely matching the expected values (Table 29). The control sample Rat Serum Albumin ran as expected and also gave the predicted multimeric complexes.

TABLE 29
SEC/MALLS results for bispecific mAb-dAbs

| | Expected MW | MW by MALLS | |
|---|---|---|---|
| Sample | (kDa) | (kDa) | Solution State |
| DMS5071 | ~175 | 174.9 | Monomer |
| DMS5072 | ~175 | 174.0 | Monomer |
| DMS5073 | ~175 | 173.9 | Monomer |
| DMS5074 | ~175 | 176.5 | Monomer |
| DMS5075 | ~175 | 175.7 | Monomer |
| DMS5076 | ~175 | 173.0 | Monomer |
| DMS5077 | ~175 | 172.4 | Monomer |
| DMS5078 | ~175 | 176.7 | Monomer |
| RSA | 65 | 141.3, 64.66 | Dimer, Monomer |

d) BIAcore affinity to cKIT and Cell Binding Properties of Humanized Anti-vMLC mAb-cKIT dAb mAb-dAbs

DMS 5071, 5072, 5073, 5074, 5075, 5076, 5077 and 5078 were diluted to 1 uM in HBS-EP buffer (GE Healthcare) and diluted 1 in 3 for a 6 point dilution series. Samples were injected across different BIAcore chips and regenerated with glycine pH 2. The BIAcore curves were fitted using a bivalent BIAcore model, as this was expected to be the biologically most relevant. All curves that did not adhere to this model were considered to be bad fits. The fits were used to generate a KD (K_{D}) value for the event of one dAb binding a single cKIT/MLC molecule.

### e) Cell Binding

Cell binding experiments (full format) were carried out on DMS5071, 5072, 5073, 5074, 5075, 5076, 5077 and 5078 following the method described above.

mAb-dAbs were also tested for binding to primary mouse bone cells. Briefly, the mouse bone marrow sample was passed through a cell strainer and then spun to pellet the cells. The cells were then washed 2 times with FACS buffer (PBS/2.5% FCS) before being enriched for Lineage negative cells using a lineage depletion Miltenyi kit (#130-090-5858). Enriched cells were labelled with the humanised MLC mAb-cKIT dAb at 500 nM for 1 hr at 4° C. and detected using the full format method as described above previously. The cells were also stained with anti-cKIT FITC (BD Pharmingen #553354) at 0.25 μg·ml⁻¹ for 30 min @ 4° C. The cells were washed in FACS buffer again and resuspended in 200 μl PBS with 50 nM Topro-3 Iodide dead-cell dye before analysis on the Canto II flow cytometer. All data was analysed using Flow Jo software.

Data is summarised in Table 30. DMS5072, DMS5074, DMS5078, DMS5102, DMS5103, DMS5104 and DMS5105 were all shown to bind to primary mouse bone marrow c-kit positive cells.

TABLE 30
Cell binding and BIAcore results for humanized anti-vMLC mAb-cKIT dAb mAb-dAbs

| | | | | Primary | BIAcore | | BIAcore | | BIAcore | | | |
|---|---|---|---|---|---|---|---|---|---|---|---|---|
| | | | | mouse | KD for mouse | | KD for rat | | KD for human | | BIAcore | |
| DMS ID | HELA | MC/9 | EML | BM | c-kit | | c-kit | | c-kit | | KD for MLC | |
| DMS5071 | NO | YES | YES | NT | 4 | μM | 5 | μM | 4 | μM [BF] | 13 | nM |
| DMS5072 | NO | YES | YES | YES | 8 | μM | 3 | μM | 8 | μM | 9 | nM |
| DMS5073 | NO | YES | YES | NT | 13 | μM | NB | | NB | | 12 | nM |
| DMS5074 | NO | YES | YES | YES | 500 | nm [BF] | NB | | NB | | 13 | nM |
| DMS5075 | NO | YES | YES | NT | 6 | μM | NB | | NB | | 36 | nM [BF] |
| DMS5076 | NO | YES | YES | NT | 8 | μM | 1.5 | μM | 4 | μM | 4 | nM |
| DMS5077 | NO | YES | YES | NT | 700 | nm [BF] | NB | | NB | | 8 | nM |
| DMS5078 | NO | YES | YES | YES | 3 | μM | 2 | μM | 1 | μM [BF] | 10 | nM |

“NT”—not tested; “NB”—no binding; “BF”—Bad fit (could not be fitted to bivalent binding model)

DOM28h-94 affinity maturations produced high affinity binders with a number of point mutations as described above. Affinity matured dAbs with combined point mutations at positions 4 (Proline), 19 (Valine), 29 (Valine or Isoleucine) and 110 (Arginine) were used for formatting into the humanized anti-vMLC mAb-cKIT dAb heavy chain. 2 of these (DOM28h-94-11 and DOM28h-94-12) were selected from affinity maturations whilst another 2 (DOM28h-94-14 and DOM28h-94-15) were generated by crossover PCR. Briefly, a mixture of templates (DOM28h-94-2, DOM28h-94-6, DOM28h-94-10, DOM28h-94-11, DOM28h-94-12 and DOM28h-94-13) which had one or more of the aforementioned point mutations were pooled and PCR was carried out with a shortened extension time of 10 seconds. The PCR product was ligated in to the mAb-dAb heavy chain using SalI and HindIII ends. Colonies were randomly picked to inoculate cultures of E. coli for plasmid DNA minipreps. Plasmid miniprep DNA (Qiagen) was then used to transfect mammalian HEK293-6E cells. Each miniprep was mixed with light chain DNA (4-1 VK—human Ck, SEQ ID NO: 365) for co-transfection. After 72 hours of expression, supernatants were harvested and tested for binders of human and mouse c-kit by BIAcore. Supernatant samples giving desired affinities were noted and minipreps which were used to transfect those wells were sequenced for identification and given new clone IDs. mAb-dAb heavy chains pDMS5102-HC, pDMS5103-HC, pDMS5104-HC and pDMS5105-HC (SEQ ID NO: 385, SEQ ID NO: 386, SEQ ID NO: 387 and SEQ ID NO: 388 respectively) with affinity matured dAb sequences DOM28h-94-11, DOM28h-94-13, DOM28h-94-14 and DOM28h-94-15 were generated.

Sequence verified clones of heavy chain constructs were selected and large scale plasmid DNA preps were made using Qiagen Mega Prep Kit following the manufacturer's protocols. mAb-dAbs were expressed in mammalian HEK293-6E cells using transient transfection techniques by co-transfection of light chain DNA (4-1 VK—human Ck) and heavy chains.

### b) Purification and SDS-PAGE Analysis

4 humanized anti-vMLC mAb-cKIT dAb mAb-dAbs (Protein DMS ID: 5102, 5103, 5104, 5105 (see Table 24) were purified from clarified expression supernatants by affinity chromatography using mAb Select HiTrap columns (GE Healthcare) according to established protocols. Concentrations of purified samples were determined by spectrophotometry from measurements of light absorbance at 280 nm and samples checked by SDS-PAGE analysis.

### c) SEC/MALLS Analysis

DMS5102, DMS5103, DMS5104 and DMS5015 were analysed by SEC/MALLS employing the method outlined above. SEC/MALLS analysis showed that all 4 bispecific mAb-dAbs DMS5102, DMS5103, DMS5104 and DMS105 had monomeric solution states with the molecular weights calculated by SEC/MALLS closely matching the expected values (Table 31). The control sample Rat Serum Albumin ran as expected and also gave the predicted multimeric complexes.

TABLE 31
SEC/MALLS results for bispecific Humanized anti-vMLC
mAb-cKIT mAb-dAbs with affinity matured DOM28h-94 dAbs

| | Expected MW | MW by MALLS | |
|---|---|---|---|
| Sample | (kDa) | (kDa) | Solution State |
| DMS5102 | ~175 | 171.1 | Monomer |
| DMS5103 | ~175 | 177.6 | Monomer |
| DMS5104 | ~175 | 181.2 | Monomer |
| DMS5105 | ~175 | 171.9 | Monomer |
| RSA | 65 | 132.8, 64.67 | Dimer/Monomer |

d) BIAcore and Cell Binding Data was obtained using the methods described above.

TABLE 32
Cell binding and BIAcore results for humanized anti-vMLC
mAb-cKIT dAb mAb-dAbs with affinity mature DOM28h-94 dAbs

| | | | | Primary | BIAcore | BIAcore | BIAcore | |
|---|---|---|---|---|---|---|---|---|
| | | | | mouse | K_{D} for mouse | K_{D} for rat | K_{D} for human | BIAcore |
| DMS ID | HELA | MC/9 | EML | BM | c-kit | c-kit | c-kit | K_{D} for MLC |
| DMS5102 | YES | YES | YES | YES | 190 nM | 125 nM | 290 nM | 5 nM |
| DMS5103 | NO | YES | YES | YES | 72 nM | 30 nM | 74 nM | 4 nM |
| DMS5104 | YES | YES | YES | YES | 4000 nM | 5000 nM | 1000 nM | 5 nM |
| DMS5105 | YES | YES | YES | YES | 12 nM | 49 nM | 79 nM | 7 nM |

### Example 6

### mAb-dAb Epitope Mapping on BIAcore

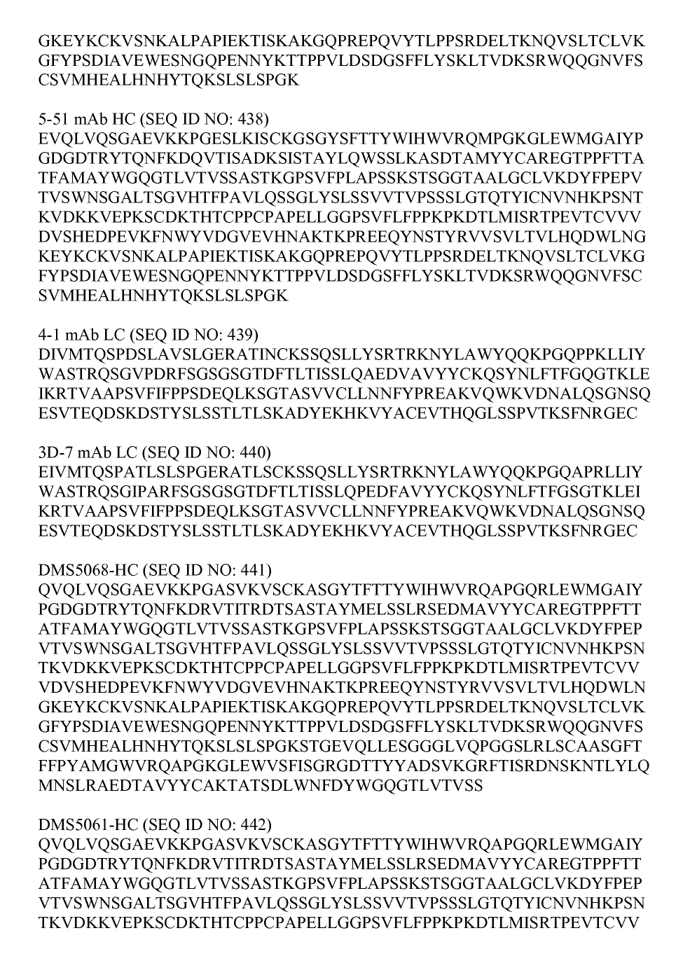

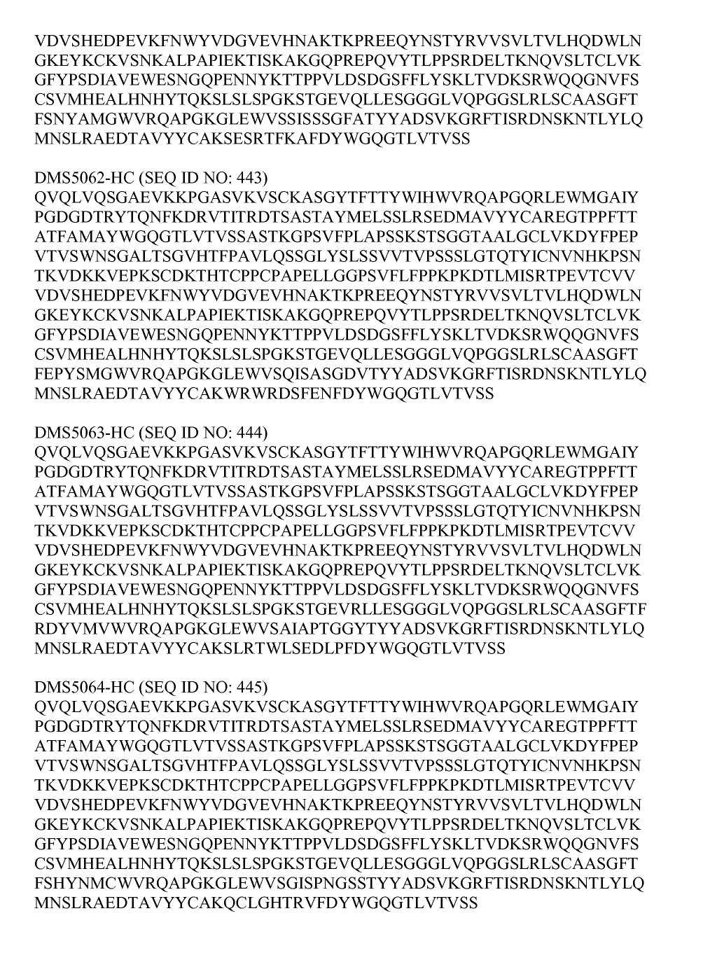

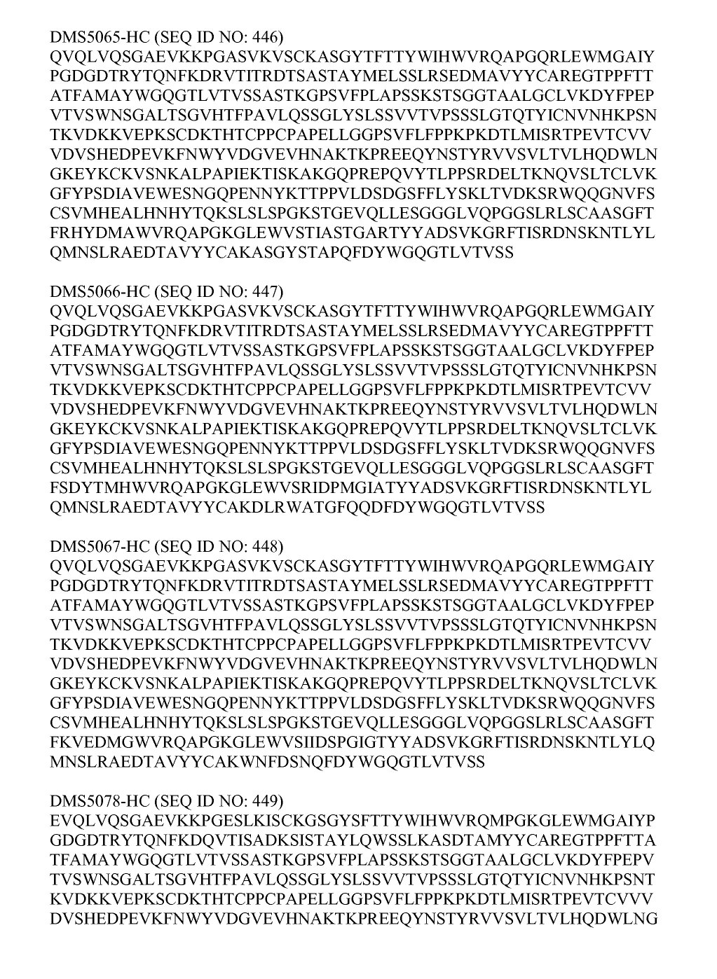

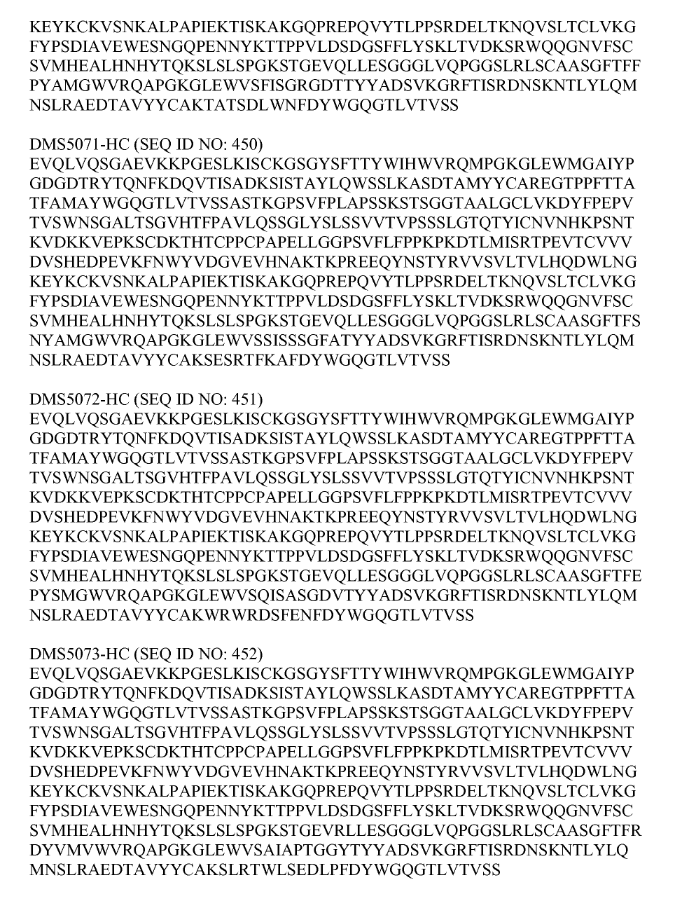

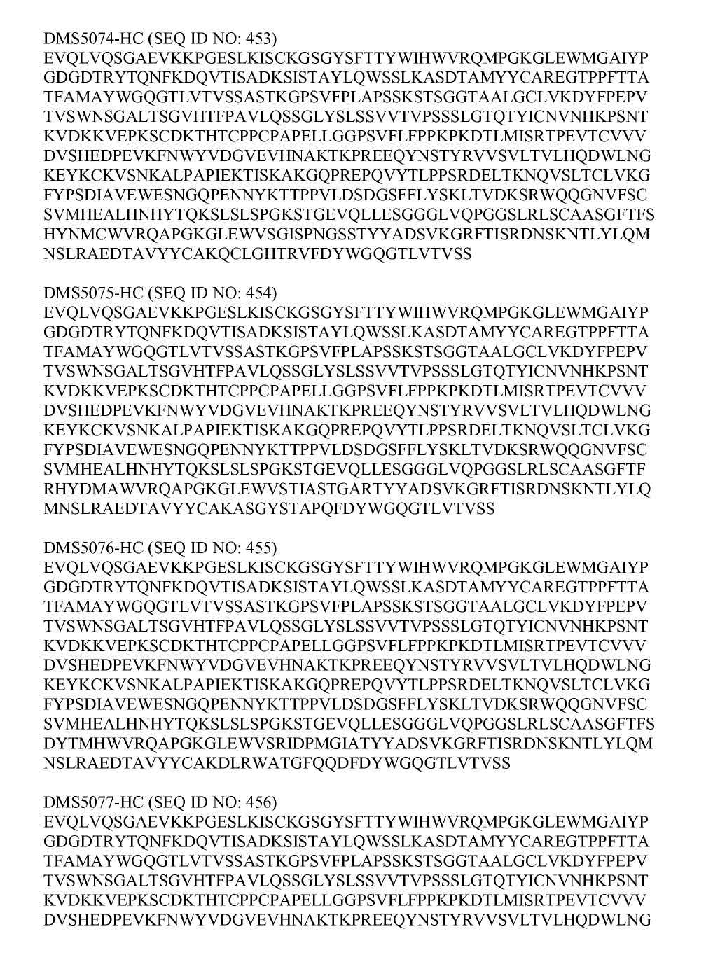

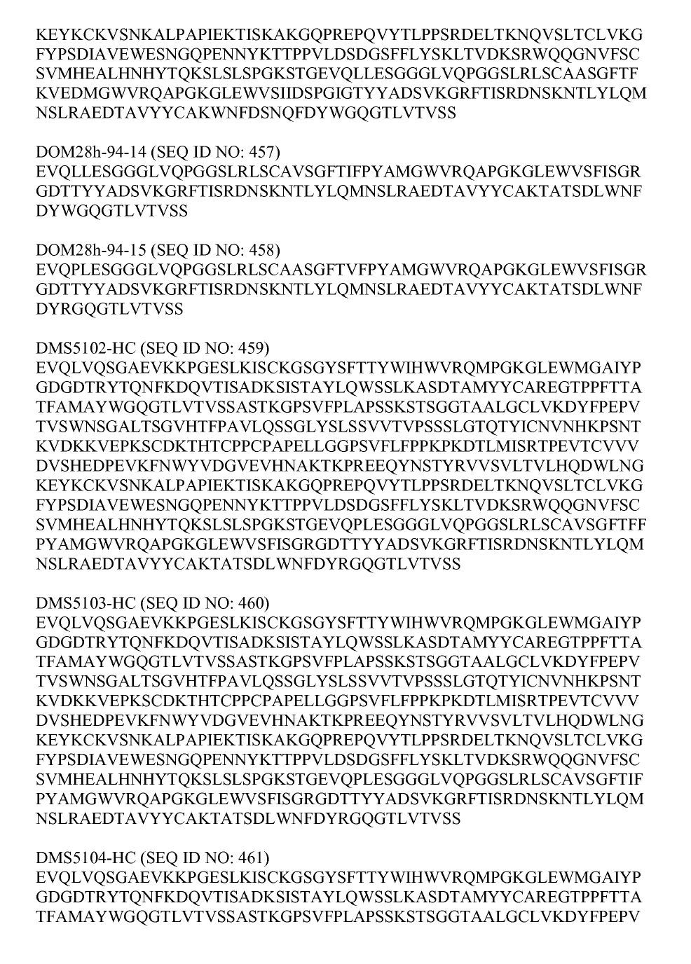

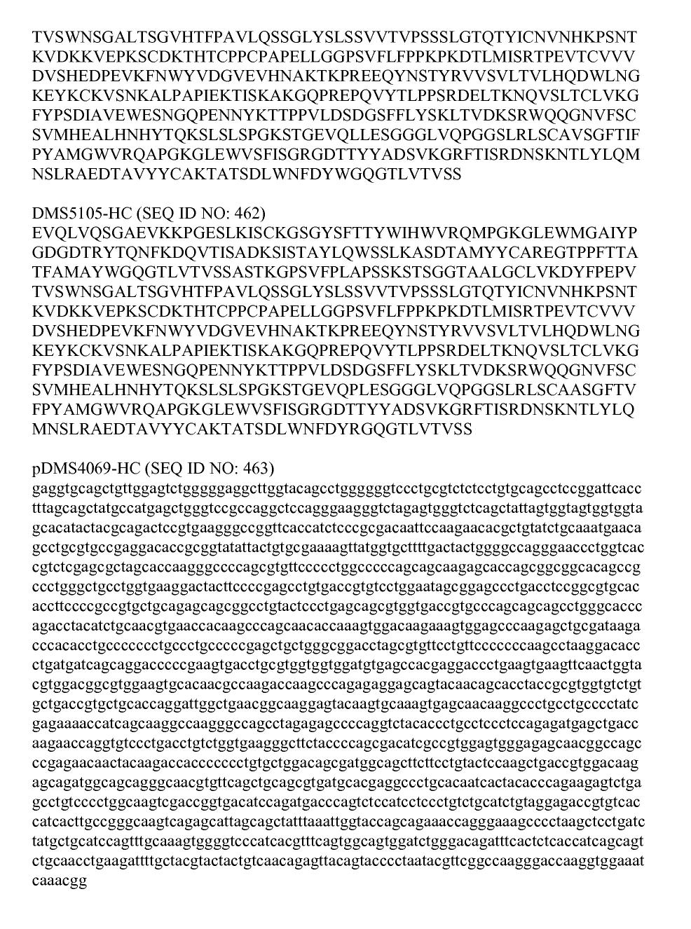

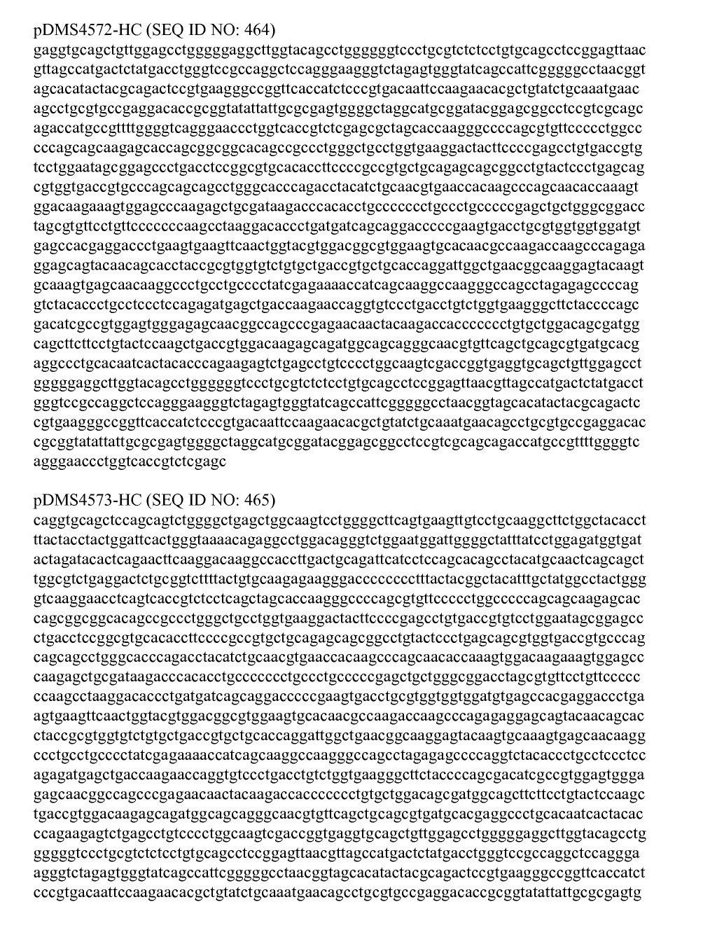

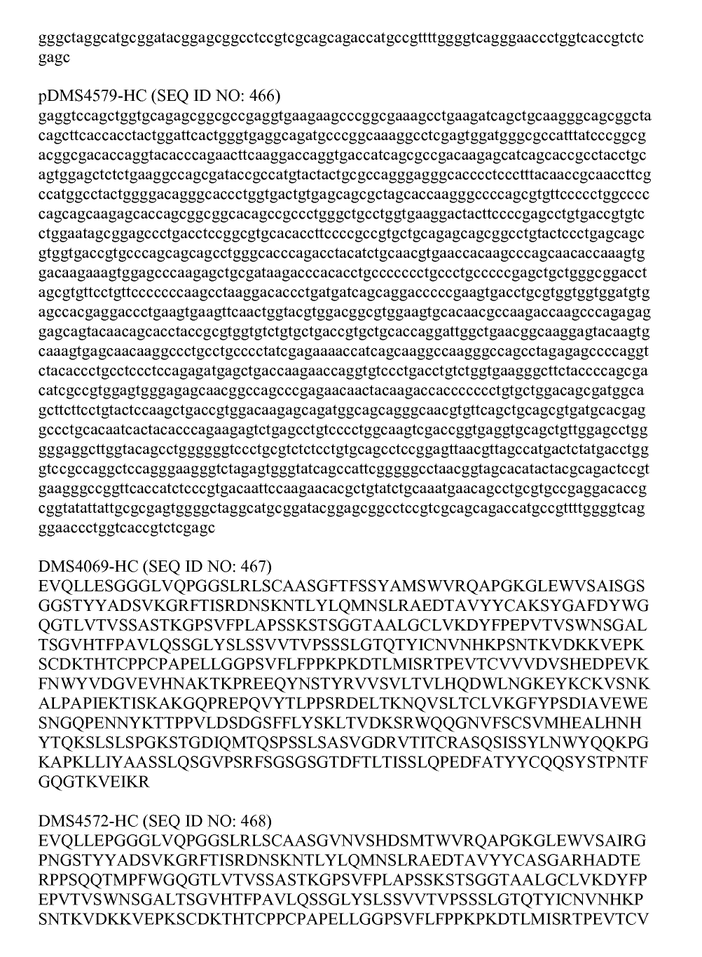

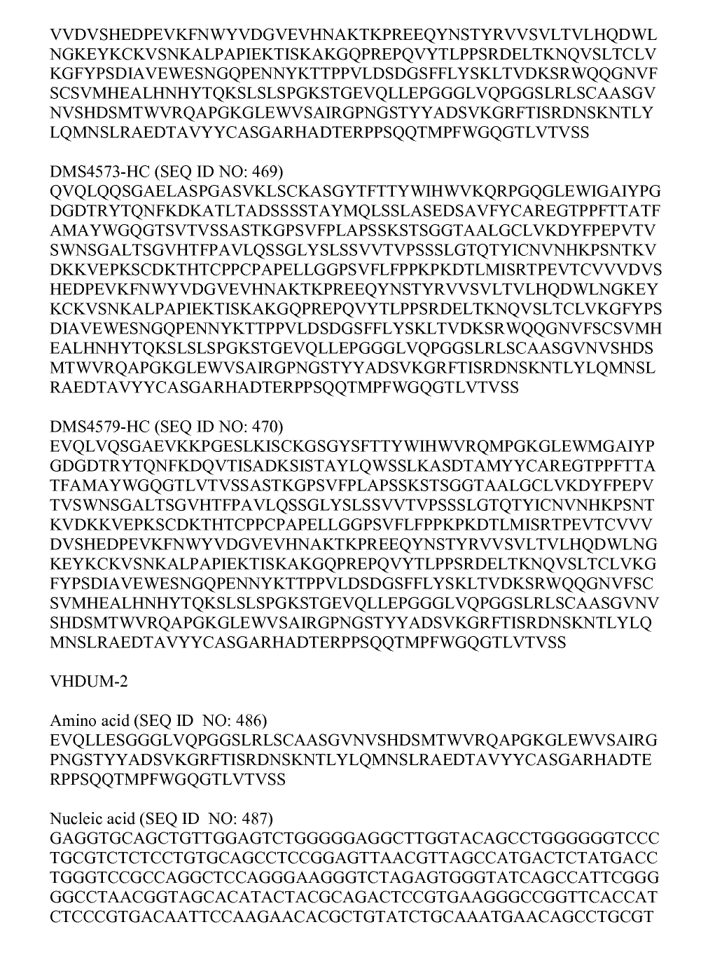

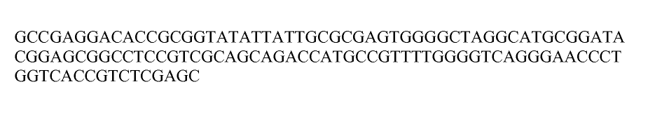

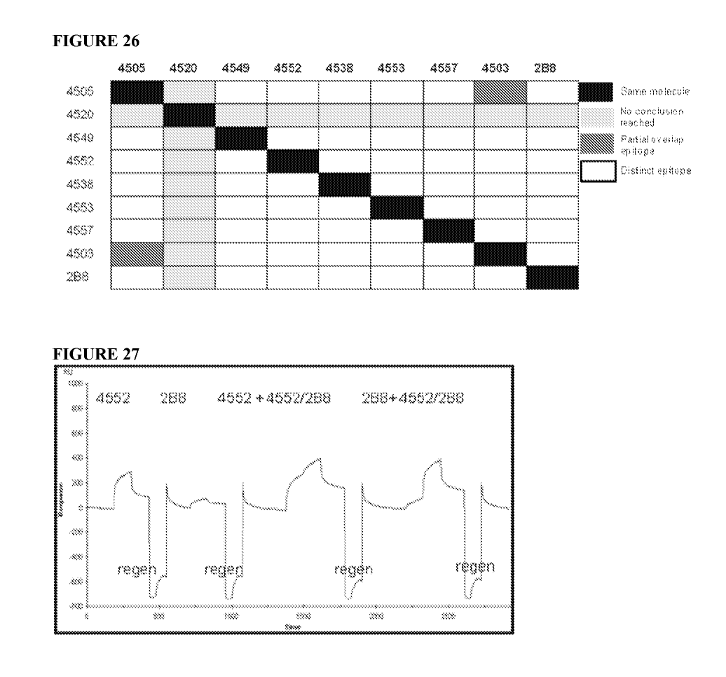

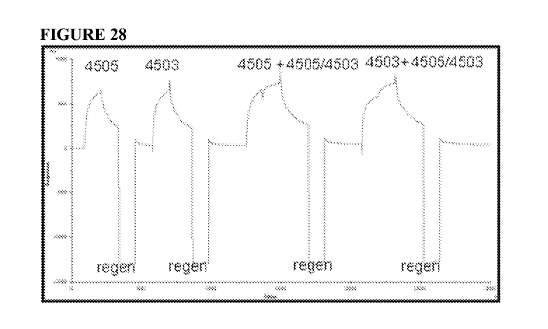

For Epitope Mapping, dummy frameworks mAb-dAbs were used to look for unique and overlapping epitopes. mAb-dAbs used were DMS 4505, 4538, 4520, 4549, 4552, 4553, 4557, 4503 and the commercial antibody 2B8. mAb-dAbs were diluted to 2 uM for analysis. Each mAb-dAb was paired with another, in all orientations, and run over a mcKIT chip coupled onto the CM5 chip. After each injection, the chip surface was regenerated with glycine pH 2. DMS 4520 bound weakly to the chip and so no meaningful epitope mapping could be determined. The FIG. 26 summarises the epitope mapping data. In addition, FIGS. 27 and 28 show examples of a typical epitope mapping experiment where the epitopes were considered to be unique and partially overlapping.

### Example 7

### Ranking of mAb-dAbs by Full-Format MSD PK Assay

Humanised MLC mAb-cKIT dAbs DM5071, DMS5072, DMS5073, DMS5074, DMS5075, DMS5076, DMS5077, DMS5078 and the affinity matured DOM28h-094 molecules DMS5102, DMS5103, DMS5104, and DMS5105 were run in an MSD PK assay as follows:

96-well MSD standard bind plates (MSD #L11XA-6) were spot-coated with 5 μL per well of cKIT-H6 (His tagged) at 50 μg/mL in spot coating buffer (Spot-coating buffer=25 mM HEPES (Sigma #H0887)+0.015% Triton-X-100 (Fisher #BP151-500)+MilliQ water)). Plates were allowed to dry for 20 hours overnight at room temperature in a laminar flow hood. Plates were washed 3 times in wash buffer containing PBS (Oxoid #BRO014G)+0.1% Tween-20 (Fisher #BPE337) and blotted onto tissue paper. Plates were then incubated with 150 μL per well of assay buffer consisting of PBS (Oxoid #BRO014G)+5% BSA (Sigma #A7030)+1% Tween-20 (Fisher #BP151-500) for 1 hour on a plate shaker at room temperature to block non-specific binding. Plates were washed as before. All test mAb-dAbs and control molecules (including DMS4579 and DMS4503 as negative controls) were diluted from 2500 ng/mL in assay buffer containing 10% control mouse serum (Sera Labs #S-808-D) serially diluted 1:2 over 11 points. A blank standard of assay buffer containing 10% mouse serum was included for each molecule. 25 μL of the prepared standards were added to the MSD plates. Triplicate replicates were run for each molecule split across 3 MSD plates, with one replicate per plate. Plates were then incubated at room temperature on a plate shaker for 1 hour. Plates were washed as before and incubated with 50 μL per well of vMLC1-sulfotag at 0.2 μg/mL in assay buffer at room temperature on a plate shaker for 1 hour. (To sulfotag vMLC1, the protein was reacted with a 5-fold molar excess of MSD Sulfo-tag (MSD #R91AN-1) (prepared as per manufacturer's instructions) and incubated in a dark drawer at room temperature for 2 hours to allow conjugation. The conjugated protein-sulfotag mixture was then purified by passing through a Zeba Spin Desalting Column (Pierce #89891) as according to manufacturer's instructions and the purified conjugated mixture was collected and stored at 4° C. until use in the assay). Plates were washed three times with wash buffer, blotted on tissue paper and 150 μL per well of MSD read buffer T with surfactant (MSD #R92TC-1) diluted to 1× with distilled water was added and plates were read immediately using the MSD Sector Imager 6000.

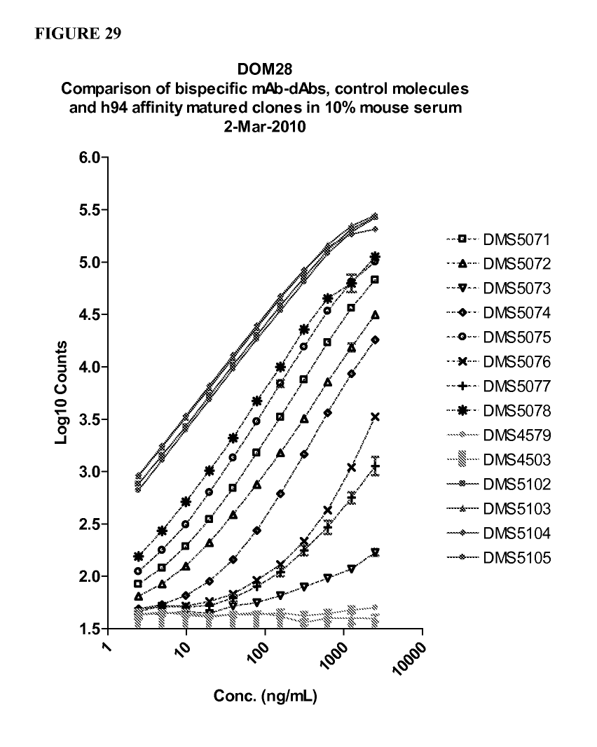

Data was analysed using GraphPad Prism 4.02 and Microsoft Excel 2007. Raw counts from the standards of individual molecules was plotted against the known concentration for the individual molecules and fitted using a 4PL non-linear regression model, subsequent to applying an X=LOG(X) and Y=LOG(Y) transformation. (as shown in FIG. 29). The raw counts of each individual replicate were then interpolated against the curve fit and the deviation from the fit was assessed in Excel by expressing the obtained values as a percentage of the expected (theoretical) concentration. The Lower Limit of Quantification (LLOQ) was determined as the lowest concentration where all three replicates fell within 70-130% of the theoretical concentration and the Upper Limit of Quantification (ULOQ) was determined as the highest concentration where all three replicate fell within 70-130% of the theoretical concentration.

Ranking of mAb-dAbs:

A summary table (Table 33) detailing the ULOQ (in ng/mL), the LLOQ (in ng/mL), the Signal-to-Noise ratio at the ULOQ, the Signal-to-Noise ratio at the LLOQ, counts obtained at the top standard concentration and background counts for each mAb-dAb tested was collated.

TABLE 33

| | DMS5071 | DMS5072 | DMS5073 | DMS5074 | DMS5075 | DMS5076 |
|---|---|---|---|---|---|---|
| ULOQ (ng/mL) | 1250 | 1250 | 1250 | 1250 | 1250 | 1250 |
| LLOQ (ng/mL) | 2.44 | 2.44 | 312.5 | 9.77 | 4.88 | 39.06 |
| S:N @ ULOQ | 811 | 361.6 | 2.9 | 198.8 | 1376 | 1099 |
| s:N @ LLOQ | 1.8 | 1.5 | 2 | 1.5 | 3.8 | 67.7 |
| Top counts | 67836 | 740.6 | 170 | 18192 | 1E+05 | 3335 |
| Background | 45 | 42.7 | 40.7 | 43.3 | 47.3 | 45 |
| | DMS5077 | DMS5078 | DMS5102 | DMS5103 | DMS5104 | DMS5105 |
| ULOQ (ng/mL) | 1250 | 625 | 1250 | 1250 | 1250 | 1250 |
| LLOQ (ng/mL) | 1250 | 4.88 | 4.88 | 4.88 | 4.88 | 4.88 |
| S:N @ ULOQ | 12.8 | 958.1 | 4315 | 5434 | 4487 | 4730 |
| s:N @ LLOQ | 12.8 | 5.8 | 29.6 | 41.9 | 42.3 | 31.4 |
| Top counts | 1172 | 1E+05 | 3E+05 | 3E+05 | 2E+05 | 3E+05 |
| Background | 44.7 | 47 | 48.3 | 41 | 41.3 | 41 |

Each mAb-dAb was assessed on the individual assay parameters stated above and assigned a numerical value for each. For each parameter, the highest ULOQ, lowest LLOQ, best signal-to-noise ratio and optimum counts was assigned a value of 1 (where the mAb-dAb results fell within the acceptable level). The next best result was assigned a value of 2 etc. Once all mAb-dAbs had been assigned values for the assay parameters, the sum of all values assigned to each was calculated. The sum was then ranked from 1-12 to provided an indication of the overall performance of the mAb-dAb in the assay. The bispecific with the lowest sum indicated the mAb-dAb which met acceptable assay parameters best and was ranked number 1 overall. All other bispecifics were then sequentially ranked up to 12. Results are shown in Table 34.

TABLE 34

| | DMS5071 | DMS5072 | DMS5073 | DMS5074 | DMS5075 | DMS5076 |
|---|---|---|---|---|---|---|
| ULOQ (ng/mL) | 1 | 1 | 1 | 1 | 1 | 1 |
| LLOQ (ng/mL) | 1 | 1 | 5 | 3 | 2 | 4 |
| S:N @ ULOQ | 8 | 9 | 12 | 10 | 5 | 6 |
| s:N @ LLOQ | 2 | 2 | 2 | 2 | 2 | 3 |
| Top counts | 7 | 11 | 12 | 8 | 6 | 9 |
| Background | 1 | 1 | 1 | 1 | 1 | 1 |
| Sum of ranking: | 20 | 25 | 33 | 25 | 17 | 24 |
| Final Rank: | 7 | 10 | 12 | 9 | 5 | 8 |
| | DMS5077 | DMS5078 | DMS5102 | DMS5103 | DMS5104 | DMS5105 |
| ULOQ (ng/mL) | 1 | 2 | 1 | 1 | 1 | 1 |
| LLOQ (ng/mL) | 6 | 2 | 2 | 2 | 2 | 2 |
| S:N @ ULOQ | 11 | 7 | 4 | 1 | 3 | 2 |
| s:N @ LLOQ | 3 | 2 | 1 | 1 | 1 | 1 |
| Top counts | 10 | 5 | 2 | 1 | 4 | 3 |
| Background | 1 | 1 | 1 | 1 | 1 | 1 |
| Sum of ranking: | 32 | 19 | 11 | 7 | 12 | 10 |
| Final Rank: | 11 | 6 | 3 | 1 | 4 | 2 |

### Conclusion:

These data show that the affinity matured DOM28h-94 molecules (DMS5102, DMS5103, DMS5104, DMS5105) were the better performing mAb-dAbs in this assay when compared to the other mAb-dAbs tested and the parent DOM28h-94 molecule (DMS5078). This result is also reflected in the graph (FIG. 29). The affinity matured DOM28h-94 mAb-dAbs showed greatest signal in the assay. They also appeared to be more potent than the other mAb-dAbs as the signal-to-noise ratio at the LLOQ was significantly higher than for the other mAb-dAbs. The background counts for these molecules were at an acceptable level for this assay and were at a level that is equal to the other bispecifics (DMS5071-DMS5078) and the control molecules (DMS4579 and DMS4503). The control mAb-dAbs DMS4579 and DMS4503 gave counts at the level of background (and produced a flat line curve) as would be expected for these molecules. All other mAb-dAbs tested gave a signal which was lower than that of the affinity matured DOM28h-94 molecules, but the level of signal varied greatly between the mAb-dAbs. Background counts for these molecules were at the level expected.

### Example 8

### Method for Immunofluorescence to Assess mAb-dAb Internalisation Properties

Dummy mAb-c-kit-dAbs were examined for internalisation using the following method:

8 well chamber slides (Lab TEK-II #154534) were washed with 1M HCL followed by two washes with dH₂0. The chamber slides were then coated with 0.05% poly-1-lysine (Sigma #P4707) and incubated for 20 minutes at room temperature. The poly-1-lysine was aspirated and the chambers were dried in an oven at 55-60° C. for 30-60 minutes then stored at 4° C. for no more than two days. MC/9 cells were then plated on the coated chambers at 1.2×10⁶ cells per well and incubated at 37° C., 5% CO₂ for 2-3 hours. The media was then aspirated from the chambers and the cells were incubated with the mAb dAbs which had been diluted in media to a final concentration of 100 nM+/−mouse stem cell factor (mSCF) at 1 μg/ml for 30 minutes at 4° C. or 37° C. 5% CO₂.

The cells were then fixed in 2% formaldehyde in PBS for 10 minutes at room temperature then washed/blocked twice in 5% FCS/PBS for approximately 7-8 minutes. The mAb-dAbs were then detected using goat anti-human IgG Alexa 488 (Molecular Probes #A11013) diluted 1:200 in 5% FCS/PBS with 0.2% saponin for permeabilisation (100 μl per well) and incubated for a minimum of 30 minutes in the dark. The antibody mixture was then aspirated and the cells were washed with PBS containing 1 μg/ml DAPI (4′6-DIAMIDINO-2-PHENYLINDOLE DIHYDROCHL Sigma #D8417) for a minimum of 5 minutes at room temperature.

The wash was then aspirated and the chamber wells were removed using the supplied equipment from the manufacturer. A large drop of fluoromount G (Southern Biotech, cat #0100-01), 100 μl between four wells, was added to the slide and a large coverslip (22 mm×50 mm Fisher Scientific UK cat #5477630) was inverted on top of the slide. The coverslip was sealed with clear nail varnish and the slides were imaged on a Leica SP2 Confocal microscope.

Results—Investigation of Internalisation Properties of Dummy IgG and cKIT dAb Molecules.

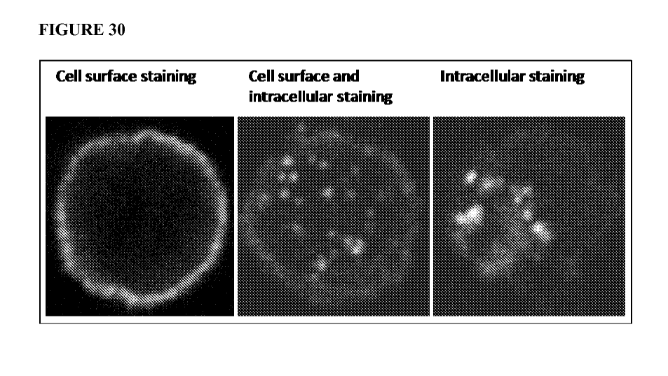

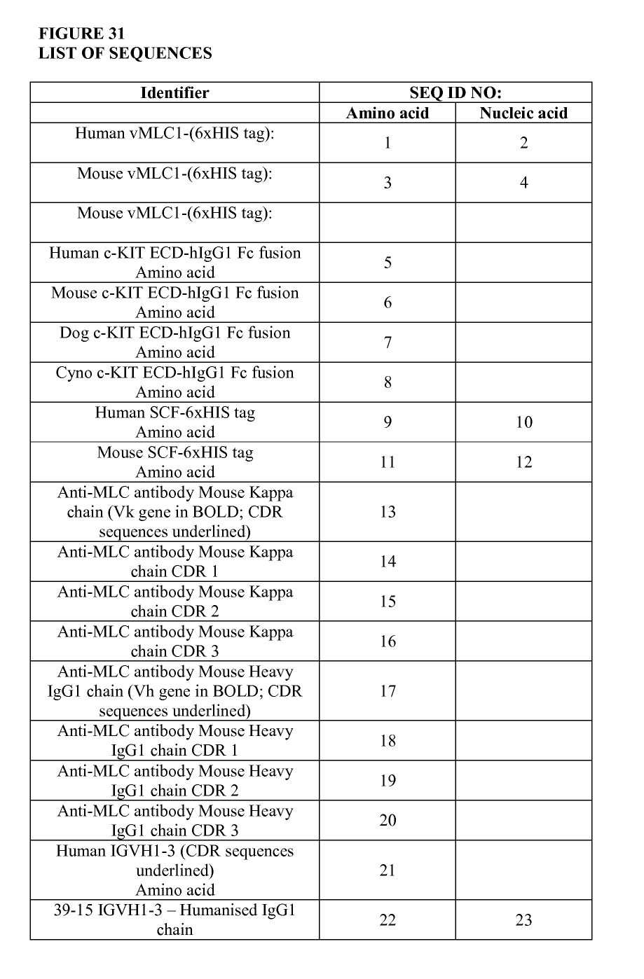

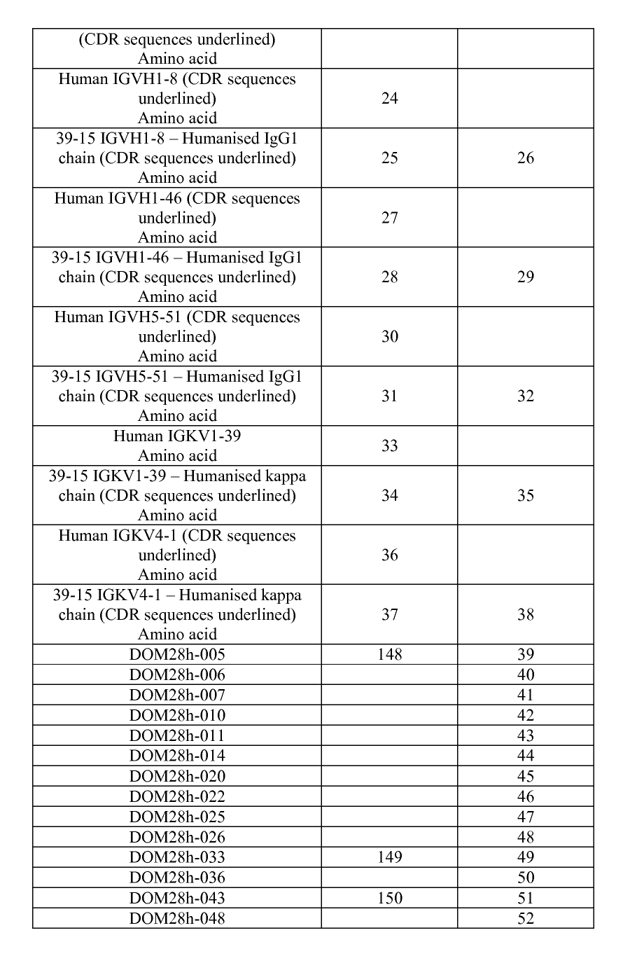

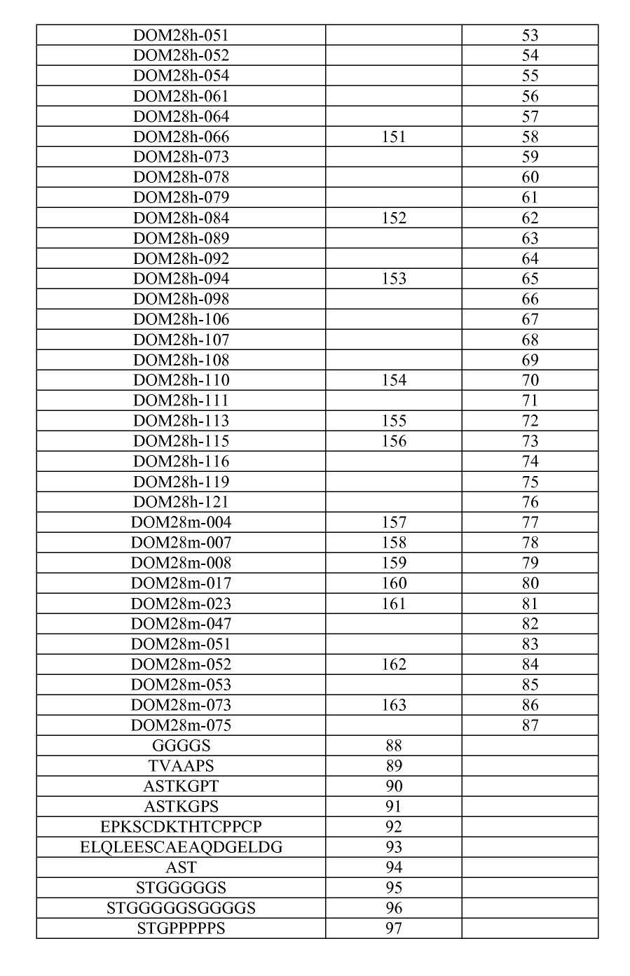

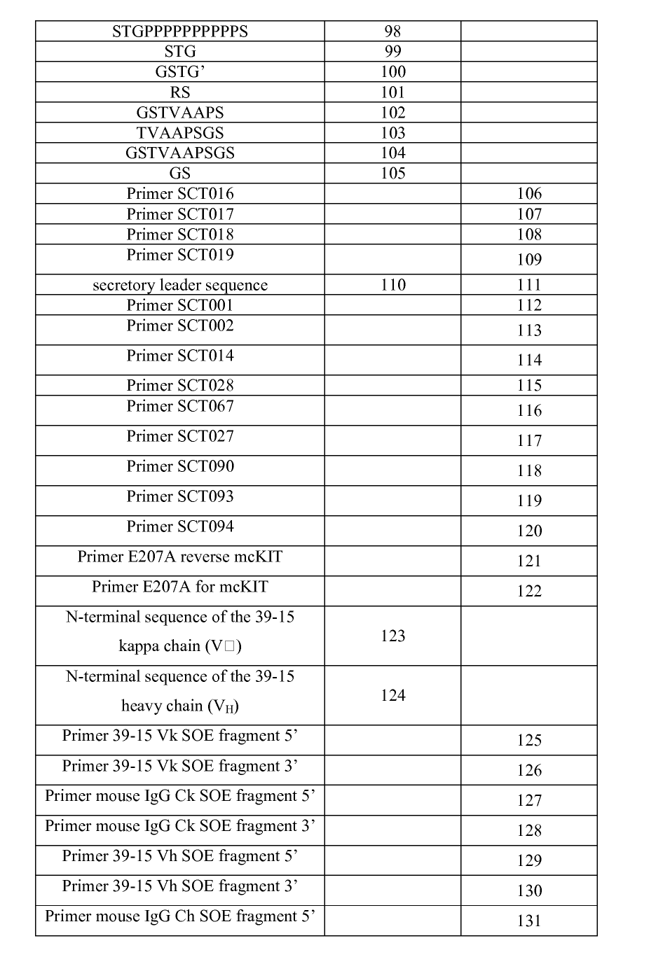

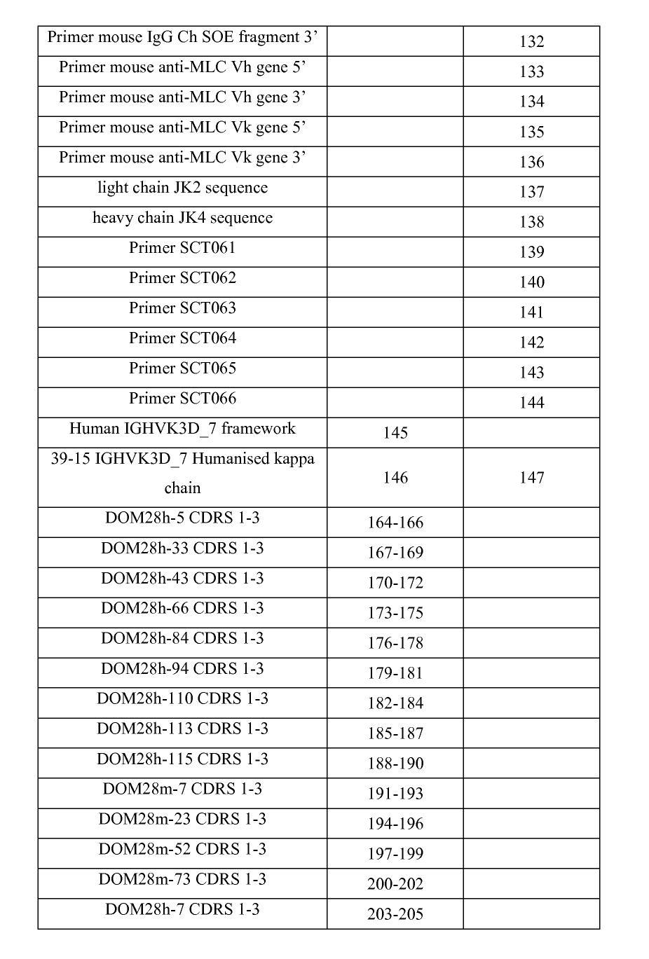

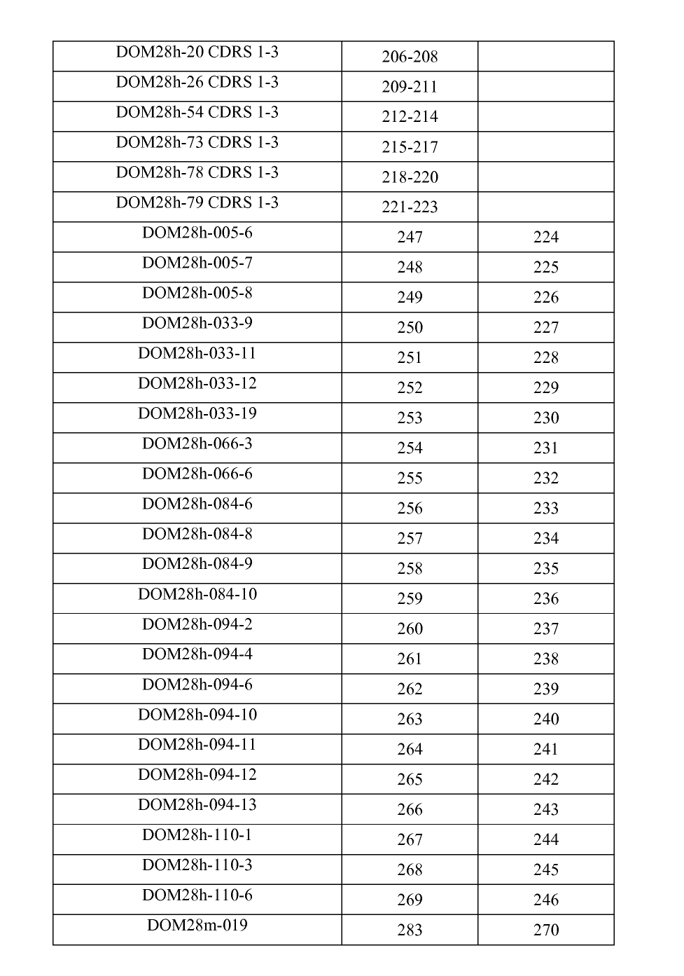

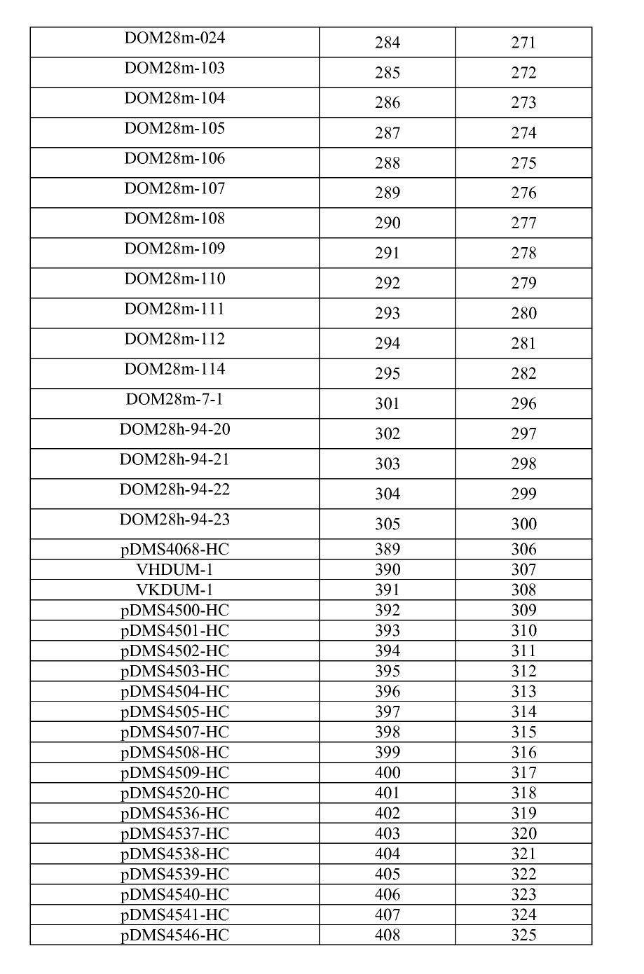

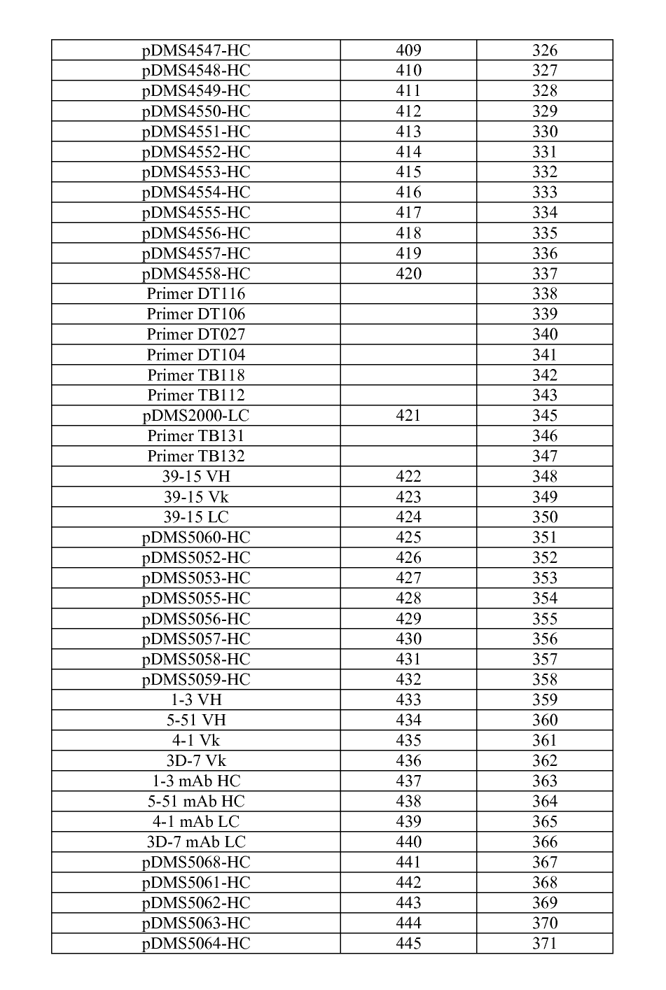

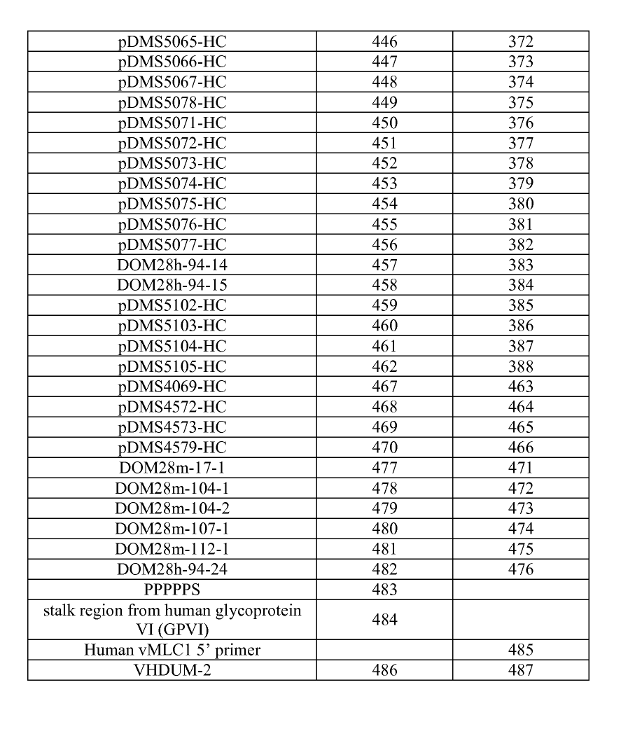

An example of the staining patterns is shown in FIG. 30. The image shows a representative example of a cell which displayed cell surface staining (CS), a cell which showed both cell surface and intracellular staining (CS & IC) and a cell with only intracellular staining (IC):

TABLE 35
Summary of the internalisation properties of
dummy molecules (ND = Not Determined):

| | Staining | Staining | Staining | Staining |
|---|---|---|---|---|
| mAb | pattern at | pattern at | pattern at | pattern at |
| dAb | 4° C. | 4° C. + mSCF | 37° C. | 37° C. + mSCF |
| DMS4503 | CS | CS | CS & IC | CS & IC |
| DMS4520 | CS | CS | CS | CS |
| DMS4538 | CS | CS | CS | CS & IC |
| DMS4539 | CS | CS | IC | IC |
| DMS4547 | CS | CS | CS & IC | IC |
| DMS4557 | CS | CS | CS | CS & IC |
| DMS4505 | ND | ND | CS | CS |
| DMS4549 | ND | ND | CS & IC | IC |
| DMS4552 | ND | ND | CS & IC | CS & IC |
| DMS4554 | ND | ND | CS | CS |
| DMS4558 | ND | ND | CS | CS |

### Example 9

### Method for Flow Cytometry to Assess mAb-dAb Internalisation Properties

MC/9 cells were counted and resuspended at 1×10⁶ cells in 100 μl of media added to a v-bottomed 96 well plate and spun down again. The media was aspirated and the cells were then resuspended in 100 μl of media containing 500 nM of mAb-dAb+/−1 μg/ml mSCF. The affinity matured clones were tested at 50 nM. The cells were then incubated at either 4° C. (on ice) for 30 minutes, 37° C. for 30 minutes or 37° C. for 60 minutes. The cells then were spun down and washed in 200 μl per well of 5% FCS/PBS twice. All wash steps were performed on ice to prevent further internalisation. The cells were then incubated in 200 μl per well of 5% FCS/PBS for 20 minutes on ice to further block. Following the blocking step the cells were then spun down and incubated with 100 μl PBS containing goat anti-human IgG Alexa 488 (Molecular Probes #A11013) at 1:1000 dilution for no more than 40 minutes on ice. The cells were then washed once in PBS and resuspended in 100 μl PBS for acquisition on the BD Canto (Becton Dickenson).

Results—Investigation of Internalisation Properties of Bispecific Humanised Anti-MLC mAb cKIT-dAb Molecules.

Table 36 shows percentage binding of mAb-dAbs after 30 and 60 minutes at 37° C. (% binding is compared to the binding observed at 4° C.):

TABLE 36

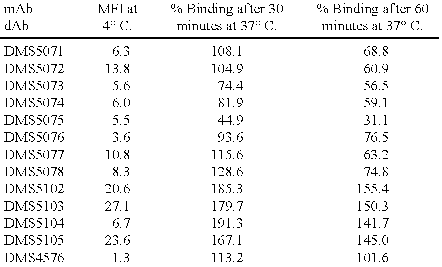

The table above shows the MFI binding of the mAbdAbs at 4° C. (second column). The relative level of binding after 30 or 60 minutes at 37° C. is shown as a percentage value compared to the 4° C. binding in the third and fourth columns respectively. 100% binding indicates that after 30 or 60 minutes at 37° C. the cell surface levels of the mAb dAb are the same for that observed at 4° C. Any value less than 100% shows that cell surface levels of the mAb dAb have decreased indicating internalisation may have occurred. Any value more than 100% indicates that cell surface levels have actually increased after increasing time at 37° C. and thus the mAb dAb has not been internalised.

Any binding observed below 100% is highlighted in shaded cells. A decrease in cell surface binding indicates that the molecule is internalised. DMS5073, 5074, 5076 show a slight decrease in signal after 30 minutes at 37° C. DMS5075 shows a larger decrease. All of the naïve molecules (DMS5071→DMS5078) show decreased binding after 60 minutes at 37° C., but only DMS5075 shows a large decrease in binding. None of the affinity matured molecules show any decreased binding after incubation at 37° C. for either 30 or 60 minutes.

### Example 10

### In Vivo Murine Studies

### Protocols:

### 1. Bone Marrow (BM) Isolation, Labeling and Injection

3-4 month-old wild-type, B6.129Sv-Gtrosa26 (Rosa-26) (Friedrich G, Soriano P. Promoter traps in embryonic stem cells: a genetic screen to identify and mutate developmental genes in mice. Genes Dev. 1991 September; 5(9):1513-23) or other genetically-engineered mice are euthanized by isoflurane or Nembutal administration. Femurs are removed and trimmed of muscle and extraossial tissue. The bones are cut proximally and distally, and the bone marrow flushed with 2% bovine serum albumin in ice-cold phosphate-buffered saline (PBS) using a 26 G needle and a 1 ml syringe. The cellular pellets are rinsed and filtered through a 40 μm nylon filter. The cellular pellets are washed and resuspended in PBS and cell concentration calculated using trypan blue and a hemocytometer.

Bone marrow cells may be labeled with Feridex (ultrasmall superparamagnetic iron oxide) for MRI imaging prior to incubation with bispecific antibodies and subsequent injection into recipient mice that have been subjected to cardiac injury (i.e. ischemia-reperfusion or permanent myocardial infarction.

### 2. Feridex Labeling of BM

Feridex (25 μg/ml) (Berlex Laboratories) is incubated with poly-L-lysine (30 ng/μl) (Sigma) for 2 hours. Meanwhile, bone marrow cells are harvested, rinsed in PBS and resuspended in Dulbecco's Modified Eagle's medium+1% penicillin and streptomycin. After 2 hours, the Feridex/PLL solution is added to the cells, which are then incubated overnight (up to 24 hours) at 37° C. and 5% CO₂. The cells are then removed from the flask by scraping.

Iron uptake is quantified using a plate-based assay Quantichrom Iron Assay Kit (BioAssay Systems, Cat # DIFE-250). Iron content of at least 20 pg/cell is considered sufficient for future detection by MRI.

3. Bone Marrow Cell Labeling with Bispecific Antibodies

Bone marrow cells from donor mice (either Rosa-26 or other genetically engineered mice, or Feridex-labeled cells from wild-type mice) are incubated with bispecific antibodies (c-kit X MLC) ex vivo prior to systemic injection into recipient mice. Briefly, bone marrow cells are incubated with the antibody (500 ng-15 μg/10⁷ cells) in PBS for 1 hour at 4° C. Cells are rinsed in >10 ml of PBS, centrifuged and resuspended in 2 ml of PBS (i.e. final concentration=10 million cells/200 μl) and kept on ice until use.

For unarmed controls, bone marrow cells from Rosa-26 mice or wild-type,

Feridex-labeled mice are harvested, rinsed, counted and resuspended to a final concentration of 10 million cells.

### 4. Systemic Bone Marrow Cell Injection

Bone marrow cells are injected into wild-type recipient mice via tail vein or jugular vein injection (10⁷ cells per mouse in a 200 μl volume of PBS) immediately following ischemia-reperfusion/coronary artery ligation, or anywhere from 1-7 days later.

### 5. Ischemia-Reperfusion Model

Mice are anesthetized with Nembutal (60 mg/kg, 0.6 ml ip) (Hanna's Pharmaceutical Supply Company) shaved and the antiseptic agents (Betadine, Purdue products LP, Stamford, Conn. and 70% alcohol) are then applied to the surgical site. The animal is placed supine and trachea is intubated with PE-90 tubing. The cannula is connected to a rodent ventilator (Harvard apparatus), at a rate of 105/min and a tide volume of 0.5 ml room air supplemented with oxygen (1 L/min). The body temperature is maintained by T/pump heat pad. The chest cavity is entered through right a midline sternotomy or left thoracotomy. An 8-0 suture is passed under the left anterior descending coronary artery, and a balloon occluder is applied to the artery. Myocardial ischemia and reperfusion are induced by inflating and then deflating the balloon occluder. The successful performance of coronary occlusion and reperfusion is verified by the apical pallor of the myocardium and typical ECG changes. A chest tube is implanted in the chest cavity in order to evacuate residual air and fluid. The incision is closed in layers (muscle and skin) using a 5-0 suture and the chest tube is withdrawn after the chest is closed.

### 6. Myocardial Infarction Model

For the MI-induced heart failure model, a similar surgical procedure is used as ischemia reperfusion procedure above, except that the coronary artery (LAD or left anterior descending artery, or other coronary artery) is permanently ligated without reperfusion.

### 7. Systemic Ab Injection

Mice are anesthetized by isoflurane to effect. Systemic Ab injection is performed by direct injection into jugular vein or tail vein using a 30 G needle 1 cc syringe (200 μl/mouse).

### 8. Functional and Histological Analyses

At completion of the study, cardiac function of the mice is evaluated by MRI. In mice receiving Feridex-labeled cells, MRI is also used to trace labeled cells in vivo. Upon completion of functional analysis, mice are sacrificed, hearts (as well as other organs, including the liver, lung and spleen) are harvested and either processed for ex vivo MRI (for higher resolution tracking of Feridex-labeled donor cells) or histology and immunohistochemistry. Histological analysis of the hearts may be used to determine infarct size or extent of Feridex labeled uptake at the infarct site (by Perl's staining). Immunohistochemistry on cardiac sections may be utilized to detect donor cells of different genetic origin (e.g. Rosa-26 cells can be detected by immunostaining for the β-galactosidase protein), to identify antibody homing to the infarct (and also to other organs) and to evaluate differentiation of donor cells into cardiac cell types, including cardiomyocytes, endothelial and smooth muscle cells. PCR may also be used to identify donor cells of different genetic origins homing to other tissues to evaluate potential safety issues and off-target effects.

### 9. Ultrasound (Echocardiography)

Echocardiography is performed as reported previously by Wyatt et al. (“Cross sectional Echocardiography I: analysis of mathematical models for quantifying mass of the left ventricle in dogs”; Circulation, Vol 60, No 5, pp 1104-1113, November 1979) and Wyatt et al. (“Cross sectional Echocardiography II: analysis of mathematical models for Quantifying Volume of the formalin-fixed left ventricle”; Circulation Vol 61, No 6, pp 1119-1125, June 1980).

### 10. In Vivo Cardiac Magnetic Resonance Imaging

Mice are anesthetized using a 1.5-2.0% isoflurane/medical air mixture at a flow rate of 1 L/min. In vivo cardiac magnetic resonance imaging is performed in a 9.4 T vertical bore magnet (Bruker Biospin; Billerica, Mass.) using a transmit/receive coil with an internal diameter of 2.9 cm. The in vivo MRI is performed using a wireless self gating intragate sequence using similar approach with navigator echoes as described in Larson et al. (Mag Res Med 51:93-102 (2004)), Kellman et al. (Mag Res Med 59:771-778 (2008) and Uribe et al. Mag Res Med 57:606-613 (2007).

Gradient echo scout images are acquired in order to obtain the long and short axis plane of the mouse heart. The tri-pilot scout sequence imaging parameters are as follows; TE/TR=1.2/67.5 ms, FOV=40 mm×40 mm, Matrix=128×128, flip angle=15 degrees, slice thickness=1 mm, 10 slices/orthogonal plane, 10 repetitions, TA=1:26 seconds.

Upon completion of the scout sequence, long axis (coronal and sagittal) and short axis (axial) gradient echo images are acquired through the mouse heart. The gradient echo cine images are acquired using the following parameters; TE/TR=1.8/6.8 ms, FOV=25 mm×25 mm, Matrix=128×128, flip angle=10 degrees, slice thickness=1 mm, 250 repetitions, TA=3:39 seconds. In vivo resolution was ˜195 microns. Images were reconstructed using 10 phases per cardiac cycle. Image analysis of cardiac function (EF %, EDV, ESV, SV, CO) and morphology (LV Mass) is performed using Analyze 8.1 software package (AnalyzeDirect, Lenexa Kans.). Upon completion of the imaging, mice are removed from the magnet and allowed to recover breathing room air.

### 11. Ex Vivo Cardiac Magnetic Resonance Imaging

Mouse hearts are excised, rinsed in phosphate buffered saline (to remove excess blood) and immediately stored in 10% formalin solution. Hearts are placed in an 8 mm internal diameter glass tube suspended in a solution of 0.2% Gd-doped water for ex vivo imaging. Ex vivo cardiac magnetic resonance imaging is performed in a 9.4 T vertical bore magnet (Bruker Biospin; Billerica, Mass.) using a transmit/receive volume coil with an internal diameter of 10 mm. Gradient echo tri-pilot scout images are acquired using the following imaging parameters; TE/TR=6/266 ms, FOV=20 mm×20 mm, Matrix=128×128, flip angle=30 degrees, slice thickness=1 mm, 8 slices/orthogonal plane, NEX=1, 156 micron resolution, TA=0:34 seconds.

Upon completion of the scout imaging, spin echo and gradient echo images are acquired in both the coronal and axial planes. Spin echo sequence parameters are as follows; TE/TR=10.5/2000 ms, FOV=10 mm×10 mm, Matrix=256×256, slice thickness=0.3 mm, NEX=4, 39 micron resolution, TA=34:08 seconds. The gradient echo sequence parameters are as follows; TE/TR=3.2/500 ms, FOV=10 mm×10 mm, Matrix=256×256, slice thickness=0.3 mm, NEX=32, 39 micron resolution, TA=1 h 08 m:16 seconds.

A T2* multi-gradient echo image (MGE) is acquired on a single slice through iron deposited cells in myocardium. The mge sequence parameters are as follows; First echo=2.55 ms, # echo images=12, min echo distance=2.55 ms, TR=500 ms, FOV=10 mm×10 mm, Matrix=256×256, slice thickness=0.3 mm, NEX=32, 39 micron resolution, TA=51:12 seconds. Image analysis of iron signal in hearts is performed using Analyze 8.1 software package (AnalyzeDirect, Lenexa Kans.). Upon completion of the imaging, hearts are removed from the magnet for histological confirmation of iron via Perl's staining.

### Results:

1. Increased Homing of Bone Marrow Cells to the Infarcted Heart after Ex Vivo Treatment with a Bivalent Antibody.

Whole bone marrow was extracted from genetically labeled ROSA-26 mice (in which each cell expressed the lacZ gene, coding for the β-galactosidase protein). Bone marrow cells were treated for one hour at 4° C. with the bivalent antibody construct c-kit×MLC-1. In this experiment, the bivalent construct was an anti-c-kit antibody (obtained from Fitzgerald, clone 2B8) conjugated to an anti-MLC antibody (39-15 mAb (ATCC HB11709)) using a chemical linker as described in Sen et al. J. Haemother. Stem Cell Res. 2001, April: 10(2): 247-60 (2001). Wild-type host (recipient) mice were subjected to 30 minutes of coronary artery ligation to induce ischemia. Upon reperfusion, the armed bone marrow cells (or unarmed control cells) were injected via the jugular vein into these host mice (10 million cells per mouse in 200 μl of saline). 5 days later, the host mice were sacrificed and their hearts harvested for histological analysis. Sections through the cardiac infarct region were immunostained to detect the b-galactosidase-positive β-gal+) donor bone marrow cells, which were quantified in a blinded fashion. The percentage of β-gal+ cells/total cells in lesion area=10.89±0.83 in control group vs. 24.73±3.50 in armed cell group. This analysis revealed that homing of armed cells to the infarct area was increased more than 2-fold compared to homing of unarmed cells (p<0.05). Thus these data demonstrate that treatment of the cells with the bivalent antibody increases their ability to home to (and be retained at) the site of myocardial injury.

### 2. Mobilization of C-Kit-Positive Cells by G-CSF.

We have demonstrated that G-CSF treatment of mice (Neupogen, 100 μg/kg/day) results in increased levels of c-kit-positive cells in the blood due to mobilization from the bone marrow. 4 days of treatment results in an 8.6-fold increase in c-kit positive cells in the blood.

3. Homing of Bivalent Antibody (biAb) to the Myocardial Infarction Site.

To investigate the optimal timing for delivery of the bivalent antibody post-myocardial infarction, we performed permanent coronary artery ligation in a set of wild-type mice to induce a myocardial infarction. At various time-points after ligation (1, 3 and 7 days later), the bivalent antibody was delivered systemically and allowed to circulate for one hour. After one hour, the mice were sacrificed and the hearts harvested and immunohistochemically stained to detect the bivalent antibody. Highest levels of antibody binding were seen in the infarct area in mice receiving the antibody 1 day post-MI. Levels were lower in mice that received the antibody at 3 days and almost no antibody was detected in mice that received it 7 days post-myocardial infarction. Thus, the optimal time-point for delivery of the antibody (i.e. when most MLC is exposed at the site of myocardial injury) is likely to be at 1-2 days post-infarction.

4. Delivery of biAb In Vivo Improves Cardiac Function Post-MI

We sought to determine whether the bivalent antibody could improve cardiac function in a model of myocardial infarction. To mimic a clinical paradigm, mice were treated with G-CSF (Neupogen, 100 n/kg/day or saline control) for 3 days beginning on the day of permanent coronary artery ligation. Two days post-ligation, the bivalent antibody was delivered via the jugular vein (15 μg/mouse; control Ab (MLC Ab alone)=7.5 μg/mouse). 127 mice were allocated at random to one of 4 treatment groups: 1) Sham surgery+vehicle treatment; 2) Coronary artery ligation+vehicle treatment; 3) Coronary artery ligation+G-CSF+control antibody (MLC mAb only); and 4) Coronary artery ligation+G-CSF+bivalent antibody. MRI analysis was performed 2, 4 and 12 post-MI to evaluate cardiac function. The findings were as follows and are summarized in Table 37 A to C:

TABLE 37A
Ejection Fraction (EF)

| | 2 weeks post-MI | | | |
|---|---|---|---|---|
| | | | | p-value vs. |
| | | | | MI + |
| | Mean | | p-value | G-CSF + control |
| | EF (%) | S.E.M. | vs. MI | mAb |
| Sham | 61.66 | 1.64 | | |
| MI | 32.34 | 2.40 | | |
| MI + G-CSF + control | 31.52 | 1.90 | | |
| mAb | | | | |
| MI + G-CSF + BiAb | 38.09 | 2.46 | 0.108 | 0.038 |
| | | | | p-value vs. |
| | | | | MI + |
| | Mean | | p-value | G-CSF + control |
| | EF (%) | S.E.M. | vs. vehicle | mAb |
| | 4 weeks post-MI | | | |
| Sham | 62.57 | 1.74 | | |
| MI | 32.81 | 2.70 | | |
| MI + G-CSF + control | 30.00 | 2.16 | | |
| mAb | | | | |
| MI + G-CSF + BiAb | 37.25 | 2.92 | 0.278 | 0.048 |
| | 12 weeks post-MI | | | |
| Sham | 62.23 | 1.48 | | |
| MI | 32.79 | 3.15 | | |
| MI + G-CSF + control | 28.84 | 2.55 | | |
| mAb | | | | |
| MI + G-CSF + BiAb | 37.14 | 3.17 | 0.34 | 0.046 |

Note:
p-values based on t-test.

TABLE 37B
Left Ventricular Mass:Body weight ratio (LVM/BW)

| | 2 weeks post-MI | | | |
|---|---|---|---|---|
| | | | | p-value vs. |
| | Mean | | | MI + G- |
| | LVM/BW | | p-value | CSF + control |
| | (mg/g) | S.E.M. | vs. MI | mAb |
| Sham | 3.38 | 0.05 | | |
| MI | 5.12 | 0.30 | | |
| MI + G-CSF + control | 5.32 | 0.22 | | |
| mAb | | | | |
| MI + G-CSF + BiAb | 4.69 | 0.18 | 0.209 | 0.033 |
| | | | | p-value vs. |
| | Mean | | p-value | MI + |
| | LVM/BW | | vs. | G-CSF + control |
| | (mg/g) | S.E.M. | vehicle | mAb |
| | 4 weeks post-MI | | | |
| Sham | 3.42 | 0.06 | | |
| MI | 5.37 | 0.54 | | |
| MI + G-CSF + control | 5.44 | 0.22 | | |
| mAb | | | | |
| MI + G-CSF + BiAb | 4.68 | 0.18 | 0.198 | 0.010 |
| | 12 weeks post-MI | | | |
| Sham | 3.24 | 0.07 | | |
| MI | 4.92 | 0.29 | | |
| MI + G-CSF + control | 5.28 | 0.27 | | |
| mAb | | | | |
| MI + G-CSF + BiAb | 4.60 | 0.21 | 0.38 | 0.062 |

Note:
p-values based on t-test.

TABLE 37C
Infarct Size (IS)

| | 2 weeks post-MI | | | |
|---|---|---|---|---|
| | | | | p-value vs. |
| | | | | MI + |
| | | | p-value | G-CSF + control |
| | IS (%) | S.E.M. | vs. MI | mAb |
| Sham | 0 | 0 | | |
| MI | 36.82 | 2.66 | | |
| MI + G-CSF + control | 39.58 | 1.86 | | |
| mAb | | | | |
| MI + G-CSF + BiAb | 32.55 | 2.62 | 0.265 | 0.032 |

Note:
p-values based on t-test.

a. Survival:
No significant difference was seen in survival among the 4 groups. (Survival at 7 weeks from the beginning of the study: Group 1=9/9; group 2: 23/36; group 3=27/34; group 4=24/38, p=n.s.)

b. Body Weight:
No significant difference in body weight was seen among the 4 groups.

c. Ejection Fraction (EF):
EF (measured by MRI) was significantly reduced in group 2 mice compared to sham controls (group 1) at 2 weeks post-MI. Group 3 mice showed comparably reduced EFs (EF in group 3 vs. group 2: p=n.s. by Student's t-test). However mice receiving the bivalent antibody (group 4) showed significantly improved EF at 2 weeks post-MI compared to those receiving control antibody (group 3; 38.1% vs. 31.5%, respectively, p<0.05) suggesting a beneficial effect of the bivalent antibody. Notably, MRI analysis at 4 and 12 weeks post-MI demonstrated that the functional benefits of the bivalent antibody were maintained at these later time-points suggesting long-term benefits of the treatment.

d. Left Ventricular Mass-to-Body Weight Ratio (LVM:BW):
LVM:BW is an indicator of a hypertrophic response to cardiac injury. In contrast to EF, LVM:BW increased in group 2 vs. group 1, as expected. This ratio was also elevated in groups 3 and 4, however group 4 showed an average LVM;BW ratio that was significantly reduced compared to group 3, indicating an attenuation of the hypertrophic response in bivalent antibody-treated mice, suggesting that there may be less cardiac injury in these mice. As for EF, results obtained at 2 weeks post-MI were similar to those obtained at 4 and 12 weeks. Biomarker analysis of plasma taken at the end of the study showed that the hypertrophic marker pro-ANP showed trends towards increased levels in groups 2, 3 and 4 versus group 1, while levels in group 4 showed a trend towards reduction versus groups 2 and 3.

e. Infarct Size:

MRI scans from 2 weeks post-MI were used to obtain a surrogate marker of infarct size (i.e. by determining the proportion of the epicardial circumference that is akinetic at a mid-papillary slice). This data demonstrated that infarct size was comparable in groups 2 and 3 but there was a significant reduction in group 4 vs. group 3 (p<0.05) suggesting that the bivalent antibody treatment was cardioprotective (in agreement with the EF data).

f. Other Markers of Cardiac Function and Remodeling (as Evaluated by MRI):

end systolic volume and end diastolic volume (indicators of ventricular dilation) showed trends towards improvement in group 4 vs. groups 2 and 3.

g. Histology:

Histological analysis of cardiac sections taken at the end of study revealed increased numbers of small capillaries (likely neovessels, positive for the vascular endothelial markers von Willebrand factor and mouse endothelial cell antigen) in the border zone surrounding the infracted region of hearts from mice in group 3 compared to group 4 (p<0.05) and a further trend towards an increase in group 4 versus group. This suggests that G-CSF treatment may be responsible for the increased angiogenesis, but that the addition of the bispecific antibody may further enhance the formation of new blood vessels.

Together these data suggest that compared to treatment with the control antibody, bivalent antibody treatment is able to improve cardiac function and attenuate adverse remodeling post-myocardial infarction. Moreover, these beneficial effects are sustained for at least 3 months post-infarction.

While this invention has been particularly shown and described with references to embodiments thereof, it will be understood by those skilled in the art that various changes in form and details may be made therein without departing from the scope of the invention encompassed by the appended claims.

1. An antigen-binding construct comprising a first agent which binds to a stem cell specific marker molecule and a second agent which binds to a tissue specific marker molecule.

2. The construct as claimed in claim 1 wherein the tissue specific marker is a muscle specific marker molecule.

3. The construct as claimed in claim 2 wherein the tissue specific marker is a myocardium-specific marker molecule.

4. The construct as claimed in any of claim 1 wherein the first or second agent is a monoclonal antibody.

5. The construct as claimed in any of claim 1 wherein the first or second agent is an epitope-binding domain.

6. The construct as claimed 5 wherein the epitope-binding domain is an immunoglobulin single variable domain.

7. The construct as claimed in any of claim 1 wherein the stem cell specific marker molecule and the tissue specific marker molecule are human.

8. The construct as claimed in any of claim 1 wherein the stem cell specific marker molecule is c-Kit.

9. The construct as claimed in any of claim 1 wherein the muscle-specific marker molecule is selected from the group consisting of a myosin-derived molecule such as Myosin Light Chain (MLC), cardiac myosin, human ventricular myosin light chain 1 (vMLC1), MLC 1, MLC 2 and MLC 3, cardiac troponin I, cardiac troponin, Tenascin C or creatine kinase.

10. The construct as claimed in claim 9 wherein the agent which binds to a muscle specific marker molecule is an anti-MLC antibody.

11. The construct as claimed in claim 10 wherein the anti-MLC antibody is a monoclonal antibody available from ATCC HB 11709.

12. The construct as claimed in claim 1 wherein the first agent is an anti-c-Kit monoclonal antibody and the second agent is an anti-MLC monoclonal antibody.

13. The construct as claimed in any of claim 1 which is a MAbdAb.

14. The construct as claimed in claim 13 wherein the first agent is an anti-c-Kit immunoglobulin single variable domain and the second agent is a monoclonal anti-MLC antibody.

15. The construct in any of claim 8 wherein the epitope binding domain which binds c-Kit is an immunoglobulin single variable domain or polypeptide.

16. The construct in any of claim 8 wherein the epitope binding domain which binds c-Kit is an immunoglobulin single variable domain or polypeptide having an amino acid as set out in any of SEQ ID NOs: 302-305, 457, 458 or 482.

17. The construct as claimed in claim 9 wherein the anti-MLC antibody is an antigen binding protein or antibody which binds human ventricular myosin light chain 1 (vMLC1).

18. The construct as claimed in claim 1 wherein the first agent and second agent are linked.

19. The construct as claimed in claim 15 wherein the linker is selected from any one of: A G4S linker (GGGGS); TVAAPS; ASTKGPT; ASTKGPS; EPKSCDKTHTCPPCP; ELQLEESCAEAQDGELDG, AST, STGGGGGS, STGGGGGSGGGGS, STGPPPPPS, STGPPPPPPPPPPS, STG, PPPPPS, STGSRDPYLWSAPSDPLELVVTGTSVTPSRLPTEPPSSVAEFSEATAELTVSFTNKVFT TETSRSITTSPKESDSPAGPARQYYTKGNGSTG, ‘STG’ (serine, threonine, glycine), ‘GSTG’ or ‘RS’.

20. A construct as claimed in claim 13 wherein the construct is selected from any of the constructs described in Table 24.

21-81. (canceled)
